(19) United States
(12) Patent Application Publication
(10) Pub. No.: US 2026/0297587 A1
(43) Pub. Date: Oct. 1, 2026

(54) Modulation of Dystrophia Myotonica-Protein Kinase (DMPK) Expression

(71) Applicants: Ionis Pharmaceuticals, Inc., Carlsbad, CA (US); University of Rochester, Rochester, NY (US)

(72) Inventors: C. Frank Bennett, Carlsbad, CA (US); Susan M. Freier, San Diego, CA (US); Robert A. MacLeod, San Diego, CA (US); Sanjay K. Pandey, Encinitas, CA (US); Charles A. Thornton, Rochester, NY (US); Thurman Wheeler, Rochester, NY (US); Seng H. Cheng, Natick, MA (US); Andrew Leger, Boston, MA (US); Bruce M. Wentworth, Northborough, MA (US)

(73) Assignees: Ionis Pharmaceuticals, Inc., Carlsbad, CA (US); University of Rochester, Rochester, NY (US)

(21) Appl. No.: 19/366,241

(22) Filed: Oct. 22, 2025

Related U.S. Application Data
(62) Division of application No. 17/814,120, filed on Jul. 21, 2022, now Pat. No. 12,480,126.
Application No. 17/814,120 is a continuation of application No. 16/722,960, filed on Dec. 20, 2019, now abandoned.
Application No. 16/722,960 is a continuation of application No. 15/657,474, filed on Jul. 24, 2017, now abandoned.
Application No. 15/657,474 is a continuation of application No. 14/814,174, filed on Jul. 30, 2015, now Pat. No. 9,765,338.
Application No. 14/814,174 is a continuation of application No. 13/811,181, filed on Jul. 19, 2011, now abandoned.
(60) Provisional application No. 61/365,775, filed on Jul. 19, 2010.
(60) Provisional application No. 61/365,762, filed on Jul. 19, 2010.
(60) Provisional application No. 61/478,021, filed on Apr. 21, 2011.

(51) Int. Cl. C12N 15/113; A61P 15/00; A61P 25/18; A61P 35/00; A61P 43/00

(52) U.S. Cl. CPC C12N 15/1137; A61P 15/00; A61P 25/18; A61P 35/00; A61P 43/00; C12N 15/113; C12Y 207/11; C12N 2310/11; C12N 2310/315; C12N 2310/3181; C12N 2310/321; C12N 2310/3231; C12N 2310/3341; C12N 2310/341; C12N 2310/346; C12N 2310/3525

(57) ABSTRACT

Provided herein are methods, compounds, and compositions for reducing expression of a DMPK mRNA and protein in an animal. Also provided herein are methods, compounds, and compositions for preferentially reducing CUGexp DMPK RNA, reducing myotonia or reducing spliceopathy in an animal. Such methods, compounds, and compositions are useful to treat, prevent, delay, or ameliorate type 1 myotonic dystrophy, or a symptom thereof.

### STATEMENT OF GOVERNMENT SUPPORT

This invention was made with government support under NS072323 awarded by the National Institutes of Health. The government has certain rights in the invention.

### SEQUENCE LISTING

The present application is being filed along with a Sequence Listing in electronic format. The Sequence Listing is provided as a file entitled 2022-07-19_01135-0068-06US_ST26.xml created Jul. 20, 2022, which is 1,114,714 bytes in size. The information in the electronic format of the sequence listing is incorporated herein by reference in its entirety.

### FIELD

Provided herein are methods, compounds, and compositions for reducing expression of DMPK mRNA and protein in an animal. Also, provided herein are methods, compounds, and compositions comprising a DMPK inhibitor for preferentially reducing CUGexp DMPK RNA, reducing myotonia, or reducing spliceopathy in an animal. Such methods, compounds, and compositions are useful, for example, to treat, prevent, or ameliorate type 1 myotonic dystrophy (DM1) in an animal.

### BACKGROUND

Myotonic dystrophy type 1 (DM1) is the most common form of muscular dystrophy in adults with an estimated frequency of 1 in 7,500 (Harper P S., Myotonic Dystrophy. London: W.B. Saunders Company; 2001). DM1 is an autosomal dominant disorder caused by expansion of a non-coding CTG repeat in DMPK1. DMPK1 is a gene encoding a cytosolic serine/threonine kinase (Brook J D, et al., Cell., 1992, 68(4):799-808). The physiologic functions and substrates of this kinase have not been fully determined. The expanded CTG repeat is located in the 3′ untranslated region (UTR) of DMPK1. This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169).

The DMPK gene normally has 5-37 CTG repeats in the 3′ untranslated region. In myotonic dystrophy type I, this number is significantly expanded and is, for example, in the range of 50 to greater than 3,500 (Harper, Myotonic Dystrophy (Saunders, London, ed. 3, 2001); Annu. Rev. Neurosci. 29: 259, 2006; EMBO J. 19: 4439, 2000; Curr Opin Neurol. 20: 572, 2007).

The CUGexp tract interacts with RNA binding proteins including muscleblind-like (MBNL) protein, a splicing factor, and causes the mutant transcript to be retained in nuclear foci. The toxicity of this RNA stems from sequestration of RNA binding proteins and activation of signaling pathways. Studies in animal models have shown that phenotypes of DM1 can be reversed if toxicity of CUGexp RNA is reduced (Wheeler T M, et al., Science., 2009, 325(5938):336-339; Mulders S A, et al., Proc Natl Acad Sci USA., 2009, 106(33):13915-13920).

In DM1, skeletal muscle is the most severely affected tissue, but the disease also has important effects on cardiac and smooth muscle, ocular lens, and brain. The cranial, distal limb, and diaphragm muscles are preferentially affected. Manual dexterity is compromised early, which causes several decades of severe disability. The median age at death is 55 years, usually from respiratory failure (de Die-Smulders C E, et al., Brain., 1998, 121(Pt 8):1557-1563).

Antisense technology is emerging as an effective means for modulating expression of certain gene products and may therefore prove to be uniquely useful in a number of therapeutic, diagnostic, and research applications for the modulation of DMPK1. Intramuscular injection of fully modified oligonucleotides targeting with the CAG-repeat were shown in mice to block formation of CUGexp-MBNL1 complexes, disperse nuclear foci of CUGexp transcripts, enhance the nucleocytoplasmic transport and translation of CUGexp transcripts, release MBNL proteins to the nucleoplasm, normalize alternative splicing of MBNL-dependent exons, and eliminate myotonia in CUGexp-expressing transgenic mice (Wheeler T M, et al., Science., 2009, 325(5938):336-339; WO2008/036406).

Presently there is no treatment that can modify the course of DM1. The burden of disease, therefore, is significant. It is, therefore, an object herein to provide compounds, compositions, and methods for treating DM1

### SUMMARY

Provided herein are methods, compounds, and compositions for inhibiting expression of DMPK and treating, preventing, delaying or ameliorating a DMPK related disease and or a symptom thereof. In certain embodiments, the compounds and compositions inhibit mutant DMPK or CUGexp DMPK.

Certain embodiments provide a method of reducing DMPK expression in an animal comprising administering to the animal a compound comprising a modified oligonucleotide as further described herein targeted to DMPK.

Certain embodiments provide a method of preferentially reducing CUGexp DMPK, reducing myotonia, or reducing spliceopathy in an animal comprising administering to the animal a compound comprising a modified oligonucleotide, as further described herein, targeted to CUGexp DMPK. CUGexp DMPK transcripts are believed to be particularly sensitive to antisense knockdown via nuclear ribonucleases, because of their longer residence time in the nucleus, and this sensitivity is thought to permit effective antisense inhibition of CUGexp DMPK transcripts in relevant tissues such as muscle despite the biodistribution barriers to tissue uptake of antisense oligonucleotides. Antisense mechanisms that do not elicit cleavage via nuclear ribonucleases, such as the CAG-repeat ASOs described in, for example, Wheeler T M, et al., Science., 2009, 325(5938):336-339 and WO2008/036406, do not provide the same therapeutic advantage.

Certain embodiments provide a method of treating an animal with type 1 myotonic dystrophy. In certain embodiments, the method includes administering to the animal a therapeutically effective amount of a compound comprising a modified oligonucleotide as further described herein targeted to DMPK. In certain embodiments, the method includes identifying an animal with type 1 myotonic dystrophy.

Certain embodiments provide a method of treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide a method of treating, preventing, delaying, or ameliorating symptoms and outcomes associated with development of DM1 in children, including, developmental delays, learning problems, language and speech issues, and personality development issues.

Certain embodiments provide a method of administering an antisense oligonucleotide to counteract RNA dominance by directing the cleavage of pathogenic transcripts.

In certain embodiments, the DMPK has a sequence as set forth in GenBank Accession No. NM_001081560.1 (incorporated herein as SEQ ID NO: 1). In certain embodiments, the DMPK has the sequence as set forth in GenBank Accession No. NT_011109.15 truncated from nucleotides 18540696 to U.S. Pat. No. 18,555,106 (incorporated herein as SEQ ID NO: 2). In certain embodiments, the DMPK has the sequence as set forth in GenBank Accession No. NT_039413.7 truncated from nucleotides 16666001 to U.S. Pat. No. 16,681,000 (incorporated herein as SEQ ID NO: 3). In certain embodiments, the DMPK has the sequence as set forth in GenBank Accession No. NM_032418.1 (incorporated herein as SEQ ID NO: 4). In certain embodiments, the DMPK has the sequence as set forth in GenBank Accession No. AI007148.1 (incorporated herein as SEQ ID NO: 5). In certain embodiments, the DMPK has the sequence as set forth in GenBank Accession No. AI304033.1 (incorporated herein as SEQ ID NO: 6). In certain embodiments, the DMPK has the sequence as set forth in GenBank Accession No. BC024150.1 (incorporated herein as SEQ ID NO: 7). In certain embodiments, the DMPK has the sequence as set forth in GenBank Accession No. BC056615.1 (incorporated herein as SEQ ID NO: 8). In certain embodiments, the DMPK has the sequence as set forth in GenBank Accession No. BC075715.1 (incorporated herein as SEQ ID NO: 793). In certain embodiments, the DMPK has the sequence as set forth in GenBank Accession No. BU519245.1 (incorporated herein as SEQ ID NO: 794). In certain embodiments, the DMPK has the sequence as set forth in GenBank Accession No. CB247909.1 (incorporated herein as SEQ ID NO: 795). In certain embodiments, the DMPK has the sequence as set forth in GenBank Accession No. CX208906.1 (incorporated herein as SEQ ID NO: 796). In certain embodiments, the DMPK has the sequence as set forth in GenBank Accession No. CX732022.1 (incorporated herein as SEQ ID NO: 797). In certain embodiments, the DMPK has the sequence as set forth in GenBank Accession No. S60315.1 (incorporated herein as SEQ ID NO: 798). In certain embodiments, the DMPK has the sequence as set forth in GenBank Accession No. S60316.1 (incorporated herein as SEQ ID NO: 799). In certain embodiments, the DMPK has the sequence as set forth in GenBank Accession No. NM_001081562.1 (incorporated herein as SEQ ID NO: 800). In certain embodiments, the DMPK has the sequence as set forth in GenBank Accession No. NM_001100.3 (incorporated herein as SEQ ID NO: 801).

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. Herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furanosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to position 5. A 5-methylcytosine is a modified nucleobase.

“About” means within ±7% of a value. For example, if it is stated, “the compound affected at least 70% inhibition of DMPK”, it is implied that the DMPK levels are inhibited within a range of 63% and 77%.

“Active pharmaceutical agent” means the substance or substances in a pharmaceutical composition that provide a therapeutic benefit when administered to an individual. For example, in certain embodiments an antisense oligonucleotide targeted to DMPK is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing an agent to an animal, and includes, but is not limited to, administering by a medical professional and self-administering.

“Agent” means an active substance that can provide a therapeutic benefit when administered to an animal. “First Agent” means a therapeutic compound of the invention. For example, a first agent can be an antisense oligonucleotide targeting DMPK. “Second agent” means a second therapeutic compound of the invention (e.g. a second antisense oligonucleotide targeting DMPK) and/or a non-DMPK therapeutic compound.

“Amelioration” refers to a lessening of at least one indicator, sign, or symptom of an associated disease, disorder, or condition. The severity of indicators can be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, snoRNAs, miRNAs, and satellite repeats.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furanosyl ring modified by the bridging of two non-geminal carbon ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“CUGexp DMPK” means mutant DMPK RNA containing an expanded CUG repeat (CUGexp). The wild-type DMPK gene has 5-37 CTG repeats in the 3′ untranslated region. In a “CUGexp DMPK” (such as in a myotonic dystrophy type I patient) this number is significantly expanded and is, for example, in the range of 50 to greater than 3,500 (Harper, Myotonic Dystrophy (Saunders, London, ed. 3, 2001); Annu. Rev. Neurosci. 29: 259, 2006; EMBO J. 19: 4439, 2000; Curr Opin Neurol. 20: 572, 2007).

“Diluent” means an ingredient in a composition that lacks pharmacological activity, but is pharmaceutically necessary or desirable. For example, the diluent in an injected composition can be a liquid, e.g. saline solution.

“DMPK” means any nucleic acid or protein of DMPK. DMPK can be a mutant DMPK including CUGexp DMPK nucleic acid.

“DMPK expression” means the level of mRNA transcribed from the gene encoding DMPK or the level of protein translated from the mRNA. DMPK expression can be determined by art known methods such as a Northern or Western blot.

“DMPK nucleic acid” means any nucleic acid encoding DMPK. For example, in certain embodiments, a DMPK nucleic acid includes a DNA sequence encoding DMPK, an RNA sequence transcribed from DNA encoding DMPK (including genomic DNA comprising introns and exons), and an mRNA or pre-mRNA sequence encoding DMPK. “DMPK mRNA” means an mRNA encoding a DMPK protein.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.” “Gap-widened” means a chimeric antisense compound having a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Identifying an animal with type 1 myotonic dystrophy” means identifying an animal having been diagnosed with a type 1 myotonic dystrophy, disorder or condition or identifying an animal predisposed to develop a type 1 myotonic dystrophy, disorder or condition. For example, individuals with a familial history can be predisposed to type 1 myotonic dystrophy, disorder or condition. Such identification can be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Linked nucleosides” means adjacent nucleosides which are bonded or linked together by an internucleoside linkage.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising at least one modified nucleotide.

“Modified sugar” refers to a substitution or change from a natural sugar.

“Motif” means the pattern of chemically distinct regions in an antisense compound.

“Myotonia” means an abnormally slow relaxation of a muscle after voluntary contraction or electrical stimulation.

“Nuclear ribonuclease” means a ribonuclease found in the nucleus. Nuclear ribonucleases include, but are not limited to, RNase H including RNase H1 and RNase H2, the double stranded RNase drosha and other double stranded RNases.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition can comprise one or more active agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Preferentially reducing CUG exp DMPK RNA” refers to a preferential reduction of RNA transcripts from a CUGexp DMPK allele relative to RNA transcripts from a normal DMPK allele.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Spliceopathy” means a change in the alternative splicing of one or more RNAs that leads to the expression of altered splice products in a particular tissue.

“Subcutaneous administration” means administration just below the skin.

“Sugar surrogate” overlaps with the slightly broader term “nucleoside mimetic” but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment.

“3′ target site” refers to the 3′-most nucleotide of a target segment.

“Therapeutically effective amount” means an amount of an agent that provides a therapeutic benefit to an individual.

“Treat” refers to administering a pharmaceutical composition to effect an alteration or improvement of a disease, disorder, or condition.

“Type 1 myotonic dystrophy” or “DM1” means an autosomal dominant disorder caused by expansion of a non-coding CTG repeat in DMPK. This mutation leads to RNA dominance, a process in which expression of RNA containing an expanded CUG repeat (CUGexp) induced cell dysfunction. The CUGexp tract interacts with RNA binding proteins and causes the mutant transcript to be retained in nuclear foci. The toxicity of this RNA stems from sequestration of RNA binding proteins and activation of signaling pathways.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods, compounds, and compositions for inhibiting DMPK expression.

Certain embodiments provide a method of reducing DMPK expression in an animal comprising administering to the animal a compound comprising a modified oligonucleotide targeting DMPK.

Certain embodiments provide a method of preferentially reducing CUGexp DMPK RNA, reducing myotonia or reducing spliceopathy in an animal comprising administering to the animal a compound comprising a modified oligonucleotide targeted to DMPK, wherein the modified oligonucleotide preferentially reduces CUGexp DMPK RNA, reduces myotonia or reduces spliceopathy in the animal.

Certain embodiments provide a method of administering an antisense oligonucleotide to counteract RNA dominance by directing the cleavage of pathogenic transcripts.

Certain embodiments provide a method of reducing spliceopathy of Serca1. In certain embodiments, methods provided herein result in exon 22 inclusion. In certain embodiments, the corrective splicing occurs in the tibialis anterior, gastrocnemius, and quadriceps muscles.

Certain embodiments provide a method of reducing spliceopathy of m-Titin. In certain embodiments, methods provided herein result in exon 5 inclusion. In certain embodiments, the corrective splicing occurs in the tibialis anterior, gastrocnemius, and quadriceps muscles.

Certain embodiments provide a method of reducing spliceopathy of Clcn1. In certain embodiments, methods provided herein result in exon 7a inclusion. In certain embodiments, the corrective splicing occurs in the tibialis anterior, gastrocnemius, and quadriceps muscles.

Certain embodiments provide a method of reducing spliceopathy of Zasp. In certain embodiments, methods provided herein result in exon 11 inclusion. In certain embodiments, the corrective splicing occurs in the tibialis anterior, gastrocnemius, and quadriceps muscles.

Certain embodiments provide a method for treating an animal with type 1 myotonic dystrophy comprising: a) identifying said animal with type 1 myotonic dystrophy, and b) administering to said animal a therapeutically effective amount of a compound comprising a modified oligonucleotide targeted to DMPK. In certain embodiments, the therapeutically effective amount of the compound administered to the animal preferentially reduces CUGexp DMPK RNA, reduces myotonia or reduces spliceopathy in the animal.

Certain embodiments provide a method of achieving a preferential reduction of CUGexp DMPK RNA, including administering to the subject suspected of having type 1 myotonic dystrophy or having a CUGexp DMPK RNA a modified antisense oligonucleotide complementary to a non-repeat region of said CUGexp DMPK RNA. The modified antisense oligonucleotide, when bound to said CUGexp DMPK RNA, achieves a preferential reduction of the CUGexp DMPK RNA.

Certain embodiments provide a method of achieving a preferential reduction of CUGexp DMPK RNA, including selecting a subject having type 1 myotonic dystrophy or having a CUGexp DMPK RNA and administering to said subject a modified antisense oligonucleotide complementary to a non-repeat region of said CUGexp DMPK RNA. The modified antisense oligonucleotide, when bound to the CUGexp DMPK RNA, activates a ribonuclease or nuclear ribonuclease, thereby achieving a preferential reduction of the CUGexp DMPK RNA in the nucleus.

Certain embodiments provide a method of achieving a preferential reduction of CUGexp DMPK RNA, including selecting a subject having type 1 myotonic dystrophy or having a mutant or CUGexp DMPK RNA and systemically administering to said subject a modified antisense oligonucleotide complementary to a non-repeat region of said CUGexp DMPK RNA. The modified antisense oligonucleotide, when bound to the mutant or CUGexp DMPK RNA, achieves a preferential reduction of the mutant or CUGexp DMPK RNA.

Certain embodiments provide a method of reducing myotonia in a subject in need thereof. The method includes administering to the subject a modified antisense oligonucleotide complementary to a non-repeat region of a DMPK RNA, wherein the modified antisense oligonucleotide, when bound to the DMPK RNA, activates a ribonuclease or nuclear ribonuclease, thereby reducing myotonia. In certain embodiments, the subject has or is suspected of having type 1 myotonic dystrophy or having a mutant DMPK RNA or CUGexp DMPK RNA. In certain embodiments, the DMPK RNA is nuclear retained.

Certain embodiments provide a method of reducing spliceopathy in a subject in need thereof. The method includes administering to the subject a modified antisense oligonucleotide complementary to a non-repeat region of a DMPK RNA, wherein the modified antisense oligonucleotide, when bound to the DMPK RNA, activates a ribonuclease or nuclear ribonuclease, thereby reducing spliceopathy. In certain embodiments, the subject has or is suspected of having type 1 myotonic dystrophy or having a nuclear retained CUGexp DMPK RNA. In certain embodiments, the DMPK RNA is nuclear retained. In certain embodiments, the spliceopathy is MBNL dependent spliceopathy.

In certain embodiments, the modified antisense oligonucleotide of the methods is chimeric. In certain embodiments, the modified antisense oligonucleotide of the methods is a gapmer.

In certain embodiments of the methods provided herein, the administering is subcutaneous. In certain embodiments, the administering is intravenous.

In certain embodiments, the modified antisense oligonucleotide of the methods targets a non-coding sequence within the non-repeat region of a DMPK RNA. In certain embodiments, the oligonucleotide targets a coding region, an intron, a 5′UTR, or a 3′UTR of the mutant DMPK RNA.

In certain embodiments of the methods provided herein, the nuclear ribonuclease is RNase H1.

In certain embodiments of the methods, the DMPK RNA is reduced in muscle tissue. In certain embodiments, the mutant DMPK RNA CUGexp DMPK RNA is preferentially reduced.

In certain embodiments, the DMPK has the sequence as set forth in GenBank Accession No. NM_001081560.1 (incorporated herein as SEQ ID NO: 1). In certain embodiments, the DMPK has the sequence as set forth in GenBank Accession No. NT_011109.15 truncated from nucleotides 18540696 to U.S. Pat. No. 18,555,106 (incorporated herein as SEQ ID NO: 2). In certain embodiments, the DMPK has the sequence as set forth in GenBank Accession No. NT_039413.7 truncated from nucleotides 16666001 to U.S. Pat. No. 16,681,000 (incorporated herein as SEQ ID NO: 3). In certain embodiments, the DMPK has the sequence as set forth in GenBank Accession No. NM_032418.1 (incorporated herein as SEQ ID NO: 4). In certain embodiments, the DMPK has the sequence as set forth in GenBank Accession No. AI007148.1 (incorporated herein as SEQ ID NO: 5). In certain embodiments, the DMPK has the sequence as set forth in GenBank Accession No. AI304033.1 (incorporated herein as SEQ ID NO: 6). In certain embodiments, the DMPK has the sequence as set forth in GenBank Accession No. BC024150.1 (incorporated herein as SEQ ID NO: 7). In certain embodiments, the DMPK has the sequence as set forth in GenBank Accession No. BC056615.1 (incorporated herein as SEQ ID NO: 8). In certain embodiments, the DMPK has the sequence as set forth in GenBank Accession No. BC075715.1 (incorporated herein as SEQ ID NO: 793). In certain embodiments, the DMPK has the sequence as set forth in GenBank Accession No. BU519245.1 (incorporated herein as SEQ ID NO: 794). In certain embodiments, the DMPK has the sequence as set forth in GenBank Accession No. CB247909.1 (incorporated herein as SEQ ID NO: 795). In certain embodiments, the DMPK has the sequence as set forth in GenBank Accession No. CX208906.1 (incorporated herein as SEQ ID NO: 796). In certain embodiments, the DMPK has the sequence as set forth in GenBank Accession No. CX732022.1 (incorporated herein as SEQ ID NO: 797). In certain embodiments, the DMPK has the sequence as set forth in GenBank Accession No. 560315.1 (incorporated herein as SEQ ID NO: 798). In certain embodiments, the DMPK has the sequence as set forth in GenBank Accession No. S60316.1 (incorporated herein as SEQ ID NO: 799). In certain embodiments, the DMPK has the sequence as set forth in GenBank Accession No. NM_001081562.1 (incorporated herein as SEQ ID NO: 800). In certain embodiments, the DMPK has the sequence as set forth in GenBank Accession No. NM_001100.3 (incorporated herein as SEQ ID NO: 801). In certain embodiments, the modified oligonucleotide has a nucleobase sequence comprising at least 8 contiguous nucleobases of a nucleobase sequence recited in any one of SEQ ID NOs: 12-156, 160-770, and 774-792. In certain embodiments, the modified oligonucleotide has a nucleobase sequence comprising at least 9, at least 10, or at least 11, contiguous nucleobases of a nucleobase sequence recited in any one of SEQ ID NOs: 12-156, 160-770, and 774-792.

In certain embodiments, the modified oligonucleotide has a nucleobase sequence comprising at least 12 contiguous nucleobases of a nucleobase sequence recited in any one of SEQ ID NOs: 12-156, 160-770, and 774-792. In certain embodiments, the modified oligonucleotide has a nucleobase sequence comprising at least 13, or at least 14, contiguous nucleobases of a nucleobase sequence recited in any one of SEQ ID NOs: 12-156, 160-770, and 774-792.

In certain embodiments, the modified oligonucleotide has a nucleobase sequence comprising at least 15 contiguous nucleobases of a nucleobase sequence recited in any one of SEQ ID NOs: 12-156, 160-770, and 774-792. In certain embodiments, the modified oligonucleotide has a nucleobase sequence comprising at least 16, or at least 17, contiguous nucleobases of a nucleobase sequence recited in any one of SEQ ID NOs: 12-156, 160-770, and 774-792.

In certain embodiments, the modified oligonucleotide has a nucleobase sequence comprising at least 18 contiguous nucleobases of a nucleobase sequence recited in any one of SEQ ID NOs: 12-156, 160-770, and 774-792. In certain embodiments, the modified oligonucleotide has a nucleobase sequence comprising at least 19 contiguous nucleobases of a nucleobase sequence recited in any one of SEQ ID NOs: 12-156, 160-770, and 774-792.

In certain embodiments, the modified oligonucleotides provided herein are targeted to any one of the following regions of SEQ ID NO: 1: 1178-1206, 2159-2182, 2174-2196, 2426-2447, 2450-2518, 2679-2704, and 2697-2725.

In certain embodiments, the modified oligonucleotides provided herein are targeted to any one of the following regions of SEQ ID NO 1: 178-223, 232-253, 279-299, 366-399, 519-541, 923-975, 1073-1105, 1171-1196, 1215-1246, 1263-1324, 1706-1734, 1743-1763, 1932-1979, 1981-2003, 2077-2108, and 2152-2173.

In certain embodiments, the modified oligonucleotides provided herein are targeted to any one of the following regions of SEQ ID NO: 2: 1251-1303, 1305-1326, 1352-1372, 3762-3795, 4170-4192, 5800-5852, 6124-6149, 6168-6199, 6216-6277, 11979-12007, 12016-12036, 12993-13042, 13044-13066, 13140-13171, and 13215-13236.

In certain embodiments, the animal is a human.

In certain embodiments, the compounds or compositions of the invention are designated as a first agent and the methods of the invention further comprise administering a second agent. In certain embodiments, the first agent and the second agent are co-administered. In certain embodiments the first agent and the second agent are co-administered sequentially or concomitantly.

In certain embodiments, administration comprises parenteral administration.

In certain embodiments, the compound is a single-stranded modified oligonucleotide. In certain embodiments, the nucleobase sequence of the modified oligonucleotide is at least 95% complementary to any one of SEQ ID NOs: 1-8 and 793-801 as measured over the entirety of said modified oligonucleotide. In certain embodiments, the nucleobase sequence of the modified oligonucleotide is 100% complementary to any one of SEQ ID NOs: 1-8 and 793-801 as measured over the entirety of said modified oligonucleotide.

In certain embodiments, at least one internucleoside linkage of said modified oligonucleotide is a modified internucleoside linkage. In certain embodiments, each internucleoside linkage is a phosphorothioate internucleoside linkage.

In certain embodiments, at least one nucleoside of said modified oligonucleotide comprises a modified sugar. In certain embodiments, at least one modified sugar is a bicyclic sugar. In certain embodiments, at least one modified sugar comprises a 2′-O-methoxyethyl or a 4′-(CH₂)ₙ—O-2′ bridge, wherein n is 1 or 2.

In certain embodiments, at least one nucleoside of said modified oligonucleotide comprises a modified nucleobase. In certain embodiments, the modified nucleobase is a 5-methylcytosine.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, and each cytosine in said modified oligonucleotide is a 5′-methylcytosine.

In certain embodiments, the modified oligonucleotide consists of 20 linked nucleosides.

Certain embodiments provide a method of preferentially reducing CUGexp DMPK RNA, reducing myotonia or reducing spliceopathy in an animal comprising administering to the animal a compound comprising a modified oligonucleotide having a gap segment consisting of ten linked deoxynucleosides, a 5′ wing segment consisting of five linked nucleosides and a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine.

Certain embodiments provide the use of any compound as described herein in the manufacture of a medicament for use in any of the therapeutic methods described herein. For example, certain embodiments provide the use of a compound as described herein in the manufacture of a medicament for treating, ameliorating, or preventing type 1 myotonic dystrophy.

Certain embodiments provide the use of a compound as described herein in the manufacture of a medicament for inhibiting expression of DMPK and treating, preventing, delaying or ameliorating a DMPK related disease and or a symptom thereof. Certain embodiments provide the use of a compound as described herein in the manufacture of a medicament for reducing DMPK expression in an animal. Certain embodiments provide the use of a compound as described herein in the manufacture of a medicament for preferentially reducing CUGexp DMPK, reducing myotonia, or reducing spliceopathy in an animal. Certain embodiments provide the use of a compound as described herein in the manufacture of a medicament for treating an animal with type 1 myotonic dystrophy. Certain embodiments provide the use of a compound as described herein in the manufacture of a medicament for treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide the use of a compound as described herein in the manufacture of a medicament for counteracting RNA dominance by directing the cleavage of pathogenic transcripts.

Certain embodiments provide a kit for treating, preventing, or ameliorating type 1 myotonic dystrophy as described herein wherein the kit comprises: a) a compound as described herein; and optionally b) an additional agent or therapy as described herein. The kit can further include instructions or a label for using the kit to treat, prevent, or ameliorate type 1 myotonic dystrophy.

Certain embodiments provide any compound or composition as described herein, for use in any of the therapeutic methods described herein. For example, certain embodiments provide a compound or composition as described herein for inhibiting expression of DMPK and treating, preventing, delaying or ameliorating a DMPK related disease and or a symptom thereof. Certain embodiments provide a compound or composition as described herein for use in reducing DMPK expression in an animal. Certain embodiments provide a compound or composition as described herein for use in preferentially reducing CUGexp DMPK, reducing myotonia, or reducing spliceopathy in an animal. Certain embodiments provide a compound or composition as described herein for use in treating an animal with type 1 myotonic dystrophy. Certain embodiments provide a compound or composition as described herein for use in treating, preventing, delaying, or ameliorating symptoms and outcomes associated with development of DM1 including muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. Certain embodiments provide a compound or composition as described herein for use in counteracting RNA dominance by directing the cleavage of pathogenic transcripts. Certain embodiments provide compounds comprising a modified oligonucleotide consisting of 12 to 30 linked nucleosides having a nucleobase sequence comprising at least 12 contiguous nucleobases of any of the nucleobase sequences of SEQ ID NOs: 12-156, 160-770, and 774-792.

Other compounds which can be used in the methods described herein are also provided.

For example, certain embodiments provide compounds comprising a modified oligonucleotide consisting of 10 to 80, 12 to 50, 12 to 30, 15 to 30, 18 to 24, 19 to 22, or 20 linked nucleosides having a nucleobase sequence comprising at least 8, at least 9, at least 10, at least 11, at least 12, at least 13, at least 14, at least 15, at least 16, at least 17, at least 18, or at least 19, contiguous nucleobases of any of the nucleobase sequences of SEQ ID NOs: 41, 44, 76, 109, 153, 320, 321, 322, 325, 329, 335, and 657.

Certain embodiments provide compounds comprising a modified oligonucleotide consisting of 10 to 80, 12 to 50, 12 to 30, 15 to 30, 18 to 24, 19 to 22, or 20, linked nucleosides having a nucleobase sequence comprising at least 8, at least 9, at least 10, at least 11, at least 12, at least 13, at least 14, at least 15, at least 16, at least 17, at least 18, at least 19, contiguous nucleobases of any of the nucleobase sequences of SEQ ID NOs: 15, 73, 77, 79, 83, 85, 130, 602, 648, 655, 674, and 680.

Certain embodiments provide compounds comprising a modified oligonucleotide consisting of 10 to 80, 12 to 50, 12 to 30, 15 to 30, 18 to 24, 19 to 22, or 20, linked nucleosides having a nucleobase sequence comprising a portion of at least 8, at least 9, at least 10, at least 11, at least 12, at least 13, at least 14, at least 15, at least 16, at least 17, at least 18, or at least 19, or more, contiguous nucleobases complementary to an equal length portion of nucleobases 664-683, 773-792, 926-945, 927-946, 928-947, 931-950, 935-954, 941-960, 2089-2108, 2163-2182, 2490-2509, 2499-2518, 2676-2695, 2685-2704, 2676-2695, 2688-2707, 2697-2716, 2764-2783, and 2770-2789 of SEQ ID NO: 1, wherein the nucleobase sequence is complementary to SEQ ID NO: 1.

Certain embodiments provide compounds comprising a modified oligonucleotide consisting of 10 to 80, 12 to 50, 12 to 30, 15 to 30, 18 to 24, 19 to 22, or 20, linked nucleosides having a nucleobase sequence comprising a portion of at least 8, at least 9, at least 10, at least 11, at least 12, at least 13, at least 14, at least 15, at least 16, at least 17, at least 18, or at least 19, or more, contiguous nucleobases complementary to an equal length portion of nucleobases 812-831, 3629-3648, 4447-4466, 4613-4632, 5803-5822, 5804-5823, 5805-5824, 5808-5827, 5818-5837, 6794-6813, 12463-12482, 13152-13171, and 13553-13572 of SEQ ID NO: 2, wherein the nucleobase sequence is complementary to SEQ ID NO: 2.

In certain embodiments, the modified oligonucleotide is a single-stranded oligonucleotide.

In certain embodiments, the nucleobase sequence of the modified oligonucleotide is at least 70%, at least 75%, at least 80%, at least 85%, at least 90%, at least 95%, or 100%, complementary to any of SEQ ID NOs: 1-8 and 793-801.

In certain embodiments, at least one internucleoside linkage is a modified internucleoside linkage.

In certain embodiments, each internucleoside linkage is a phosphorothioate internucleoside linkage.

In certain embodiments, at least one nucleoside comprises a modified sugar.

In certain embodiments, at least one modified sugar is a bicyclic sugar.

In certain embodiments, at least one modified sugar comprises a 2′-O-methoxyethyl.

In certain embodiments, at least one nucleoside comprises a modified nucleobase.

In certain embodiments, the modified nucleobase is a 5-methylcytosine.

In certain embodiments, the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

In certain embodiments, the modified oligonucleotide comprises:
a gap segment consisting of ten linked deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide consists of 14 linked nucleosides.

In certain embodiments, the modified oligonucleotide consists of 16 linked nucleosides.

In certain embodiments, the modified oligonucleotide consists of 20 linked nucleosides.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to DMPK as described herein is 10 to 30 nucleotides in length. In other words, the antisense compounds are in some embodiments from 10 to 30 linked nucleobases. In other embodiments, the antisense compound comprises a modified oligonucleotide consisting of 8 to 80, 10 to 80, 12 to 30, 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In certain embodiments, antisense compounds of any of these lengths contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 12-156, 160-770, and 774-792.

In certain embodiments, the antisense compound comprises a shortened or truncated modified oligonucleotide. The shortened or truncated modified oligonucleotide can have a single nucleoside deleted from the 5′ end (5′ truncation), or alternatively from the 3′ end (3′ truncation). A shortened or truncated oligonucleotide can have two nucleosides deleted from the 5′ end, or alternatively can have two subunits deleted from the 3′ end. Alternatively, the deleted nucleosides can be dispersed throughout the modified oligonucleotide, for example, in an antisense compound having one nucleoside deleted from the 5′ end and one nucleoside deleted from the 3′ end.

When a single additional nucleoside is present in a lengthened oligonucleotide, the additional nucleoside can be located at the 5′ or 3′ end of the oligonucleotide. When two or more additional nucleosides are present, the added nucleosides can be adjacent to each other, for example, in an oligonucleotide having two nucleosides added to the 5′ end (5′ addition), or alternatively to the 3′ end (3′ addition), of the oligonucleotide. Alternatively, the added nucleoside can be dispersed throughout the antisense compound, for example, in an oligonucleotide having one nucleoside added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a DMPK nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH₂)ₙ—O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, or 2-6-2.

In certain embodiments, the antisense compound as a “wingmer” motif, having a wing-gap or gap-wing configuration, i.e. an X-Y or Y-Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a DMPK nucleic acid possess a 5-10-5 gapmer motif.

In certain embodiments, an antisense compound targeted to a DMPK nucleic acid has a gap-widened motif.

In certain embodiments, antisense compounds of any of these gapmer or wingmer motifs contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 12-156, 160-770, and 774-792.

### Target Nucleic Acids, Target Regions and Nucleotide Sequences

Nucleotide sequences that encode DMPK include, without limitation, the following sequences as set forth in GenBank Accession No. NM_001081560.1 (incorporated herein as SEQ ID NO: 1), GenBank Accession No. NT_011109.15 truncated from nucleotides 18540696 to U.S. Pat. No. 18,555,106 (incorporated herein as SEQ ID NO: 2), GenBank Accession No. NT_039413.7 truncated from nucleotides 16666001 to U.S. Pat. No. 16,681,000 (incorporated herein as SEQ ID NO: 3), GenBank Accession No. NM_032418.1 (incorporated herein as SEQ ID NO: 4), GenBank Accession No. AI007148.1 (incorporated herein as SEQ ID NO: 5), GenBank Accession No. AI304033.1 (incorporated herein as SEQ ID NO: 6), GenBank Accession No. BC024150.1 (incorporated herein as SEQ ID NO: 7), GenBank Accession No. BC056615.1 (incorporated herein as SEQ ID NO: 8), GenBank Accession No. BC075715.1 (incorporated herein as SEQ ID NO: 793), GenBank Accession No. BU519245.1 (incorporated herein as SEQ ID NO: 794), GenBank Accession No. CB247909.1 (incorporated herein as SEQ ID NO: 795), GenBank Accession No. CX208906.1 (incorporated herein as SEQ ID NO: 796), GenBank Accession No. CX732022.1 (incorporated herein as SEQ ID NO: 797), GenBank Accession No. S60315.1 (incorporated herein as SEQ ID NO: 798), GenBank Accession No. S60316.1 (incorporated herein as SEQ ID NO: 799), GenBank Accession No. NM_001081562.1 (incorporated herein as SEQ ID NO: 800), and GenBank Accession No. NM_001100.3 (incorporated herein as SEQ ID NO: 801). It is understood that the sequence set forth in each SEQ ID NO in the Examples contained herein is independent of any modification to a sugar moiety, an internucleoside linkage, or a nucleobase. As such, antisense compounds defined by a SEQ ID NO can comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for DMPK can be obtained by accession number from sequence databases such as NCBI and such information is incorporated herein by reference. In certain embodiments, a target region can encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense compound sequences that can hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in DMPK mRNA levels are indicative of inhibition of DMPK protein expression. Reductions in levels of a DMPK protein are also indicative of inhibition of target mRNA expression. Further, phenotypic changes, such as a reducing myotonia or reducing spliceopathy, can be indicative of inhibition of DMPK mRNA and/or protein expression.

### Hybridization

In some embodiments, hybridization occurs between an antisense compound disclosed herein and a DMPK nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrooke and Russell, Molecular Cloning: A Laboratory Manual, 3^{rd} Ed., 2001). In certain embodiments, the antisense compounds provided herein are specifically hybridizable with a DMPK nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a DMPK nucleic acid).

An antisense compound can hybridize over one or more segments of a DMPK nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a DMPK nucleic acid, a target region, target segment, or specified portion thereof. In certain embodiments, the antisense compounds are at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to a DMPK nucleic acid, a target region, target segment, or specified portion thereof, and contain at least 8, at least 9, at least 10, at least 11, at least 12, at least 13, at least 14, at least 15, at least 16, at least 17, at least 18, or at least 19, contiguous nucleobases of the nucleobase sequence of any of the exemplary antisense compounds described herein (e.g., at least 8 contiguous nucleobases of a nucleobase sequence recited in any one of SEQ ID NOs: 12-156, 160-770, and 774-792). Percent complementarity of an antisense compound with a target nucleic acid can be determined using routine methods, and is measured over the entirety of the antisense compound.

For example, an antisense compound in which 18 of 20 nucleobases of the antisense compound are complementary to a target region, and would therefore specifically hybridize, would represent 90 percent complementarity. In this example, the remaining noncomplementary nucleobases can be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound can be fully complementary to a DMPK nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound can be fully complementary to the target sequence, depending on whether the remaining 10 nucleobases of the antisense compound are also complementary to the target sequence. The location of a non-complementary nucleobase can be at the 5′ end or 3′ end of the antisense compound. Alternatively, the non-complementary nucleobase or nucleobases can be at an internal position of the antisense compound. When two or more non-complementary nucleobases are present, they can be either contiguous (i.e. linked) or non-contiguous. In one embodiment, a non-complementary nucleobase is located in the wing segment of a gapmer antisense oligonucleotide.

In certain embodiments, antisense compounds that are, or are up to 10, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a DMPK nucleic acid, or specified portion thereof.

In certain embodiments, antisense compounds that are, or are up to 10, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a DMPK nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 10 nucleobase portion of a target segment. In certain embodiments, the antisense compounds are complementary to at least a 15 nucleobase portion of a target segment. Also contemplated are antisense compounds that are complementary to at least an 8, at least a 9, at least a 10, at least an 11, at least a 12, at least a 13, at least a 14, at least a 15, at least a 16, at least a 17, at least an 18, at least a 19, at least a 20, or more nucleobase portion of a target segment, or a range defined by any two of these values.

### Identity

The antisense compounds provided herein can also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases can be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, at least 75%, at least 80%, at least 85%, at least 90%, at least 95%, at least 96%, at least 97%, at least 98%, at least 99% or 100% identical to one or more of the exemplary antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a DMPK nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, 0-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each Rₗ, Rₘ and Rₐ is, independently, H or substituted or unsubstituted C₁-C₁₀ alkyl.

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C(═CH₂)-2′ (and analogs thereof see PCT/US2008/066154 published as WO 2008/154401, published on Dec. 8, 2008).

Further bicyclic nucleosides have been reported in published literature (see for example: Srivastava et al., J. Am. Chem. Soc., 2007, 129(26) 8362-8379; Frieden et al., Nucleic Acids Research, 2003, 21, 6365-6372; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wahlestedt et al., Proc. Natl. Acad. Sci. U.S.A, 2000, 97, 5633-5638; Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; U.S. Pat. Nos. 7,399,845; 7,053,207; 7,034,133; 6,794,499; 6,770,748; 6,670,461; 6,525,191; 6,268,490; U.S. Patent Publication Nos.: US2008-0039618; US2007-0287831; US2004-0171570; U.S. patent application Ser. Nos. 12/129,154; 61/099,844; 61/097,787; 61/086,231; 61/056,564; 61/026,998; 61/026,995; 60/989,574; International applications WO 2007/134181; WO 2005/021570; WO 2004/106356; WO 94/14226; and PCT International Applications Nos.: PCT/US2008/068922; PCT/US2008/066154; and PCT/US2008/064591). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic nucleosides comprise a bridge between the 4′ and the 2′ carbon atoms of the pentofuranosyl sugar moiety including without limitation, bridges comprising 1 or from 1 to 4 linked groups independently selected from —[C(Rₐ)(R_{b})]ₙ—, —C(Rₐ)═C(R_{b})—, —C(R)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-(CH₂)—O-2′ bridge, may be in the α-L configuration or in the j-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include those having a 4′ to 2′ bridge wherein such bridges include without limitation, α-L-4′-(CH₂)—O-2′, 3-D-4′-CH₂—O-2′, 4′-(CH₂)₂—O-2′, 4′-CH₂—O—N(R)-2′, 4′-CH₂—N(R)—O-2′, 4′-CH(CH₃)—O-2′, 4′-CH₂—S-2′, 4′-CH₂—N(R)-2′, 4′-CH₂—CH(CH₃)-2′, and 4′-(CH₂)₃-2′, wherein R is H, a protecting group or C₁-C₁₂ alkyl.

In certain embodiments, bicyclic nucleosides have the formula:

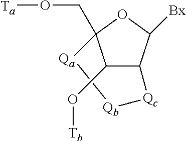

wherein:
Bx is a heterocyclic base moiety;
-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
R_{c} is C₁-C₁₂ alkyl or an amino protecting group; and
Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides have the formula:

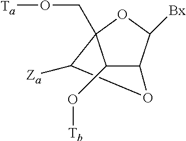

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thiol.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides have the formula:

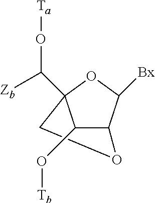

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides have the formula:

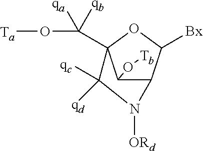

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides have the formula:

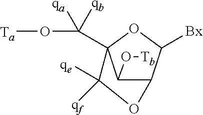

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
or qₑ and q_{f} together are ═C(q_{g})(qₕ);
q_{g} and qₕ are each, independently, H, halogen, C₁-C₁₂ alkyl or substituted C₁-C₁₂ alkyl.

The synthesis and preparation of adenine, cytosine, guanine, 5-methyl-cytosine, thymine and uracil bicyclic nucleosides having a 4′-CH₂—O-2′ bridge, along with their oligomerization, and nucleic acid recognition properties have been described (Koshkin et al., Tetrahedron, 1998, 54, 3607-3630). The synthesis of bicyclic nucleosides has also been described in WO 98/39352 and WO 99/14226.

Analogs of various bicyclic nucleosides that have 4′ to 2′ bridging groups such as 4′-CH₂—O-2′ and 4′-CH₂—S-2′, have also been prepared (Kumar et al., Bioorg. Med. Chem. Lett., 1998, 8, 2219-2222). Preparation of oligodeoxyribonucleotide duplexes comprising bicyclic nucleosides for use as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported. In certain embodiments, bicyclic nucleosides have the formula:

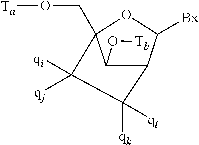

wherein:
Bx is a heterocyclic base moiety;
Tₐ and T_{b} are each, independently H, a hydroxyl protecting group, a conjugate group, a reactive phosphorus group, a phosphorus moiety or a covalent attachment to a support medium;
each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Frier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129(26), 8362-8379).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, (F) methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) vinyl BNA as depicted below.

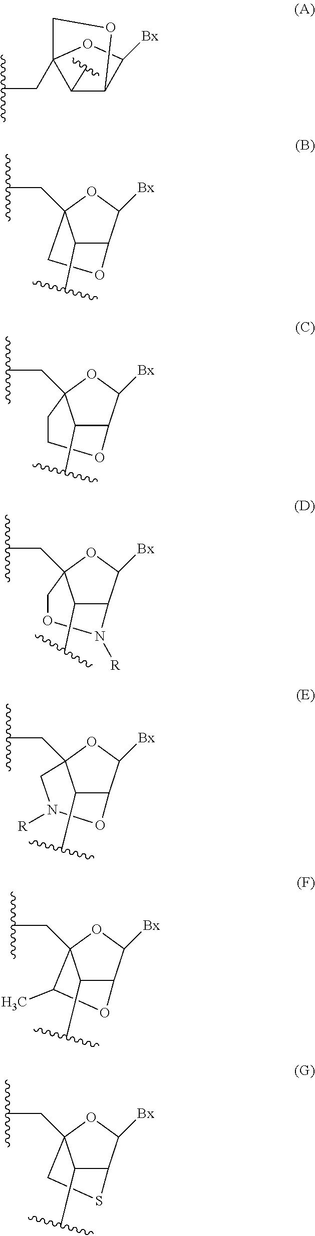

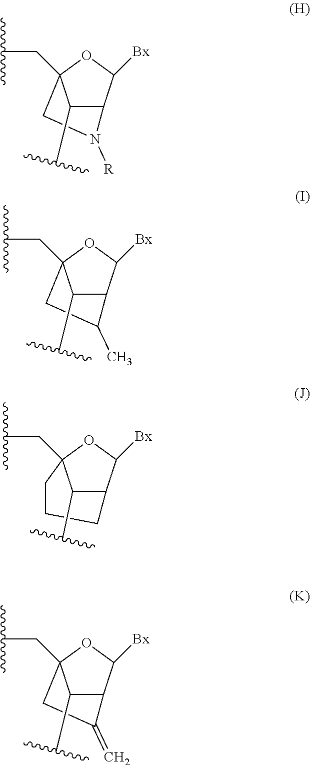

wherein Bx is the base moiety and R is, independently, H, a protecting group, C₁-C₆ alkyl or C₁-C₆ alkoxy.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

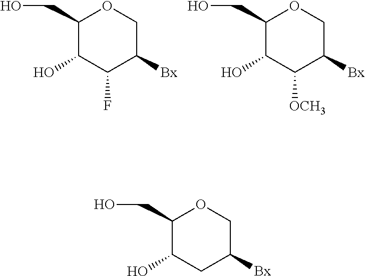

In certain embodiments, sugar surrogates are selected having the formula:

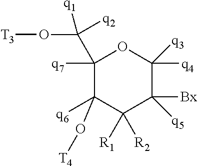

wherein:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to an oligomeric compound or oligonucleotide and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and
one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides are provided wherein one of R₁ and R₂ is F. In certain embodiments, R₁ is fluoro and R₂ is H; R₁ is methoxy and R₂ is H, and R₁ is methoxyethoxy and R₂ is H.

Such sugar surrogates include, but are not limited to, what is referred to in the art as hexitol nucleic acid (HNA), altritol nucleic acid (ANA), and mannitol nucleic acid (MNA) (see Leumann, C. J., Bioorg. & Med. Chem., 2002, 10, 841-854).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horvith et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have the formula:

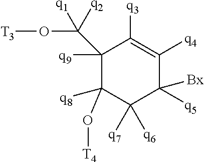

wherein:
Bx is a heterocyclic base moiety;
T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

Many other bicyclic and tricyclic sugar surrogate ring systems are also known in the art that can be used to modify nucleosides for incorporation into antisense compounds (see for example review article: Leumann, Christian J., Bioorg. & Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds targeted to a DMPK nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense compounds targeted to a DMPK nucleic acid comprise one or more modified nucleobases. In certain embodiments, gap-widened antisense oligonucleotides targeted to a DMPK nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a DMPK nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a DMPK nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of DMPK nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and cells are cultured according to the vendor's instructions using commercially available reagents (e.g. Invitrogen Life Technologies, Carlsbad, CA). Illustrative cell types include, but are not limited to, HepG2 cells, Hep3B cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, CA). Antisense oligonucleotides are mixed with LIPOFECTIN® in OPTI-MEM® 1 (Invitrogen, Carlsbad, CA) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE 2000® (Invitrogen, Carlsbad, CA). Antisense oligonucleotide is mixed with LIPOFECTAMINE 2000® in OPTI-MEM® 1 reduced serum medium (Invitrogen, Carlsbad, CA) to achieve the desired concentration of antisense oligonucleotide and a LIPOFECTAMINE® concentration that typically ranges 2 to 12 μg/mL per 100 nM antisense oligonucleotide. Another reagent used to introduce antisense oligonucleotides into cultured cells includes Cytofectin® (Invitrogen, Carlsbad, CA). Antisense oligonucleotide is mixed with Cytofectin® in OPTI-MEM® 1 reduced serum medium (Invitrogen, Carlsbad, CA) to achieve the desired concentration of antisense oligonucleotide and a Cytofectin® concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE2000®, Lipofectin or Cytofectin. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art. RNA is prepared using methods well known in the art, for example, using the TRIZOL® Reagent (Invitrogen, Carlsbad, CA) according to the manufacturer's recommended protocols.

### Analysis of Inhibition of Target Levels or Expression

Inhibition of levels or expression of a DMPK nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels can be accomplished by quantitative real-time PCR using the ABI PRISM® 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, CA). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, CA). Cyclophilin A expression is quantified by real time PCR, by being run simultaneously with the target, multiplexing, or separately. Total RNA is quantified using RIBOGREEN® RNA quantification reagent (Invitrogen, Inc. Eugene, OR). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a DMPK nucleic acid. Methods for designing real-time PCR probes and primers are well known in the art, and can include the use of software such as PRIMER EXPRESS® Software (Applied Biosystems, Foster City, CA).

### Analysis of Protein Levels

Antisense inhibition of DMPK nucleic acids can be assessed by measuring DMPK protein levels. Protein levels of DMPK can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of DMPK and produce phenotypic changes. Testing can be performed in normal animals, or in experimental disease models, for example, the HSA^{LR} mouse model of myotonic dystrophy (DM1).

The HSA^{LR} mouse model is an established model for DM1 (Mankodi, A. et al. Science. 289: 1769, 2000). The mice carry a human skeletal actin (hACTA1) transgene with 220 CTG repeats inserted in the 3′ UTR of the gene. The hACTA1-CUG^{exp} transcript accumulates in nuclear foci in skeletal muscles and results in myotonia similar to that in human DM1 (Mankodi, A. et al. Mol. Cell 10: 35, 2002; Lin, X. et al. Hum. Mol. Genet. 15: 2087, 2006). Hence, it is expected that amelioration of DM1 symptoms in the HSA^{LR} mouse by antisense inhibition of the hACTA1 transgene would predict amelioration of similar symptoms in human patients by antisense inhibition of the DMPK transcript.

Expression of CUG^{exp} RNA in mice causes extensive remodeling of the muscle transcriptome, much of which is reproduced by ablation of MBNL1. Hence, it is expected that normalization of the transcriptome in HSA^{LR} mice would predict normalization of the human transcriptome in DM1 patients by antisense inhibition of the DMPK transcript.

For administration to animals, antisense oligonucleotides are formulated in a pharmaceutically acceptable diluent, such as phosphate-buffered saline. Administration includes parenteral routes of administration. Following a period of treatment with antisense oligonucleotides, RNA is isolated from tissue and changes in DMPK nucleic acid expression are measured. Changes in DMPK protein levels are also measured.

### Splicing

Myotonic dystrophy (DM1) is caused by CTG repeat expansions in the 3′ untranslated region of the DMPK gene (Brook, J. D. et al. Cell. 68: 799, 1992). This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169). Such CUGexp are retained in the nuclear foci of skeletal muscles (Davis, B. M. et al. Proc. Natl. Acad. Sci. U.S.A. 94:7388, 1997). The accumulation of CUGexp in the nuclear foci leads to the sequestration of poly(CUG)-binding proteins, such as, Muscleblind-like 1 (MBLN1) (Miller, J. W. et al. EMBO J. 19: 4439, 2000). MBLN1 is a splicing factor and regulates the splicing of genes such as Serca1, CIC-1, Titin, and Zasp. Therefore, sequestration of MBLN1 by CUGexp triggers misregulated alternative splicing of the exons of genes that MBLN1 normally controls (Lin, X. et al. Hum. Mol. Genet. 15: 2087, 2006). Correction of alternative splicing in an animal displaying such disregulation, such as, for example, in a DM1 patient and the HSALR mouse model, is a useful indicator for the efficacy of a treatment, including treatment with an antisense oligonucleotide.

### Certain Biomarkers

DM1 severity in mouse models is determined, at least in part, by the level of CUG^{exp} transcript accumulation in the nucleus or nuclear foci. A useful physiological marker for DM1 severity is the development of high-frequency runs of involuntary action potentials (myotonia).

### Certain Indications

In certain embodiments, provided herein are methods of treating an individual comprising administering one or more pharmaceutical compositions as described herein. In certain embodiments, the individual has type 1 myotonic dystrophy (DM1).

Accordingly, provided herein are methods for ameliorating a symptom associated with type 1 myotonic dystrophy in a subject in need thereof. In certain embodiments, provided is a method for reducing the rate of onset of a symptom associated with type 1 myotonic dystrophy. In certain embodiments, provided is a method for reducing the severity of a symptom associated with type 1 myotonic dystrophy. In certain embodiments, symptoms associated with DM1 include muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, the symptoms may also be developmental delays, learning problems, language and speech issues, and personality development issues.

In certain embodiments, the methods comprise administering to an individual in need thereof a therapeutically effective amount of a compound targeted to a DMPK nucleic acid.

In certain embodiments, administration of an antisense compound targeted to a DMPK nucleic acid results in reduction of DMPK expression by at least about 15%, by at least about 20%, by at least about 25%, by at least about 30%, by at least about 35%, by at least about 40%, by at least about 45%, by at least about 50%, by at least about 55%, by at least about 60%, by least about 65%, by least about 70%, by least about 75%, by least about 80%, by at least about 85%, by at least about 90%, by at least about 95% or by at least about 99%, or a range defined by any two of these values.

In certain embodiments, pharmaceutical compositions comprising an antisense compound targeted to DMPK are used for the preparation of a medicament for treating a patient suffering or susceptible to type 1 myotonic dystrophy.

In certain embodiments, the methods described herein include administering a compound comprising a modified oligonucleotide having a contiguous nucleobases portion as described herein of a sequence recited in SEQ ID NO: 12-156, 160-770, and 774-792.

### Administration

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump. In certain embodiments, parenteral administration is by injection (e.g., bolus injection). The injection can be delivered with a syringe.

Parenteral administration includes subcutaneous administration, intravenous administration, intramuscular administration, intraarterial administration, intraperitoneal administration, or intracranial administration, e.g., intrathecal or intracerebroventricular administration. Administration can be continuous, or chronic, or short, or intermittent.

In certain embodiments, the administering is subcutaneous, intravenous, intracerebral, intracerebroventricular, intrathecal or another administration that results in a systemic effect of the oligonucleotide (systemic administration is characterized by a systemic effect, i.e., an effect in more than one tissue) or delivery to the CNS or to the CSF.

The duration of action as measured by inhibition of alpha 1 actin and reduction of myotonia in the HSA^{LR} mouse model of DM1 is prolonged in muscle tissue including quadriceps, gastrocnemius, and the tibialis anterior (see Examples, below). Subcutaneous injections of antisense oligonucleotide for 4 weeks results in inhibition of alpha 1 actin by at least 70% in quadriceps, gastrocnemius, and the tibialis anterior in HSA^{LR} mice for at least 11 weeks (77 days) after termination of dosing. Subcutaneous injections of antisense oligonucleotide for 4 weeks results in elimination of myotonia in quadriceps, gastrocnemius, and the tibialis anterior in HSA^{LR} mice for at least 11 weeks (77 days) after termination of dosing.

In certain embodiments, delivery of a compound of composition, as described herein, results in at least 70% down-regulation of a target mRNA and/or target protein for at least 77 days. In certain embodiments, delivery of a compound or composition, as described herein, results in 50%, 55%, 60%, 65%, 70%, 75%, 80%, 85%, 90%, 95%, or 100% down-regulation of a target mRNA and/or target protein for at least 30 days, at least 35 days, at least 40 days, at least 45 days, at least 50 days, at least 55 days, at least 60 days, at least 65 days, at least 70 days, at least 75 days, at least 76 days, at least 77 days, at least 78 days, at least 79 days, at least 80 days, at least 85 days, at least 90 days, at least 95 days, at least 100 days, at least 105 days, at least 110 days, at least 115 days, at least 120 days, at least 1 year.

In certain embodiments, an antisense oligonucleotide is delivered by injection or infusion once every 77 days. In certain embodiments, an antisense oligonucleotide is delivered by injection or infusion once every month, every two months, every three months, every 6 months, twice a year or once a year.

### Certain Combination Therapies

In certain embodiments, a first agent comprising the modified oligonucleotide of the invention is co-administered with one or more secondary agents. In certain embodiments, such second agents are designed to treat the same type 1 myotonic dystrophy as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, second agents are co-administered with the first agent to treat an undesired effect of the first agent. In certain embodiments, second agents are co-administered with the first agent to produce a combinational effect. In certain embodiments, second agents are co-administered with the first agent to produce a synergistic effect.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human Dystrophia Myotonica Protein Kinase (DMPK) in Human Skeletal Muscle Cells (hSKMC)

Antisense oligonucleotides targeted to a human DMPK nucleic acid were tested for their effect on DMPK RNA transcript in vitro. Cultured hSKM cells at a density of 20,000 cells per well were transfected using electroporation with 100 nM antisense oligonucleotide. After approximately 24 hours, RNA was isolated from the cells and DMPK RNA transcript levels were measured by quantitative real-time PCR with human primer probe set RTS3164 (forward sequence AGCCTGAGCCGGGAGATG, designated herein as SEQ ID NO: 9; reverse sequence GCGTAGTTGACTGGCGAAGTT, designated herein as SEQ ID NO: 10; probe sequence AGGCCATCCGCACGGACAACCX, designated herein as SEQ ID NO: 11). DMPK RNA transcript levels were adjusted according to total RNA content, as measured by RIBOGREEN®. Results are presented as percent inhibition of hDMPK, relative to untreated control cells.

The antisense oligonucleotides in Tables 1 and 2 are 5-10-5 gapmers, where the gap segment comprises ten 2′-deoxynucleosides and each wing segment comprises five 2′-MOE nucleosides. The internucleoside linkages throughout each gapmer are phosphorothioate (P═S) linkages. All cytosine residues throughout each gapmer are 5-methylcytosines. ‘Target start site’ indicates the 5′-most nucleoside to which the antisense oligonucleotide is targeted. ‘Target stop site’ indicates the 3′-most nucleoside to which the antisense oligonucleotide is targeted. All the antisense oligonucleotides listed in Table 1 target SEQ ID NO: 1 (GENBANK Accession No. NM_001081560.1). All the antisense oligonucleotides listed in Table 2 target SEQ ID NO: 2 (the complement of GENBANK Accession No. NT_011109.15 truncated from nucleotides 18540696 to 18555106).

Several antisense oligonucleotides demonstrated significant inhibition of human DMPK mRNA levels under the conditions specified above.

TABLE 1
Inhibition of human DMPK RNA transcript in hSKMC by 5-10-5
gapmers targeting SEQ ID NO: 1

| Target | Target | | | | |
|---|---|---|---|---|---|
| Start | Stop | | | % | SEQ ID |
| Site | Site | ISIS No | Sequence | inhibition | NO. |
| 93 | 112 | 299476 | CTGGCTGCATGTCTGCCTGT | 81 | 12 |
| 277 | 296 | 299479 | CCAGGAGAAGGTCGAGCAGG | 57 | 13 |
| 737 | 756 | 299493 | TCTATGGCCATGACAATCTC | 57 | 14 |
| 773 | 792 | 299494 | ATGTCCCTGTGCACGTAGCC | 77 | 15 |
| 1194 | 1213 | 299501 | ATGTGTCCGGAAGTCGCCTG | 50 | 16 |
| 1628 | 1647 | 299511 | CTCAGGCTCTGCCGGGTGAG | 70 | 17 |
| 1855 | 1874 | 299517 | GGCACTGGCCCACAGCCACG | 78 | 18 |
| 2379 | 2398 | 299526 | CCTGGCCGAAAGAAAGAAAT | 31 | 19 |
| 2367 | 2386 | 444380 | AAAGAAATGGTCTGTGATCC | 56 | 20 |
| 2370 | 2389 | 444381 | AAGAAAGAAATGGTCTGTGA | 77 | 21 |
| 2376 | 2395 | 444382 | GGCCGAAAGAAAGAAATGGT | 61 | 22 |
| 2385 | 2404 | 444383 | CCTCAGCCTGGCCGAAAGAA | 57 | 23 |
| 2388 | 2407 | 444384 | GGGCCTCAGCCTGGCCGAAA | 65 | 24 |
| 2391 | 2410 | 444385 | TCAGGGCCTCAGCCTGGCCG | 61 | 25 |
| 2411 | 2430 | 444386 | CTGCAGTTTGCCCATCCACG | 68 | 26 |
| 2414 | 2433 | 444387 | GGCCTGCAGTTTGCCCATCC | 77 | 27 |
| 2417 | 2436 | 444388 | CCAGGCCTGCAGTTTGCCCA | 54 | 28 |
| 2423 | 2442 | 444389 | GCCTTCCCAGGCCTGCAGTT | 77 | 29 |
| 2426 | 2445 | 444390 | GCTGCCTTCCCAGGCCTGCA | 83 | 30 |
| 2429 | 2448 | 444391 | CTTGCTGCCTTCCCAGGCCT | 69 | 31 |
| 2435 | 2454 | 444392 | GCCCGGCTTGCTGCCTTCCC | 82 | 32 |
| 2438 | 2457 | 444393 | ACGGCCCGGCTTGCTGCCTT | 78 | 33 |
| 2441 | 2460 | 444394 | CGGACGGCCCGGCTTGCTGC | 57 | 34 |
| 2444 | 2463 | 444395 | ACACGGACGGCCCGGCTTGC | 73 | 35 |
| 2450 | 2469 | 444396 | GATGGAACACGGACGGCCCG | 80 | 36 |
| 2453 | 2472 | 444397 | GAGGATGGAACACGGACGGC | 86 | 37 |
| 2456 | 2475 | 444398 | GTGGAGGATGGAACACGGAC | 84 | 38 |
| 2481 | 2500 | 444399 | GCGAACCAACGATAGGTGGG | 80 | 39 |
| 2484 | 2503 | 444400 | TTTGCGAACCAACGATAGGT | 86 | 40 |
| 2490 | 2509 | 444401 | TTGCACTTTGCGAACCAACG | 89 | 41 |
| 2493 | 2512 | 444402 | GCTTTGCACTTTGCGAACCA | 89 | 42 |
| 2496 | 2515 | 444403 | AAAGCTTTGCACTTTGCGAA | 83 | 43 |
| 2499 | 2518 | 444404 | AAGAAAGCTTTGCACTTTGC | 91 | 44 |
| 2502 | 2521 | 444405 | CACAAGAAAGCTTTGCACTT | 70 | 45 |
| 2508 | 2527 | 444406 | GTCATGCACAAGAAAGCTTT | 34 | 46 |
| 2527 | 2546 | 444407 | ACGCTCCCCAGAGCAGGGCG | 39 | 47 |
| 2543 | 2562 | 444408 | GCAGAGATCGCGCCAGACGC | 85 | 48 |
| 2546 | 2565 | 444409 | CAGGCAGAGATCGCGCCAGA | 65 | 49 |
| 2549 | 2568 | 444410 | AAGCAGGCAGAGATCGCGCC | 84 | 50 |
| 2555 | 2574 | 444411 | CCGAGTAAGCAGGCAGAGAT | 58 | 51 |
| 2558 | 2577 | 444412 | TTCCCGAGTAAGCAGGCAGA | 70 | 52 |
| 2564 | 2583 | 444413 | GCAAATTTCCCGAGTAAGCA | 62 | 53 |
| 2567 | 2586 | 444414 | AAAGCAAATTTCCCGAGTAA | 53 | 54 |
| 2573 | 2592 | 444415 | TTGGCAAAAGCAAATTTCCC | 64 | 55 |
| 2576 | 2595 | 444416 | GGTTTGGCAAAAGCAAATTT | 23 | 56 |
| 2579 | 2598 | 444417 | GCGGGTTTGGCAAAAGCAAA | 70 | 57 |
| 2582 | 2601 | 444418 | AAAGCGGGTTTGGCAAAAGC | 43 | 58 |
| 2588 | 2607 | 444419 | CCCGAAAAAGCGGGTTTGGC | 71 | 59 |
| 2591 | 2610 | 444420 | ATCCCCGAAAAAGCGGGTTT | 53 | 60 |
| 2595 | 2614 | 444421 | CGGGATCCCCGAAAAAGCGG | 45 | 61 |
| 2598 | 2617 | 444422 | GCGCGGGATCCCCGAAAAAG | 48 | 62 |
| 2623 | 2642 | 444423 | GAGAGCAGCGCAAGTGAGGA | 77 | 63 |
| 2626 | 2645 | 444424 | TCCGAGAGCAGCGCAAGTGA | 62 | 64 |
| 2629 | 2648 | 444425 | GGCTCCGAGAGCAGCGCAAG | 79 | 65 |
| 2649 | 2668 | 444426 | AAGCGGGCGGAGCCGGCTGG | 20 | 66 |
| 2652 | 2671 | 444427 | CCGAAGCGGGCGGAGCCGGC | 0 | 67 |
| 2658 | 2677 | 444428 | AAACCGCCGAAGCGGGCGGA | 0 | 68 |
| 2661 | 2680 | 444429 | TCCAAACCGCCGAAGCGGGC | 45 | 69 |
| 2664 | 2683 | 444430 | ATATCCAAACCGCCGAAGCG | 31 | 70 |
| 2667 | 2686 | 444431 | TAAATATCCAAACCGCCGAA | 42 | 71 |
| 2670 | 2689 | 444432 | CAATAAATATCCAAACCGCC | 53 | 72 |
| 2676 | 2695 | 444433 | CGAGGTCAATAAATATCCAA | 63 | 73 |
| 2679 | 2698 | 444434 | GGACGAGGTCAATAAATATC | 83 | 74 |
| 2682 | 2701 | 444435 | GGAGGACGAGGTCAATAAAT | 82 | 75 |
| 2685 | 2704 | 444436 | GTCGGAGGACGAGGTCAATA | 86 | 76 |
| 2688 | 2707 | 444437 | CGAGTCGGAGGACGAGGTCA | 73 | 77 |
| 2694 | 2713 | 444438 | TGTCAGCGAGTCGGAGGACG | 79 | 78 |
| 2697 | 2716 | 444439 | GCCTGTCAGCGAGTCGGAGG | 83 | 79 |
| 2700 | 2719 | 444440 | GTAGCCTGTCAGCGAGTCGG | 94 | 80 |
| 2703 | 2722 | 444441 | CCTGTAGCCTGTCAGCGAGT | 90 | 81 |
| 2706 | 2725 | 444442 | GGTCCTGTAGCCTGTCAGCG | 90 | 82 |
| 2764 | 2783 | 444443 | AAATACCGAGGAATGTCGGG | 82 | 83 |
| 2767 | 2786 | 444444 | AATAAATACCGAGGAATGTC | 66 | 84 |
| 2770 | 2789 | 444445 | GACAATAAATACCGAGGAAT | 67 | 85 |
| 2093 | 2112 | 445546 | CGGGGCCCCGGAGTCGAAGA | 0 | 86 |
| 2097 | 2116 | 445547 | CCAACGGGGCCCCGGAGTCG | 38 | 87 |
| 2099 | 2118 | 445548 | TTCCAACGGGGCCCCGGAGT | 22 | 88 |
| 2102 | 2121 | 445549 | GTCTTCCAACGGGGCCCCGG | 50 | 89 |
| 2104 | 2123 | 445550 | CAGTCTTCCAACGGGGCCCC | 27 | 90 |
| 2106 | 2125 | 445551 | CTCAGTCTTCCAACGGGGCC | 57 | 91 |
| 2109 | 2128 | 445552 | GCACTCAGTCTTCCAACGGG | 69 | 92 |
| 2115 | 2134 | 445553 | CCCCGGGCACTCAGTCTTCC | 76 | 93 |
| 2117 | 2136 | 445554 | TGCCCCGGGCACTCAGTCTT | 59 | 94 |
| 2119 | 2138 | 445555 | CGTGCCCCGGGCACTCAGTC | 61 | 95 |
| 2123 | 2142 | 445556 | GTGCCGTGCCCCGGGCACTC | 26 | 96 |
| 2126 | 2145 | 445557 | TCTGTGCCGTGCCCCGGGCA | 50 | 97 |
| 2129 | 2148 | 445558 | GCTTCTGTGCCGTGCCCCGG | 57 | 98 |
| 2132 | 2151 | 445559 | GCGGCTTCTGTGCCGTGCCC | 27 | 99 |
| 2134 | 2153 | 445560 | GCGCGGCTTCTGTGCCGTGC | 0 | 100 |
| 2136 | 2155 | 445561 | GGGCGCGGCTTCTGTGCCGT | 8 | 101 |
| 2142 | 2161 | 445562 | GGCGGTGGGCGCGGCTTCTG | 62 | 102 |
| 2146 | 2165 | 445563 | GGCAGGCGGTGGGCGCGGCT | 49 | 103 |
| 2148 | 2167 | 445564 | CTGGCAGGCGGTGGGCGCGG | 51 | 104 |
| 2150 | 2169 | 445565 | AACTGGCAGGCGGTGGGCGC | 38 | 105 |
| 2153 | 2172 | 445566 | GTGAACTGGCAGGCGGTGGG | 64 | 106 |
| 2157 | 2176 | 445567 | GGTTGTGAACTGGCAGGCGG | 66 | 107 |
| 2159 | 2178 | 445568 | GCGGTTGTGAACTGGCAGGC | 85 | 108 |
| 2163 | 2182 | 445569 | CGGAGCGGTTGTGAACTGGC | 92 | 109 |
| 2167 | 2186 | 445570 | CGCTCGGAGCGGTTGTGAAC | 51 | 110 |
| 2171 | 2190 | 445571 | CCCACGCTCGGAGCGGTTGT | 74 | 111 |
| 2174 | 2193 | 445572 | AGACCCACGCTCGGAGCGGT | 80 | 112 |
| 2177 | 2196 | 445573 | CGGAGACCCACGCTCGGAGC | 83 | 113 |
| 2180 | 2199 | 445574 | GGGCGGAGACCCACGCTCGG | 62 | 114 |
| 2183 | 2202 | 445575 | GCTGGGCGGAGACCCACGCT | 11 | 115 |
| 2186 | 2205 | 445576 | GGAGCTGGGCGGAGACCCAC | 42 | 116 |
| 2188 | 2207 | 445577 | CTGGAGCTGGGCGGAGACCC | 17 | 117 |
| 2191 | 2210 | 445578 | GGACTGGAGCTGGGCGGAGA | 53 | 118 |
| 2193 | 2212 | 445579 | CAGGACTGGAGCTGGGCGGA | 46 | 119 |
| 2197 | 2216 | 445580 | ATCACAGGACTGGAGCTGGG | 66 | 120 |
| 2209 | 2228 | 445581 | GGGCGGGCCCGGATCACAGG | 85 | 121 |
| 2211 | 2230 | 445582 | GGGGGCGGGCCCGGATCACA | 96 | 122 |
| 179 | 198 | 445583 | AGGCAGCACCATGGCCCCTC | 88 | 123 |
| 235 | 254 | 445584 | GGTCCAACACCAGCTGCTGG | 84 | 124 |
| 418 | 437 | 445585 | CGATCACCTTCAGAATCTCG | 11 | 125 |
| 498 | 517 | 445586 | CTTGTTCATGATCTTCATGG | 0 | 126 |
| 565 | 584 | 445587 | CCCCATTCACCAACACGTCC | 83 | 127 |
| 583 | 602 | 445588 | GCGTGATCCACCGCCGGTCC | 59 | 128 |
| 639 | 658 | 445589 | GTAATACTCCATGACCAGGT | 86 | 129 |
| 664 | 683 | 445590 | GCAGTGTCAGCAGGTCCCCG | 83 | 130 |
| 744 | 763 | 445591 | CACCGAGTCTATGGCCATGA | 60 | 131 |
| 761 | 780 | 445592 | ACGTAGCCAAGCCGGTGCAC | 68 | 132 |
| 812 | 831 | 445593 | ATGTGGCCACAGCGGTCCAG | 56 | 133 |
| 1099 | 1118 | 445594 | CTTCGTCCACCAGCGGCAGA | 32 | 134 |
| 1104 | 1123 | 445595 | GACCCCTTCGTCCACCAGCG | 83 | 135 |
| 1178 | 1197 | 445596 | CCTGCTCCACCCCGGCCCAG | 82 | 136 |
| 1187 | 1206 | 445597 | CGGAAGTCGCCTGCTCCACC | 81 | 137 |
| 1229 | 1248 | 445598 | CGGAGACCATCCCAGTCGAG | 67 | 138 |
| 1402 | 1421 | 445599 | TGAGGGCCATGCAGGAGTAG | 26 | 139 |
| 1443 | 1462 | 445600 | CTCCAGTTCCATGGGTGTGG | 80 | 140 |
| 1477 | 1496 | 445601 | GCGCTTGCACGTGTGGCTCA | 94 | 141 |
| 1526 | 1545 | 445602 | GCCACTTCAGCTGTTTCATC | 54 | 142 |
| 1562 | 1581 | 445603 | GCCTCAGCCTCTGCCGCAGG | 71 | 143 |
| 1576 | 1595 | 445604 | GCAGCGTCACCTCGGCCTCA | 31 | 144 |
| 1630 | 1649 | 445605 | GGCTCAGGCTCTGCCGGGTG | 86 | 145 |
| 1700 | 1719 | 445606 | TTCCGAGCCTCTGCCTCGCG | 73 | 146 |
| 1708 | 1727 | 445607 | GGTCCCGGTTCCGAGCCTCT | 76 | 147 |
| 1742 | 1761 | 445608 | ATCCGCTCCTGCAACTGCCG | 93 | 148 |
| 1750 | 1769 | 445609 | GCAACTCCATCCGCTCCTGC | 60 | 149 |
| 1812 | 1831 | 445610 | AGGTGGATCCGTGGCCCGGG | 48 | 150 |
| 2133 | 2152 | 445611 | CGCGGCTTCTGTGCCGTGCC | 24 | 151 |
| 2428 | 2447 | 445612 | TTGCTGCCTTCCCAGGCCTG | 80 | 152 |

TABLE 2
Inhibition of human DMPK RNA transcript in hSKMC by 5-10-5
gapmers targeting SEQ ID NO: 2

| Target | Target | | | | |
|---|---|---|---|---|---|
| Start | Stop | | | % | SEQ ID |
| Site | Site | ISIS No | Sequence | inhibition | NO. |
| 812 | 831 | 299471 | TGCTCCCGACAAGCTCCAGA | 95 | 153 |
| 876 | 895 | 299473 | AGAACCTGCCCATTGCTGAA | 68 | 154 |
| 2381 | 2400 | 299535 | CACTGAGGGCCAGACATATG | 68 | 155 |
| 3289 | 3308 | 299544 | CTCTAGATTCAGATGCAGGT | 88 | 156 |

The antisense oligonucleotides from Tables 1 and 2 were also tested in an assay with similar conditions as described above, and mRNA levels measured with the human primer probe RTS3162 (forward sequence CGGGCCGTCCGTGTT, designated herein as SEQ ID NO: 157; reverse sequence CTTTGCACTTTGCGAACCAA, designated herein as SEQ ID NO: 158; probe sequence CATCCTCCACGCACCCCCACCX, designated herein as SEQ ID NO: 159). The results are presented in Table 3. DMPK mRNA expression was also assessed by RTS3162 which targets the DMPK gene near the 3′UTR. The use of a second primer probe was employed to confirm that the expression of the entire DMPK gene had been inhibited

TABLE 3
Inhibition of human DMPK RNA transcript
in hSKMC by 5-10-5 gapmers measured
using primer probe set RTS3162

| ISIS | % |
|---|---|
| No | inhibition |
| 299471 | 91 |
| 299473 | 65 |
| 299476 | 76 |
| 299479 | 53 |
| 299493 | 60 |
| 299494 | 66 |
| 299501 | 44 |
| 299511 | 39 |
| 299517 | 71 |
| 299526 | 39 |
| 299535 | 75 |
| 299544 | 84 |
| 444380 | 72 |
| 444381 | 82 |
| 444382 | 67 |
| 444383 | 63 |
| 444384 | 66 |
| 444385 | 66 |
| 444386 | 74 |
| 444387 | 85 |
| 444388 | 60 |
| 444389 | 81 |
| 444390 | 88 |
| 444391 | 79 |
| 444392 | 94 |
| 444393 | 88 |
| 444394 | 94 |
| 444395 | 96 |
| 444396 | 96 |
| 444397 | 95 |
| 444398 | 96 |
| 444399 | 95 |
| 444400 | 95 |
| 444401 | 95 |
| 444402 | 91 |
| 444403 | 84 |
| 444404 | 89 |
| 444405 | 71 |
| 444406 | 47 |
| 444407 | 42 |
| 444408 | 80 |
| 444409 | 56 |
| 444410 | 79 |
| 444411 | 66 |
| 444412 | 67 |
| 444413 | 55 |
| 444414 | 45 |
| 444415 | 57 |
| 444416 | 18 |
| 444417 | 64 |
| 444418 | 51 |
| 444419 | 66 |
| 444420 | 0 |
| 444421 | 46 |
| 444422 | 33 |
| 444423 | 74 |
| 444424 | 73 |
| 444425 | 78 |
| 444426 | 0 |
| 444427 | 0 |
| 444428 | 0 |
| 444429 | 75 |
| 444430 | 28 |
| 444431 | 58 |
| 444432 | 52 |
| 444433 | 60 |
| 444434 | 87 |
| 444435 | 76 |
| 444436 | 83 |
| 444437 | 71 |
| 444438 | 76 |
| 444439 | 73 |
| 444440 | 91 |
| 444441 | 87 |
| 444442 | 93 |
| 444443 | 77 |
| 444444 | 64 |
| 444445 | 67 |
| 445546 | 0 |
| 445547 | 59 |
| 445548 | 49 |
| 445549 | 77 |
| 445550 | 62 |
| 445551 | 74 |
| 445552 | 84 |
| 445553 | 70 |
| 445554 | 63 |
| 445555 | 75 |
| 445556 | 52 |
| 445557 | 78 |
| 445558 | 81 |
| 445559 | 58 |
| 445560 | 12 |
| 445561 | 42 |
| 445562 | 70 |
| 445563 | 76 |
| 445564 | 69 |
| 445565 | 60 |
| 445566 | 86 |
| 445567 | 84 |
| 445568 | 92 |
| 445569 | 93 |
| 445570 | 59 |
| 445571 | 84 |
| 445572 | 88 |
| 445573 | 84 |
| 445574 | 74 |
| 445575 | 26 |
| 445576 | 56 |
| 445577 | 38 |
| 445578 | 69 |
| 445579 | 70 |
| 445580 | 75 |
| 445581 | 85 |
| 445582 | 95 |
| 445583 | 88 |
| 445584 | 87 |
| 445585 | 34 |
| 445586 | 0 |
| 445587 | 82 |
| 445588 | 66 |
| 445589 | 87 |
| 445590 | 82 |
| 445591 | 68 |
| 445592 | 64 |
| 445593 | 54 |
| 445594 | 52 |
| 445595 | 77 |
| 445596 | 84 |
| 445597 | 78 |
| 445598 | 73 |
| 445599 | 29 |
| 445600 | 68 |
| 445601 | 92 |
| 445602 | 53 |
| 445603 | 70 |
| 445604 | 32 |
| 445605 | 61 |
| 445606 | 84 |
| 445607 | 80 |
| 445608 | 91 |
| 445609 | 68 |
| 445610 | 63 |
| 445611 | 44 |
| 445612 | 91 |

### Example 2: Design of Antisense Oligonucleotides Targeting CUG Repeats

Antisense oligonucleotides were designed targeting mRNA transcripts that contain multiple CUG repeats. The chemistry of these oligonucleotides as well as their sequence is shown in Table 4. The symbols designated to the sugar type are shown after the base in subscript and are as follows: b=2′-O—N-[2-(dimethylamino)ethyl]acetamido ribose; d=2′-deoxyribose; e=2′-O-methoxyethyl ribose; f=2′-alpha-fluoro-2′-deoxyribose; g=2′-O-2[2-(2-methoxyethoxy)ethoxy]ethyl ribose; h=3′-fluoro-HNA; k=(S)-cEt; 1=LNA (Locked Nucleic Acids); n=2′-O—(N-methylacetamide) ribose; o=2′-O-dimethylaminooxyethyl (DMAOE) ribose; p=PNA; r=propylribose; and x=amino acid core. The heterocycle names are defined with standard symbols for adenine, cytosine, thymine and guanine, ‘mC’ for 5-methylcytosine, and ‘K’ for Lysine Side Chain. Linkers are shown after the sugar type in subscript and designated with the following symbols: g=PNA-glycine full; a=amino acid; and s=thioate ester.

TABLE 4
Design of antiseₙₛe oligonucleotides targeting CUG repeats

| ISIS | | | | SEQ |
|---|---|---|---|---|
| No | Sequence | Chemistry | Backbone | ID NO |
| 431896 | G_{ds}C_{ds}AₗₛG_{ds}C_{ds}AₗₛG_{ds}C_{ds}Aₗₛ | Deoxy and LNA units | Phosphorothioate | 802 |
| | G_{ds}C_{ds}AₗₛG_{ds}C_{ds}AₗₛG_{ds}C_{ds}AₗₛG_{d} | | | |
| 433804 | KₓₐG_{pg}C_{pg}A_{pg}G_{pg}C_{pg}A_{pg}G_{pg}C_{pg}A_{pg}G_{pg}C_{pg}A_{pg}G_{pg} | PNA and Amino Acid | mixed | 803 |
| | C_{pg}A_{pg}G_{pg}C_{pg}A_{pg}G_{pg}KₓₐKₓₐKₓₐKₓₐKₓₐKₓₐKₓₐKₓₐ | Core units with a | | |
| | | Carboxy-amide endcap | | |
| 444745 | AₑₛGₑₛmCₑₛAₑₛGₑₛmCₑₛAₑₛGₑₛmCₑₛAₑₛGₑₛmCₑₛ | Uniform MOE | Phosphorothioate | 789 |
| | AₑₛGₑₛmCₑₛAₑₛGₑₛmCₑₛAₑₛGₑₛmCₑₛAₑₛGₑₛmCₑₛAₑ | | | |
| 444746 | AₑₛGₑₛmCₑₛAₑₛGₑₛmCₑₛAₑₛGₑₛmCₑₛAₑₛ | Uniform MOE | Phosphorothioate | 804 |
| | GₑₛmCₑₛAₑₛGₑₛmCₑₛAₑₛGₑₛmCₑₛAₑₛGₑ | | | |
| 444747 | GₑₛmCₑₛAₑₛGₑₛmCₑₛAₑₛGₑₛmCₑₛAₑₛ | Uniform MOE | Phosphorothioate | 802 |
| | GₑₛmCₑₛAₑₛGₑₛmCₑₛAₑₛGₑₛmCₑₛAₑₛGₑₛ | | | |
| 444748 | GₑₛmCₑₛAₑₛGₑₛmCₑₛAₑₛGₑₛmCₑₛAₑₛ | Uniform MOE | Phosphorothioate | 805 |
| | GₑₛmCₑₛAₑₛGₑₛmCₑₛAₑₛGₑₛmCₑₛAₑ | | | |
| 444750 | GₖₛCₖₛA_{ds}G_{ds}CₖₛA_{ds}G_{ds}CₖₛA_{ds} | Deoxy and (S)-cEt units | Phosphorothioate | 805 |
| | G_{ds}CₖₛA_{ds}G_{ds}CₖₛA_{ds}G_{ds}CₖₛAₖ | | | |
| 444752 | GₖₛCₖₛAₑₛGₑₛCₖₛAₑₛGₑₛCₖₛAₑₛ | MOE and (S)-cEt units | Phosphorothioate | 805 |
| | GₑₛCₖₛAₑₛGₑₛCₖₛAₑₛGₑₛCₖₛAₖ | | | |
| 444754 | GₑₛmCₑₛA_{fs}G_{fs}C_{fs}A_{fs}G_{fs}C_{fs}A_{fs} | MOE and | Phosphorothioate | 805 |
| | G_{fs}C_{fs}A_{fs}G_{fs}C_{fs}A_{fs}G_{fs}mCₑₛAₑₛ | 2′-alpha-flouro units | | |
| 444759 | GₕₛmCₕₛAₕₛGₕₛmCₕₛAₕₛGₕₛmCₕₛAₕₛ | Uniform 3′-fluoro-HNA | Phosphorothioate | 805 |
| | GₕₛmCₕₛAₕₛGₕₛmCₕₛAₕₛGₕₛmCₕₛAₕ | | | |
| 444761 | GᵣₛmCᵣₛAᵣₛGᵣₛmCᵣₛAᵣₛGᵣₛmCᵣₛAᵣₛ | Uniform 2′-O- | Phosphorothioate | 805 |
| | GᵣₛmCᵣₛAᵣₛGᵣₛmCᵣₛAᵣₛGᵣₛmCᵣₛAᵣ | propylribose | | |
| 444762 | GₙₛmCₙₛAₙₛGₙₛmCₙₛAₙₛGₙₛmCₙₛAₙₛ | Uniform 2′-O-(N- | Phosphorothioate | 805 |
| | GₙₛmCₙₛAₙₛGₙₛmCₙₛAₙₛGₙₛmCₙₛAₙ | methylacetamide) ribose | | |
| 444763 | GₒₛmCₑₛAₒₛGₒₛmCₑₛAₒₛGₒₛmCₑₛAₒₛ | MOE and 2′-O- | Phosphorothioate | 805 |
| | GₒₛmCₑₛAₒₛGₒₛmCₑₛAₒₛGₒₛmCₑₛAₒ | dimethylaminooxyethyl | | |
| | | (DMAOE) ribose units | | |
| 444764 | G_{gs}mCₑₛAₑₛG_{gs}mCₑₛAₑₛGₑₛmCₑₛAₑₛ | MOE and 2′-O-2[2-(2- | Phosphorothioate | 802 |
| | G_{gs}mCₑₛAₑₛG_{gs}mCₑₛAₑₛG_{gs}mCₑₛAₑₛG_{g} | methoxyethoxy)ethoxy] | | |
| | | ethyl ribose units | | |
| 444765 | G_{bs}mCₑₛAₑₛG_{bs}mCₑₛAₑₛG_{bs}mCₑₛAₑₛ | MOE and 2′-O-N-[2- | Phosphorothioate | 802 |
| | G_{bs}mCₑₛAₑₛG_{bs}mCₑₛAₑₛG_{bs}mCₑₛAₑₛG_{b} | (dimethylamino)ethyl]ac | | |
| | | etamido ribose units | | |
| 473810 | AₖₛG_{ds}mC_{ds}AₖₛG_{ds}mC_{ds}AₖₛG_{ds}mC_{ds} | Deoxy and (S)-cEt units | Phosphorothioate | 806 |
| | AₖₛG_{ds}mC_{ds}AₖₛG_{ds}mC_{ds}AₖₛG_{ds}mC_{ds}Aₖ | | | |
| 473811 | AₖₛG_{ds}mC_{ds}AₖₛG_{ds}mC_{ds}AₖₛG_{ds} | Deoxy and (S)-cEt units | Phosphorothioate | 807 |
| | mC_{ds}AₖₛG_{ds}mC_{ds}AₖₛG_{ds}mC_{ds}Aₖ | | | |

### Example 3: Dose-Dependent Antisense Inhibition of Human DMPK in Human Skeletal Muscle Cells

Several of the antisense oligonucleotides exhibiting in vitro inhibition of DMPK in hSKMC (see Example 1) were tested at various doses. Cells were plated at a density of 20,000 cells per well and transfected using electroporation with 1,250 nM, 2,500 nM, 5,000 nM, 10,000 nM and 20,000 nM concentrations of each antisense oligonucleotide. After approximately 16 hours, RNA was isolated from the cells and DMPK mRNA transcript levels were measured by quantitative real-time PCR using primer probe set RTS3164, described hereinabove. DMPK mRNA transcript levels were normalized to total RNA content, as measured by RIBOGREEN®. Results are presented in Table 5 as percent inhibition of DMPK, relative to untreated control cells.

The tested antisense oligonucleotides demonstrated dose-dependent inhibition of DMPK mRNA levels under the conditions specified above.

TABLE 5
Dose-dependent antisense inhibition of human DMPK in hSKMC
tested with primer probe set RTS3164

| ISIS | 1,250 | 2,500 | 5,000 | 10,000 | 20,000 | IC₅₀ |
|---|---|---|---|---|---|---|
| No. | nM | nM | nM | nM | nM | (μM) |
| 299471 | 34 | 65 | 87 | 91 | 94 | 1.60 |
| 299473 | 2 | 33 | 60 | 89 | 92 | 4.31 |
| 299476 | 15 | 17 | 49 | 81 | 91 | 4.89 |
| 299535 | 0 | 12 | 34 | 62 | 59 | 9.95 |
| 299535 | 20 | 33 | 47 | 67 | 80 | 5.11 |
| 299544 | 32 | 63 | 81 | 85 | 87 | 1.82 |
| 444397 | 10 | 30 | 58 | 85 | 82 | 4.51 |
| 444398 | 33 | 57 | 74 | 85 | 87 | 2.07 |
| 444400 | 52 | 46 | 63 | 82 | 88 | 1.76 |
| 444401 | 51 | 71 | 84 | 89 | 91 | 0.71 |
| 444402 | 53 | 79 | 83 | 87 | 84 | <1.25 |
| 444404 | 48 | 68 | 77 | 86 | 90 | 0.95 |
| 444408 | 26 | 47 | 70 | 87 | 87 | 2.80 |
| 444410 | 22 | 47 | 67 | 83 | 87 | 3.12 |
| 444436 | 28 | 67 | 76 | 89 | 92 | 1.94 |
| 444440 | 70 | 77 | 83 | 89 | 85 | <1.25 |
| 444441 | 33 | 55 | 81 | 87 | 86 | 1.99 |
| 444442 | 54 | 73 | 84 | 89 | 88 | <1.25 |
| 445568 | 65 | 83 | 85 | 84 | 76 | <1.25 |
| 445569 | 60 | 77 | 87 | 93 | 91 | <1.25 |
| 445581 | 16 | 44 | 78 | 86 | 94 | 3.13 |
| 445582 | 0 | 7 | 26 | 96 | 99 | 5.60 |
| 445583 | 39 | 53 | 73 | 89 | 94 | 2.00 |
| 445584 | 20 | 26 | 61 | 81 | 93 | 4.02 |
| 445589 | 42 | 61 | 81 | 91 | 87 | 1.36 |
| 445601 | 49 | 79 | 87 | 93 | 94 | 0.66 |
| 445608 | 26 | 59 | 71 | 85 | 97 | 2.41 |
| 445612 | 46 | 59 | 72 | 88 | 93 | 1.51 |

The antisense oligonucleotides from Table 5 were also tested with primer probe set RTS3162, described hereinabove. The results are presented in Table 6. DMPK mRNA expression was also assessed by RTS3162 which targets the DMPK gene near the 3′UTR. The use of a second primer probe was employed to confirm that the expression of the entire DMPK gene had been inhibited.

TABLE 6
Dose-dependent antisense inhibition of human DMPK in hSKMC
tested with primer probe set RTS3164

| ISIS | 1,250 | 2,500 | 5,000 | 10,000 | 20,000 | IC₅₀ |
|---|---|---|---|---|---|---|
| No. | nM | nM | nM | nM | nM | (μM) |
| 299471 | 40 | 72 | 86 | 91 | 93 | 1.17 |
| 299473 | 6 | 43 | 63 | 87 | 89 | 3.86 |
| 299476 | 3 | 21 | 48 | 74 | 86 | 5.58 |
| 299535 | 9 | 22 | 36 | 62 | 77 | 7.05 |
| 299535 | 6 | 19 | 49 | 68 | 70 | 6.70 |
| 299544 | 35 | 66 | 81 | 84 | 87 | 1.52 |
| 444397 | 88 | 90 | 95 | 97 | 96 | <1.25 |
| 444398 | 91 | 97 | 97 | 97 | 98 | <1.25 |
| 444400 | 72 | 87 | 93 | 96 | 96 | <1.25 |
| 444401 | 86 | 92 | 97 | 98 | 97 | <1.25 |
| 444402 | 83 | 91 | 94 | 95 | 95 | <1.25 |
| 444404 | 49 | 69 | 81 | 90 | 93 | 0.92 |
| 444408 | 21 | 46 | 70 | 84 | 86 | 3.10 |
| 444410 | 35 | 55 | 77 | 89 | 91 | 2.02 |
| 444436 | 37 | 66 | 81 | 89 | 92 | 1.50 |
| 444440 | 66 | 79 | 89 | 92 | 89 | <1.25 |
| 444441 | 40 | 62 | 85 | 89 | 89 | 1.40 |
| 444442 | 55 | 75 | 86 | 90 | 91 | <1.25 |
| 445568 | 74 | 92 | 91 | 92 | 91 | <1.25 |
| 445569 | 68 | 83 | 90 | 94 | 93 | <1.25 |
| 445581 | 8 | 48 | 77 | 85 | 92 | 3.33 |
| 445582 | 15 | 22 | 44 | 97 | 99 | 4.29 |
| 445583 | 36 | 58 | 71 | 87 | 92 | 1.96 |
| 445584 | 25 | 43 | 66 | 86 | 94 | 3.05 |
| 445589 | 38 | 56 | 77 | 85 | 81 | 1.74 |
| 445601 | 55 | 76 | 84 | 93 | 93 | <1.25 |
| 445608 | 22 | 56 | 72 | 86 | 94 | 2.66 |
| 445612 | 61 | 75 | 85 | 91 | 94 | <1.25 |

### Example 4: Dose-Dependent Antisense Inhibition of Human DMPK in Human Skeletal Muscle Cells

Several of the antisense oligonucleotides exhibiting in vitro inhibition of DMPK in hSKMC (see Example 3) were tested at various doses. Cells were plated at a density of 20,000 cells per well and transfected using electroporation with 1,250 nM, 2,500 nM, 5,000 nM, 10,000 nM and 20,000 nM concentrations of each antisense oligonucleotide. After approximately 16 hours, RNA was isolated from the cells and DMPK mRNA transcript levels were measured by quantitative real-time PCR using primer probe set RTS3164, described hereinabove. DMPK mRNA transcript levels were normalized to total RNA content, as measured by RIBOGREEN®. Results are presented in Table 7 as percent inhibition of DMPK, relative to untreated control cells.

The majority of the tested antisense oligonucleotides demonstrated dose-dependent inhibition of DMPK mRNA levels under the conditions specified above.

TABLE 7
Dose-dependent antisense inhibition of human DMPK in
hSKMC tested with primer probe set RTS3164

| ISIS | 1,250 | 2,500 | 5,000 | 10,000 | 20,000 | IC₅₀ |
|---|---|---|---|---|---|---|
| No. | nM | nM | nM | nM | nM | (μM) |
| 299471 | 34 | 65 | 87 | 91 | 94 | 1.59 |
| 299473 | 2 | 33 | 60 | 89 | 92 | 4.31 |
| 299476 | 15 | 17 | 49 | 81 | 91 | 4.89 |
| 299535 | 0 | 12 | 34 | 62 | 59 | 9.95 |
| 299535 | 20 | 33 | 47 | 67 | 80 | 5.11 |
| 299544 | 32 | 63 | 81 | 85 | 87 | 1.82 |
| 444397 | 10 | 30 | 58 | 85 | 82 | 4.51 |
| 444398 | 33 | 57 | 74 | 85 | 87 | 2.07 |
| 444400 | 52 | 46 | 63 | 82 | 88 | 1.76 |
| 444401 | 51 | 71 | 84 | 89 | 91 | <1.25 |
| 444402 | 53 | 79 | 83 | 87 | 84 | <1.25 |
| 444404 | 48 | 68 | 77 | 86 | 90 | 0.95 |
| 444408 | 26 | 47 | 70 | 87 | 87 | 2.80 |
| 444410 | 22 | 47 | 67 | 83 | 87 | 3.12 |
| 444436 | 28 | 67 | 76 | 89 | 92 | 1.94 |
| 444440 | 66 | 77 | 83 | 89 | 85 | <1.25 |
| 444441 | 33 | 55 | 81 | 87 | 86 | 1.99 |
| 444442 | 54 | 73 | 84 | 89 | 88 | <1.25 |
| 445568 | 65 | 83 | 85 | 84 | 76 | <1.25 |
| 445569 | 60 | 77 | 87 | 93 | 91 | <1.25 |
| 445581 | 16 | 44 | 78 | 86 | 94 | 3.13 |
| 445582 | 0 | 7 | 26 | 96 | 99 | 5.62 |
| 445583 | 39 | 53 | 73 | 89 | 94 | 1.97 |
| 445584 | 20 | 26 | 61 | 81 | 93 | 4.20 |
| 445589 | 42 | 61 | 81 | 91 | 87 | 1.36 |
| 445601 | 49 | 79 | 87 | 93 | 94 | 0.66 |
| 445608 | 26 | 59 | 71 | 85 | 97 | 2.41 |
| 445612 | 46 | 59 | 72 | 88 | 93 | 1.51 |

### Example 5: Dose-Dependent Antisense Inhibition of Human DMPK in Human Skeletal Muscle Cells

Several antisense oligonucleotides were designed to target human DMPK mRNA and were tested in hSKMC at various doses. Several other antisense oligonucleotides were designed to target human actin mRNA and were also tested in hSKMC at various doses. The newly designed gapmers are 2-10-2 MOE or 3-10-3 MOE gapmers. The 2-10-2 MOE gapmers are 14 nucleosides in length and where the gap segment comprises ten 2′-deoxynucleosides and each wing segment comprises two 2′-MOE nucleosides. The 3-10-3 MOE gapmers are 16 nucleosides in length and where the gap segment comprises ten 2′-deoxynucleosides and each wing segment comprises three 2′-MOE nucleosides. The internucleoside linkages throughout each gapmer are phosphorothioate (P═S) linkages. All cytosine residues throughout each gapmer are 5-methylcytosines. ‘Target start site’ indicates the 5′-most nucleoside to which the antisense oligonucleotide is targeted. ‘Target stop site’ indicates the 3′-most nucleoside to which the antisense oligonucleotide is targeted. The antisense oligonucleotides listed in Table 8 target either the human DMPK genomic sequence, designated herein as SEQ ID NO: 2 (the complement of GENBANK Accession No. NT_011109.15 truncated from nucleotides 18540696 to 18555106) or the human actin sequence, designated herein as SEQ ID NO: 801 (GENBANK Accession No. NM_001100.3).

Cells were plated at a density of 20,000 cells per well and transfected using electroporation with 1,250 nM, 2,500 nM, 5,000 nM, 10,000 nM and 20,000 nM concentrations of each antisense oligonucleotide. After approximately 16 hours, RNA was isolated from the cells and DMPK mRNA transcript levels were measured by quantitative real-time PCR using primer probe set RTS3162, described hereinabove. DMPK mRNA transcript levels were normalized to total RNA content, as measured by RIBOGREEN®. Results are presented in Table 8 as percent inhibition of DMPK, relative to untreated control cells. The antisense oligonucleotides were also tested under similar conditions with RTS3164. The results are presented in Table 9.

Many of the tested antisense oligonucleotides demonstrated dose-dependent inhibition of DMPK mRNA levels under the conditions specified above.

TABLE 8
Dose-dependent antisense inhibition of human DMPK and human actin
in hSKMC tested with primer probe set RTS3162

| | | | Target | | | | | | | | SEQ |
|---|---|---|---|---|---|---|---|---|---|---|---|
| | | | SEQ ID | Start | | | | | | IC₅₀ | ID |
| ISIS No | Sequence | Motif | NO | Site | 1,250 nM | 2,500 nM | 5,000 nM | 10,000 nM | 20,000 nM | (nM) | NO |
| 468787 | CTCCCGACAAGCTCCA | 3-10-3 | 2 | 814 | 28 | 47 | 51 | 84 | 88 | 3.27 | 808 |
| 468772 | TCCCGACAAGCTCC | 2-10-2 | 2 | 815 | 17 | 39 | 67 | 72 | 80 | 4.04 | 809 |
| 468795 | GCTTGCACGTGTGGCT | 3-10-3 | 2 | 10935 | 32 | 58 | 77 | 85 | 75 | 1.94 | 810 |
| 468780 | CTTGCACGTGTGGC | 2-10-2 | 2 | 10936 | 22 | 17 | 43 | 66 | 77 | 6.23 | 811 |
| 468793 | GGTTGTGAACTGGCAG | 3-10-3 | 2 | 13224 | 69 | 77 | 93 | 96 | 96 | <1.25 | 812 |
| 468778 | GTTGTGAACTGGCA | 2-10-2 | 2 | 13225 | 60 | 69 | 89 | 95 | 97 | <1.25 | 813 |
| 468794 | GAGCGGTTGTGAACTG | 3-10-3 | 2 | 13228 | 21 | 32 | 61 | 70 | 86 | 4.27 | 814 |
| 468779 | AGCGGTTGTGAACT | 2-10-2 | 2 | 13229 | 40 | 45 | 72 | 91 | 97 | 2.20 | 815 |
| 468796 | GCTGCCTTCCCAGGCC | 3-10-3 | 2 | 13493 | 73 | 79 | 91 | 96 | 95 | <1.25 | 816 |
| 468781 | CTGCCTTCCCAGGC | 2-10-2 | 2 | 13494 | 36 | 53 | 66 | 86 | 90 | 2.28 | 817 |
| 468788 | GCACTTTGCGAACCAA | 3-10-3 | 2 | 13555 | 55 | 80 | 84 | 94 | 96 | <1.25 | 818 |
| 468773 | CACTTTGCGAACCA | 2-10-2 | 2 | 13556 | 31 | 52 | 82 | 91 | 93 | 2.16 | 819 |
| 468789 | GAAAGCTTTGCACTTT | 3-10-3 | 2 | 13564 | 42 | 66 | 83 | 91 | 98 | 1.31 | 820 |
| 468774 | AAAGCTTTGCACTT | 2-10-2 | 2 | 13565 | 21 | 0 | 31 | 41 | 55 | 1.87 | 821 |
| 468790 | CGGAGGACGAGGTCAA | 3-10-3 | 2 | 13750 | 43 | 57 | 79 | 87 | 89 | 1.51 | 822 |
| 468775 | GGAGGACGAGGTCA | 2-10-2 | 2 | 13751 | 27 | 51 | 58 | 78 | 81 | 3.18 | 823 |
| 468791 | AGCCTGTCAGCGAGTC | 3-10-3 | 2 | 13765 | 49 | 63 | 85 | 62 | 95 | 1.04 | 824 |
| 468776 | GCCTGTCAGCGAGT | 2-10-2 | 2 | 13766 | 65 | 47 | 81 | 88 | 93 | <1.25 | 825 |
| 468792 | TCCTGTAGCCTGTCAG | 3-10-3 | 2 | 13771 | 38 | 57 | 73 | 85 | 93 | 1.91 | 826 |
| 468777 | CCTGTAGCCTGTCA | 2-10-2 | 2 | 13772 | 15 | 58 | 66 | 85 | 92 | 2.99 | 827 |
| 468783 | GAAGCGAGGCTTCACT | 3-10-3 | 801 | 22 | 0 | 20 | 5 | 0 | 0 | >20.00 | 828 |
| 468768 | AAGCGAGGCTTCAC | 2-10-2 | 801 | 23 | 25 | 22 | 5 | 17 | 0 | >20.00 | 829 |
| 468784 | ACCTGCCCGTCTGGCA | 3-10-3 | 801 | 836 | 15 | 25 | 32 | 18 | 25 | >20.00 | 830 |
| 468769 | CCTGCCCGTCTGGC | 2-10-2 | 801 | 837 | 32 | 11 | 11 | 20 | 32 | >20.00 | 831 |
| 468782 | GGTCAGCGATCCCAGG | 3-10-3 | 801 | 1030 | 0 | 0 | 0 | 0 | 0 | >20.00 | 832 |
| 468767 | GTCAGCGATCCCAG | 2-10-2 | 801 | 1031 | 15 | 0 | 11 | 0 | 0 | >20.00 | 833 |
| 468785 | ATTTTCTTCCACAGGG | 3-10-3 | 801 | 1432 | 12 | 0 | 0 | 0 | 0 | >20.00 | 834 |
| 468770 | TTTTCTTCCACAGG | 2-10-2 | 801 | 1433 | 36 | 2 | 0 | 0 | 28 | >20.00 | 835 |
| 468786 | GAATGACTTTAATGCT | 3-10-3 | 801 | 1462 | 0 | 0 | 0 | 4 | 0 | >20.00 | 836 |
| 468771 | AATGACTTTAATGC | 2-10-2 | 801 | 1463 | 8 | 16 | 0 | 5 | 0 | >20.00 | 837 |

TABLE 9
Dose-dependent antisense inhibition of human DMPK in
hSKMC tested with primer probe set RTS3164

| ISIS | 1,250 | 2,500 | 5,000 | 10,000 | 20,000 | IC₅₀ |
|---|---|---|---|---|---|---|
| No | nM | nM | nM | nM | nM | (μM) |
| 468777 | 20 | 66 | 72 | 87 | 96 | 2.41 |
| 468776 | 68 | 48 | 86 | 90 | 96 | <1.25 |
| 468794 | 18 | 23 | 58 | 65 | 86 | 4.97 |
| 468787 | 36 | 50 | 51 | 88 | 92 | 2.69 |
| 468772 | 12 | 47 | 69 | 80 | 86 | 3.57 |
| 468773 | 33 | 48 | 82 | 91 | 96 | 2.21 |
| 468774 | 21 | 0 | 30 | 42 | 59 | 1.60 |
| 468790 | 50 | 57 | 77 | 91 | 91 | 1.26 |
| 468780 | 23 | 22 | 55 | 73 | 85 | 4.69 |
| 468775 | 29 | 52 | 55 | 79 | 84 | 3.03 |
| 468782 | 9 | 0 | 0 | 0 | 0 | >20.00 |
| 468786 | 2 | 0 | 0 | 0 | 0 | >20.00 |
| 468785 | 15 | 0 | 1 | 0 | 5 | >20.00 |
| 468788 | 57 | 74 | 76 | 94 | 96 | <1.25 |
| 468791 | 45 | 66 | 88 | 61 | 97 | 1.10 |
| 468789 | 26 | 65 | 82 | 90 | 97 | 2.02 |
| 468781 | 28 | 46 | 59 | 82 | 84 | 3.08 |
| 468779 | 26 | 31 | 66 | 90 | 97 | 3.29 |
| 468784 | 7 | 23 | 26 | 7 | 18 | >20.00 |
| 468783 | 0 | 16 | 8 | 0 | 0 | >20.00 |
| 468792 | 26 | 49 | 73 | 84 | 92 | 2.72 |
| 468795 | 30 | 53 | 83 | 86 | 85 | 2.14 |
| 468793 | 49 | 66 | 90 | 96 | 95 | 0.93 |
| 468768 | 23 | 3 | 5 | 9 | 0 | >20.00 |
| 468767 | 0 | 0 | 14 | 0 | 0 | >20.00 |
| 468769 | 31 | 0 | 0 | 16 | 25 | >20.00 |
| 468771 | 4 | 0 | 0 | 0 | 0 | >20.00 |
| 468770 | 33 | 0 | 0 | 0 | 32 | >20.00 |
| 468796 | 62 | 72 | 84 | 96 | 95 | <1.25 |
| 468778 | 44 | 58 | 86 | 96 | 98 | 1.44 |

### Example 6: Dose Response Studies with Antisense Oligonucleotides Targeting Human Dystrophia Myotonica-Protein Kinase (DMPK) in DM1 Fibroblast Cells

The mutant form of the DMPK mRNA, harboring large CUG repeats, are fully transcribed and polyadenylated, but remain trapped in the nucleus (Davis et al, 1997, Proc. Natl. Acad. Sci. U.S.A 94, 7388 7393). These mutant nuclear-retained mRNAs are one of the most important pathological features of myotonic dystrophy 1 (DM1). Antisense inhibition of mutant DMPK mRNA in DM1 fibroblast cells was studied.

The DMPK gene normally has 5-37 CTG repeats in the 3′ untranslated region. In myotonic dystrophy type I, this number is significantly expanded and may be in the range of 50 to greater than 3,500 (Harper, Myotonic Dystrophy (Saunders, London, ed. 3, 2001); Annu. Rev. Neurosci. 29: 259, 2006; EMBO J. 19: 4439, 2000; Curr Opin Neurol. 20: 572, 2007). DM1 fibroblast cells were plated at a density of 4,500 cells per well and transfected using Cytofectin reagent with 9.4 nM, 18.8 nM, 37.5 nM, 75.0 nM, 150.0 nM, and 300.0 nM concentrations of each antisense oligonucleotide. After approximately 16 hours, RNA was isolated from the cells and DMPK RNA transcript levels were measured by quantitative real-time PCR using primer probe set RTS3164, described hereinabove. DMPK RNA transcript levels were normalized to total RNA content, as measured by RIBOGREEN®. Results are presented in Table 10 as percent inhibition of DMPK, relative to untreated control cells.

An assay with similar conditions was also performed with primer probe set RTS3162, described hereinabove, which targets the 3′-end of the DMPK transcript. Results are presented in Table 11 as percent inhibition of DMPK, relative to untreated control cells.

The tested antisense oligonucleotides demonstrated dose-dependent inhibition of DMPK mRNA levels under the conditions specified above.

TABLE 10
Dose-dependent antisense inhibition of DMPK mRNA
in DM1 fibroblast cells with RTS3164

| ISIS | 9.4 | 18.8 | 37.5 | 75.0 | 150.0 | 300.0 | IC₅₀ |
|---|---|---|---|---|---|---|---|
| No. | nM | nM | nM | nM | nM | nM | (nM) |
| 299471 | 10 | 25 | 31 | 47 | 61 | 73 | 86.3 |
| 444401 | 8 | 27 | 41 | 60 | 67 | 74 | 64.3 |
| 444404 | 10 | 21 | 31 | 43 | 55 | 73 | 100.0 |
| 444436 | 7 | 17 | 36 | 64 | 68 | 70 | 72.3 |
| 445569 | 19 | 31 | 41 | 59 | 46 | 77 | 72.2 |

TABLE 11
Dose-dependent antisense inhibition of DMPK mRNA
in DM1 fibroblast cells with RTS3162

| ISIS | 9.4 | 18.8 | 37.5 | 75.0 | 150.0 | 300.0 | IC₅₀ |
|---|---|---|---|---|---|---|---|
| No | nM | nM | nM | nM | nM | nM | (nM) |
| 299471 | 7 | 25 | 29 | 46 | 48 | 69 | 115.3 |
| 444401 | 20 | 34 | 52 | 72 | 83 | 89 | 35.8 |
| 444404 | 5 | 20 | 28 | 42 | 54 | 77 | 98.8 |
| 444436 | 12 | 15 | 27 | 61 | 68 | 75 | 74.3 |
| 445569 | 5 | 25 | 33 | 53 | 50 | 76 | 89.6 |

### Example 7: Antisense Inhibition of Human DMPK in Human Skeletal Muscle Cells (hSKMc)

Antisense oligonucleotides targeted to a human DMPK nucleic acid were tested for their effect on DMPK RNA transcript in vitro. Cultured hSKMc at a density of 20,000 cells per well were transfected using electroporation with 10,000 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent inhibition of DMPK, relative to untreated control cells.

The antisense oligonucleotides in Tables 12 and 13 are 5-10-5 gapmers, where the gap segment comprises ten 2′-deoxynucleosides and each wing segment comprises five 2′-MOE nucleosides. The internucleoside linkages throughout each gapmer are phosphorothioate (P═S) linkages. All cytsoine residues throughout each gapmer are 5-methylcytosines. ‘Target start site’ indicates the 5′-most nucleoside to which the antisense oligonucleotide is targeted in the human genomic gene sequence. ‘Target stop site’ indicates the 3′-most nucleoside to which the antisense oligonucleotide is targeted in the human genomic sequence. All the antisense oligonucleotides listed in Table 12 target SEQ ID NO: 1 (GENBANK Accession No. NM_001081560.1). All the antisense oligonucleotides listed in Table 13 target SEQ ID NO: 2 (the complement of GENBANK Accession No. NT_011109.15 truncated from nucleotides 18540696 to 18555106).

Several of the antisense oligonucleotides demonstrated significant inhibition of DMPK mRNA levels under the conditions specified above.

TABLE 12
Inhibition of human DMPK RNA transcript in hSKMc by 5-10-5
gapmers targeting SEQ ID NO: 1

| Target | Target | | | | |
|---|---|---|---|---|---|
| Start | Stop | ISIS | | % | |
| Site | Site | No | Sequence | inhibition | SEQ ID NO. |
| 124 | 143 | 502369 | GCCTGGCAGCCCCTGTCCAG | 16 | 160 |
| 125 | 144 | 502370 | GGCCTGGCAGCCCCTGTCCA | 58 | 161 |
| 126 | 145 | 502371 | GGGCCTGGCAGCCCCTGTCC | 62 | 162 |
| 169 | 188 | 502372 | ATGGCCCCTCCCCGGGCCGG | 41 | 163 |
| 170 | 189 | 502373 | CATGGCCCCTCCCCGGGCCG | 29 | 164 |
| 171 | 190 | 502374 | CCATGGCCCCTCCCCGGGCC | 34 | 165 |
| 172 | 191 | 502375 | ACCATGGCCCCTCCCCGGGC | 60 | 166 |
| 173 | 192 | 502376 | CACCATGGCCCCTCCCCGGG | 68 | 167 |
| 174 | 193 | 502377 | GCACCATGGCCCCTCCCCGG | 75 | 168 |
| 175 | 194 | 502378 | AGCACCATGGCCCCTCCCCG | 65 | 169 |
| 176 | 195 | 502379 | CAGCACCATGGCCCCTCCCC | 63 | 170 |
| 177 | 196 | 502380 | GCAGCACCATGGCCCCTCCC | 73 | 171 |
| 178 | 197 | 502381 | GGCAGCACCATGGCCCCTCC | 80 | 172 |
| 180 | 199 | 502382 | CAGGCAGCACCATGGCCCCT | 82 | 173 |
| 181 | 200 | 502383 | ACAGGCAGCACCATGGCCCC | 72 | 174 |
| 183 | 202 | 502384 | GGACAGGCAGCACCATGGCC | 70 | 175 |
| 184 | 203 | 502385 | TGGACAGGCAGCACCATGGC | 71 | 176 |
| 185 | 204 | 502386 | TTGGACAGGCAGCACCATGG | 73 | 177 |
| 186 | 205 | 502387 | GTTGGACAGGCAGCACCATG | 73 | 178 |
| 187 | 206 | 502388 | TGTTGGACAGGCAGCACCAT | 60 | 179 |
| 188 | 207 | 502389 | ATGTTGGACAGGCAGCACCA | 75 | 180 |
| 189 | 208 | 502390 | CATGTTGGACAGGCAGCACC | 81 | 181 |
| 190 | 209 | 502391 | ACATGTTGGACAGGCAGCAC | 67 | 182 |
| 191 | 210 | 502392 | GACATGTTGGACAGGCAGCA | 71 | 183 |
| 192 | 211 | 502393 | TGACATGTTGGACAGGCAGC | 81 | 184 |
| 193 | 212 | 502394 | CTGACATGTTGGACAGGCAG | 76 | 185 |
| 194 | 213 | 502395 | GCTGACATGTTGGACAGGCA | 70 | 186 |
| 195 | 214 | 502396 | GGCTGACATGTTGGACAGGC | 77 | 187 |
| 196 | 215 | 502397 | CGGCTGACATGTTGGACAGG | 74 | 188 |
| 197 | 216 | 502398 | TCGGCTGACATGTTGGACAG | 63 | 189 |
| 198 | 217 | 502399 | CTCGGCTGACATGTTGGACA | 80 | 190 |
| 199 | 218 | 502400 | CCTCGGCTGACATGTTGGAC | 71 | 191 |
| 200 | 219 | 502401 | ACCTCGGCTGACATGTTGGA | 64 | 192 |
| 201 | 220 | 502402 | CACCTCGGCTGACATGTTGG | 71 | 193 |
| 202 | 221 | 502403 | GCACCTCGGCTGACATGTTG | 77 | 194 |
| 203 | 222 | 502404 | CGCACCTCGGCTGACATGTT | 80 | 195 |
| 204 | 223 | 502405 | CCGCACCTCGGCTGACATGT | 80 | 196 |
| 205 | 224 | 502406 | GCCGCACCTCGGCTGACATG | 79 | 197 |
| 206 | 225 | 502407 | AGCCGCACCTCGGCTGACAT | 74 | 198 |
| 207 | 226 | 502408 | CAGCCGCACCTCGGCTGACA | 66 | 199 |
| 208 | 227 | 502409 | TCAGCCGCACCTCGGCTGAC | 15 | 200 |
| 209 | 228 | 502410 | CTCAGCCGCACCTCGGCTGA | 32 | 201 |
| 210 | 229 | 502411 | CCTCAGCCGCACCTCGGCTG | 65 | 202 |
| 211 | 230 | 502412 | GCCTCAGCCGCACCTCGGCT | 81 | 203 |
| 232 | 251 | 502413 | CCAACACCAGCTGCTGGAGC | 90 | 204 |
| 233 | 252 | 502414 | TCCAACACCAGCTGCTGGAG | 78 | 205 |
| 234 | 253 | 502415 | GTCCAACACCAGCTGCTGGA | 84 | 206 |
| 236 | 255 | 502416 | GGGTCCAACACCAGCTGCTG | 69 | 207 |
| 257 | 276 | 502417 | GGCTCCAGCCCCAGGAAGCC | 46 | 208 |
| 258 | 277 | 502418 | GGGCTCCAGCCCCAGGAAGC | 28 | 209 |
| 276 | 295 | 502419 | CAGGAGAAGGTCGAGCAGGG | 41 | 210 |
| 278 | 297 | 502420 | CCCAGGAGAAGGTCGAGCAG | 71 | 211 |
| 279 | 298 | 502421 | GCCCAGGAGAAGGTCGAGCA | 85 | 212 |
| 280 | 299 | 451363 | CGCCCAGGAGAAGGTCGAGC | 84 | 213 |
| 281 | 300 | 502422 | ACGCCCAGGAGAAGGTCGAG | 67 | 214 |
| 317 | 336 | 502423 | TCCTGGGCCAGTTCGGAGGC | 58 | 215 |
| 318 | 337 | 502424 | GTCCTGGGCCAGTTCGGAGG | 71 | 216 |
| 319 | 338 | 502425 | TGTCCTGGGCCAGTTCGGAG | 69 | 217 |
| 320 | 339 | 502426 | TTGTCCTGGGCCAGTTCGGA | 71 | 218 |
| 321 | 340 | 502427 | CTTGTCCTGGGCCAGTTCGG | 66 | 219 |
| 322 | 341 | 502428 | ACTTGTCCTGGGCCAGTTCG | 59 | 220 |
| 323 | 342 | 502429 | TACTTGTCCTGGGCCAGTTC | 75 | 221 |
| 324 | 343 | 502430 | GTACTTGTCCTGGGCCAGTT | 78 | 222 |
| 325 | 344 | 502431 | CGTACTTGTCCTGGGCCAGT | 74 | 223 |
| 343 | 362 | 502432 | ACTGCAAGAAGTCGGCCACG | 73 | 224 |
| 345 | 364 | 502433 | CCACTGCAAGAAGTCGGCCA | 65 | 225 |
| 346 | 365 | 451364 | CCCACTGCAAGAAGTCGGCC | 32 | 226 |
| 347 | 366 | 502434 | GCCCACTGCAAGAAGTCGGC | 70 | 227 |
| 348 | 367 | 502435 | CGCCCACTGCAAGAAGTCGG | 61 | 228 |
| 349 | 368 | 502436 | CCGCCCACTGCAAGAAGTCG | 54 | 229 |
| 350 | 369 | 502437 | TCCGCCCACTGCAAGAAGTC | 40 | 230 |
| 351 | 370 | 502438 | CTCCGCCCACTGCAAGAAGT | 33 | 231 |
| 352 | 371 | 502439 | GCTCCGCCCACTGCAAGAAG | 23 | 232 |
| 353 | 372 | 502440 | GGCTCCGCCCACTGCAAGAA | 23 | 233 |
| 354 | 373 | 502441 | GGGCTCCGCCCACTGCAAGA | 17 | 234 |
| 355 | 374 | 502442 | TGGGCTCCGCCCACTGCAAG | 22 | 235 |
| 356 | 375 | 502443 | ATGGGCTCCGCCCACTGCAA | 14 | 236 |
| 357 | 376 | 502444 | GATGGGCTCCGCCCACTGCA | 43 | 237 |
| 358 | 377 | 502445 | CGATGGGCTCCGCCCACTGC | 37 | 238 |
| 359 | 378 | 502446 | ACGATGGGCTCCGCCCACTG | 0 | 239 |
| 360 | 379 | 502447 | CACGATGGGCTCCGCCCACT | 59 | 240 |
| 361 | 380 | 502448 | CCACGATGGGCTCCGCCCAC | 69 | 241 |
| 362 | 381 | 502449 | ACCACGATGGGCTCCGCCCA | 63 | 242 |
| 363 | 382 | 502450 | CACCACGATGGGCTCCGCCC | 73 | 243 |
| 364 | 383 | 502451 | TCACCACGATGGGCTCCGCC | 77 | 244 |
| 365 | 384 | 502452 | CTCACCACGATGGGCTCCGC | 66 | 245 |
| 366 | 385 | 502453 | CCTCACCACGATGGGCTCCG | 81 | 246 |
| 367 | 386 | 502454 | GCCTCACCACGATGGGCTCC | 77 | 247 |
| 368 | 387 | 502455 | AGCCTCACCACGATGGGCTC | 63 | 248 |
| 369 | 388 | 502456 | AAGCCTCACCACGATGGGCT | 70 | 249 |
| 370 | 389 | 502457 | TAAGCCTCACCACGATGGGC | 78 | 250 |
| 371 | 390 | 502458 | TTAAGCCTCACCACGATGGG | 76 | 251 |
| 372 | 391 | 502459 | CTTAAGCCTCACCACGATGG | 78 | 252 |
| 373 | 392 | 502460 | CCTTAAGCCTCACCACGATG | 68 | 253 |
| 374 | 393 | 502461 | TCCTTAAGCCTCACCACGAT | 67 | 254 |
| 375 | 394 | 502462 | CTCCTTAAGCCTCACCACGA | 84 | 255 |
| 376 | 395 | 502463 | CCTCCTTAAGCCTCACCACG | 76 | 256 |
| 377 | 396 | 502464 | ACCTCCTTAAGCCTCACCAC | 64 | 257 |
| 378 | 397 | 502465 | GACCTCCTTAAGCCTCACCA | 72 | 258 |
| 379 | 398 | 502466 | GGACCTCCTTAAGCCTCACC | 69 | 259 |
| 380 | 399 | 502467 | CGGACCTCCTTAAGCCTCAC | 81 | 260 |
| 381 | 400 | 502468 | TCGGACCTCCTTAAGCCTCA | 78 | 261 |
| 382 | 401 | 502469 | GTCGGACCTCCTTAAGCCTC | 57 | 262 |
| 384 | 403 | 502470 | CAGTCGGACCTCCTTAAGCC | 62 | 263 |
| 385 | 404 | 502471 | GCAGTCGGACCTCCTTAAGC | 45 | 264 |
| 386 | 405 | 502472 | TGCAGTCGGACCTCCTTAAG | 60 | 265 |
| 412 | 431 | 502473 | CCTTCAGAATCTCGAAGTCG | 67 | 266 |
| 413 | 432 | 502474 | ACCTTCAGAATCTCGAAGTC | 50 | 267 |
| 415 | 434 | 502475 | TCACCTTCAGAATCTCGAAG | 54 | 268 |
| 416 | 435 | 502476 | ATCACCTTCAGAATCTCGAA | 38 | 269 |
| 417 | 436 | 502477 | GATCACCTTCAGAATCTCGA | 35 | 270 |
| 419 | 438 | 502478 | CCGATCACCTTCAGAATCTC | 52 | 271 |
| 420 | 439 | 502479 | TCCGATCACCTTCAGAATCT | 50 | 272 |
| 421 | 440 | 502480 | GTCCGATCACCTTCAGAATC | 44 | 273 |
| 422 | 441 | 502481 | CGTCCGATCACCTTCAGAAT | 41 | 274 |
| 467 | 486 | 502482 | CCCGTCTGCTTCATCTTCAC | 67 | 275 |
| 468 | 487 | 502483 | GCCCGTCTGCTTCATCTTCA | 76 | 276 |
| 469 | 488 | 502484 | GGCCCGTCTGCTTCATCTTC | 57 | 277 |
| 470 | 489 | 502485 | TGGCCCGTCTGCTTCATCTT | 64 | 278 |
| 471 | 490 | 502486 | CTGGCCCGTCTGCTTCATCT | 64 | 279 |
| 472 | 491 | 502487 | CCTGGCCCGTCTGCTTCATC | 73 | 280 |
| 473 | 492 | 502488 | ACCTGGCCCGTCTGCTTCAT | 64 | 281 |
| 474 | 493 | 502489 | CACCTGGCCCGTCTGCTTCA | 80 | 282 |
| 475 | 494 | 502490 | ACACCTGGCCCGTCTGCTTC | 71 | 283 |
| 476 | 495 | 502491 | TACACCTGGCCCGTCTGCTT | 74 | 284 |
| 497 | 516 | 502492 | TTGTTCATGATCTTCATGGC | 56 | 285 |
| 499 | 518 | 502493 | ACTTGTTCATGATCTTCATG | 23 | 286 |
| 500 | 519 | 502494 | CACTTGTTCATGATCTTCAT | 43 | 287 |
| 501 | 520 | 502495 | CCACTTGTTCATGATCTTCA | 43 | 288 |
| 502 | 521 | 502496 | CCCACTTGTTCATGATCTTC | 47 | 289 |
| 503 | 522 | 502497 | TCCCACTTGTTCATGATCTT | 34 | 290 |
| 504 | 523 | 502498 | GTCCCACTTGTTCATGATCT | 34 | 291 |
| 505 | 524 | 502499 | TGTCCCACTTGTTCATGATC | 27 | 292 |
| 506 | 525 | 502500 | ATGTCCCACTTGTTCATGAT | 23 | 293 |
| 507 | 526 | 502501 | CATGTCCCACTTGTTCATGA | 51 | 294 |
| 508 | 527 | 502502 | GCATGTCCCACTTGTTCATG | 20 | 295 |
| 509 | 528 | 502503 | AGCATGTCCCACTTGTTCAT | 52 | 296 |
| 510 | 529 | 502504 | CAGCATGTCCCACTTGTTCA | 72 | 297 |
| 511 | 530 | 502505 | TCAGCATGTCCCACTTGTTC | 70 | 298 |
| 512 | 531 | 502506 | TTCAGCATGTCCCACTTGTT | 53 | 299 |
| 513 | 532 | 502507 | CTTCAGCATGTCCCACTTGT | 52 | 300 |
| 514 | 533 | 502508 | TCTTCAGCATGTCCCACTTG | 45 | 301 |
| 516 | 535 | 502509 | CCTCTTCAGCATGTCCCACT | 68 | 302 |
| 517 | 536 | 502510 | CCCTCTTCAGCATGTCCCAC | 68 | 303 |
| 518 | 537 | 502511 | CCCCTCTTCAGCATGTCCCA | 79 | 304 |
| 519 | 538 | 502512 | GCCCCTCTTCAGCATGTCCC | 85 | 305 |
| 520 | 539 | 502513 | CGCCCCTCTTCAGCATGTCC | 84 | 306 |
| 521 | 540 | 502514 | TCGCCCCTCTTCAGCATGTC | 80 | 307 |
| 522 | 541 | 502515 | CTCGCCCCTCTTCAGCATGT | 82 | 308 |
| 523 | 542 | 502516 | CCTCGCCCCTCTTCAGCATG | 78 | 309 |
| 524 | 543 | 502517 | ACCTCGCCCCTCTTCAGCAT | 73 | 310 |
| 525 | 544 | 502518 | CACCTCGCCCCTCTTCAGCA | 76 | 311 |
| 526 | 545 | 502519 | ACACCTCGCCCCTCTTCAGC | 79 | 312 |
| 527 | 546 | 502520 | GACACCTCGCCCCTCTTCAG | 73 | 313 |
| 821 | 840 | 502521 | GCCAGGCGGATGTGGCCACA | 57 | 314 |
| 868 | 887 | 502522 | ACCGCACCGTTCCATCTGCC | 62 | 315 |
| 869 | 888 | 502523 | GACCGCACCGTTCCATCTGC | 29 | 316 |
| 923 | 942 | 502524 | ACAGCCTGCAGGATCTCGGG | 86 | 317 |
| 924 | 943 | 502525 | CACAGCCTGCAGGATCTCGG | 81 | 318 |
| 925 | 944 | 502526 | CCACAGCCTGCAGGATCTCG | 83 | 319 |
| 926 | 945 | 502527 | CCCACAGCCTGCAGGATCTC | 84 | 320 |
| 927 | 946 | 502528 | GCCCACAGCCTGCAGGATCT | 91 | 321 |
| 928 | 947 | 502529 | CGCCCACAGCCTGCAGGATC | 90 | 322 |
| 929 | 948 | 502530 | CCGCCCACAGCCTGCAGGAT | 82 | 323 |
| 930 | 949 | 502531 | ACCGCCCACAGCCTGCAGGA | 83 | 324 |
| 931 | 950 | 502532 | CACCGCCCACAGCCTGCAGG | 85 | 325 |
| 932 | 951 | 502533 | CCACCGCCCACAGCCTGCAG | 84 | 326 |
| 933 | 952 | 502534 | CCCACCGCCCACAGCCTGCA | 80 | 327 |
| 934 | 953 | 502535 | GCCCACCGCCCACAGCCTGC | 90 | 328 |
| 935 | 954 | 502536 | GGCCCACCGCCCACAGCCTG | 94 | 329 |
| 936 | 955 | 502537 | AGGCCCACCGCCCACAGCCT | 88 | 330 |
| 937 | 956 | 502538 | CAGGCCCACCGCCCACAGCC | 91 | 331 |
| 938 | 957 | 502539 | CCAGGCCCACCGCCCACAGC | 73 | 332 |
| 939 | 958 | 502540 | CCCAGGCCCACCGCCCACAG | 86 | 333 |
| 940 | 959 | 502541 | TCCCAGGCCCACCGCCCACA | 88 | 334 |
| 941 | 960 | 502542 | GTCCCAGGCCCACCGCCCAC | 84 | 335 |
| 942 | 961 | 502543 | TGTCCCAGGCCCACCGCCCA | 85 | 336 |
| 943 | 962 | 502544 | CTGTCCCAGGCCCACCGCCC | 65 | 337 |
| 944 | 963 | 502545 | CCTGTCCCAGGCCCACCGCC | 81 | 338 |
| 945 | 964 | 502546 | GCCTGTCCCAGGCCCACCGC | 90 | 339 |
| 946 | 965 | 502547 | TGCCTGTCCCAGGCCCACCG | 85 | 340 |
| 947 | 966 | 502548 | CTGCCTGTCCCAGGCCCACC | 89 | 341 |
| 948 | 967 | 502549 | GCTGCCTGTCCCAGGCCCAC | 91 | 342 |
| 949 | 968 | 502550 | AGCTGCCTGTCCCAGGCCCA | 94 | 343 |
| 950 | 969 | 502551 | TAGCTGCCTGTCCCAGGCCC | 92 | 344 |
| 951 | 970 | 502552 | GTAGCTGCCTGTCCCAGGCC | 88 | 345 |
| 952 | 971 | 502553 | CGTAGCTGCCTGTCCCAGGC | 85 | 346 |
| 953 | 972 | 502554 | CCGTAGCTGCCTGTCCCAGG | 83 | 347 |
| 954 | 973 | 502555 | CCCGTAGCTGCCTGTCCCAG | 64 | 348 |
| 955 | 974 | 502556 | GCCCGTAGCTGCCTGTCCCA | 83 | 349 |
| 956 | 975 | 502557 | GGCCCGTAGCTGCCTGTCCC | 89 | 350 |
| 1004 | 1023 | 502558 | TAGAACATTTCATAGGCGAA | 68 | 351 |
| 1042 | 1061 | 502559 | TCTCCGCCGTGGAATCCGCG | 75 | 352 |
| 1043 | 1062 | 502560 | GTCTCCGCCGTGGAATCCGC | 79 | 353 |
| 1044 | 1063 | 502561 | GGTCTCCGCCGTGGAATCCG | 66 | 354 |
| 1045 | 1064 | 502562 | AGGTCTCCGCCGTGGAATCC | 50 | 355 |
| 1046 | 1065 | 502563 | TAGGTCTCCGCCGTGGAATC | 71 | 356 |
| 1067 | 1086 | 502564 | TTGTAGTGGACGATCTTGCC | 68 | 357 |
| 1068 | 1087 | 502565 | CTTGTAGTGGACGATCTTGC | 70 | 358 |
| 1069 | 1088 | 502566 | CCTTGTAGTGGACGATCTTG | 61 | 359 |
| 1070 | 1089 | 502567 | TCCTTGTAGTGGACGATCTT | 72 | 360 |
| 1071 | 1090 | 502568 | CTCCTTGTAGTGGACGATCT | 75 | 361 |
| 1072 | 1091 | 502569 | GCTCCTTGTAGTGGACGATC | 75 | 362 |
| 1073 | 1092 | 502570 | TGCTCCTTGTAGTGGACGAT | 83 | 363 |
| 1074 | 1093 | 502571 | GTGCTCCTTGTAGTGGACGA | 72 | 364 |
| 1075 | 1094 | 502572 | GGTGCTCCTTGTAGTGGACG | 66 | 365 |
| 1076 | 1095 | 502573 | AGGTGCTCCTTGTAGTGGAC | 51 | 366 |
| 1077 | 1096 | 502574 | GAGGTGCTCCTTGTAGTGGA | 46 | 367 |
| 1078 | 1097 | 502575 | AGAGGTGCTCCTTGTAGTGG | 70 | 368 |
| 1079 | 1098 | 502576 | GAGAGGTGCTCCTTGTAGTG | 47 | 369 |
| 1080 | 1099 | 502577 | AGAGAGGTGCTCCTTGTAGT | 65 | 370 |
| 1081 | 1100 | 502578 | GAGAGAGGTGCTCCTTGTAG | 45 | 371 |
| 1082 | 1101 | 502579 | AGAGAGAGGTGCTCCTTGTA | 63 | 372 |
| 1083 | 1102 | 502580 | CAGAGAGAGGTGCTCCTTGT | 77 | 373 |
| 1085 | 1104 | 502581 | GGCAGAGAGAGGTGCTCCTT | 70 | 374 |
| 1086 | 1105 | 502582 | CGGCAGAGAGAGGTGCTCCT | 80 | 375 |
| 1087 | 1106 | 502583 | GCGGCAGAGAGAGGTGCTCC | 62 | 376 |
| 1088 | 1107 | 502584 | AGCGGCAGAGAGAGGTGCTC | 44 | 377 |
| 1089 | 1108 | 502585 | CAGCGGCAGAGAGAGGTGCT | 78 | 378 |
| 1090 | 1109 | 502586 | CCAGCGGCAGAGAGAGGTGC | 71 | 379 |
| 1165 | 1184 | 502587 | GGCCCAGCCGTGTCTCCGGG | 77 | 380 |
| 1166 | 1185 | 502588 | CGGCCCAGCCGTGTCTCCGG | 69 | 381 |
| 1167 | 1186 | 502589 | CCGGCCCAGCCGTGTCTCCG | 70 | 382 |
| 1168 | 1187 | 502590 | CCCGGCCCAGCCGTGTCTCC | 75 | 383 |
| 1169 | 1188 | 502591 | CCCCGGCCCAGCCGTGTCTC | 77 | 384 |
| 1170 | 1189 | 502592 | ACCCCGGCCCAGCCGTGTCT | 73 | 385 |
| 1171 | 1190 | 502593 | CACCCCGGCCCAGCCGTGTC | 84 | 386 |
| 1172 | 1191 | 502594 | CCACCCCGGCCCAGCCGTGT | 78 | 387 |
| 1173 | 1192 | 502595 | TCCACCCCGGCCCAGCCGTG | 71 | 388 |
| 1174 | 1193 | 502596 | CTCCACCCCGGCCCAGCCGT | 81 | 389 |
| 1175 | 1194 | 502597 | GCTCCACCCCGGCCCAGCCG | 86 | 390 |
| 1176 | 1195 | 502598 | TGCTCCACCCCGGCCCAGCC | 83 | 391 |
| 1177 | 1196 | 502599 | CTGCTCCACCCCGGCCCAGC | 88 | 392 |
| 1199 | 1218 | 502600 | AAGGGATGTGTCCGGAAGTC | 60 | 393 |
| 1200 | 1219 | 502601 | GAAGGGATGTGTCCGGAAGT | 58 | 394 |
| 1201 | 1220 | 502602 | AGAAGGGATGTGTCCGGAAG | 63 | 395 |
| 1202 | 1221 | 502603 | AAGAAGGGATGTGTCCGGAA | 62 | 396 |
| 1203 | 1222 | 502604 | GAAGAAGGGATGTGTCCGGA | 61 | 397 |
| 1204 | 1223 | 502605 | AGAAGAAGGGATGTGTCCGG | 62 | 398 |
| 1205 | 1224 | 502606 | AAGAAGAAGGGATGTGTCCG | 56 | 399 |
| 1206 | 1225 | 502607 | AAAGAAGAAGGGATGTGTCC | 58 | 400 |
| 1207 | 1226 | 502608 | CAAAGAAGAAGGGATGTGTC | 50 | 401 |
| 1208 | 1227 | 502609 | CCAAAGAAGAAGGGATGTGT | 61 | 402 |
| 1210 | 1229 | 502610 | GGCCAAAGAAGAAGGGATGT | 73 | 403 |
| 1211 | 1230 | 502611 | AGGCCAAAGAAGAAGGGATG | 56 | 404 |
| 1212 | 1231 | 502612 | GAGGCCAAAGAAGAAGGGAT | 73 | 405 |
| 1213 | 1232 | 502613 | CGAGGCCAAAGAAGAAGGGA | 75 | 406 |
| 1214 | 1233 | 502614 | TCGAGGCCAAAGAAGAAGGG | 75 | 407 |
| 1215 | 1234 | 502615 | GTCGAGGCCAAAGAAGAAGG | 83 | 408 |
| 1216 | 1235 | 502616 | AGTCGAGGCCAAAGAAGAAG | 58 | 409 |
| 1217 | 1236 | 502617 | CAGTCGAGGCCAAAGAAGAA | 52 | 410 |
| 1218 | 1237 | 502618 | CCAGTCGAGGCCAAAGAAGA | 68 | 411 |
| 1219 | 1238 | 502619 | CCCAGTCGAGGCCAAAGAAG | 78 | 412 |
| 1220 | 1239 | 502620 | TCCCAGTCGAGGCCAAAGAA | 66 | 413 |
| 1221 | 1240 | 502621 | ATCCCAGTCGAGGCCAAAGA | 75 | 414 |
| 1222 | 1241 | 502622 | CATCCCAGTCGAGGCCAAAG | 70 | 415 |
| 1223 | 1242 | 502623 | CCATCCCAGTCGAGGCCAAA | 81 | 416 |
| 1224 | 1243 | 502624 | ACCATCCCAGTCGAGGCCAA | 82 | 417 |
| 1225 | 1244 | 502625 | GACCATCCCAGTCGAGGCCA | 88 | 418 |
| 1226 | 1245 | 502626 | AGACCATCCCAGTCGAGGCC | 79 | 419 |
| 1227 | 1246 | 502627 | GAGACCATCCCAGTCGAGGC | 82 | 420 |
| 1228 | 1247 | 502628 | GGAGACCATCCCAGTCGAGG | 60 | 421 |
| 1263 | 1282 | 502629 | TTCGAAATCCGGTGTAAAGG | 84 | 422 |
| 1264 | 1283 | 502630 | CTTCGAAATCCGGTGTAAAG | 57 | 423 |
| 1265 | 1284 | 502631 | CCTTCGAAATCCGGTGTAAA | 64 | 424 |
| 1266 | 1285 | 502632 | ACCTTCGAAATCCGGTGTAA | 73 | 425 |
| 1267 | 1286 | 502633 | CACCTTCGAAATCCGGTGTA | 77 | 426 |
| 1268 | 1287 | 502634 | GCACCTTCGAAATCCGGTGT | 59 | 427 |
| 1269 | 1288 | 502635 | GGCACCTTCGAAATCCGGTG | 85 | 428 |
| 1270 | 1289 | 502636 | TGGCACCTTCGAAATCCGGT | 86 | 429 |
| 1271 | 1290 | 502637 | GTGGCACCTTCGAAATCCGG | 74 | 430 |
| 1272 | 1291 | 502638 | GGTGGCACCTTCGAAATCCG | 79 | 431 |
| 1273 | 1292 | 502639 | CGGTGGCACCTTCGAAATCC | 85 | 432 |
| 1274 | 1293 | 502640 | TCGGTGGCACCTTCGAAATC | 71 | 433 |
| 1275 | 1294 | 502641 | GTCGGTGGCACCTTCGAAAT | 88 | 434 |
| 1276 | 1295 | 502642 | TGTCGGTGGCACCTTCGAAA | 89 | 435 |
| 1277 | 1296 | 502643 | GTGTCGGTGGCACCTTCGAA | 88 | 436 |
| 1278 | 1297 | 502644 | TGTGTCGGTGGCACCTTCGA | 87 | 437 |
| 1279 | 1298 | 502645 | ATGTGTCGGTGGCACCTTCG | 88 | 438 |
| 1280 | 1299 | 502646 | CATGTGTCGGTGGCACCTTC | 88 | 439 |
| 1281 | 1300 | 502647 | GCATGTGTCGGTGGCACCTT | 91 | 440 |
| 1282 | 1301 | 502648 | TGCATGTGTCGGTGGCACCT | 87 | 441 |
| 1283 | 1302 | 502649 | TTGCATGTGTCGGTGGCACC | 86 | 442 |
| 1284 | 1303 | 502650 | GTTGCATGTGTCGGTGGCAC | 83 | 443 |
| 1285 | 1304 | 502651 | AGTTGCATGTGTCGGTGGCA | 81 | 444 |
| 1286 | 1305 | 502652 | AAGTTGCATGTGTCGGTGGC | 79 | 445 |
| 1287 | 1306 | 502653 | GAAGTTGCATGTGTCGGTGG | 58 | 446 |
| 1288 | 1307 | 502654 | CGAAGTTGCATGTGTCGGTG | 85 | 447 |
| 1290 | 1309 | 502655 | GTCGAAGTTGCATGTGTCGG | 77 | 448 |
| 1291 | 1310 | 502656 | AGTCGAAGTTGCATGTGTCG | 79 | 449 |
| 1292 | 1311 | 502657 | AAGTCGAAGTTGCATGTGTC | 74 | 450 |
| 1293 | 1312 | 502658 | CAAGTCGAAGTTGCATGTGT | 82 | 451 |
| 1294 | 1313 | 502659 | CCAAGTCGAAGTTGCATGTG | 82 | 452 |
| 1295 | 1314 | 502660 | ACCAAGTCGAAGTTGCATGT | 70 | 453 |
| 1296 | 1315 | 502661 | CACCAAGTCGAAGTTGCATG | 76 | 454 |
| 1297 | 1316 | 502662 | CCACCAAGTCGAAGTTGCAT | 79 | 455 |
| 1298 | 1317 | 502663 | TCCACCAAGTCGAAGTTGCA | 68 | 456 |
| 1299 | 1318 | 502664 | CTCCACCAAGTCGAAGTTGC | 71 | 457 |
| 1300 | 1319 | 502665 | CCTCCACCAAGTCGAAGTTG | 67 | 458 |
| 1301 | 1320 | 502666 | TCCTCCACCAAGTCGAAGTT | 70 | 459 |
| 1302 | 1321 | 502667 | GTCCTCCACCAAGTCGAAGT | 80 | 460 |
| 1303 | 1322 | 502668 | CGTCCTCCACCAAGTCGAAG | 76 | 461 |
| 1304 | 1323 | 502669 | CCGTCCTCCACCAAGTCGAA | 78 | 462 |
| 1305 | 1324 | 502670 | CCCGTCCTCCACCAAGTCGA | 83 | 463 |
| 1306 | 1325 | 502671 | GCCCGTCCTCCACCAAGTCG | 76 | 464 |
| 1307 | 1326 | 502672 | AGCCCGTCCTCCACCAAGTC | 72 | 465 |
| 1308 | 1327 | 502673 | GAGCCCGTCCTCCACCAAGT | 71 | 466 |
| 1309 | 1328 | 502674 | TGAGCCCGTCCTCCACCAAG | 60 | 467 |
| 1702 | 1721 | 502675 | GGTTCCGAGCCTCTGCCTCG | 44 | 468 |
| 1703 | 1722 | 502676 | CGGTTCCGAGCCTCTGCCTC | 74 | 469 |
| 1704 | 1723 | 502677 | CCGGTTCCGAGCCTCTGCCT | 72 | 470 |
| 1705 | 1724 | 502678 | CCCGGTTCCGAGCCTCTGCC | 73 | 471 |
| 1706 | 1725 | 502679 | TCCCGGTTCCGAGCCTCTGC | 84 | 472 |
| 1707 | 1726 | 502680 | GTCCCGGTTCCGAGCCTCTG | 66 | 473 |
| 1709 | 1728 | 502681 | AGGTCCCGGTTCCGAGCCTC | 82 | 474 |
| 1710 | 1729 | 502682 | TAGGTCCCGGTTCCGAGCCT | 83 | 475 |
| 1711 | 1730 | 502683 | CTAGGTCCCGGTTCCGAGCC | 81 | 476 |
| 1712 | 1731 | 502684 | TCTAGGTCCCGGTTCCGAGC | 74 | 477 |
| 1713 | 1732 | 502685 | CTCTAGGTCCCGGTTCCGAG | 78 | 478 |
| 1714 | 1733 | 502686 | CCTCTAGGTCCCGGTTCCGA | 75 | 479 |
| 1715 | 1734 | 502687 | GCCTCTAGGTCCCGGTTCCG | 80 | 480 |
| 1743 | 1762 | 502688 | CATCCGCTCCTGCAACTGCC | 89 | 481 |
| 1744 | 1763 | 502689 | CCATCCGCTCCTGCAACTGC | 81 | 482 |
| 1745 | 1764 | 502690 | TCCATCCGCTCCTGCAACTG | 71 | 483 |
| 1746 | 1765 | 502691 | CTCCATCCGCTCCTGCAACT | 75 | 484 |
| 1747 | 1766 | 502692 | ACTCCATCCGCTCCTGCAAC | 64 | 485 |
| 1748 | 1767 | 502693 | AACTCCATCCGCTCCTGCAA | 52 | 486 |
| 1749 | 1768 | 502694 | CAACTCCATCCGCTCCTGCA | 45 | 487 |
| 1751 | 1770 | 502695 | AGCAACTCCATCCGCTCCTG | 78 | 488 |
| 1752 | 1771 | 502696 | CAGCAACTCCATCCGCTCCT | 64 | 489 |
| 1753 | 1772 | 502697 | GCAGCAACTCCATCCGCTCC | 56 | 490 |
| 1774 | 1793 | 502698 | CAGCTGTGGCTCCCTCTGCC | 60 | 491 |
| 1775 | 1794 | 502699 | ACAGCTGTGGCTCCCTCTGC | 45 | 492 |
| 1776 | 1795 | 502700 | GACAGCTGTGGCTCCCTCTG | 49 | 493 |
| 1777 | 1796 | 502701 | TGACAGCTGTGGCTCCCTCT | 26 | 494 |
| 1778 | 1797 | 502702 | GTGACAGCTGTGGCTCCCTC | 32 | 495 |
| 1779 | 1798 | 502703 | CGTGACAGCTGTGGCTCCCT | 28 | 496 |
| 1780 | 1799 | 502704 | CCGTGACAGCTGTGGCTCCC | 35 | 497 |
| 1781 | 1800 | 502705 | CCCGTGACAGCTGTGGCTCC | 33 | 498 |
| 1782 | 1801 | 502706 | CCCCGTGACAGCTGTGGCTC | 53 | 499 |
| 1783 | 1802 | 502707 | CCCCCGTGACAGCTGTGGCT | 39 | 500 |
| 1784 | 1803 | 502708 | ACCCCCGTGACAGCTGTGGC | 53 | 501 |
| 1785 | 1804 | 502709 | GACCCCCGTGACAGCTGTGG | 51 | 502 |
| 1786 | 1805 | 502710 | GGACCCCCGTGACAGCTGTG | 58 | 503 |
| 1787 | 1806 | 502711 | GGGACCCCCGTGACAGCTGT | 71 | 504 |
| 1814 | 1833 | 502712 | GAAGGTGGATCCGTGGCCCG | 73 | 505 |
| 1815 | 1834 | 502713 | GGAAGGTGGATCCGTGGCCC | 70 | 506 |
| 1816 | 1835 | 502714 | GGGAAGGTGGATCCGTGGCC | 72 | 507 |
| 1817 | 1836 | 502715 | TGGGAAGGTGGATCCGTGGC | 50 | 508 |
| 1818 | 1837 | 502716 | ATGGGAAGGTGGATCCGTGG | 62 | 509 |
| 1819 | 1838 | 502717 | GATGGGAAGGTGGATCCGTG | 75 | 510 |
| 1821 | 1840 | 502718 | TAGATGGGAAGGTGGATCCG | 52 | 511 |
| 1822 | 1841 | 502719 | CTAGATGGGAAGGTGGATCC | 56 | 512 |
| 1823 | 1842 | 502720 | TCTAGATGGGAAGGTGGATC | 21 | 513 |
| 1824 | 1843 | 502721 | ATCTAGATGGGAAGGTGGAT | 34 | 514 |
| 1826 | 1845 | 502722 | CCATCTAGATGGGAAGGTGG | 43 | 515 |
| 1827 | 1846 | 502723 | GCCATCTAGATGGGAAGGTG | 17 | 516 |
| 1828 | 1847 | 451383 | GGCCATCTAGATGGGAAGGT | 0 | 517 |
| 1863 | 1882 | 502724 | CACCAGCGGGCACTGGCCCA | 51 | 518 |
| 1864 | 1883 | 502725 | CCACCAGCGGGCACTGGCCC | 55 | 519 |
| 1865 | 1884 | 502726 | CCCACCAGCGGGCACTGGCC | 61 | 520 |
| 1866 | 1885 | 502727 | CCCCACCAGCGGGCACTGGC | 43 | 521 |
| 1868 | 1887 | 502728 | GGCCCCACCAGCGGGCACTG | 16 | 522 |
| 1869 | 1888 | 502729 | TGGCCCCACCAGCGGGCACT | 43 | 523 |
| 1870 | 1889 | 502730 | CTGGCCCCACCAGCGGGCAC | 43 | 524 |
| 1871 | 1890 | 502731 | CCTGGCCCCACCAGCGGGCA | 41 | 525 |
| 1872 | 1891 | 502732 | GCCTGGCCCCACCAGCGGGC | 30 | 526 |
| 1874 | 1893 | 502733 | GGGCCTGGCCCCACCAGCGG | 66 | 527 |
| 1892 | 1911 | 502734 | AGGTGGCGGCGGTGCATGGG | 31 | 528 |
| 1893 | 1912 | 502735 | CAGGTGGCGGCGGTGCATGG | 23 | 529 |
| 1894 | 1913 | 502736 | GCAGGTGGCGGCGGTGCATG | 57 | 530 |
| 1895 | 1914 | 502737 | AGCAGGTGGCGGCGGTGCAT | 54 | 531 |
| 1896 | 1915 | 502738 | CAGCAGGTGGCGGCGGTGCA | 61 | 532 |
| 1897 | 1916 | 502739 | GCAGCAGGTGGCGGCGGTGC | 57 | 533 |
| 1898 | 1917 | 502740 | AGCAGCAGGTGGCGGCGGTG | 36 | 534 |
| 1899 | 1918 | 502741 | GAGCAGCAGGTGGCGGCGGT | 53 | 535 |
| 1900 | 1919 | 502742 | GGAGCAGCAGGTGGCGGCGG | 39 | 536 |
| 1901 | 1920 | 502743 | GGGAGCAGCAGGTGGCGGCG | 36 | 537 |
| 1902 | 1921 | 502744 | AGGGAGCAGCAGGTGGCGGC | 62 | 538 |
| 1903 | 1922 | 502745 | CAGGGAGCAGCAGGTGGCGG | 56 | 539 |
| 1904 | 1923 | 502746 | GCAGGGAGCAGCAGGTGGCG | 58 | 540 |
| 1905 | 1924 | 502747 | GGCAGGGAGCAGCAGGTGGC | 65 | 541 |
| 1906 | 1925 | 502748 | TGGCAGGGAGCAGCAGGTGG | 47 | 542 |
| 1907 | 1926 | 502749 | CTGGCAGGGAGCAGCAGGTG | 41 | 543 |
| 1909 | 1928 | 451432 | CCCTGGCAGGGAGCAGCAGG | 53 | 544 |
| 1910 | 1929 | 502750 | ACCCTGGCAGGGAGCAGCAG | 52 | 545 |
| 1911 | 1930 | 502751 | GACCCTGGCAGGGAGCAGCA | 77 | 546 |
| 1912 | 1931 | 502752 | GGACCCTGGCAGGGAGCAGC | 0 | 547 |
| 1919 | 1938 | 502753 | GGCCTAGGGACCCTGGCAGG | 39 | 548 |
| 1920 | 1939 | 502754 | AGGCCTAGGGACCCTGGCAG | 35 | 549 |
| 1922 | 1941 | 502755 | CCAGGCCTAGGGACCCTGGC | 44 | 550 |
| 1923 | 1942 | 502756 | GCCAGGCCTAGGGACCCTGG | 60 | 551 |
| 1924 | 1943 | 502757 | GGCCAGGCCTAGGGACCCTG | 58 | 552 |
| 1925 | 1944 | 502758 | AGGCCAGGCCTAGGGACCCT | 57 | 553 |
| 1926 | 1945 | 502759 | TAGGCCAGGCCTAGGGACCC | 52 | 554 |
| 1927 | 1946 | 502760 | ATAGGCCAGGCCTAGGGACC | 51 | 555 |
| 1928 | 1947 | 502761 | GATAGGCCAGGCCTAGGGAC | 41 | 556 |
| 1929 | 1948 | 502762 | CGATAGGCCAGGCCTAGGGA | 69 | 557 |
| 1930 | 1949 | 502763 | CCGATAGGCCAGGCCTAGGG | 80 | 558 |
| 1931 | 1950 | 502764 | TCCGATAGGCCAGGCCTAGG | 78 | 559 |
| 1932 | 1951 | 502765 | CTCCGATAGGCCAGGCCTAG | 89 | 560 |
| 1933 | 1952 | 502766 | CCTCCGATAGGCCAGGCCTA | 79 | 561 |
| 1934 | 1953 | 502767 | GCCTCCGATAGGCCAGGCCT | 73 | 562 |
| 1936 | 1955 | 502768 | GCGCCTCCGATAGGCCAGGC | 83 | 563 |
| 1952 | 1971 | 502769 | AACAGGAGCAGGGAAAGCGC | 83 | 564 |
| 1953 | 1972 | 502770 | GAACAGGAGCAGGGAAAGCG | 70 | 565 |
| 1954 | 1973 | 502771 | CGAACAGGAGCAGGGAAAGC | 43 | 566 |
| 1955 | 1974 | 502772 | GCGAACAGGAGCAGGGAAAG | 47 | 567 |
| 1956 | 1975 | 502773 | GGCGAACAGGAGCAGGGAAA | 61 | 568 |
| 1957 | 1976 | 502774 | CGGCGAACAGGAGCAGGGAA | 74 | 569 |
| 1958 | 1977 | 502775 | ACGGCGAACAGGAGCAGGGA | 60 | 570 |
| 1959 | 1978 | 502776 | AACGGCGAACAGGAGCAGGG | 86 | 571 |
| 1960 | 1979 | 502777 | CAACGGCGAACAGGAGCAGG | 84 | 572 |
| 1981 | 2000 | 502778 | GGGCGGCGGCACGAGACAGA | 80 | 573 |
| 1982 | 2001 | 502779 | AGGGCGGCGGCACGAGACAG | 76 | 574 |
| 1983 | 2002 | 502780 | CAGGGCGGCGGCACGAGACA | 58 | 575 |
| 1984 | 2003 | 502781 | CCAGGGCGGCGGCACGAGAC | 80 | 576 |
| 1985 | 2004 | 502782 | CCCAGGGCGGCGGCACGAGA | 59 | 577 |
| 1986 | 2005 | 502783 | GCCCAGGGCGGCGGCACGAG | 68 | 578 |
| 1987 | 2006 | 502784 | AGCCCAGGGCGGCGGCACGA | 75 | 579 |
| 1988 | 2007 | 502785 | CAGCCCAGGGCGGCGGCACG | 76 | 580 |
| 1989 | 2008 | 502786 | GCAGCCCAGGGCGGCGGCAC | 70 | 581 |
| 2026 | 2045 | 502787 | CTGCGGTGAGTTGGCCGGCG | 68 | 582 |
| 2027 | 2046 | 502788 | ACTGCGGTGAGTTGGCCGGC | 67 | 583 |
| 2028 | 2047 | 502789 | GACTGCGGTGAGTTGGCCGG | 58 | 584 |
| 2029 | 2048 | 502790 | AGACTGCGGTGAGTTGGCCG | 71 | 585 |
| 2030 | 2049 | 502791 | CAGACTGCGGTGAGTTGGCC | 70 | 586 |
| 2031 | 2050 | 502792 | CCAGACTGCGGTGAGTTGGC | 79 | 587 |
| 2032 | 2051 | 502793 | GCCAGACTGCGGTGAGTTGG | 76 | 588 |
| 2033 | 2052 | 502794 | CGCCAGACTGCGGTGAGTTG | 66 | 589 |
| 2077 | 2096 | 502795 | AAGACAGTTCTAGGGTTCAG | 87 | 590 |
| 2078 | 2097 | 502796 | GAAGACAGTTCTAGGGTTCA | 78 | 591 |
| 2079 | 2098 | 502797 | CGAAGACAGTTCTAGGGTTC | 85 | 592 |
| 2080 | 2099 | 502798 | TCGAAGACAGTTCTAGGGTT | 78 | 593 |
| 2081 | 2100 | 502799 | GTCGAAGACAGTTCTAGGGT | 92 | 594 |
| 2082 | 2101 | 502800 | AGTCGAAGACAGTTCTAGGG | 85 | 595 |
| 2083 | 2102 | 502801 | GAGTCGAAGACAGTTCTAGG | 83 | 596 |
| 2084 | 2103 | 502802 | GGAGTCGAAGACAGTTCTAG | 86 | 597 |
| 2085 | 2104 | 502803 | CGGAGTCGAAGACAGTTCTA | 91 | 598 |
| 2086 | 2105 | 502804 | CCGGAGTCGAAGACAGTTCT | 76 | 599 |
| 2087 | 2106 | 502805 | CCCGGAGTCGAAGACAGTTC | 90 | 600 |
| 2088 | 2107 | 502806 | CCCCGGAGTCGAAGACAGTT | 83 | 601 |
| 2089 | 2108 | 502807 | GCCCCGGAGTCGAAGACAGT | 82 | 602 |
| 2090 | 2109 | 502808 | GGCCCCGGAGTCGAAGACAG | 73 | 603 |
| 2091 | 2110 | 502809 | GGGCCCCGGAGTCGAAGACA | 67 | 604 |
| 2143 | 2162 | 502810 | AGGCGGTGGGCGCGGCTTCT | 73 | 605 |
| 2144 | 2163 | 502811 | CAGGCGGTGGGCGCGGCTTC | 57 | 606 |
| 2145 | 2164 | 502812 | GCAGGCGGTGGGCGCGGCTT | 69 | 607 |
| 2147 | 2166 | 502813 | TGGCAGGCGGTGGGCGCGGC | 73 | 608 |
| 2149 | 2168 | 502814 | ACTGGCAGGCGGTGGGCGCG | 56 | 609 |
| 2151 | 2170 | 502815 | GAACTGGCAGGCGGTGGGCG | 71 | 610 |
| 2152 | 2171 | 502816 | TGAACTGGCAGGCGGTGGGC | 80 | 611 |
| 2154 | 2173 | 502817 | TGTGAACTGGCAGGCGGTGG | 85 | 612 |
| 2187 | 2206 | 502818 | TGGAGCTGGGCGGAGACCCA | 55 | 613 |
| 2189 | 2208 | 502819 | ACTGGAGCTGGGCGGAGACC | 53 | 614 |
| 2190 | 2209 | 502820 | GACTGGAGCTGGGCGGAGAC | 55 | 615 |
| 2192 | 2211 | 502821 | AGGACTGGAGCTGGGCGGAG | 76 | 616 |
| 2194 | 2213 | 502822 | ACAGGACTGGAGCTGGGCGG | 77 | 617 |
| 2195 | 2214 | 502823 | CACAGGACTGGAGCTGGGCG | 74 | 618 |
| 2196 | 2215 | 502824 | TCACAGGACTGGAGCTGGGC | 90 | 619 |
| 2386 | 2405 | 502825 | GCCTCAGCCTGGCCGAAAGA | 80 | 620 |
| 2387 | 2406 | 502826 | GGCCTCAGCCTGGCCGAAAG | 72 | 621 |
| 2490 | 2509 | 444401 | TTGCACTTTGCGAACCAACG | 97 | 41 |

TABLE 13
Inhibition of human DMPK RNA transcript in hSKMc by
5-10-5 gapmers targeting SEQ ID NO: 2

| Target | Target | | | | |
|---|---|---|---|---|---|
| Start | Stop | ISIS | | | SEQ ID |
| Site | Site | No | Sequence | % inhibition | NO. |
| 503 | 522 | 502983 | TGGTGGAGCCAAGCCCTCCC | 83 | 622 |
| 561 | 580 | 502984 | GGGCACCCTCAGAGCCTGAA | 82 | 623 |
| 1197 | 1216 | 502369 | GCCTGGCAGCCCCTGTCCAG | 16 | 160 |
| 1198 | 1217 | 502370 | GGCCTGGCAGCCCCTGTCCA | 58 | 161 |
| 1199 | 1218 | 502371 | GGGCCTGGCAGCCCCTGTCC | 62 | 162 |
| 1242 | 1261 | 502372 | ATGGCCCCTCCCCGGGCCGG | 41 | 163 |
| 1243 | 1262 | 502373 | CATGGCCCCTCCCCGGGCCG | 29 | 164 |
| 1244 | 1263 | 502374 | CCATGGCCCCTCCCCGGGCC | 34 | 165 |
| 1245 | 1264 | 502375 | ACCATGGCCCCTCCCCGGGC | 60 | 166 |
| 1246 | 1265 | 502376 | CACCATGGCCCCTCCCCGGG | 68 | 167 |
| 1247 | 1266 | 502377 | GCACCATGGCCCCTCCCCGG | 75 | 168 |
| 1248 | 1267 | 502378 | AGCACCATGGCCCCTCCCCG | 65 | 169 |
| 1249 | 1268 | 502379 | CAGCACCATGGCCCCTCCCC | 63 | 170 |
| 1250 | 1269 | 502380 | GCAGCACCATGGCCCCTCCC | 73 | 171 |
| 1251 | 1270 | 502381 | GGCAGCACCATGGCCCCTCC | 80 | 172 |
| 1253 | 1272 | 502382 | CAGGCAGCACCATGGCCCCT | 82 | 173 |
| 1254 | 1273 | 502383 | ACAGGCAGCACCATGGCCCC | 72 | 174 |
| 1256 | 1275 | 502384 | GGACAGGCAGCACCATGGCC | 70 | 175 |
| 1257 | 1276 | 502385 | TGGACAGGCAGCACCATGGC | 71 | 176 |
| 1258 | 1277 | 502386 | TTGGACAGGCAGCACCATGG | 73 | 177 |
| 1259 | 1278 | 502387 | GTTGGACAGGCAGCACCATG | 73 | 178 |
| 1260 | 1279 | 502388 | TGTTGGACAGGCAGCACCAT | 60 | 179 |
| 1261 | 1280 | 502389 | ATGTTGGACAGGCAGCACCA | 75 | 180 |
| 1262 | 1281 | 502390 | CATGTTGGACAGGCAGCACC | 81 | 181 |
| 1263 | 1282 | 502391 | ACATGTTGGACAGGCAGCAC | 67 | 182 |
| 1264 | 1283 | 502392 | GACATGTTGGACAGGCAGCA | 71 | 183 |
| 1265 | 1284 | 502393 | TGACATGTTGGACAGGCAGC | 81 | 184 |
| 1266 | 1285 | 502394 | CTGACATGTTGGACAGGCAG | 76 | 185 |
| 1267 | 1286 | 502395 | GCTGACATGTTGGACAGGCA | 70 | 186 |
| 1268 | 1287 | 502396 | GGCTGACATGTTGGACAGGC | 77 | 187 |
| 1269 | 1288 | 502397 | CGGCTGACATGTTGGACAGG | 74 | 188 |
| 1270 | 1289 | 502398 | TCGGCTGACATGTTGGACAG | 63 | 189 |
| 1271 | 1290 | 502399 | CTCGGCTGACATGTTGGACA | 80 | 190 |
| 1272 | 1291 | 502400 | CCTCGGCTGACATGTTGGAC | 71 | 191 |
| 1273 | 1292 | 502401 | ACCTCGGCTGACATGTTGGA | 64 | 192 |
| 1274 | 1293 | 502402 | CACCTCGGCTGACATGTTGG | 71 | 193 |
| 1275 | 1294 | 502403 | GCACCTCGGCTGACATGTTG | 77 | 194 |
| 1276 | 1295 | 502404 | CGCACCTCGGCTGACATGTT | 80 | 195 |
| 1277 | 1296 | 502405 | CCGCACCTCGGCTGACATGT | 80 | 196 |
| 1278 | 1297 | 502406 | GCCGCACCTCGGCTGACATG | 79 | 197 |
| 1279 | 1298 | 502407 | AGCCGCACCTCGGCTGACAT | 74 | 198 |
| 1280 | 1299 | 502408 | CAGCCGCACCTCGGCTGACA | 66 | 199 |
| 1281 | 1300 | 502409 | TCAGCCGCACCTCGGCTGAC | 15 | 200 |
| 1282 | 1301 | 502410 | CTCAGCCGCACCTCGGCTGA | 32 | 201 |
| 1283 | 1302 | 502411 | CCTCAGCCGCACCTCGGCTG | 65 | 202 |
| 1284 | 1303 | 502412 | GCCTCAGCCGCACCTCGGCT | 81 | 203 |
| 1305 | 1324 | 502413 | CCAACACCAGCTGCTGGAGC | 90 | 204 |
| 1306 | 1325 | 502414 | TCCAACACCAGCTGCTGGAG | 78 | 205 |
| 1307 | 1326 | 502415 | GTCCAACACCAGCTGCTGGA | 84 | 206 |
| 1309 | 1328 | 502416 | GGGTCCAACACCAGCTGCTG | 69 | 207 |
| 1330 | 1349 | 502417 | GGCTCCAGCCCCAGGAAGCC | 46 | 208 |
| 1331 | 1350 | 502418 | GGGCTCCAGCCCCAGGAAGC | 28 | 209 |
| 1349 | 1368 | 502419 | CAGGAGAAGGTCGAGCAGGG | 41 | 210 |
| 1351 | 1370 | 502420 | CCCAGGAGAAGGTCGAGCAG | 71 | 211 |
| 1352 | 1371 | 502421 | GCCCAGGAGAAGGTCGAGCA | 85 | 212 |
| 1353 | 1372 | 451363 | CGCCCAGGAGAAGGTCGAGC | 84 | 213 |
| 1354 | 1373 | 502422 | ACGCCCAGGAGAAGGTCGAG | 67 | 214 |
| 1390 | 1409 | 502423 | TCCTGGGCCAGTTCGGAGGC | 58 | 215 |
| 1391 | 1410 | 502424 | GTCCTGGGCCAGTTCGGAGG | 71 | 216 |
| 1392 | 1411 | 502425 | TGTCCTGGGCCAGTTCGGAG | 69 | 217 |
| 1393 | 1412 | 502426 | TTGTCCTGGGCCAGTTCGGA | 71 | 218 |
| 1394 | 1413 | 502427 | CTTGTCCTGGGCCAGTTCGG | 66 | 219 |
| 1395 | 1414 | 502428 | ACTTGTCCTGGGCCAGTTCG | 59 | 220 |
| 1396 | 1415 | 502429 | TACTTGTCCTGGGCCAGTTC | 75 | 221 |
| 1397 | 1416 | 502430 | GTACTTGTCCTGGGCCAGTT | 78 | 222 |
| 1398 | 1417 | 502431 | CGTACTTGTCCTGGGCCAGT | 74 | 223 |
| 1416 | 1435 | 502432 | ACTGCAAGAAGTCGGCCACG | 73 | 224 |
| 1418 | 1437 | 502433 | CCACTGCAAGAAGTCGGCCA | 65 | 225 |
| 1419 | 1438 | 451364 | CCCACTGCAAGAAGTCGGCC | 32 | 226 |
| 1421 | 1440 | 502985 | ACCCCACTGCAAGAAGTCGG | 60 | 624 |
| 1551 | 1570 | 502986 | GCCCCAGGATGGGAGGATCT | 58 | 625 |
| 1597 | 1616 | 502987 | CATAGGACAGAGAAATGTTG | 70 | 626 |
| 1630 | 1649 | 502988 | TGCTGACCTTACTCTGCCCC | 86 | 627 |
| 1666 | 1685 | 502989 | TAAGCCATGGCTCTGAGTCA | 51 | 628 |
| 1712 | 1731 | 502990 | AGAGAGGCCATGGGAGGCTG | 42 | 629 |
| 1841 | 1860 | 502991 | CTGGCCCTCCTGGCTTGCCC | 72 | 630 |
| 1853 | 1872 | 502992 | AGCTGCCCCATGCTGGCCCT | 76 | 631 |
| 1862 | 1881 | 502993 | GCCCCTGGCAGCTGCCCCAT | 70 | 632 |
| 1873 | 1892 | 502994 | CTGTCGGCTGCGCCCCTGGC | 78 | 633 |
| 1887 | 1906 | 502995 | CGCCGAACACCTGCCTGTCG | 68 | 634 |
| 1931 | 1950 | 502996 | CCTCCCAGTGCCTGGGCACC | 52 | 635 |
| 1981 | 2000 | 502998 | GCGCCTGTCTGCAAAGCTGG | 84 | 636 |
| 2025 | 2044 | 502999 | CCCAAAGTTGTCCCTCCTGG | 83 | 637 |
| 2038 | 2057 | 503000 | ACACCCAGAAGAACCCAAAG | 75 | 638 |
| 2117 | 2136 | 503001 | CTGACCCACACGGCTCATAG | 65 | 639 |
| 2235 | 2254 | 503002 | TGGCCCCAGGCCCTGGAAAG | 67 | 640 |
| 2278 | 2297 | 503003 | GACAAGGCAGCTGGCAGAAG | 79 | 641 |
| 2331 | 2350 | 503004 | AAGAAACCAGTGACCAGTGA | 85 | 642 |
| 2523 | 2542 | 503005 | CTGTGAAATGGGAGGAGGAG | 0 | 643 |
| 2578 | 2597 | 503006 | GAAGGTTTTTCCAGAGGCTG | 88 | 644 |
| 2615 | 2634 | 503007 | GGCCAGGAGAGTCATTAGGG | 84 | 645 |
| 2710 | 2729 | 503008 | CCACAAAAGGAGTGCTCCTC | 79 | 646 |
| 2789 | 2808 | 503009 | CCTTTTAAGGCAGCAGGAAC | 78 | 647 |
| 3629 | 3648 | 503010 | CTAGGACTGTCTGCTTCCCA | 88 | 648 |
| 3761 | 3780 | 502452 | CTCACCACGATGGGCTCCGC | 66 | 245 |
| 3762 | 3781 | 502453 | CCTCACCACGATGGGCTCCG | 81 | 246 |
| 3763 | 3782 | 502454 | GCCTCACCACGATGGGCTCC | 77 | 247 |
| 3764 | 3783 | 502455 | AGCCTCACCACGATGGGCTC | 63 | 248 |
| 3765 | 3784 | 502456 | AAGCCTCACCACGATGGGCT | 70 | 249 |
| 3766 | 3785 | 502457 | TAAGCCTCACCACGATGGGC | 78 | 250 |
| 3767 | 3786 | 502458 | TTAAGCCTCACCACGATGGG | 76 | 251 |
| 3768 | 3787 | 502459 | CTTAAGCCTCACCACGATGG | 78 | 252 |
| 3769 | 3788 | 502460 | CCTTAAGCCTCACCACGATG | 68 | 253 |
| 3770 | 3789 | 502461 | TCCTTAAGCCTCACCACGAT | 67 | 254 |
| 3771 | 3790 | 502462 | CTCCTTAAGCCTCACCACGA | 84 | 255 |
| 3772 | 3791 | 502463 | CCTCCTTAAGCCTCACCACG | 76 | 256 |
| 3773 | 3792 | 502464 | ACCTCCTTAAGCCTCACCAC | 64 | 257 |
| 3774 | 3793 | 502465 | GACCTCCTTAAGCCTCACCA | 72 | 258 |
| 3775 | 3794 | 502466 | GGACCTCCTTAAGCCTCACC | 69 | 259 |
| 3776 | 3795 | 502467 | CGGACCTCCTTAAGCCTCAC | 81 | 260 |
| 3777 | 3796 | 502468 | TCGGACCTCCTTAAGCCTCA | 78 | 261 |
| 3778 | 3797 | 502469 | GTCGGACCTCCTTAAGCCTC | 57 | 262 |
| 3780 | 3799 | 502470 | CAGTCGGACCTCCTTAAGCC | 62 | 263 |
| 3781 | 3800 | 502471 | GCAGTCGGACCTCCTTAAGC | 45 | 264 |
| 3782 | 3801 | 502472 | TGCAGTCGGACCTCCTTAAG | 60 | 265 |
| 3808 | 3827 | 502473 | CCTTCAGAATCTCGAAGTCG | 67 | 266 |
| 3809 | 3828 | 502474 | ACCTTCAGAATCTCGAAGTC | 50 | 267 |
| 3811 | 3830 | 502475 | TCACCTTCAGAATCTCGAAG | 54 | 268 |
| 3812 | 3831 | 502476 | ATCACCTTCAGAATCTCGAA | 38 | 269 |
| 3813 | 3832 | 502477 | GATCACCTTCAGAATCTCGA | 35 | 270 |
| 3815 | 3834 | 502478 | CCGATCACCTTCAGAATCTC | 52 | 271 |
| 3816 | 3835 | 502479 | TCCGATCACCTTCAGAATCT | 50 | 272 |
| 3817 | 3836 | 502480 | GTCCGATCACCTTCAGAATC | 44 | 273 |
| 3818 | 3837 | 502481 | CGTCCGATCACCTTCAGAAT | 41 | 274 |
| 3921 | 3940 | 503011 | GTCATTCATCAATTTCTAAG | 44 | 649 |
| 4118 | 4137 | 502482 | CCCGTCTGCTTCATCTTCAC | 67 | 275 |
| 4119 | 4138 | 502483 | GCCCGTCTGCTTCATCTTCA | 76 | 276 |
| 4120 | 4139 | 502484 | GGCCCGTCTGCTTCATCTTC | 57 | 277 |
| 4121 | 4140 | 502485 | TGGCCCGTCTGCTTCATCTT | 64 | 278 |
| 4122 | 4141 | 502486 | CTGGCCCGTCTGCTTCATCT | 64 | 279 |
| 4123 | 4142 | 502487 | CCTGGCCCGTCTGCTTCATC | 73 | 280 |
| 4124 | 4143 | 502488 | ACCTGGCCCGTCTGCTTCAT | 64 | 281 |
| 4125 | 4144 | 502489 | CACCTGGCCCGTCTGCTTCA | 80 | 282 |
| 4126 | 4145 | 502490 | ACACCTGGCCCGTCTGCTTC | 71 | 283 |
| 4127 | 4146 | 502491 | TACACCTGGCCCGTCTGCTT | 74 | 284 |
| 4148 | 4167 | 502492 | TTGTTCATGATCTTCATGGC | 56 | 285 |
| 4150 | 4169 | 502493 | ACTTGTTCATGATCTTCATG | 23 | 286 |
| 4151 | 4170 | 502494 | CACTTGTTCATGATCTTCAT | 43 | 287 |
| 4152 | 4171 | 502495 | CCACTTGTTCATGATCTTCA | 43 | 288 |
| 4153 | 4172 | 502496 | CCCACTTGTTCATGATCTTC | 47 | 289 |
| 4154 | 4173 | 502497 | TCCCACTTGTTCATGATCTT | 34 | 290 |
| 4155 | 4174 | 502498 | GTCCCACTTGTTCATGATCT | 34 | 291 |
| 4156 | 4175 | 502499 | TGTCCCACTTGTTCATGATC | 27 | 292 |
| 4157 | 4176 | 502500 | ATGTCCCACTTGTTCATGAT | 23 | 293 |
| 4158 | 4177 | 502501 | CATGTCCCACTTGTTCATGA | 51 | 294 |
| 4159 | 4178 | 502502 | GCATGTCCCACTTGTTCATG | 20 | 295 |
| 4160 | 4179 | 502503 | AGCATGTCCCACTTGTTCAT | 52 | 296 |
| 4161 | 4180 | 502504 | CAGCATGTCCCACTTGTTCA | 72 | 297 |
| 4162 | 4181 | 502505 | TCAGCATGTCCCACTTGTTC | 70 | 298 |
| 4163 | 4182 | 502506 | TTCAGCATGTCCCACTTGTT | 53 | 299 |
| 4164 | 4183 | 502507 | CTTCAGCATGTCCCACTTGT | 52 | 300 |
| 4165 | 4184 | 502508 | TCTTCAGCATGTCCCACTTG | 45 | 301 |
| 4167 | 4186 | 502509 | CCTCTTCAGCATGTCCCACT | 68 | 302 |
| 4168 | 4187 | 502510 | CCCTCTTCAGCATGTCCCAC | 68 | 303 |
| 4169 | 4188 | 502511 | CCCCTCTTCAGCATGTCCCA | 79 | 304 |
| 4170 | 4189 | 502512 | GCCCCTCTTCAGCATGTCCC | 85 | 305 |
| 4171 | 4190 | 502513 | CGCCCCTCTTCAGCATGTCC | 84 | 306 |
| 4172 | 4191 | 502514 | TCGCCCCTCTTCAGCATGTC | 80 | 307 |
| 4173 | 4192 | 502515 | CTCGCCCCTCTTCAGCATGT | 82 | 308 |
| 4174 | 4193 | 502516 | CCTCGCCCCTCTTCAGCATG | 78 | 309 |
| 4175 | 4194 | 502517 | ACCTCGCCCCTCTTCAGCAT | 73 | 310 |
| 4176 | 4195 | 502518 | CACCTCGCCCCTCTTCAGCA | 76 | 311 |
| 4239 | 4258 | 503012 | GGAGGAGCTGCAGCCGGAGA | 7 | 650 |
| 4245 | 4264 | 503013 | GCACCCGGAGGAGCTGCAGC | 0 | 651 |
| 4261 | 4280 | 503014 | GCACGACACCTGCAGGGCAC | 23 | 652 |
| 4355 | 4374 | 503015 | AGCTCACCAGGTAGTTCTCA | 49 | 653 |
| 4427 | 4446 | 503016 | GCTTCCTCTCCCCACCTCCT | 65 | 654 |
| 4447 | 4466 | 503017 | GCAGCACCCCCAATCCTAGA | 67 | 655 |
| 4508 | 4527 | 503018 | GCCCCTCATCCACCTGACAC | 62 | 656 |
| 4613 | 4632 | 503019 | TTCCAGGTAAGAGACCCCCC | 87 | 657 |
| 4679 | 4698 | 503020 | AGAATAGGTCCCAGACACTC | 81 | 658 |
| 4731 | 4750 | 503021 | CTCCCCCTGAGATGTTCTGG | 53 | 659 |
| 4858 | 4877 | 503022 | CCCCAGCCCAGAGATAACCA | 74 | 660 |
| 4927 | 4946 | 503023 | CCTGATCCATCACGGATGGC | 69 | 661 |
| 4987 | 5006 | 503024 | TACTCCATGACCAGGTACTG | 81 | 662 |
| 5185 | 5204 | 503025 | GCTCTGACCTTCCAAGAACC | 56 | 663 |
| 5354 | 5373 | 503026 | CTCCCTTCTGTGGTCCCACC | 0 | 664 |
| 5407 | 5426 | 503027 | GTCGGGTTTGATGTCCCTGC | 75 | 665 |
| 5445 | 5464 | 502521 | GCCAGGCGGATGTGGCCACA | 57 | 314 |
| 5500 | 5519 | 503028 | AGGGCACTGGCTCACCGTTC | 45 | 666 |
| 5681 | 5700 | 503029 | GGGCCCTCCTTCCAACCACT | 28 | 667 |
| 5708 | 5727 | 503030 | GCCCACCCCTCTGGGCCCAC | 45 | 668 |
| 5728 | 5747 | 503031 | AGGAGCAGAGCGAGGCTTGG | 38 | 669 |
| 5800 | 5819 | 502524 | ACAGCCTGCAGGATCTCGGG | 86 | 317 |
| 5801 | 5820 | 502525 | CACAGCCTGCAGGATCTCGG | 81 | 318 |
| 5802 | 5821 | 502526 | CCACAGCCTGCAGGATCTCG | 83 | 319 |
| 5803 | 5822 | 502527 | CCCACAGCCTGCAGGATCTC | 84 | 320 |
| 5804 | 5823 | 502528 | GCCCACAGCCTGCAGGATCT | 91 | 321 |
| 5805 | 5824 | 502529 | CGCCCACAGCCTGCAGGATC | 90 | 322 |
| 5806 | 5825 | 502530 | CCGCCCACAGCCTGCAGGAT | 82 | 323 |
| 5807 | 5826 | 502531 | ACCGCCCACAGCCTGCAGGA | 83 | 324 |
| 5808 | 5827 | 502532 | CACCGCCCACAGCCTGCAGG | 85 | 325 |
| 5809 | 5828 | 502533 | CCACCGCCCACAGCCTGCAG | 84 | 326 |
| 5810 | 5829 | 502534 | CCCACCGCCCACAGCCTGCA | 80 | 327 |
| 5811 | 5830 | 502535 | GCCCACCGCCCACAGCCTGC | 90 | 328 |
| 5812 | 5831 | 502536 | GGCCCACCGCCCACAGCCTG | 94 | 329 |
| 5813 | 5832 | 502537 | AGGCCCACCGCCCACAGCCT | 88 | 330 |
| 5814 | 5833 | 502538 | CAGGCCCACCGCCCACAGCC | 91 | 331 |
| 5815 | 5834 | 502539 | CCAGGCCCACCGCCCACAGC | 73 | 332 |
| 5816 | 5835 | 502540 | CCCAGGCCCACCGCCCACAG | 86 | 333 |
| 5817 | 5836 | 502541 | TCCCAGGCCCACCGCCCACA | 88 | 334 |
| 5818 | 5837 | 502542 | GTCCCAGGCCCACCGCCCAC | 84 | 335 |
| 5819 | 5838 | 502543 | TGTCCCAGGCCCACCGCCCA | 85 | 336 |
| 5820 | 5839 | 502544 | CTGTCCCAGGCCCACCGCCC | 65 | 337 |
| 5821 | 5840 | 502545 | CCTGTCCCAGGCCCACCGCC | 81 | 338 |
| 5822 | 5841 | 502546 | GCCTGTCCCAGGCCCACCGC | 90 | 339 |
| 5823 | 5842 | 502547 | TGCCTGTCCCAGGCCCACCG | 85 | 340 |
| 5824 | 5843 | 502548 | CTGCCTGTCCCAGGCCCACC | 89 | 341 |
| 5825 | 5844 | 502549 | GCTGCCTGTCCCAGGCCCAC | 91 | 342 |
| 5826 | 5845 | 502550 | AGCTGCCTGTCCCAGGCCCA | 94 | 343 |
| 5827 | 5846 | 502551 | TAGCTGCCTGTCCCAGGCCC | 92 | 344 |
| 5828 | 5847 | 502552 | GTAGCTGCCTGTCCCAGGCC | 88 | 345 |
| 5829 | 5848 | 502553 | CGTAGCTGCCTGTCCCAGGC | 85 | 346 |
| 5830 | 5849 | 502554 | CCGTAGCTGCCTGTCCCAGG | 83 | 347 |
| 5831 | 5850 | 502555 | CCCGTAGCTGCCTGTCCCAG | 64 | 348 |
| 5832 | 5851 | 502556 | GCCCGTAGCTGCCTGTCCCA | 83 | 349 |
| 5833 | 5852 | 502557 | GGCCCGTAGCTGCCTGTCCC | 89 | 350 |
| 5881 | 5900 | 502558 | TAGAACATTTCATAGGCGAA | 68 | 351 |
| 5919 | 5938 | 502559 | TCTCCGCCGTGGAATCCGCG | 75 | 352 |
| 5920 | 5939 | 502560 | GTCTCCGCCGTGGAATCCGC | 79 | 353 |
| 5921 | 5940 | 502561 | GGTCTCCGCCGTGGAATCCG | 66 | 354 |
| 5922 | 5941 | 502562 | AGGTCTCCGCCGTGGAATCC | 50 | 355 |
| 5923 | 5942 | 502563 | TAGGTCTCCGCCGTGGAATC | 71 | 356 |
| 5944 | 5963 | 502564 | TTGTAGTGGACGATCTTGCC | 68 | 357 |
| 5945 | 5964 | 502565 | CTTGTAGTGGACGATCTTGC | 70 | 358 |
| 5946 | 5965 | 502566 | CCTTGTAGTGGACGATCTTG | 61 | 359 |
| 5948 | 5967 | 503032 | CACCTTGTAGTGGACGATCT | 62 | 670 |
| 6039 | 6058 | 502582 | CGGCAGAGAGAGGTGCTCCT | 80 | 375 |
| 6040 | 6059 | 502583 | GCGGCAGAGAGAGGTGCTCC | 62 | 376 |
| 6041 | 6060 | 502584 | AGCGGCAGAGAGAGGTGCTC | 44 | 377 |
| 6042 | 6061 | 502585 | CAGCGGCAGAGAGAGGTGCT | 78 | 378 |
| 6043 | 6062 | 502586 | CCAGCGGCAGAGAGAGGTGC | 71 | 379 |
| 6118 | 6137 | 502587 | GGCCCAGCCGTGTCTCCGGG | 77 | 380 |
| 6119 | 6138 | 502588 | CGGCCCAGCCGTGTCTCCGG | 69 | 381 |
| 6120 | 6139 | 502589 | CCGGCCCAGCCGTGTCTCCG | 70 | 382 |
| 6121 | 6140 | 502590 | CCCGGCCCAGCCGTGTCTCC | 75 | 383 |
| 6122 | 6141 | 502591 | CCCCGGCCCAGCCGTGTCTC | 77 | 384 |
| 6123 | 6142 | 502592 | ACCCCGGCCCAGCCGTGTCT | 73 | 385 |
| 6124 | 6143 | 502593 | CACCCCGGCCCAGCCGTGTC | 84 | 386 |
| 6125 | 6144 | 502594 | CCACCCCGGCCCAGCCGTGT | 78 | 387 |
| 6126 | 6145 | 502595 | TCCACCCCGGCCCAGCCGTG | 71 | 388 |
| 6127 | 6146 | 502596 | CTCCACCCCGGCCCAGCCGT | 81 | 389 |
| 6128 | 6147 | 502597 | GCTCCACCCCGGCCCAGCCG | 86 | 390 |
| 6129 | 6148 | 502598 | TGCTCCACCCCGGCCCAGCC | 83 | 391 |
| 6130 | 6149 | 502599 | CTGCTCCACCCCGGCCCAGC | 88 | 392 |
| 6152 | 6171 | 502600 | AAGGGATGTGTCCGGAAGTC | 60 | 393 |
| 6153 | 6172 | 502601 | GAAGGGATGTGTCCGGAAGT | 58 | 394 |
| 6154 | 6173 | 502602 | AGAAGGGATGTGTCCGGAAG | 63 | 395 |
| 6155 | 6174 | 502603 | AAGAAGGGATGTGTCCGGAA | 62 | 396 |
| 6156 | 6175 | 502604 | GAAGAAGGGATGTGTCCGGA | 61 | 397 |
| 6157 | 6176 | 502605 | AGAAGAAGGGATGTGTCCGG | 62 | 398 |
| 6158 | 6177 | 502606 | AAGAAGAAGGGATGTGTCCG | 56 | 399 |
| 6159 | 6178 | 502607 | AAAGAAGAAGGGATGTGTCC | 58 | 400 |
| 6160 | 6179 | 502608 | CAAAGAAGAAGGGATGTGTC | 50 | 401 |
| 6161 | 6180 | 502609 | CCAAAGAAGAAGGGATGTGT | 61 | 402 |
| 6163 | 6182 | 502610 | GGCCAAAGAAGAAGGGATGT | 73 | 403 |
| 6164 | 6183 | 502611 | AGGCCAAAGAAGAAGGGATG | 56 | 404 |
| 6165 | 6184 | 502612 | GAGGCCAAAGAAGAAGGGAT | 73 | 405 |
| 6166 | 6185 | 502613 | CGAGGCCAAAGAAGAAGGGA | 75 | 406 |
| 6167 | 6186 | 502614 | TCGAGGCCAAAGAAGAAGGG | 75 | 407 |
| 6168 | 6187 | 502615 | GTCGAGGCCAAAGAAGAAGG | 83 | 408 |
| 6169 | 6188 | 502616 | AGTCGAGGCCAAAGAAGAAG | 58 | 409 |
| 6170 | 6189 | 502617 | CAGTCGAGGCCAAAGAAGAA | 52 | 410 |
| 6171 | 6190 | 502618 | CCAGTCGAGGCCAAAGAAGA | 68 | 411 |
| 6172 | 6191 | 502619 | CCCAGTCGAGGCCAAAGAAG | 78 | 412 |
| 6173 | 6192 | 502620 | TCCCAGTCGAGGCCAAAGAA | 66 | 413 |
| 6174 | 6193 | 502621 | ATCCCAGTCGAGGCCAAAGA | 75 | 414 |
| 6175 | 6194 | 502622 | CATCCCAGTCGAGGCCAAAG | 70 | 415 |
| 6176 | 6195 | 502623 | CCATCCCAGTCGAGGCCAAA | 81 | 416 |
| 6177 | 6196 | 502624 | ACCATCCCAGTCGAGGCCAA | 82 | 417 |
| 6178 | 6197 | 502625 | GACCATCCCAGTCGAGGCCA | 88 | 418 |
| 6179 | 6198 | 502626 | AGACCATCCCAGTCGAGGCC | 79 | 419 |
| 6180 | 6199 | 502627 | GAGACCATCCCAGTCGAGGC | 82 | 420 |
| 6181 | 6200 | 502628 | GGAGACCATCCCAGTCGAGG | 60 | 421 |
| 6216 | 6235 | 502629 | TTCGAAATCCGGTGTAAAGG | 84 | 422 |
| 6217 | 6236 | 502630 | CTTCGAAATCCGGTGTAAAG | 57 | 423 |
| 6218 | 6237 | 502631 | CCTTCGAAATCCGGTGTAAA | 64 | 424 |
| 6219 | 6238 | 502632 | ACCTTCGAAATCCGGTGTAA | 73 | 425 |
| 6220 | 6239 | 502633 | CACCTTCGAAATCCGGTGTA | 77 | 426 |
| 6221 | 6240 | 502634 | GCACCTTCGAAATCCGGTGT | 59 | 427 |
| 6222 | 6241 | 502635 | GGCACCTTCGAAATCCGGTG | 85 | 428 |
| 6223 | 6242 | 502636 | TGGCACCTTCGAAATCCGGT | 86 | 429 |
| 6224 | 6243 | 502637 | GTGGCACCTTCGAAATCCGG | 74 | 430 |
| 6225 | 6244 | 502638 | GGTGGCACCTTCGAAATCCG | 79 | 431 |
| 6226 | 6245 | 502639 | CGGTGGCACCTTCGAAATCC | 85 | 432 |
| 6227 | 6246 | 502640 | TCGGTGGCACCTTCGAAATC | 71 | 433 |
| 6228 | 6247 | 502641 | GTCGGTGGCACCTTCGAAAT | 88 | 434 |
| 6229 | 6248 | 502642 | TGTCGGTGGCACCTTCGAAA | 89 | 435 |
| 6230 | 6249 | 502643 | GTGTCGGTGGCACCTTCGAA | 88 | 436 |
| 6231 | 6250 | 502644 | TGTGTCGGTGGCACCTTCGA | 87 | 437 |
| 6232 | 6251 | 502645 | ATGTGTCGGTGGCACCTTCG | 88 | 438 |
| 6233 | 6252 | 502646 | CATGTGTCGGTGGCACCTTC | 88 | 439 |
| 6234 | 6253 | 502647 | GCATGTGTCGGTGGCACCTT | 91 | 440 |
| 6235 | 6254 | 502648 | TGCATGTGTCGGTGGCACCT | 87 | 441 |
| 6236 | 6255 | 502649 | TTGCATGTGTCGGTGGCACC | 86 | 442 |
| 6237 | 6256 | 502650 | GTTGCATGTGTCGGTGGCAC | 83 | 443 |
| 6238 | 6257 | 502651 | AGTTGCATGTGTCGGTGGCA | 81 | 444 |
| 6239 | 6258 | 502652 | AAGTTGCATGTGTCGGTGGC | 79 | 445 |
| 6240 | 6259 | 502653 | GAAGTTGCATGTGTCGGTGG | 58 | 446 |
| 6241 | 6260 | 502654 | CGAAGTTGCATGTGTCGGTG | 85 | 447 |
| 6243 | 6262 | 502655 | GTCGAAGTTGCATGTGTCGG | 77 | 448 |
| 6244 | 6263 | 502656 | AGTCGAAGTTGCATGTGTCG | 79 | 449 |
| 6245 | 6264 | 502657 | AAGTCGAAGTTGCATGTGTC | 74 | 450 |
| 6246 | 6265 | 502658 | CAAGTCGAAGTTGCATGTGT | 82 | 451 |
| 6247 | 6266 | 502659 | CCAAGTCGAAGTTGCATGTG | 82 | 452 |
| 6248 | 6267 | 502660 | ACCAAGTCGAAGTTGCATGT | 70 | 453 |
| 6249 | 6268 | 502661 | CACCAAGTCGAAGTTGCATG | 76 | 454 |
| 6250 | 6269 | 502662 | CCACCAAGTCGAAGTTGCAT | 79 | 455 |
| 6251 | 6270 | 502663 | TCCACCAAGTCGAAGTTGCA | 68 | 456 |
| 6252 | 6271 | 502664 | CTCCACCAAGTCGAAGTTGC | 71 | 457 |
| 6253 | 6272 | 502665 | CCTCCACCAAGTCGAAGTTG | 67 | 458 |
| 6254 | 6273 | 502666 | TCCTCCACCAAGTCGAAGTT | 70 | 459 |
| 6255 | 6274 | 502667 | GTCCTCCACCAAGTCGAAGT | 80 | 460 |
| 6256 | 6275 | 502668 | CGTCCTCCACCAAGTCGAAG | 76 | 461 |
| 6257 | 6276 | 502669 | CCGTCCTCCACCAAGTCGAA | 78 | 462 |
| 6258 | 6277 | 502670 | CCCGTCCTCCACCAAGTCGA | 83 | 463 |
| 6259 | 6278 | 502671 | GCCCGTCCTCCACCAAGTCG | 76 | 464 |
| 6260 | 6279 | 502672 | AGCCCGTCCTCCACCAAGTC | 72 | 465 |
| 6261 | 6280 | 502673 | GAGCCCGTCCTCCACCAAGT | 71 | 466 |
| 6262 | 6281 | 502674 | TGAGCCCGTCCTCCACCAAG | 60 | 467 |
| 6289 | 6308 | 503033 | CTACCCCGCCCCCGCTCACC | 60 | 671 |
| 6445 | 6464 | 503034 | CTAGGTCACTGCTGGGTCCT | 86 | 672 |
| 6596 | 6615 | 503035 | CTCAGATAGCTCCCCACTCC | 55 | 673 |
| 6794 | 6813 | 503036 | AATTCTCTAATTCTCTAGAC | 19 | 674 |
| 8666 | 8685 | 503037 | TACCTGAGGGCCATGCAGGA | 51 | 675 |
| 8765 | 8784 | 503038 | GTTCCAAGACTGATCCTGCA | 69 | 676 |
| 11975 | 11994 | 502675 | GGTTCCGAGCCTCTGCCTCG | 44 | 468 |
| 11976 | 11995 | 502676 | CGGTTCCGAGCCTCTGCCTC | 74 | 469 |
| 11977 | 11996 | 502677 | CCGGTTCCGAGCCTCTGCCT | 72 | 470 |
| 11978 | 11997 | 502678 | CCCGGTTCCGAGCCTCTGCC | 73 | 471 |
| 11979 | 11998 | 502679 | TCCCGGTTCCGAGCCTCTGC | 84 | 472 |
| 11980 | 11999 | 502680 | GTCCCGGTTCCGAGCCTCTG | 66 | 473 |
| 11982 | 12001 | 502681 | AGGTCCCGGTTCCGAGCCTC | 82 | 474 |
| 11983 | 12002 | 502682 | TAGGTCCCGGTTCCGAGCCT | 83 | 475 |
| 11984 | 12003 | 502683 | CTAGGTCCCGGTTCCGAGCC | 81 | 476 |
| 11985 | 12004 | 502684 | TCTAGGTCCCGGTTCCGAGC | 74 | 477 |
| 11986 | 12005 | 502685 | CTCTAGGTCCCGGTTCCGAG | 78 | 478 |
| 11987 | 12006 | 502686 | CCTCTAGGTCCCGGTTCCGA | 75 | 479 |
| 11988 | 12007 | 502687 | GCCTCTAGGTCCCGGTTCCG | 80 | 480 |
| 12016 | 12035 | 502688 | CATCCGCTCCTGCAACTGCC | 89 | 481 |
| 12017 | 12036 | 502689 | CCATCCGCTCCTGCAACTGC | 81 | 482 |
| 12018 | 12037 | 502690 | TCCATCCGCTCCTGCAACTG | 71 | 483 |
| 12019 | 12038 | 502691 | CTCCATCCGCTCCTGCAACT | 75 | 484 |
| 12020 | 12039 | 502692 | ACTCCATCCGCTCCTGCAAC | 64 | 485 |
| 12021 | 12040 | 502693 | AACTCCATCCGCTCCTGCAA | 52 | 486 |
| 12022 | 12041 | 502694 | CAACTCCATCCGCTCCTGCA | 45 | 487 |
| 12024 | 12043 | 502695 | AGCAACTCCATCCGCTCCTG | 78 | 488 |
| 12025 | 12044 | 502696 | CAGCAACTCCATCCGCTCCT | 64 | 489 |
| 12026 | 12045 | 502697 | GCAGCAACTCCATCCGCTCC | 56 | 490 |
| 12173 | 12192 | 503039 | AGGAGGGCGGTGGCGCGGCG | 0 | 677 |
| 12221 | 12240 | 503040 | TGACAGCTGGAAGGAGAAGA | 41 | 678 |
| 12258 | 12277 | 502712 | GAAGGTGGATCCGTGGCCCG | 73 | 505 |
| 12259 | 12278 | 502713 | GGAAGGTGGATCCGTGGCCC | 70 | 506 |
| 12260 | 12279 | 502714 | GGGAAGGTGGATCCGTGGCC | 72 | 507 |
| 12261 | 12280 | 502715 | TGGGAAGGTGGATCCGTGGC | 50 | 508 |
| 12262 | 12281 | 502716 | ATGGGAAGGTGGATCCGTGG | 62 | 509 |
| 12263 | 12282 | 451417 | CATGGGAAGGTGGATCCGTG | 77 | 679 |
| 12463 | 12482 | 503041 | GGAGGTTATCTAGGGAGATC | 42 | 680 |
| 12542 | 12561 | 503042 | GAAGGGACAGGTGACCCGAT | 69 | 681 |
| 12596 | 12615 | 502724 | CACCAGCGGGCACTGGCCCA | 51 | 518 |
| 12597 | 12616 | 502725 | CCACCAGCGGGCACTGGCCC | 55 | 519 |
| 12598 | 12617 | 502726 | CCCACCAGCGGGCACTGGCC | 61 | 520 |
| 12599 | 12618 | 502727 | CCCCACCAGCGGGCACTGGC | 43 | 521 |
| 12601 | 12620 | 502728 | GGCCCCACCAGCGGGCACTG | 16 | 522 |
| 12602 | 12621 | 502729 | TGGCCCCACCAGCGGGCACT | 43 | 523 |
| 12603 | 12622 | 502730 | CTGGCCCCACCAGCGGGCAC | 43 | 524 |
| 12604 | 12623 | 502731 | CCTGGCCCCACCAGCGGGCA | 41 | 525 |
| 12605 | 12624 | 502732 | GCCTGGCCCCACCAGCGGGC | 30 | 526 |
| 12607 | 12626 | 502733 | GGGCCTGGCCCCACCAGCGG | 66 | 527 |
| 12625 | 12644 | 502734 | AGGTGGCGGCGGTGCATGGG | 31 | 528 |
| 12626 | 12645 | 502735 | CAGGTGGCGGCGGTGCATGG | 23 | 529 |
| 12627 | 12646 | 502736 | GCAGGTGGCGGCGGTGCATG | 57 | 530 |
| 12628 | 12647 | 502737 | AGCAGGTGGCGGCGGTGCAT | 54 | 531 |
| 12629 | 12648 | 502738 | CAGCAGGTGGCGGCGGTGCA | 61 | 532 |
| 12630 | 12649 | 502739 | GCAGCAGGTGGCGGCGGTGC | 57 | 533 |
| 12631 | 12650 | 502740 | AGCAGCAGGTGGCGGCGGTG | 36 | 534 |
| 12632 | 12651 | 502741 | GAGCAGCAGGTGGCGGCGGT | 53 | 535 |
| 12633 | 12652 | 502742 | GGAGCAGCAGGTGGCGGCGG | 39 | 536 |
| 12634 | 12653 | 502743 | GGGAGCAGCAGGTGGCGGCG | 36 | 537 |
| 12635 | 12654 | 502744 | AGGGAGCAGCAGGTGGCGGC | 62 | 538 |
| 12636 | 12655 | 502745 | CAGGGAGCAGCAGGTGGCGG | 56 | 539 |
| 12637 | 12656 | 502746 | GCAGGGAGCAGCAGGTGGCG | 58 | 540 |
| 12638 | 12657 | 502747 | GGCAGGGAGCAGCAGGTGGC | 65 | 541 |
| 12639 | 12658 | 502748 | TGGCAGGGAGCAGCAGGTGG | 47 | 542 |
| 12640 | 12659 | 502749 | CTGGCAGGGAGCAGCAGGTG | 41 | 543 |
| 12642 | 12661 | 451432 | CCCTGGCAGGGAGCAGCAGG | 53 | 544 |
| 12643 | 12662 | 502750 | ACCCTGGCAGGGAGCAGCAG | 52 | 545 |
| 12646 | 12665 | 503043 | CGTACCCTGGCAGGGAGCAG | 59 | 682 |
| 12918 | 12937 | 502977 | GGACTCGCCCCGCCTACGCC | 71 | 683 |
| 12924 | 12943 | 502978 | CTCCTGGGACTCGCCCCGCC | 67 | 684 |
| 12925 | 12944 | 503044 | GCTCCTGGGACTCGCCCCGC | 66 | 685 |
| 12929 | 12948 | 503045 | ATTGGCTCCTGGGACTCGCC | 77 | 686 |
| 12930 | 12949 | 502979 | GATTGGCTCCTGGGACTCGC | 70 | 687 |
| 12936 | 12955 | 502980 | GCCTCTGATTGGCTCCTGGG | 56 | 688 |
| 12942 | 12961 | 502981 | GCATGGGCCTCTGATTGGCT | 20 | 689 |
| 12948 | 12967 | 502982 | CACCCGGCATGGGCCTCTGA | 20 | 690 |
| 12986 | 13005 | 503046 | GCCAGGCCTAGGGACCTGCG | 58 | 691 |
| 12990 | 13009 | 502760 | ATAGGCCAGGCCTAGGGACC | 51 | 555 |
| 12991 | 13010 | 502761 | GATAGGCCAGGCCTAGGGAC | 41 | 556 |
| 12992 | 13011 | 502762 | CGATAGGCCAGGCCTAGGGA | 69 | 557 |
| 12993 | 13012 | 502763 | CCGATAGGCCAGGCCTAGGG | 80 | 558 |
| 12994 | 13013 | 502764 | TCCGATAGGCCAGGCCTAGG | 78 | 559 |
| 12995 | 13014 | 502765 | CTCCGATAGGCCAGGCCTAG | 89 | 560 |
| 12996 | 13015 | 502766 | CCTCCGATAGGCCAGGCCTA | 79 | 561 |
| 12997 | 13016 | 502767 | GCCTCCGATAGGCCAGGCCT | 73 | 562 |
| 12999 | 13018 | 502768 | GCGCCTCCGATAGGCCAGGC | 83 | 563 |
| 13015 | 13034 | 502769 | AACAGGAGCAGGGAAAGCGC | 83 | 564 |
| 13016 | 13035 | 502770 | GAACAGGAGCAGGGAAAGCG | 70 | 565 |
| 13017 | 13036 | 502771 | CGAACAGGAGCAGGGAAAGC | 43 | 566 |
| 13018 | 13037 | 502772 | GCGAACAGGAGCAGGGAAAG | 47 | 567 |
| 13019 | 13038 | 502773 | GGCGAACAGGAGCAGGGAAA | 61 | 568 |
| 13020 | 13039 | 502774 | CGGCGAACAGGAGCAGGGAA | 74 | 569 |
| 13021 | 13040 | 502775 | ACGGCGAACAGGAGCAGGGA | 60 | 570 |
| 13022 | 13041 | 502776 | AACGGCGAACAGGAGCAGGG | 86 | 571 |
| 13023 | 13042 | 502777 | CAACGGCGAACAGGAGCAGG | 84 | 572 |
| 13044 | 13063 | 502778 | GGGCGGCGGCACGAGACAGA | 80 | 573 |
| 13045 | 13064 | 502779 | AGGGCGGCGGCACGAGACAG | 76 | 574 |
| 13046 | 13065 | 502780 | CAGGGCGGCGGCACGAGACA | 58 | 575 |
| 13047 | 13066 | 502781 | CCAGGGCGGCGGCACGAGAC | 80 | 576 |
| 13048 | 13067 | 502782 | CCCAGGGCGGCGGCACGAGA | 59 | 577 |
| 13049 | 13068 | 502783 | GCCCAGGGCGGCGGCACGAG | 68 | 578 |
| 13050 | 13069 | 502784 | AGCCCAGGGCGGCGGCACGA | 75 | 579 |
| 13051 | 13070 | 502785 | CAGCCCAGGGCGGCGGCACG | 76 | 580 |
| 13052 | 13071 | 502786 | GCAGCCCAGGGCGGCGGCAC | 70 | 581 |
| 13089 | 13108 | 502787 | CTGCGGTGAGTTGGCCGGCG | 68 | 582 |
| 13090 | 13109 | 502788 | ACTGCGGTGAGTTGGCCGGC | 67 | 583 |
| 13091 | 13110 | 502789 | GACTGCGGTGAGTTGGCCGG | 58 | 584 |
| 13092 | 13111 | 502790 | AGACTGCGGTGAGTTGGCCG | 71 | 585 |
| 13093 | 13112 | 502791 | CAGACTGCGGTGAGTTGGCC | 70 | 586 |
| 13094 | 13113 | 502792 | CCAGACTGCGGTGAGTTGGC | 79 | 587 |
| 13095 | 13114 | 502793 | GCCAGACTGCGGTGAGTTGG | 76 | 588 |
| 13096 | 13115 | 502794 | CGCCAGACTGCGGTGAGTTG | 66 | 589 |
| 13140 | 13159 | 502795 | AAGACAGTTCTAGGGTTCAG | 87 | 590 |
| 13141 | 13160 | 502796 | GAAGACAGTTCTAGGGTTCA | 78 | 591 |
| 13142 | 13161 | 502797 | CGAAGACAGTTCTAGGGTTC | 85 | 592 |
| 13143 | 13162 | 502798 | TCGAAGACAGTTCTAGGGTT | 78 | 593 |
| 13144 | 13163 | 502799 | GTCGAAGACAGTTCTAGGGT | 92 | 594 |
| 13145 | 13164 | 502800 | AGTCGAAGACAGTTCTAGGG | 85 | 595 |
| 13146 | 13165 | 502801 | GAGTCGAAGACAGTTCTAGG | 83 | 596 |
| 13147 | 13166 | 502802 | GGAGTCGAAGACAGTTCTAG | 86 | 597 |
| 13148 | 13167 | 502803 | CGGAGTCGAAGACAGTTCTA | 91 | 598 |
| 13149 | 13168 | 502804 | CCGGAGTCGAAGACAGTTCT | 76 | 599 |
| 13150 | 13169 | 502805 | CCCGGAGTCGAAGACAGTTC | 90 | 600 |
| 13151 | 13170 | 502806 | CCCCGGAGTCGAAGACAGTT | 83 | 601 |
| 13152 | 13171 | 502807 | GCCCCGGAGTCGAAGACAGT | 82 | 602 |
| 13153 | 13172 | 502808 | GGCCCCGGAGTCGAAGACAG | 73 | 603 |
| 13154 | 13173 | 502809 | GGGCCCCGGAGTCGAAGACA | 67 | 604 |
| 13206 | 13225 | 502810 | AGGCGGTGGGCGCGGCTTCT | 73 | 605 |
| 13207 | 13226 | 502811 | CAGGCGGTGGGCGCGGCTTC | 57 | 606 |
| 13208 | 13227 | 502812 | GCAGGCGGTGGGCGCGGCTT | 69 | 607 |
| 13210 | 13229 | 502813 | TGGCAGGCGGTGGGCGCGGC | 73 | 608 |
| 13212 | 13231 | 502814 | ACTGGCAGGCGGTGGGCGCG | 56 | 609 |
| 13214 | 13233 | 502815 | GAACTGGCAGGCGGTGGGCG | 71 | 610 |
| 13215 | 13234 | 502816 | TGAACTGGCAGGCGGTGGGC | 80 | 611 |
| 13217 | 13236 | 502817 | TGTGAACTGGCAGGCGGTGG | 85 | 612 |
| 13250 | 13269 | 502818 | TGGAGCTGGGCGGAGACCCA | 55 | 613 |
| 13252 | 13271 | 502819 | ACTGGAGCTGGGCGGAGACC | 53 | 614 |
| 13253 | 13272 | 502820 | GACTGGAGCTGGGCGGAGAC | 55 | 615 |
| 13255 | 13274 | 502821 | AGGACTGGAGCTGGGCGGAG | 76 | 616 |
| 13257 | 13276 | 502822 | ACAGGACTGGAGCTGGGCGG | 77 | 617 |
| 13258 | 13277 | 502823 | CACAGGACTGGAGCTGGGCG | 74 | 618 |
| 13259 | 13278 | 502824 | TCACAGGACTGGAGCTGGGC | 90 | 619 |
| 13449 | 13468 | 502825 | GCCTCAGCCTGGCCGAAAGA | 80 | 620 |
| 13450 | 13469 | 502826 | GGCCTCAGCCTGGCCGAAAG | 72 | 621 |
| 13553 | 13572 | 444401 | TTGCACTTTGCGAACCAACG | 97 | 41 |
| 14037 | 14056 | 503047 | TTCCTCCCCCAACCCTGATT | 34 | 692 |
| 14255 | 14274 | 503048 | AAGTTTGCAGCAACTTTTCT | 0 | 693 |
| 14325 | 14344 | 503049 | GCCCCTCGGAATTCCCGGCT | 0 | 694 |
| 14343 | 14362 | 503050 | CATCTCGGCCTGCGCTCCGC | 39 | 695 |
| 14361 | 14380 | 503051 | GCAGGCCCCCACATTCCCCA | 0 | 696 |
| 14392 | 14411 | 503052 | CTTCTGCACGCCTCCGTCTC | 30 | 697 |

### Example 8: Antisense Inhibition of Murine DMPK in Mouse Primary Hepatocytes

Antisense oligonucleotides targeted to a murine DMPK nucleic acid were tested for their effect on DMPK RNA transcript in vitro. Cultured mouse primary hepatocytes at a density of 35,000 cells per well were transfected using electroporation with 8,000 nM antisense oligonucleotide. After approximately 24 hours, RNA was isolated from the cells and DMPK transcript levels were measured by quantitative real-time PCR. DMPK RNA transcript levels were adjusted according to total RNA content, as measured by RIBOGREEN®. Results are presented as percent inhibition of DMPK, relative to untreated control cells.

The antisense oligonucleotides in Tables 14, 15, and 16 are 5-10-5 gapmers, where the gap segment comprises ten 2′-deoxynucleosides and each wing segment comprises five 2′-MOE nucleosides. The internucleoside linkages throughout each gapmer are phosphorothioate (P═S) linkages. All cytosine residues throughout each gapmer are 5-methylcytosines. ‘Murine Target start site’ indicates the 5′-most nucleoside to which the antisense oligonucleotide is targeted in the murine gene sequence. ‘Murine Target stop site’ indicates the 3′-most nucleoside to which the antisense oligonucleotide is targeted in the murine gene sequence. All the antisense oligonucleotides listed in Table 12 target SEQ ID NO: 3 (GENBANK Accession No. NT_039413.7 truncated from nucleotides 16666001 to 16681000). All the antisense oligonucleotides listed in Table 13 target SEQ ID NO: 4 (GENBANK Accession No. NM_032418.1). The antisense oligonucleotides of Table 14 target SEQ ID NO: 5 (GENBANK Accession No. A1007148.1), SEQ ID NO: 6 (GENBANK Accession No. A1304033.1), SEQ ID NO: 7 (GENBANK Accession No. BC024150.1), SEQ ID NO: 8 (GENBANK Accession No. BC056615.1), SEQ ID NO: 793 (GENBANK Accession No. BC075715.1), SEQ ID NO: 794 (GENBANK Accession No. BU519245.1), SEQ ID NO: 795 (GENBANK Accession No. CB247909.1), SEQ ID NO: 796 (GENBANK Accession No. CX208906.1), SEQ ID NO: 797 (GENBANK Accession No. CX732022.1), SEQ ID NO: 798 (GENBANK Accession No. S60315.1), or SEQ ID NO: 799 (GENBANK Accession No. S60316.1). In addition, the human antisense oligonucleotide ISIS 451421 targeting SEQ ID NO: 800(GENBANK Accession No. NM_001081562.1) was also included in this assay and is listed in Table 14.

The murine oligonucleotides of Tables 14, 15, and 16 may also be cross-reactive with human gene sequences. ‘Mismatches’ indicate the number of nucleobases by which the murine oligonucleotide is mismatched with a human gene sequence. The greater the complementarity between the murine oligonucleotide and the human sequence, the more likely the murine oligonucleotide can cross-react with the human sequence. The murine oligonucleotides in Tables 14, 15, and 16 were compared to SEQ ID NO: 800 (GENBANK Accession No. NM_001081562.1). “Human Target start site” indicates the 5′-most nucleoside to which the gapmer is targeted in the human gene sequence. “Human Target stop site” indicates the 3′-most nucleoside to which the gapmer is targeted human gene sequence.

Several of the tested antisense oligonucleotides demonstrated significant inhibition of DMPK mRNA levels under the conditions specified above. Certain of the tested antisense oligonucleotides are cross-reactive with human gene sequences.

TABLE 14
Inhibition of murine DMPK RNA transcript in mouse primary hepatocytes by 5-10-5
gapmers targeting SEQ ID NO: 800

| Murine | Murine | | | | | Human | Human | |
|---|---|---|---|---|---|---|---|---|
| Target | Target | | | | SEQ | Target | Target | |
| Start | Stop | | | % | ID | Start | Stop | |
| Site | Site | ISIS No | Sequence | inhibition | NO. | Site | Site | Mismatches |
| 11904 | 11923 | 299516 | TGGCCCACAGCCACGGCCGG | 47 | 698 | 1850 | 1869 | 0 |
| 11927 | 11946 | 299520 | GGCCTGGCCCCACCAGCGGG | 58 | 699 | 1873 | 1892 | 0 |
| 11962 | 11981 | 299521 | CCTGGCAGGGAGCAGCAGGT | 44 | 700 | 1908 | 1927 | 0 |
| 3345 | 3364 | 451360 | CAGCCGCACTTCGGCTGACA | 29 | 701 | 207 | 226 | 1 |
| 3378 | 3397 | 451361 | GCCTGGGTCCAGCACCAGCT | 67 | 702 | 240 | 259 | 2 |
| 3388 | 3407 | 451362 | GTCCCAGGAAGCCTGGGTCC | 62 | 703 | 250 | 269 | 2 |
| 3418 | 3437 | 451363 | CGCCCAGGAGAAGGTCGAGC | 69 | 213 | 280 | 299 | 0 |
| 3484 | 3503 | 451364 | CCCACTGCAAGAAGTCGGCC | 69 | 226 | 346 | 365 | 0 |
| 6264 | 6283 | 451366 | CGTTAGCAGGTCCCCGCCCA | 73 | 704 | 660 | 679 | 2 |
| 6342 | 6361 | 451367 | GTCTATGGCCATGACAATCT | 61 | 705 | 738 | 757 | 0 |
| 6363 | 6382 | 451368 | GTAGCCCAGCCGGTGCACGG | 54 | 706 | 759 | 778 | 2 |
| 6851 | 6870 | 451370 | GGGTGCCCACAGCCACCAGC | 72 | 707 | 889 | 908 | 0 |
| 6919 | 6938 | 451371 | TGGCCCGTAGCTGCCTGCCC | 80 | 708 | 957 | 976 | 2 |
| 7448 | 7467 | 451373 | GGAAATCACCTGCCCCACCT | 80 | 709 | n/a | n/a | n/a |
| 7458 | 7477 | 451374 | GGATGTTTCTGGAAATCACC | 84 | 710 | n/a | n/a | n/a |
| 7533 | 7552 | 451375 | GTGGCACCCTCGAAGTCTGG | 77 | 711 | 1271 | 1290 | 3 |
| 7589 | 7608 | 451376 | CCCCGCTCACCATGGCAGTG | 31 | 712 | n/a | n/a | n/a |
| 10278 | 10297 | 451378 | GGTCCGGGACCTGATTGTCT | 85 | 713 | n/a | n/a | n/a |
| 3229 | 3248 | 451385 | GCTGCATGTCTGCCCGTCCC | 74 | 714 | 90 | 109 | 1 |
| 3244 | 3263 | 451386 | GGCCCCAGAACCCTAGCTGC | 73 | 715 | n/a | n/a | n/a |
| 3270 | 3289 | 451387 | TCACAGGGCCTGGCTGCCCC | 62 | 716 | 131 | 150 | 1 |
| 3333 | 3352 | 451388 | GGCTGACATGTTGGGCAGGC | 60 | 717 | 195 | 214 | 1 |
| 3250 | 3269 | 451389 | TGTCCAGGCCCCAGAACCCT | 68 | 718 | 111 | 130 | 3 |
| 12295 | 12314 | 451391 | GGCCAGGCCTAGGGATCTGC | 51 | 719 | n/a | n/a | n/a |
| 12306 | 12325 | 451392 | CGCCTCGGATAGGCCAGGCC | 52 | 720 | 1935 | 1954 | 1 |
| 12450 | 12469 | 451393 | GGCTTGGAGTCTTAGGGTTC | 85 | 721 | n/a | n/a | n/a |
| 12623 | 12642 | 451394 | TCCCCGGCCGCCAGGTGGCA | 43 | 722 | 2224 | 2243 | 3 |
| 12651 | 12670 | 451395 | GGTGCTGGGCACGAGCCCTG | 62 | 723 | n/a | n/a | n/a |
| 12698 | 12717 | 451396 | GCCCAGCTGCTGCAGCAGCG | 66 | 724 | n/a | n/a | n/a |
| 12876 | 12895 | 451397 | CCGTGTGTGCTGGCAGAGGT | 76 | 725 | n/a | n/a | n/a |
| 13084 | 13103 | 451398 | ATAAATACCGAGGAATGTCG | 77 | 726 | 2766 | 2785 | 0 |
| 13094 | 13113 | 451399 | GGGACAGACAATAAATACCG | 80 | 727 | 2776 | 2795 | 0 |
| 12362 | 12381 | 451405 | GTGCAGCCCAGTGTGGCGGC | 69 | 728 | 1991 | 2010 | 3 |
| 11175 | 11194 | 451415 | CCTGGAGAAGTTCTGGTTGG | 48 | 729 | 1674 | 1693 | 3 |
| 11585 | 11604 | 451417 | CATGGGAAGGTGGATCCGTG | 65 | 679 | 1819 | 1838 | 1 |
| 11854 | 11873 | 451419 | GGTGACCCGATCGGAGCCCA | 11 | 730 | n/a | n/a | n/a |
| 11874 | 11893 | 451420 | AGCTGGAGAGAGAAGGGACA | 37 | 731 | n/a | n/a | n/a |
| 11379 | 11398 | 451422 | GTGAGGGACTCGCCTGCGGC | 36 | 732 | n/a | n/a | n/a |
| 11479 | 11498 | 451423 | GCGGCTGCGGTGCCCCAGCC | 50 | 733 | n/a | n/a | n/a |
| 11883 | 11902 | 451424 | GGGCCATCTAGCTGGAGAGA | 45 | 734 | n/a | n/a | n/a |
| 3485 | 3504 | 451427 | CCCCACTGCAAGAAGTCGGC | 57 | 735 | 347 | 366 | 1 |
| 4621 | 4640 | 451428 | TTGAGCCCTTTTAAGGCAGC | 43 | 736 | n/a | n/a | n/a |
| 6232 | 6251 | 451429 | TGACCAGGTACTGGGAGCGG | 47 | 737 | n/a | n/a | n/a |
| 10985 | 11004 | 451430 | CCTGGAGCTGGATCAGTCCC | 6 | 738 | n/a | n/a | n/a |
| 11586 | 11605 | 451431 | ACATGGGAAGGTGGATCCGT | 70 | 739 | 1820 | 1839 | 1 |
| 11963 | 11982 | 451432 | CCCTGGCAGGGAGCAGCAGG | 42 | 544 | 1909 | 1928 | 0 |
| 11973 | 11992 | 451433 | GTGGGACATACCCTGGCAGG | 34 | 740 | n/a | n/a | n/a |
| 12294 | 12313 | 451434 | GCCAGGCCTAGGGATCTGCA | 35 | 741 | n/a | n/a | n/a |

TABLE 15
Inhibition of murine DMPK RNA transcript in mouse primary
hepatocytes by 5-10-5 gapmers targeting SEQ ID NO: 800

| Murine | Murine | | | | | Human | Human | |
|---|---|---|---|---|---|---|---|---|
| Target | Target | | | | SEQ | Target | Target | |
| Start | Stop | ISIS | | % | ID | Start | Stop | |
| Site | Site | No | Sequence | inhibition | NO. | Site | Site | Mismatches |
| 330 | 349 | 451365 | GGAAGCACGACACCTCGCCT | 67 | 742 | 535 | 554 | 1 |
| 662 | 681 | 451369 | CCTCACCATTCCATCAGGCT | 81 | 743 | n/a | n/a | n/a |
| 881 | 900 | 451372 | CGGCAGCGACAAGTGTTCCC | 90 | 744 | n/a | n/a | n/a |
| 1217 | 1236 | 451377 | GTCTCTGAAGGCCATGCAGC | 69 | 745 | 1407 | 1426 | 3 |
| 1329 | 1348 | 451379 | CAGCCACTTGATCCGGTGGG | 62 | 746 | n/a | n/a | n/a |
| 1342 | 1361 | 451380 | AGGTCGGCCTCTTCAGCCAC | 74 | 747 | n/a | n/a | n/a |
| 1494 | 1513 | 451381 | GTTGGCTGGAGAAGTTCTGG | 39 | 748 | 1678 | 1697 | 2 |
| 1598 | 1617 | 451382 | CCCCGTGATGGCTGCGGCTC | 54 | 749 | 1782 | 1801 | 3 |
| 1644 | 1663 | 451383 | GGCCATCTAGATGGGAAGGT | 21 | 517 | 1828 | 1847 | 0 |
| 1741 | 1760 | 451384 | AGGCCAGGCCTAGGGATCCT | 39 | 750 | 1925 | 1944 | 1 |

TABLE 16
Inhibition of murine DMPK RNA transcript in mouse primary hepatocytes by 5-10-5
gapmers targeting SEQ ID NOs: 5-8 and 793-799

| Murine | Murine | Murine | | | | | Human | Human | |
|---|---|---|---|---|---|---|---|---|---|
| Target | Target | Target | | | | SEQ | Target | Target | |
| Start | Stop | SEQ | ISIS | | % | ID | Start | Stop | |
| Site | Site | ID NO | No | Sequence | inhibition | NO. | Site | Site | Mismatches |
| 324 | 343 | 5 | 451410 | GGCGCGGTGCCCCAGCCTGG | 67 | 751 | n/a | n/a | n/a |
| 485 | 504 | 5 | 451411 | GTCCTGGCCCCACCAGCGGG | 66 | 752 | 1873 | 1892 | 1 |
| 534 | 553 | 5 | 451412 | CCAGGCCTAGGAATCCTGGC | 17 | 753 | 1922 | 1941 | 2 |
| 547 | 566 | 5 | 451413 | GCGCCTCGGATAGCCAGGCC | 51 | 754 | n/a | n/a | n/a |
| 594 | 613 | 5 | 451414 | CCCAGTGTGGCGCAGCAGCC | 65 | 755 | n/a | n/a | n/a |
| 393 | 412 | 6 | 451402 | GTGTTTCATCTTCACCACCG | 80 | 756 | 462 | 481 | 3 |
| 1475 | 1494 | 7 | 451390 | AGGTCAGCCTCTTCAGCCAC | 60 | 757 | n/a | n/a | n/a |
| n/a | n/a | n/a | 451425 | GGCCATATGGGAAGGTGGAT | 48 | 758 | 1824 | 1843 | 0 |
| 1763 | 1782 | 8 | 451418 | GGAGGATTTGGCGAGAAGCA | 48 | 759 | n/a | n/a | n/a |
| 1032 | 1051 | 793 | 451403 | CGAAGTCTGCCCCACCTCGA | 58 | 760 | n/a | n/a | n/a |
| 1042 | 1061 | 793 | 451404 | GTGGCACCCTCGAAGTCTGC | 72 | 761 | n/a | n/a | n/a |
| 217 | 236 | 794 | 451400 | GGGTCCATTGTAAGGAAGCT | 4 | 762 | n/a | n/a | n/a |
| 754 | 773 | 794 | 451401 | GGTGCCCACAGCCACCAGGG | 82 | 763 | 888 | 907 | 1 |
| 322 | 341 | 795 | 451406 | TCCATGGCAGTGAGCCGGTC | 55 | 764 | 1319 | 1338 | 1 |
| 523 | 542 | 795 | 451407 | GGGACCACTTGATCCGGTGG | 63 | 765 | n/a | n/a | n/a |
| 534 | 553 | 795 | 451408 | GGATCAGAGTTGGGACCACT | 0 | 766 | n/a | n/a | n/a |
| 492 | 511 | 796 | 451416 | CCCCGTGATGGCTGCGGTTC | 49 | 767 | n/a | n/a | n/a |
| 469 | 488 | 797 | 451409 | GTGTGTCCTCATACCCCGCC | 60 | 768 | n/a | n/a | n/a |
| 629 | 648 | 798 | 451421 | GCACCCTCGAAGTCTCGACC | 72 | 769 | n/a | n/a | n/a |
| 854 | 873 | 799 | 451426 | GCTCTGAAGGCCATGCAGCA | 52 | 770 | n/a | n/a | n/a |

### Example 9: Dose-Dependent Antisense Inhibition of Murine DMPK in Mouse Primary Hepatocytes

Several of the antisense oligonucleotides exhibiting in vitro inhibition of DMPK in mouse primary hepatocytes (see Example 8) were tested at various doses. Cells were plated at a density of 35,000 cells per well and transfected using electroporation with 1,000 nM, 2,000 nM, 4,000 nM, 8,000 nM, and 16,000 nM concentrations of each antisense oligonucleotide. After approximately 16 hours, RNA was isolated from the cells and DMPK transcript levels were measured by quantitative real-time PCR using primer probe set RTS3181 (forward sequence GACATATGCCAAGATTGTGCACTAC, designated herein as SEQ ID NO: 771; reverse sequence CACGAATGAGGTCCTGAGCTT, designated herein as SEQ ID NO: 772; probe sequence AACACTTGTCGCTGCCGCTGGCX, designated herein as SEQ ID NO: 773). DMPK transcript levels were normalized to total RNA content, as measured by RIBOGREEN®. Results are presented in Table 17 as percent inhibition of DMPK, relative to untreated control cells.

The majority of the tested antisense oligonucleotides demonstrated dose-dependent inhibition of DMPK mRNA levels under the conditions specified above.

TABLE 17
Dose-dependent antisense inhibition of murine
DMPK in mouse primary hepatocytes

| ISIS | 1,000 | 2,000 | 4,000 | 8,000 | 16,000 | IC₅₀ |
|---|---|---|---|---|---|---|
| No | nM | nM | nM | nM | nM | (nM) |
| 451369 | 33 | 59 | 78 | 87 | 94 | 1.57 |
| 451371 | 60 | 77 | 84 | 90 | 91 | 0.24 |
| 451373 | 53 | 62 | 82 | 89 | 92 | 0.74 |
| 451374 | 33 | 42 | 76 | 88 | 94 | 2.00 |
| 451375 | 43 | 62 | 81 | 89 | 88 | 1.05 |
| 451378 | 39 | 79 | 80 | 87 | 94 | 0.87 |
| 451385 | 22 | 57 | 80 | 78 | 93 | 2.01 |
| 451393 | 49 | 63 | 86 | 80 | 80 | 0.59 |
| 451397 | 63 | 75 | 74 | 81 | 92 | 0.22 |
| 451398 | 29 | 72 | 84 | 83 | 90 | 1.29 |
| 451399 | 27 | 53 | 81 | 68 | 80 | 2.07 |
| 451401 | 34 | 71 | 87 | 86 | 92 | 1.12 |
| 451402 | 34 | 69 | 75 | 86 | 74 | 1.14 |

### Example 10: Antisense Inhibition of Human Alpha1 Skeletal Actin in HepG2 Cells

Antisense oligonucleotides targeted to a human alpha1 skeletal actin nucleic acid, a gene which may carry an expanded CTG repeat capable of causing symptoms of DM1 when inserted into mouse models, were tested for their effect on alpha1 actin RNA transcript in vitro. Cultured HepG2 cells at a density of 20,000 cells per well were transfected using electroporation with 10,000 nM antisense oligonucleotide. After approximately 24 hours, RNA was isolated from the cells and alpha1 actin RNA transcript levels were measured by quantitative real-time PCR. Alpha1 actin RNA transcript levels were adjusted according to total RNA content, as measured by RIBOGREEN®. Results are presented as percent inhibition of alpha1 actin, relative to untreated control cells.

The antisense oligonucleotides in Table 18 are 5-10-5 gapmers, where the gap segment comprises ten 2′-deoxynucleosides and each wing segment comprises five 2′-MOE nucleosides. The internucleoside linkages throughout each gapmer are phosphorothioate (P═S) linkages. All cytosine residues throughout each gapmer are 5-methylcytosines. ‘Target start site’ indicates the 5′-most nucleoside to which the antisense oligonucleotide is targeted. ‘Target stop site’ indicates the 3′-most nucleoside to which the antisense oligonucleotide is targeted. All the antisense oligonucleotides listed in Table 18 target SEQ ID NO: 801 (GENBANK Accession No. NM_001100.3).

The tested antisense oligonucleotide sequences demonstrated dose-dependent inhibition of alpha 1 actin mRNA levels under the conditions specified above.

TABLE 18
Inhibition of human alpha 1 actin RNA transcript in
HepG2 cells by 5-10-5 gapmers targeting SEQ ID NO: 801

| Target | Target | | | | |
|---|---|---|---|---|---|
| Start | Stop | | | % | SEQ ID |
| Site | Site | ISIS No | Sequence | inhibition | NO. |
| 16 | 35 | 445205 | AGCGAGGCTTCACTTGGCGC | 74 | 774 |
| 20 | 39 | 190403 | GGGAAGCGAGGCTTCACTTG | 75 | 775 |
| 1028 | 1047 | 190401 | GCGGTCAGCGATCCCAGGGT | 78 | 776 |
| 1058 | 1077 | 445225 | GGGTGCCAGCGCGGTGATCT | 73 | 777 |
| 1320 | 1339 | 445231 | TGTTACAAAGAAAGTGACTG | 74 | 778 |
| 1339 | 1358 | 445232 | CGATGGCAGCAACGGAAGTT | 96 | 779 |
| 1348 | 1367 | 445233 | GTCAGTTTACGATGGCAGCA | 100 | 780 |
| 1417 | 1436 | 445235 | CAGGGCTTTGTTTCGAAAAA | 91 | 781 |
| 1430 | 1449 | 445236 | CCATTTTCTTCCACAGGGCT | 99 | 782 |
| 1447 | 1466 | 445237 | ATGCTTCTTCAAGTTTTCCA | 97 | 783 |
| 1460 | 1479 | 445238 | CAGAATGACTTTAATGCTTC | 95 | 784 |

### Example 11: Dose-Dependent Antisense Inhibition of Human Alpha1 Actin in HepG2 Cells

Several of the antisense oligonucleotides exhibiting in vitro inhibition of alpha1 actin in HepG2 cells (see Example 8) were tested at various doses. Cells were plated at a density of 20,000 cells per well and transfected using electroporation with 625 nM, 1,250 nM, 2,500 nM, 5,000 nM, 10,000 nM and 20,000 nM concentrations of each antisense oligonucleotide. After approximately 16 hours, RNA was isolated from the cells and alpha1 actin RNA transcript levels were measured by quantitative real-time PCR using primer probe set RTS3154 (forward sequence CCACCGCAAATGCTTCTAGAC, designated herein as SEQ ID NO: 785; reverse sequence CCCCCCCATTGAGAAGATTC, designated herein as SEQ ID NO: 786; probe sequence CTCCACCTCCAGCACGCGACTTCTX, designated herein as SEQ ID NO: 787). Alpha1 actin RNA transcript levels were normalized to total RNA content, as measured by RIBOGREEN®. Results are presented in Table 19 as percent inhibition of alpha1 actin, relative to untreated control cells.

Several of the antisense oligonucleotides demonstrated dose-dependent inhibition of alpha 1 actin mRNA levels under the conditions specified above.

TABLE 19
Dose-dependent antisense inhibition of
human alpha1 actin in HepG2 cells

| ISIS | 625 | 1,250 | 2,500 | 5,000 | 10,000 | 20,000 | IC₅₀ |
|---|---|---|---|---|---|---|---|
| No. | nM | nM | nM | nM | nM | nM | (nM) |
| 445233 | 21 | 72 | 63 | 82 | 96 | 83 | 1.1 |
| 445236 | 26 | 68 | 82 | 91 | 90 | 91 | 0.8 |
| 445237 | 36 | 59 | 76 | 84 | 83 | 90 | 0.8 |
| 445232 | 14 | 42 | 54 | 59 | 80 | 91 | 2.6 |
| 445238 | 27 | 43 | 54 | 73 | 76 | 90 | 2.0 |
| 445235 | 26 | 52 | 29 | 58 | 59 | 24 | 0.7 |
| 190403 | 25 | 29 | 36 | 25 | 61 | 54 | 11.9 |
| 190401 | 17 | 14 | 40 | 68 | 76 | 72 | 3.9 |
| 445225 | 25 | 23 | 49 | 28 | 52 | 50 | 15.8 |
| 445205 | 26 | 31 | 34 | 28 | 55 | 36 | 7.6 |
| 445231 | 30 | 25 | 39 | 26 | 42 | 36 | >20.0 |

### Example 12: In Vivo Antisense Inhibition of Human Alpha1 Actin by Intramuscular Administration in Transgenic Mice

To test the effect of antisense inhibition for the treatment of myotonic dystrophy, an appropriate mouse model was required. The HSA^{LR} mouse model is an established model for DM1 (Mankodi, A. et al. Science. 289: 1769, 2000). The mice carry a human skeletal actin (hACTA1) transgene with 220 CTG repeats inserted in the 3′ UTR of the gene. The hACTA1-CUGexp transcript accumulates in nuclear foci in skeletal muscles and results in myotonia similar to that in human DM1 (Mankodi, A. et al. Mol. Cell 10: 35, 2002; Lin, X. et al. Hum. Mol. Genet. 15: 2087, 2006). Hence, it was expected that amelioration of DM1 symptoms in the HSA^{LR} mouse by antisense inhibition of the hACTA1 transgene would predict amelioration of similar symptoms in human patients by antisense inhibition of the DMPK transcript.

HSA (human skeletal actin)^{LR} (long repeat) DM1 mice were generated by insertion in FVB/N mice of a transgene with 250 CUG repeats in the 3′ UTR of human skeletal actin. The transgene is expressed in the mice as a CUG repeat RNA, which is retained in the nucleus, forming nuclear inclusions or foci, similar to that seen in human tissue samples of patients with myotonic dystrophy (DM1).

ISIS 190403 and ISIS 445238, which demonstrated statistically significant dose-dependent inhibition in vitro (see Example 11), were evaluated for their ability to reduce human alpha1 actin RNA transcript in vivo.

### Treatment

HSA^{LR} mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into two treatment groups. The two groups received direct intramuscular injections of ISIS 190403 or ISIS 445238 at a dose of 0.8 nM into the tibialis anterior muscle on one side. The contralateral tibialis anterior muscle in each mouse received a single dose intramuscular injection of PBS. The PBS-injected muscle acted as the control.

### Inhibition of Alpha1 Actin RNA

Twenty four hours after the final dose, the animals were sacrificed and tissue from the tibialis anterior muscles of both sides was isolated. RNA was isolated for real-time PCR analysis of alpha1 actin and normalized to 18s RNA. As presented in Table 20, treatment with antisense oligonucleotides reduced human alpha1 actin RNA transcript expression. The results are expressed as percent inhibition of alpha1 actin transcript, relative to the PBS control. The results indicate that treatment with ISIS 190403 and ISIS 445238 resulted in inhibition of alpha 1 actin RNA levels in the mice.

TABLE 20
Percent inhibition of human alpha1 actin
RNA transcript in HSA^{LR} mice

| ISIS | % |
|---|---|
| No. | inhibition |
| 190403 | 38 |
| 445238 | 40 |

### Example 13: Dose Dependent Antisense Inhibition of Human Alpha1 Actin by Intramuscular Administration in Transgenic Mice

ISIS 445236, which demonstrated statistically significant dose-dependent inhibition in vitro (see Example 11), was evaluated for its ability to reduce human alpha1 actin RNA transcript in vivo.

### Treatment

HSA^{LR} mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into three treatment groups. The groups received direct intramuscular injections of ISIS 445236 at doses of 0.2 nM, 0.4 nM or 0.8 nM into the tibialis anterior muscle of one side. The contralateral tibialis anterior muscle in each mouse received a single dose intramuscular injection of PBS. The PBS-injected muscle acted as the control.

### Inhibition of Alpha1 Actin RNA

Twenty four hours after the final dose, the animals were sacrificed and tissue from the tibialis anterior muscles of both sides was isolated. RNA was isolated for real-time PCR analysis of alpha1 actin and normalized to 18s RNA. As presented in Table 21, treatment with ISIS 445236 reduced human alpha1 actin RNA transcript expression at all dosages. The results are expressed as percent inhibition of alpha1 actin transcript, relative to the control.

The results indicate that treatment with ISIS 445236 resulted in significant inhibition of alpha 1 actin mRNA levels under the conditions specified above.

TABLE 21
Inhibition of human alpha1 actin
RNA transcript by ISIS 445236
in HSA^{LR} mice

| Dose | % |
|---|---|
| (nM) | inhibition |
| 0.2 | 70 |
| 0.4 | 54 |
| 0.8 | 78 |

### Assessment of Myotonia by Electromyography

Myotonia refers to repetitive action potential that is due to delayed relaxation of muscle fibers. This phenomenon is observed in patients of myotonic dystrophy as well as in the HSA^{LR} mice. When the EMG needle is inserted into a myotonic muscle, the electrical activity is prolonged for up to several seconds past when the insertional activity should normally cease. The frequency of myotonic discharges ranges from 50 to 100 impulses per second.

Myotonia was measured via electromyography and graded in the following manner: grade 0 refers to no myotonia elicited by any needle insertion (0%); grade 1 refers to myotonia elicited by less than 50% needle insertions; grade 2 refers to myotonia elicited by more than 50% needle insertions; and grade 3 refers to mytonia elicited by 100% needle insertions.

Before electromyography, mice were anesthetized by using i.p. a cocktail of 100 mg/kg ketamine, 10 mg/kg xylazine, and 3 mg/kg acepromazine. Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 22 as the average myotonia grade observed in four mice of each group and demonstrates significant reduction of myotonia in mice treated with ISIS 445236.

TABLE 22
Average reduction of myotonia in various muscles
of antisense oligonucleotide-treated HSA^{LR} mice

| | Dose | Myotonia |
|---|---|---|
| Treatment | (nM) | grade |
| PBS | | 2.7 |
| ISIS | 0.2 | 1.3 |
| 455236 | 0.4 | 1.0 |
| | 0.8 | 1.0 |

### Correction of Alternative Splicing

In DM1/HSA^{LR} mouse model, the accumulation of expanded CUG RNA in the nucleus leads to the sequestration of poly(CUG)-binding proteins, such as Muscleblind-like 1 (MBLN1) (Miller, J. W. et al. EMBO J. 19: 4439, 2000). The splicing factor MBNL1, which controls alternative splicing of the Serca1 gene is sequestered in expanded CUG foci. This triggers dysregulation of the alternative splicing of this gene. To evaluate the effect of antisense inhibition of human alpha 1 actin on such alternative splicing, total RNA was purified from the tibialis anterior, gastrocnemius, and quadriceps muscle using RNeasy Lipid Tissue Mini Kit (Qiagen), according to the manufacturer's instructions. RT-PCR was performed with the SuperScript III One-Step RT-PCR System and Platinum Taq Polymerase (Invitrogen), using gene-specific primers for cDNA synthesis and PCR amplification. The forward and reverse primers for Serca-1 have been described in Bennett and Swayze (Annu. Rev. Pharmacol. 2010; 50: 259-93). PCR products were separated on agarose gels, stained with SybrGreen I Nucleic Acid Gel Stain (Invitrogen), and imaged using a Fujifilm LAS-3000 Intelligent Dark Box.

The PCR products of Serca1 splicing in the PBS control demonstrated exon 22 exclusion as a result of dysregulation of MBLN1. Treatment with ISIS 445236 resulted in exon 22 inclusion and normalization of alternative splicing of the Serca1 gene in the tibialis anterior, gastrocnemius, and quadriceps muscles.

Therefore, antisense inhibition of alpha1 actin corrected Serca1 splicing dysregulation, which indicates that treatment with antisense oligonucleotide reduced accumulation of CUGexp in the nuclear foci. Reduced accumulation of CUGexp in the nuclear foci corrects MBLN1 sequestration thereby allowing normal splicing to occur.

### Example 14: In Vivo Antisense Inhibition of Human Alpha1 Actin by Subcutaneous Administration in Transgenic Mice

ISIS 190403, ISIS 445236 and ISIS 445238 were evaluated for their ability to reduce human alpha1 actin RNA transcript in vivo.

### Treatment

HSA^{LR} mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into four treatment groups. The first three groups received subcutaneous injections of ISIS 190403, ISIS 445236 or ISIS 445238 at a dose of 25 mg/kg twice per week for 4 weeks. The fourth group received subcutaneous injections of PBS twice weekly for 4 weeks. The PBS-injected group served as the control group to which the oligonucleotide-treated group was compared.

### Inhibition of Alpha1 Actin RNA

Twenty four hours after the final dose, the animals were sacrificed and tissue from the quadriceps muscles (left and right), gastrocnemius muscles (left and right), and tibialis anterior muscles (left and right) was isolated. RNA was isolated for real-time PCR analysis of alpha1 actin and normalized to 18s RNA. As presented in Table 23, treatment with antisense oligonucleotides reduced human alpha1 actin RNA transcript expression. The results are expressed as percent inhibition of alpha1 actin transcript, relative to the control.

Both ISIS 445236 and ISIS 445238 demonstrated significant inhibition of alpha1 actin mRNA levels under the conditions specified above.

TABLE 23
Percent inhibition of human alpha1 actin
RNA transcript in HSA^{LR} mice

| | ISIS | ISIS | ISIS |
|---|---|---|---|
| Muscle Type | 190403 | 445236 | 445238 |
| Quadriceps | 16 | 83 | 72 |
| Gastrocnemius | 0 | 85 | 73 |
| Tibialis anterior | 2 | 81 | 71 |

### Fluorescence In Situ Hybridization of Alpha1 Actin in Muscles

Frozen muscle tissue sections were fixed in fresh 3% paraformaldehyde in PBS solution for 15-20 minutes, after which they were rinsed twice with PBS for 5 minutes. The nuclei were permeabilized with 0.5% Triton X-100 for 5 minutes after which the tissue was blocked with normal goat serum for 30 minutes. The sections were incubated a 2′-O-methyl RNA targeted to alpha1 actin that is 5′-labeled with Texas Red (Integrated DNA Technologies). The sections were counter-stained with DAPI to label the nuclei. The sections were mounted and viewed with a standard fluorescence microscope. Image acquisition was by Metavue software and deconvolution was achieved by Autoquant software.

All muscle tissue sections from mice treated with ISIS 445236 and ISIS 445238 displayed reduced fluorescent intensity of alpha1 actin signal at the ribonuclear foci, indicating antisense inhibition of human alpha1 actin mRNA and reduction of the RNA in the nuclear foci.

### Assessment of Myotonia by Electromyography

Myotonia refers to repetitive action potential that is due to delayed relaxation of muscle fibers. This phenomenon is observed in patients of myotonic dystrophy as well as in the HSA^{LR} mice. When the EMG needle is inserted into a myotonic muscle, the electrical activity is prolonged for up to several seconds past when the insertional activity should normally cease. The frequency of myotonic discharges ranges from 50 to 100 impulses per second.

Myotonia may be measured via electromyography and is graded in the following manner: grade 0 refers to no myotonia elicited by any needle insertion (0%); grade 1 refers to myotonia elicited by less than 50% needle insertions; grade 2 refers to myotonia elicited by more than 50% needle insertions; and grade 3 refers to mytonia elicited by 100% needle insertions.

Before electromyography, mice were anesthetized by using i.p. 100 mg/kg ketamine, 10 mg/kg xylazine, and 3 mg/kg acepromazine or 250 mg/kg 2,2,2-tribromoethanol. Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 24 as the average myotonia grade observed in four mice of each group and demonstrates significant reduction of myotonia in mice treated with ISIS 445236 and ISIS 445238.

TABLE 24
Average reduction of myotonia in various muscles of
antisense oligonucleotide-treated HSA^{LR} mice

| | | ISIS | ISIS | ISIS |
|---|---|---|---|---|
| | PBS | 190403 | 445236 | 445238 |
| Left quadriceps | 3.00 | 3.00 | 0.00 | 0.25 |
| Right quadriceps | 3.00 | 3.00 | 0.00 | 0.00 |
| Left gastrocnemius | 3.00 | 3.00 | 0.00 | 0.25 |
| Right gastrocnemius | 3.00 | 3.00 | 0.00 | 0.25 |
| Left Tibialis anterior | 2.75 | 2.50 | 0.00 | 0.00 |
| Right Tibialis anterior | 2.75 | 2.50 | 0.00 | 0.00 |
| Lumbar paraspinals | 3.00 | 3.00 | 0.00 | 0.75 |

### Correction of Alternative Splicing

The splicing factor MBNL1, which controls Serca1 splicing, m-Titin splicing, CIC-1 chloride channel gene (Clcn1) splicing, and Zasp splicing, is sequestered in expanded CUG foci. MBNL1 sequestration triggers dysregulated splicing in each of these genes. To evaluate the effect of antisense inhibition of human alpha 1 actin on splicing, total RNA was purified from the tibialis anterior, gastrocnemius, and quadriceps muscle and RT-PCR was performed, as described in Example 13. The forward and reverse primers for Serca-1, m-Titin, Clcn1, and ZASP have been described in Bennett and Swayze, Annu. Rev. Pharmacol. 2010; 50: 259-93.

In PBS treated HSA^{LR} mice, Serca1 splicing is dysregulated as demonstrated by exon 22 exclusion. Treatment with each of ISIS 445236 and ISIS 445238 resulted in exon 22 inclusion and normalization of alternative splicing of the Serca1 gene in the tibialis anterior, gastrocnemius, and quadriceps muscles.

In PBS treated HSA^{LR} mice, m-Titin splicing is dysregulated as demonstrated by exon 5 inclusion. Treatment with each of ISIS 445236 and ISIS 445238 resulted in skipping of exon 5 and normalization of alternative splicing of the m-Titin gene in the tibialis anterior, gastrocnemius, and quadriceps muscles.

In PBS treated HSA^{LR} mice, Clcn1 splicing is dysregulated as demonstrated by exon 7a inclusion. Treatment with each of ISIS 445236 and ISIS 445238 resulted in skipping of exon 7a and normalization of alternative splicing of the Clcn1 gene in the tibialis anterior, gastrocnemius, and quadriceps muscles.

In PBS treated HSA^{LR} mice, Zasp splicing is dysregulated as demonstrated by exon 11 inclusion. Treatment with each of ISIS 445236 and ISIS 445238 resulted in skipping of exon 11 and normalization of alternative splicing of the Zasp gene in the tibialis anterior, gastrocnemius, and quadriceps muscles.

Therefore, antisense inhibition of alpha1 actin corrected Serca1, m-Titin, Clcn1, and Zasp splicing dysregulation, which indicates that treatment with antisense oligonucleotide reduced accumulation of CUGexp in the nuclear foci. Reduced accumulation of CUGexp in the nuclear foci correct MBLN1 sequestration thereby allowing normal splicing to occur.

### Example 15: In Vivo Antisense Inhibition of Human Alpha1 Actin in Transgenic Mice

Antisense inhibition of human alpha1 actin RNA transcript by ISIS 445236 and ISIS 445238 on myotonia in HSA^{LR} mice was further evaluated.

### Treatment

HSA^{LR} mice were divided into three treatment groups. The first two groups received subcutaneous injections of ISIS 445236 or ISIS 445238 at a dose of 25 mg/kg twice per week for 2 weeks. The third group received subcutaneous injections of PBS twice per week for 2 weeks. The PBS-injected group served as the control group to which the oligonucleotide-treated group was compared.

### Inhibition of Alpha1 Actin RNA

Twenty four hours after the final dose, the animals were sacrificed and tissue from the quadriceps muscles, gastrocnemius muscles, and tibialis anterior muscles was isolated. RNA was isolated for real-time PCR analysis of alpha1 actin and normalized to 18s RNA. As presented in Table 25, treatment with antisense oligonucleotides reduced human alpha1 actin RNA transcript expression. The results are expressed as percent inhibition of alpha1 actin transcript, relative to the PBS control.

Both ISIS 445236 and ISIS 445238 demonstrated significant inhibition of alpha1 actin mRNA levels under the conditions specified above.

TABLE 25
Percent inhibition of human alpha1 actin
RNA transcript in HSA^{LR} mice

| | ISIS | ISIS |
|---|---|---|
| Muscle Type | 445236 | 445238 |
| Quadriceps | 61 | 64 |
| Gastrocnemius | 68 | 37 |
| Tibialis anterior | 68 | 41 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 26 as the average myotonia grade observed in four mice of each group and demonstrates significant reduction of myotonia in mice treated with ISIS 445236 and ISIS 445238.

TABLE 26
Average reduction of myotonia in various muscles
of antisense oligonucleotide-treated HSA^{LR} mice

| | | ISIS | ISIS |
|---|---|---|---|
| | PBS | 445236 | 445238 |
| Left quadriceps | 3.00 | 0.00 | 1.75 |
| Right quadriceps | 3.00 | 0.00 | 1.75 |
| Left gastrocnemius | 3.00 | 0.25 | 1.5 |
| Right gastrocnemius | 3.00 | 0.25 | 1.00 |
| Left Tibialis anterior | 2.75 | 0.00 | 0.00 |
| Right Tibialis anterior | 2.75 | 0.00 | 0.00 |
| Lumbar paraspinals | 3.00 | 0.50 | 2.00 |

### Correction of Alternative Splicing

To evaluate the effect of ISIS 190401 on alternative splicing of Serca1, total RNA purified from the tibialis anterior gastrocnemius, and quadriceps muscle was analyzed in a procedure similar to that described in Example 13.

In PBS treated HSA^{LR} mice, Serca1 splicing is dysregulated as demonstrated by exon 22 exclusion, as a result of MBLN1 dysregulation. Treatment with each of ISIS 445236 and ISIS 445238 resulted in near-complete inclusion and normalization of alternative splicing of exon 22 of the Serca1 gene in the tibialis anterior and quadriceps muscles.

Therefore, antisense inhibition of alpha1 actin corrected Serca1 splicing dysregulation, which indicates that treatment with antisense oligonucleotide reduced accumulation of CUGexp in the nuclear foci. Reduced accumulation of CUGexp in the nuclear foci correct MBLN1 sequestration thereby allowing normal splicing to occur.

### Example 16: Dose-Dependent Antisense Inhibition of Human Alpha1 Actin in Transgenic Mice

Dose-dependent inhibition of human alpha1 actin RNA transcript by ISIS 445236 and ISIS 445238 on myotonia in HSA^{LR} mice was evaluated.

### Treatment

HSA^{LR} mice were subcutaneously injected with ISIS 445236 or ISIS 445238 at doses of 2.5 mg/kg, 8.5 mg/kg or 25.0 mg/kg twice per week for 4 weeks. The control group received subcutaneous injections of PBS twice per week for 4 weeks. The PBS-injected group served as the control group to which the oligonucleotide-treated group was compared.

### Inhibition of Alpha1 Actin RNA

Twenty four hours after the final dose, the animals were sacrificed and tissue from the quadriceps muscles (Quad), gastrocnemius muscles (Gastroc), and tibialis anterior muscles (TA) was isolated. RNA was isolated for real-time PCR analysis of alpha1 actin and normalized to 18s RNA. As presented in Table 27, treatment with antisense oligonucleotides reduced human alpha1 actin RNA transcript expression. The results are expressed as percent inhibition of alpha1 actin transcript, relative to the PBS control.

Both the antisense oligonucleotides demonstrated dose-dependent inhibition of alpha1 actin mRNA levels in quadriceps muscles, gastrocnemius muscles, and tibialis anterior muscles under the conditions specified above.

TABLE 27
Dose-dependent inhibition of human alpha1
actin RNA transcript in HSA^{LR} mice

| | mg/kg/wk | Quad | Gastroc | TA |
|---|---|---|---|---|
| ISIS 445236 | 5 | 24 | 36 | 46 |
| | 17 | 53 | 57 | 59 |
| | 50 | 86 | 86 | 90 |
| ISIS 445238 | 5 | 21 | 37 | 3 |
| | 17 | 30 | 39 | 60 |
| | 50 | 59 | 81 | 70 |

### Assessment of Myotonia by Electromyography

Electromyography on left and right quadriceps (Quad), left and right gastrocnemius muscles (Gastroc), left and right tibialis anterior (TA) muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 28 as the average myotonia grade observed in four mice of each group and demonstrates significant dose-dependent reduction of myotonia in mice treated with ISIS 445236 and ISIS 445238.

TABLE 28
Average reduction of myotonia in various muscles
of antisense oligonucleotide-treated HSA^{LR} mice

| | mg/kg/ | Left | Right | Left | Right | Left | Right | Lumbar |
|---|---|---|---|---|---|---|---|---|
| | wk | Quad | Quad | Gastroc | Gastroc | TA | TA | paraspinals |
| PBS | — | 3.00 | 3.00 | 3.00 | 3.00 | 2.75 | 2.75 | 3.00 |
| ISIS | 5 | 3.00 | 3.00 | 3.00 | 3.00 | 2.25 | 2.25 | 3.00 |
| 445236 | 17 | 0.75 | 0.75 | 0.75 | 1.00 | 0.00 | 0.00 | 1.75 |
| | 50 | 0.00 | 0.00 | 0.00 | 0.00 | 0.00 | 0.00 | 0.00 |
| ISIS | 5 | 2.75 | 2.75 | 2.50 | 2.50 | 2.00 | 1.75 | 2.75 |
| 445238 | 17 | 3.00 | 3.00 | 2.00 | 2.25 | 0.00 | 0.00 | 2.75 |
| | 50 | 0.75 | 0.75 | 0.25 | 0.25 | 0.00 | 0.00 | 1.00 |

### Correction of Alternative Splicing

To evaluate the effect of ISIS 190401 on alternative splicing of Serca1, total RNA purified from the tibialis anterior gastrocnemius, and quadriceps muscle was analyzed in a procedure similar to that described in Example 13.

In PBS treated HSA^{LR} mice, Serca1 splicing is dysregulated as demonstrated by exon 22 exclusion, as a result of MBLN1 dysregulation. Treatment with either ISIS 445236 or ISIS 445238 at doses of 8.5 mg/kg or 25.0 mg/kg twice a week (or 17.0 mg/kg/week and 50.0 mg/kg/week) resulted in complete inclusion and normalization of alternative splicing of exon 22 of the Serca1 gene in all three muscle types.

Therefore, antisense inhibition of alpha1 actin corrected Serca1 splicing dysregulation, which indicates that treatment with antisense oligonucleotide reduced accumulation of CUGexp in the nuclear foci. Reduced accumulation of CUGexp in the nuclear foci correct MBLN1 sequestration thereby allowing normal splicing to occur.

### Example 17: In Vivo Antisense Inhibition by an Oligonucleotide Targeting the HSA Coding Region of Human Alpha1 Actin in Transgenic Mice

Antisense inhibition of human alpha1 actin RNA transcript by ISIS 190401 (5′-GCGGTCAGCGATCCCAGGGT-3′ (SEQ ID NO: 788), target start site 1028 of SEQ ID NO: 1) on myotonia in HSA^{LR} mice was evaluated.

### Treatment

HSA^{LR} mice received subcutaneous injections of ISIS 190401 at a dose of 25 mg/kg twice per week for 4 weeks. A control group received subcutaneous injections of PBS twice per week for 2 weeks. The PBS-injected group served as the control group to which the oligonucleotide-treated group was compared.

### Inhibition of Alpha1 Actin RNA

Twenty four hours after the final dose, the animals were sacrificed and tissue from the quadriceps muscles, gastrocnemius muscles, and tibialis anterior muscles was isolated. RNA was isolated for real-time PCR analysis of alpha1 actin and normalized to 18s RNA. As presented in Table 29, treatment with antisense oligonucleotides reduced human alpha1 actin RNA transcript expression. The results are expressed as percent inhibition of alpha1 actin transcript, relative to the PBS control.

Treatment with ISIS 190401 resulted in significant inhibition of alpha1 actin mRNA levels in quadriceps muscle, gastrocnemius muscle, and tibialis anterior muscle under the conditions specified above.

TABLE 29
Antisense inhibition of human alpha1
actin RNA transcript in HSA^{LR} mice

| | % |
|---|---|
| Muscle Type | inhibition |
| Quadriceps | 85 |
| Gastrocnemius | 86 |
| Tibialis anterior | 89 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 30 as the average myotonia grade observed in four mice of each group and demonstrates significant reduction of myotonia in mice treated with ISIS 190401.

TABLE 30
Average reduction of myotonia in various muscles
of antisense oligonucleotide-treated HSA^{LR} mice

| | | ISIS |
|---|---|---|
| | PBS | 190401 |
| Left quadriceps | 3.00 | 0.00 |
| Right quadriceps | 3.00 | 0.00 |
| Left gastrocnemius | 3.00 | 0.00 |
| Right gastrocnemius | 3.00 | 0.00 |
| Left Tibialis anterior | 2.50 | 0.00 |
| Right Tibialis anterior | 2.50 | 0.00 |
| Lumbar paraspinals | 3.00 | 0.50 |

### Correction of Alternative Splicing

To evaluate the effect of ISIS 190401 on alternative splicing of Serca1, total RNA purified from the tibialis anterior gastrocnemius, and quadriceps muscle was analyzed in a procedure similar to that described in Example 13.

In PBS treated HSA^{LR} mice, Serca1 splicing is dysregulated as demonstrated by exon 22 exclusion, as a result of MBLN1 dysregulation. Treatment with ISIS 190401 resulted in complete inclusion and normalization of alternative splicing of exon 22 of the Serca1 gene in all three muscle types.

Therefore, antisense inhibition of alpha1 actin corrected Serca1 splicing dysregulation, which indicates that treatment with antisense oligonucleotide reduced accumulation of CUGexp in the nuclear foci. Reduced accumulation of CUGexp in the nuclear foci corrects MBLN1 sequestration thereby allowing normal splicing to occur.

### Example 18: Duration of Action of Antisense Inhibition by an Oligonucleotide Targeting Human Alpha1 Actin in Transgenic Mice

The duration of action of antisense inhibition of human alpha1 actin RNA transcript by ISIS 445236 in HSA^{LR} mice was evaluated.

### Treatment

HSA^{LR} mice received subcutaneous injections of ISIS 445236 at a dose of 25 mg/kg twice per week for 4 weeks. A control group received subcutaneous injections of PBS twice per week for 2 weeks. The PBS-injected group served as the control group to which the oligonucleotide-treated group was compared. The mice were analyzed 6 weeks after administration of the last dose.

### Inhibition of Alpha1 Actin RNA

Six weeks after the final dose, the animals were sacrificed and tissue from the quadriceps muscles, gastrocnemius muscles, and tibialis anterior muscles was isolated. RNA was isolated for real-time PCR analysis of alpha1 actin and normalized to 18s RNA. As presented in Table 31, treatment with ISIS 445236 reduced human alpha1 actin RNA transcript expression, and this effect was sustained at least for 6 weeks. The results are expressed as percent inhibition of alpha1 actin transcript, relative to the PBS control.

Treatment with ISIS 445236 resulted in significant inhibition of alpha1 actin mRNA levels in quadriceps muscle, gastrocnemius muscle, and tibialis anterior muscle under the conditions specified above.

TABLE 31
Antisense inhibition of human alpha1
actin RNA transcript in HSA^{LR} mice

| | % |
|---|---|
| Muscle Type | inhibition |
| Quadriceps | 88 |
| Gastrocnemius | 76 |
| Tibialis anterior | 67 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 32 as the average myotonia grade observed in four mice of each group and demonstrates significant reduction of myotonia in mice treated with ISIS 445236. Therefore, the effect of antisense inhibition of alpha actin by ISIS 445236 was sustained at least for 6 weeks.

TABLE 32
Average reduction of myotonia in various muscles
of antisense oligonucleotide-treated HSA^{LR} mice

| | | ISIS |
|---|---|---|
| | PBS | 445236 |
| Left quadriceps | 3.00 | 0.00 |
| Right quadriceps | 3.00 | 0.00 |
| Left gastrocnemius | 3.00 | 0.00 |
| Right gastrocnemius | 3.00 | 0.00 |
| Left Tibialis anterior | 2.50 | 0.00 |
| Right Tibialis anterior | 2.50 | 0.00 |
| Lumbar paraspinals | 3.00 | 0.00 |

### Example 19: In Vivo Effect of Antisense Inhibition of mRNA with CUG Repeats by Intramuscular Administration in Transgenic Mice

The effect of antisense inhibition of mRNA transcripts containing multiple CUG repeats on myotonia in HSA^{LR} mice was evaluated. Three antisense oligonucleotides targeting the CUG repeats and with varying lengths were assayed for their effectiveness in inhibiting myotonia in the mice. ISIS 444745 (AGCAGCAGCAGCAGCAGCAGCAGCA (SEQ ID NO: 789) is a uniform 2′-O-methoxyethyl oligonucleotide, 25 nucleotides in length and with a phosphorothioate backbone. ISIS 444746 (AGCAGCAGCAGCAGCAGCAG (SEQ ID NO: 790) is a uniform 2′-O-methoxyethyl oligonucleotide, 20 nucleotides in length and with a phosphorothioate backbone. ISIS 444749 (GCAGCAGCAGCAGCA (SEQ ID NO: 791) is a uniform 2′-O-methoxyethyl oligonucleotide, 15 nucleotides in length and with a phosphorothioate backbone. ISIS 445236 was included in the assay as a positive control.

### Treatment

HSA^{LR} mice were divided into three treatment groups. The groups received direct intramuscular injections of ISIS 444745, ISIS 444746 or ISIS 444749 at a dose of 0.4 nM into the tibialis anterior muscle. The contralateral tibialis anterior muscle in each mouse received a single dose intramuscular injection of PBS. The PBS-injected muscle acted as the control.

### Inhibition of Alpha1 Actin RNA

Twenty four hours after the final dose, the animals were sacrificed and tissue from the tibialis anterior (left and right) was isolated. RNA was isolated for real-time PCR analysis of alpha1 actin and normalized to 18s RNA. As presented in Table 33, only treatment with ISIS 444745 reduced human alpha1 actin RNA transcript expression. The results are expressed as percent inhibition of alpha1 actin transcript, relative to the PBS control.

TABLE 33
Percent inhibition of human alpha1
actin RNA transcript in HSA^{LR} mice

| ISIS | % |
|---|---|
| No. | inhibition |
| 444745 | 51 |
| 444746 | 0 |
| 444749 | 12 |

### Example 20: In Vivo Dose Dependent Inhibition of mRNA with CUG Repeats by Intramuscular Administration in Transgenic Mice

ISIS 444745 and ISIS 444746 were further evaluated for their ability to reduce human alpha 1 actin mRNA in vivo.

### Treatment

HSA^{LR} mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into 6 treatment groups. Three of the groups received direct intramuscular injections of ISIS 444745 at doses of 0.2 nM, 0.5 nM, or 1.0 nM into the tibialis anterior muscle on one side. Another three groups direct intramuscular injections of ISIS 444746 at doses of 0.2 nM, 0.5 nM, or 1.0 nM into the tibialis anterior muscle on one side. The contralateral tibialis anterior muscle in each mouse received a single dose intramuscular injection of PBS. The PBS-injected muscle acted as the control for the corresponding muscle treated with ISIS oligonucleotide.

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 34 as the average myotonia grade observed in four mice of each group and demonstrates significant reduction of myotonia in mice treated with either ISIS 444745 or ISIS 444746. The effect of antisense inhibition of alpha actin by ISIS 444745 and 444746 was sustained at least for 6 weeks.

TABLE 34
Dose-dependent reduction of myotonia in muscles
of antisense oligonucleotide-treated HSA^{LR} mice

| | 0.2 nM | 0.5 nM | 1.0 nM |
|---|---|---|---|
| PBS | 3.00 | 3.00 | 2.33 |
| ISIS 444745 | 1.67 | 1.00 | 0.33 |
| PBS | 2.50 | 2.00 | 3.00 |
| ISIS444746 | 2.00 | 0.00 | 1.00 |

### Example 21: In Vivo Effect of Antisense Inhibition of mRNA with CUG Repeats by Subcutaneous Administration in Transgenic Mice

The effect of antisense inhibition of mRNA transcripts containing multiple CUG repeats on myotonia in HSA^{LR} mice was evaluated. ISIS 445236 was included in the assay as a positive control.

### Treatment

HSA^{LR} mice were divided into five treatment groups. The first three groups received subcutaneous injections of ISIS 444745, ISIS 444746 or ISIS 444749 at a dose of 25 mg/kg twice per week for 4 weeks. The fourth group received subcutaneous injections of PBS twice per week for 4 weeks. The fifth group received subcutaneous injections of ISIS 445236 at a dose of 25 mg/kg twice per week for 4 weeks. The PBS-injected group served as the control group to which the oligonucleotide-treated group was compared.

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 35 as the average myotonia grade observed in four mice of each group.

Treatment with ISIS 445236 led to significant reduction in myotonia. Treatment with ISIS 444745 and ISIS 444746 also resulted in reduced myotonia in some of the tissues tested.

TABLE 35
Average reduction of myotonia in various muscles
of antisense oligonucleotide-treated HSA^{LR} mice

| | | ISIS | ISIS | ISIS | ISIS |
|---|---|---|---|---|---|
| | PBS | 444745 | 444746 | 444749 | 445236 |
| Left quadriceps | 3.00 | 3.00 | 3.00 | 3.00 | 0.00 |
| Right quadriceps | 3.00 | 3.00 | 3.00 | 3.00 | 0.00 |
| Left gastrocnemius | 3.00 | 2.75 | 3.00 | 3.00 | 0.00 |
| Right gastrocnemius | 3.00 | 2.75 | 2.75 | 3.00 | 0.00 |
| Left Tibialis anterior | 3.00 | 2.25 | 2.75 | 2.75 | 0.00 |
| Right Tibialis anterior | 3.00 | 2.25 | 2.50 | 2.75 | 0.00 |
| Lumbar paraspinals | 3.00 | 3.00 | 3.00 | 3.00 | 0.00 |

### Example 22: Dose-Dependent Inhibition of Long CUG Repeat mRNA (HSA^{LR} Mice) and a Short CUG Repeat (HSA^{SR} Mice) by Subcutaneous Administration in Transgenic Mice

Dose-dependent inhibition of mRNA transcripts containing a long CUG repeat (HSALR mice) and a short CUG repeat (HSA^{SR} mice), was evaluated. HSA-short repeat (HSA^{SR}) mice express the identical transgene as the HSA^{LR} mice, except that 5 instead of 250 CUG repeats are inserted in the 3′ UTR. HSA^{SR} mice do not have myotonia, splicing changes, or any other observable myotonia phenotype. ISIS 445236 was used in this assay.

### Treatment

HSA^{LR} mice were divided into four treatment groups. The first three groups received subcutaneous injections of ISIS 445236 at doses of 2.5 mg/kg, 8.5 mg/kg or 25.0 mg/kg twice per week for 4 weeks. The fourth group received subcutaneous injections of PBS twice per week for 4 weeks. The PBS-injected group served as the control group to which the oligonucleotide-treated group was compared. HSA^{SR} mice were also divided into four groups and similarly treated.

### Inhibition of Alpha1 Actin RNA

Twenty four hours after the final dose, the animals were sacrificed and tissue from the quadriceps muscles (left and right), gastrocnemius muscles (left and right), and tibialis anterior muscles (left and right) was isolated. RNA was isolated for real-time PCR analysis of alpha1 actin and normalized to 18s RNA. The results are presented in Tables 36 and 37 and are expressed as percent inhibition of alpha1 actin transcript, relative to the control. Greater inhibition of the nuclear-retained long repeat in the muscle of HSA^{LR} mice was achieved compared with the non-nuclear-retained short repeat in the muscle of HSA^{SR} mice.

TABLE 36
Percent inhibition of human alpha1 actin
RNA transcript in HSA^{LR} mice

| Dose | | | Tibialis |
|---|---|---|---|
| (mg/kg) | Quadriceps | Gastrocnemius | anterior |
| 2.5 | 24 | 36 | 46 |
| 8.5 | 53 | 66 | 59 |
| 25 | 86 | 86 | 90 |

TABLE 37
Percent inhibition of human alpha1 actin
RNA transcript in HSA^{SR} mice

| Dose | | | Tibialis |
|---|---|---|---|
| (mg/kg) | Quadriceps | Gastrocnemius | anterior |
| 2.5 | 15 | 14 | 0 |
| 8.5 | 30 | 11 | 0 |
| 25 | 59 | 48 | 54 |

### Example 23: In Vivo Antisense Inhibition of Human DMPK in Transgenic Mice

LC15 mice, Line A, are transgenic mice containing the entire human DMPK 3′UTR (developed by Wheeler et al, University of Rochester). The mice are the second generation of mice backcrossed to an FVB background. The transgene is expressed in the mice as a CUG repeat RNA, which is retained in the nucleus, forming nuclear inclusions or foci, similar to that seen in human tissue samples of patients with myotonic dystrophy (DM1). There are 350-400 CUG repeats in the DMPK transgene. These mice display early signs of DM1 and do not display any myotonia in their muscle tissues.

ISIS 445569, ISIS 444404, ISIS 444436 and ISIS 473810, which demonstrated statistically significant dose-dependent inhibition in vitro (see Example 5), were evaluated for their ability to reduce human DMPK RNA transcript in vivo.

### Treatment

LC15, Line A mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into five treatment groups. The first three groups received subcutaneous injections of ISIS 445569, ISIS 444404 or ISIS 444436 at a dose of 25 mg/kg twice per week for 4 weeks. The fourth group received subcutaneous injections of ISIS 473810 at a dose of 12.5 mg/kg twice per week for 4 weeks. The fifth group received subcutaneous injections of PBS twice weekly for 4 weeks. The PBS-injected group served as the control group to which the oligonucleotide-treated group was compared.

### Inhibition of DMPK RNA

Twenty four hours after the final dose, the animals were sacrificed and tissue from the quadriceps muscles was isolated. RNA was isolated for real-time PCR analysis of DMPK and normalized to 18s RNA. As presented in Table 38, treatment with antisense oligonucleotides reduced human DMPK RNA transcript expression. The results are expressed as percent inhibition of DMPK transcript, relative to the PBS control.

TABLE 38
Antisense inhibition of human DMPK
RNA transcript in LC15 mice

| | | % |
|---|---|---|
| ISIS No | mg/kg/wk | inhibition |
| 444404 | 50 | 20 |
| 444404 | 50 | 55 |
| 444436 | 50 | 41 |
| 473810 | 25 | 56 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. Since LC15 mice do not have myotonia, neither the control group nor the treatment groups displayed any myotonia in any muscle tested.

### Example 24: In Vivo Antisense Inhibition of Human DMPK in Transgenic Mice

LC15 mice, Line D, are transgenic mice containing the entire human DMPK 3′UTR (developed by Wheeler et al, University of Rochester). The mice are the third generation of mice backcrossed to an FVB background. The transgene is expressed in the mice as a CUG repeat RNA, which is retained in the nucleus, forming nuclear inclusions or foci, similar to that seen in human tissue samples of patients with myotonic dystrophy (DM1). There are 350-400 CUG repeats in the DMPK transgene. These mice display early signs of DM1 and do not display any myotonia in their muscle tissues.

ISIS 445569, ISIS 444404, ISIS 444436 and ISIS 473810 were further evaluated for their ability to reduce human DMPK RNA transcript in vivo.

### Treatment

LC15, Line D mice were maintained on a 12-hour light/dark cycle and fed ad libitum normal Purina mouse chow. Animals were acclimated for at least 7 days in the research facility before initiation of the experiment. Antisense oligonucleotides (ASOs) were prepared in PBS and sterilized by filtering through a 0.2 micron filter. Oligonucleotides were dissolved in 0.9% PBS for injection.

The mice were divided into six treatment groups. The first three groups received subcutaneous injections of ISIS 445569, ISIS 444404 or ISIS 444436 at a dose of 25.00 mg/kg twice per week for 4 weeks. The fourth group received subcutaneous injections of ISIS 473810 at a dose of 12.50 mg/kg twice per week for 4 weeks. The fifth group received subcutaneous injections of ISIS 473810 at a dose of 6.25 mg/kg twice per week for 4 weeks. The sixth group received subcutaneous injections of PBS twice weekly for 4 weeks. The PBS-injected group served as the control group to which the oligonucleotide-treated group was compared.

### Inhibition of DMPK RNA

Twenty four hours after the final dose, the animals were sacrificed and tissue from the quadriceps muscles was isolated. RNA was isolated for real-time PCR analysis of DMPK and normalized to 18s RNA. As presented in Table 39, treatment with antisense oligonucleotides reduced human DMPK RNA transcript expression. The results are expressed as percent inhibition of DMPK transcript, relative to the PBS control.

The results indicate that treatment with the antisense oligonucleotides resulted in inhibition of DMPK mRNA in the mice.

TABLE 39
Antisense inhibition of human DMPK
RNA transcript in LC15 mice

| | | % |
|---|---|---|
| ISIS No | mg/kg/wk | inhibition |
| 444404 | 50.00 | 24 |
| 444404 | 50.00 | 30 |
| 444436 | 50.00 | 17 |
| 473810 | 25.00 | 7 |
| 473810 | 12.50 | 18 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. Since LC15 mice do not have myotonia, neither the control group nor the treatment groups displayed any myotonia in any muscle tested.

### Example 25: In Vivo Antisense Inhibition of Human DMPK in SXL Transgenic Mouse Model

Using hDMPK-targeting ASOs 444401 and 299471 target knockdown in soleus muscle was measured in SXL mice. The SXL mouse is transgenic for the entire DMPK gene and promoter and contains a 1000 CUG repeat sequence in the 3′UTR of DMPK gene. Mice were dosed 50 mg/kg twice weekly for 4 weeks (n=3 mice per group, except n=2 for saline-injected controls). Results of Taqman assays demonstrated that treatment with either ISISI 444401 or ISIS 299471 significantly reduced mut-hDMPK mRNA levels but had negligible effect on endogenous mouse Dmpk mRNA levels.

Therefore, ISIS 444401 and ISIS 299471 selectively target human DMPK mRNA transcript.

### Example 26: Duration of Action of Antisense Inhibition by an Oligonucleotide Targeting Human Alpha1 Actin in Transgenic Mice

The duration of action of antisense inhibition of human alpha1 actin RNA transcript by ISIS 190401 in HSA^{LR} mice was evaluated.

### Treatment

HSA^{LR} mice received subcutaneous injections of ISIS 190401 at a dose of 25 mg/kg twice per week for 4 weeks. A control group received subcutaneous injections of PBS twice per week for 4 weeks. The PBS-injected group served as the control group to which the oligonucleotide-treated group was compared. The mice were analyzed 15 weeks after administration of the last dose.

### Inhibition of Alpha1 Actin RNA

Fifteen weeks after the final dose, the animals were sacrificed and tissue from the quadriceps muscles, gastrocnemius muscles, and tibialis anterior muscles was isolated. RNA was isolated for real-time PCR analysis of alpha1 actin and normalized to 18s RNA. As presented in Table 40, treatment with ISIS 190401 reduced human alpha1 actin RNA transcript expression, and this effect was sustained at least for 15 weeks. The results are expressed as percent inhibition of alpha1 actin transcript, relative to the PBS control.

Treatment with ISIS 190401 resulted in significant inhibition of alpha1 actin mRNA levels under the conditions specified above.

TABLE 40
Antisense inhibition of human alpha1
actin RNA transcript in HSA^{LR} mice

| | % |
|---|---|
| Muscle Type | inhibition |
| Quadriceps | 74 |
| Gastrocnemius | 81 |
| Tibialis anterior | 75 |

### Assessment of Myotonia by Electromyography

Electromyography on left and right quadriceps, left and right gastrocnemius muscles, left and right tibialis anterior muscles and lumbar paraspinals muscles was performed as previously described (Kanadia et al, 2003, Science, 302: 1978-1980) by using 30 gauge concentric needle electrodes and a minimum of 10 needle insertions for each muscle. The data is presented in Table 41 as the average myotonia grade observed in four mice of each group and demonstrates significant reduction of myotonia in mice treated with ISIS 190401. Therefore, the effect of antisense inhibition of alpha actin by ISIS 190401 was sustained at least for 15 weeks.

TABLE 41
Average reduction of myotonia in various muscles
of antisense oligonucleotide-treated HSA^{LR} mice

| | | ISIS |
|---|---|---|
| | PBS | 190401 |
| Left quadriceps | 3.0 | 0.0 |
| Right quadriceps | 3.0 | 0.0 |
| Left gastrocnemius | 2.5 | 0.0 |
| Right gastrocnemius | 2.5 | 0.0 |
| Left Tibialis anterior | 2.5 | 0.0 |
| Right Tibialis anterior | 2.5 | 0.0 |
| Lumbar paraspinals | 2.5 | 0.0 |

### Correction of Alternative Splicing

To evaluate the effect of ISIS 190401 on alternative splicing of Serca1, total RNA purified from the tibialis anterior gastrocnemius, and quadriceps muscle was analyzed in a procedure similar to that described in Example 13.

In PBS treated HSA^{LR} mice, Serca1 splicing is dysregulated as demonstrated by exon 22 exclusion. Treatment with ISIS 190401 resulted in complete inclusion and normalization of alternative splicing of exon 22 of the Serca1 gene in all three muscle types, which was sustained even after 15 weeks.

Therefore, antisense inhibition of alpha1 actin corrected Serca1 splicing dysregulation, which indicates that treatment with antisense oligonucleotide reduced accumulation of CUGexp in the nuclear foci. Reduced accumulation of CUGexp in the nuclear foci corrects MBLN1 sequestration thereby allowing normal splicing to occur.

### Example 27: Microarray Analysis of Transcriptomic Effect of Antisense Inhibition of Human Actin

Expression of actin mRNA with expanded CUG repeats causes extensive remodeling of the muscle transcriptome. To evaluate the overall transcriptomic effects of ISIS 190401 and ISIS 445236, microarray analyses was utilized in HSA^{LR} mice.

### Treatment

HSA^{LR} mice received subcutaneous injections of ISIS 190401 or ISIS 445236 at a dose of 25 mg/kg twice per week for 4 weeks. A control group received subcutaneous injections of PBS twice per week for 4 weeks. The PBS-injected group served as the control group to which the oligonucleotide-treated group was compared.

### Transcriptome Analysis by Microarray

RNA was isolated from the quadriceps muscle of wild-type or HSA^{LR} mice. RNA integrity was verified using an Agilent Bioanalyzer (RNA integrity number >7.5). RNA was processed to complementary RNA (cRNA) and hybridized on microbeads using MouseRef-8 v2.0 Expression BeadChip Kits (Illumina, San Diego), according to the manufacturer's recommendations. Image data were quantified using BeadStudio software (Illumina). Signal intensities were quantile normalized. Row-specific offsets were used to avoid any values of less than 2 prior to normalization. Data from all probe sets with 6 or more nucleotides of CUG, UGC, or GCU repeats was suppressed to eliminate the possibility that expanded repeats in the hybridization mixture (CAG repeats in cRNA originating from CUG repeats in the mRNA) could cross-hybridize with repeat sequences in the probes. To eliminate genes whose expression was not readily quantified on the arrays, probes showing a P value for detection probability of <0.1 were suppressed in all samples. Comparisons between groups were summarized and rank-ordered by fold-changes of mean expression level and t tests. The software package R (Butler et al. Diabetes. 2002; 51: 1028-34) was used to perform principal components analysis (Levin et al. In Antisense Drug Technology: Principles, Strategies, and Applications, S. T. Crooke, Ed. (CRC Press, Boca Raton, 2008), pp 183-215; Geary et al. Drug Metab. Dispos. 2003; 31: 1419-28) on wild-type, ISIS oligonucleotide-treated, and PBS-treated microarray samples. The principle components allowed the capture of the majority of the expression variation in each sample within 3 dimensions. The first three principal components of each sample were plotted.

The principle component analysis of untreated wild-type and HSA^{LR} mice demonstrated segregation of HSA^{LR} away from wild-type mice, in widely separated clusters. In contrast, antisense oligonucleotide-treated HSA^{LR} mice clustered more closely to wild-type mice, suggesting an overall trend for transcriptome normalization. Comparisons of HSA^{LR} transgenic mice with wild-type mice identified 93 transcripts whose expression levels were altered more than two-fold (P<0.0001), as presented in Table 42, below. The extent of dysregulation for these transcripts was reduced or normalized for antisense oligonucleotides (88% dysregulated transcripts responded to ISIS 445236, P<0.05 for ISIS 445236 vs. PBS control, whereas 90% responded to ISIS 190401).

In order to consider transcripts that have off-target knockdown, all transcripts whose expression was reduced in antisense oligonucleotide-treated HSALR mice were identified (>two-fold reduction by either oligonucleotide, P<0.0001, n=41 transcripts). All transcripts that were down-regulated by these criteria demonstrated upregulation in HSA^{LR} mice. The only exception, collagen 6 alpha2, is unlikely to result from off-target cleavage because it was down-regulated by the two antisense oligonucleotides with non-overlapping sequences.

These results indicate that treatment with antisense oligonucleotides for 4 weeks resulted in a general improvement of the muscle transcriptome without any evidence for off-target effects.

TABLE 42
Comparisons of HSA^{LR} transgenic mice with wild-type mice identified 93 transcripts

| | Fold- | | Fold- | t test | Fold- | | Fold- | t test | Fold- | |
|---|---|---|---|---|---|---|---|---|---|---|
| | change | t test | change | HASLR | change | t test | change | HSALR- | change | t test |
| | HSALR- | HSALR- | HSALR- | 190401 | HSALR- | HSALR- | HSALR- | 445236 | HSALR- | HSALR- |
| | saline | Saline | 190104 vs. | vs. | 190401 | 190401 | 445236 vs. | vs. | 445236 | 445236 |
| | vs. | vs. | HSALR- | HSALR- | vs. | vs. | HSALR- | HSALR- | vs. | vs. |
| Transcript | WT | WT | saline | saline | WT | WT | saline | saline | WT | WT |
| OSBPL10 | 15.11 | 0.0000 | 0.46 | 0.0023 | 6.95 | 0.0008 | 0.39 | 0.0007 | 5.92 | 0.0002 |
| FBXL13 | 12.12 | 0.0000 | 0.49 | 0.0159 | 5.91 | 0.0385 | 0.65 | 0.0255 | 7.93 | 0.0026 |
| NGFR | 11.57 | 0.0000 | 0.23 | 0.0001 | 2.66 | 0.0314 | 0.16 | 0.0000 | 1.84 | 0.0133 |
| SLC1A1 | 9.39 | 0.0000 | 0.39 | 0.0001 | 3.66 | 0.0001 | 0.30 | 0.0001 | 2.85 | 0.0116 |
| CXADR | 9.13 | 0.0000 | 0.14 | 0.0000 | 1.30 | 0.6119 | 0.21 | 0.0001 | 1.94 | 0.2244 |
| NFATC2 | 8.48 | 0.0000 | 0.32 | 0.0002 | 2.67 | 0.0043 | 0.22 | 0.0001 | 1.84 | 0.0394 |
| ATP1B4 | 7.02 | 0.0000 | 0.24 | 0.0000 | 1.68 | 0.0021 | 0.24 | 0.0000 | 1.70 | 0.0091 |
| UCHL1 | 6.80 | 0.0000 | 0.71 | 0.0168 | 4.86 | 0.0005 | 0.72 | 0.1187 | 4.91 | 0.0090 |
| TEAD4 | 6.76 | 0.0000 | 0.50 | 0.0030 | 3.39 | 0.0085 | 0.30 | 0.0004 | 2.06 | 0.1213 |
| TAS1R1 | 6.72 | 0.0000 | 0.28 | 0.0003 | 1.91 | 0.1857 | 0.43 | 0.0002 | 2.88 | 0.0047 |
| MUSTN1 | 6.52 | 0.0000 | 0.31 | 0.0000 | 2.01 | 0.0006 | 0.33 | 0.0000 | 2.15 | 0.0115 |
| IRF5 | 6.01 | 0.0000 | 0.21 | 0.0000 | 1.28 | 0.0556 | 0.33 | 0.0001 | 1.96 | 0.0035 |
| CRIP3 | 5.82 | 0.0000 | 0.33 | 0.0000 | 1.92 | 0.0151 | 0.29 | 0.0001 | 1.67 | 0.1470 |
| TAL2 | 5.75 | 0.0000 | 0.20 | 0.0001 | 1.13 | 0.7717 | 0.36 | 0.0002 | 2.08 | 0.0274 |
| ORF63 | 5.39 | 0.0000 | 0.27 | 0.0001 | 1.45 | 0.0206 | 0.47 | 0.0018 | 2.51 | 0.0066 |
| COPG | 5.05 | 0.0000 | 0.30 | 0.0000 | 1.53 | 0.0218 | 0.25 | 0.0001 | 1.25 | 0.3617 |
| CAMK1D | 4.92 | 0.0000 | 0.23 | 0.0002 | 1.12 | 0.8157 | 0.27 | 0.0000 | 1.32 | 0.2449 |
| HSPA2 | 4.76 | 0.0000 | 0.43 | 0.0000 | 2.02 | 0.0079 | 0.42 | 0.0000 | 2.02 | 0.0197 |
| CAMK2D | 4.70 | 0.0000 | 0.36 | 0.0001 | 1.70 | 0.0493 | 0.45 | 0.0004 | 2.12 | 0.0095 |
| CNTNAP2 | 4.49 | 0.0000 | 0.58 | 0.0001 | 2.59 | 0.0000 | 0.67 | 0.0007 | 3.02 | 0.0000 |
| TTC7 | 4.33 | 0.0000 | 0.38 | 0.0000 | 1.63 | 0.0085 | 0.68 | 0.0468 | 2.96 | 0.0126 |
| CD276 | 4.08 | 0.0001 | 0.36 | 0.0001 | 1.47 | 0.1613 | 0.59 | 0.0029 | 2.39 | 0.0072 |
| USH1C | 4.07 | 0.0000 | 0.50 | 0.0011 | 2.04 | 0.0077 | 0.38 | 0.0029 | 1.55 | 0.2881 |
| LRP11 | 4.03 | 0.0000 | 0.55 | 0.0017 | 2.24 | 0.0011 | 0.55 | 0.0006 | 2.23 | 0.0000 |
| PHLDA3 | 3.96 | 0.0000 | 0.40 | 0.0001 | 1.60 | 0.0019 | 0.36 | 0.0001 | 1.42 | 0.0609 |
| HSPB7 | 3.80 | 0.0000 | 0.30 | 0.0000 | 1.14 | 0.5358 | 0.30 | 0.0000 | 1.15 | 0.4474 |
| TRIT1 | 3.74 | 0.0000 | 0.43 | 0.0000 | 1.62 | 0.0003 | 0.31 | 0.0000 | 1.16 | 0.1043 |
| PCNX | 3.66 | 0.0000 | 0.37 | 0.0002 | 1.34 | 0.1628 | 0.42 | 0.0001 | 1.53 | 0.0105 |
| 3632451O06RIK | 3.51 | 0.0000 | 0.81 | 0.1094 | 2.83 | 0.0025 | 0.71 | 0.0015 | 2.51 | 0.0002 |
| AMHR2 | 3.46 | 0.0000 | 0.45 | 0.0001 | 1.56 | 0.0037 | 0.52 | 0.0003 | 1.79 | 0.0016 |
| SNX13 | 3.27 | 0.0000 | 0.47 | 0.0000 | 1.55 | 0.0007 | 0.44 | 0.0000 | 1.42 | 0.0003 |
| ATP9A | 3.26 | 0.0000 | 0.60 | 0.0001 | 1.96 | 0.0024 | 0.42 | 0.0002 | 1.38 | 0.2009 |
| D030028O16RIK | 3.22 | 0.0000 | 0.53 | 0.0011 | 1.70 | 0.0104 | 0.48 | 0.0001 | 1.56 | 0.0007 |
| RPS6KA3 | 3.09 | 0.0000 | 0.38 | 0.0000 | 1.17 | 0.1845 | 0.44 | 0.0001 | 1.37 | 0.0321 |
| GCA | 3.00 | 0.0000 | 0.70 | 0.0031 | 2.09 | 0.0005 | 0.74 | 0.0103 | 2.22 | 0.0006 |
| PACRG | 2.89 | 0.0001 | 0.51 | 0.0002 | 1.46 | 0.0063 | 0.46 | 0.0001 | 1.34 | 0.0229 |
| SPSB2 | 2.88 | 0.0001 | 0.33 | 0.0000 | 0.95 | 0.6599 | 0.37 | 0.0000 | 1.07 | 0.6216 |
| POU4F1 | 2.83 | 0.0000 | 0.42 | 0.0000 | 1.19 | 0.2046 | 0.60 | 0.0007 | 1.68 | 0.0074 |
| STRN4 | 2.72 | 0.0000 | 0.38 | 0.0000 | 1.03 | 0.8900 | 0.46 | 0.0000 | 1.25 | 0.2128 |
| NCAM1 | 2.67 | 0.0001 | 0.70 | 0.0259 | 1.87 | 0.0135 | 0.54 | 0.0006 | 1.43 | 0.0343 |
| A930018M24Rik | 2.65 | 0.0001 | 0.58 | 0.0058 | 1.53 | 0.0727 | 0.43 | 0.0002 | 1.13 | 0.3919 |
| TUBA4A | 2.60 | 0.0000 | 0.42 | 0.0000 | 1.09 | 0.1806 | 0.50 | 0.0000 | 1.31 | 0.0041 |
| 1AP | 2.57 | 0.0000 | 0.57 | 0.0002 | 1.46 | 0.0108 | 0.59 | 0.0016 | 1.52 | 0.0333 |
| ANKRD40 | 2.56 | 0.0000 | 0.63 | 0.0155 | 1.60 | 0.0683 | 0.57 | 0.0002 | 1.46 | 0.0047 |
| UVRAG | 2.48 | 0.0000 | 0.59 | 0.0000 | 1.48 | 0.0005 | 0.52 | 0.0000 | 1.28 | 0.0165 |
| HIST1H4H | 2.46 | 0.0001 | 0.55 | 0.0001 | 1.34 | 0.0474 | 0.65 | 0.0014 | 1.60 | 0.0125 |
| EPS15 | 2.44 | 0.0000 | 0.61 | 0.0001 | 1.50 | 0.0057 | 0.77 | 0.0043 | 1.87 | 0.0007 |
| PANX1 | 2.41 | 0.0001 | 0.46 | 0.0004 | 1.11 | 0.4311 | 0.36 | 0.0000 | 0.87 | 0.0561 |
| CALML4 | 2.41 | 0.0001 | 0.45 | 0.0008 | 1.10 | 0.6994 | 0.67 | 0.0154 | 1.62 | 0.0538 |
| ASPH | 2.40 | 0.0000 | 0.40 | 0.0000 | 0.95 | 0.6969 | 0.44 | 0.0000 | 1.05 | 0.7267 |
| CREB3L2 | 2.37 | 0.0001 | 0.71 | 0.0287 | 1.67 | 0.0416 | 0.65 | 0.0051 | 1.54 | 0.0410 |
| TRAF3 | 2.32 | 0.0001 | 0.50 | 0.0001 | 1.16 | 0.2851 | 0.57 | 0.0001 | 1.32 | 0.0481 |
| CMYA1 | 2.30 | 0.0000 | 0.44 | 0.0007 | 1.02 | 0.9450 | 0.44 | 0.0000 | 1.01 | 0.9265 |
| ADAMTSL5 | 2.30 | 0.0001 | 0.48 | 0.0000 | 1.11 | 0.3365 | 0.53 | 0.0004 | 1.22 | 0.1827 |
| HS2ST1 | 2.27 | 0.0001 | 0.64 | 0.0002 | 1.44 | 0.0223 | 0.74 | 0.0041 | 1.68 | 0.0062 |
| HIST1H4J | 2.21 | 0.0000 | 0.59 | 0.0000 | 1.31 | 0.0283 | 0.72 | 0.0002 | 1.60 | 0.0023 |
| SPSB1 | 2.20 | 0.0000 | 0.53 | 0.0005 | 1.16 | 0.2409 | 0.48 | 0.0000 | 1.05 | 0.3088 |
| LANCL1 | 2.20 | 0.0000 | 0.63 | 0.0002 | 1.39 | 0.0002 | 0.66 | 0.0006 | 1.46 | 0.0005 |
| KCNC4 | 2.16 | 0.0000 | 0.91 | 0.3892 | 1.96 | 0.0036 | 0.98 | 0.8712 | 2.12 | 0.0029 |
| PRRC1 | 2.16 | 0.0000 | 0.57 | 0.0001 | 1.23 | 0.0324 | 0.59 | 0.0000 | 1.26 | 0.0070 |
| MID1IP1 | 2.13 | 0.0001 | 1.27 | 0.0161 | 2.70 | 0.0001 | 1.09 | 0.4336 | 2.32 | 0.0014 |
| DICER1 | 2.13 | 0.0000 | 0.65 | 0.0006 | 1.39 | 0.0051 | 0.69 | 0.0018 | 1.47 | 0.0035 |
| IKBKB | 2.10 | 0.0001 | 0.74 | 0.0240 | 1.56 | 0.0262 | 0.78 | 0.0039 | 1.64 | 0.0015 |
| D5WSU178E | 2.10 | 0.0000 | 0.86 | 0.1447 | 1.80 | 0.0049 | 0.88 | 0.0352 | 1.84 | 0.0002 |
| ZFP106 | 2.08 | 0.0000 | 0.53 | 0.0000 | 1.11 | 0.1324 | 0.58 | 0.0002 | 1.20 | 0.0706 |
| B930041F14RIK | 2.06 | 0.0000 | 0.71 | 0.0002 | 1.47 | 0.0000 | 0.72 | 0.0030 | 1.49 | 0.0025 |
| FHL1 | 2.04 | 0.0000 | 0.58 | 0.0000 | 1.17 | 0.1332 | 0.40 | 0.0000 | 0.81 | 0.0815 |
| UHRFIBP1L | 2.04 | 0.0001 | 0.78 | 0.0315 | 1.59 | 0.0071 | 0.68 | 0.0024 | 1.38 | 0.0151 |
| PHCA | 2.02 | 0.0000 | 0.64 | 0.0001 | 1.29 | 0.0354 | 0.74 | 0.0070 | 1.50 | 0.0145 |
| B230312A22RIK | 2.02 | 0.0000 | 0.79 | 0.0022 | 1.59 | 0.0004 | 0.77 | 0.0019 | 1.56 | 0.0007 |
| PPP2R5C | 2.01 | 0.0000 | 0.59 | 0.0001 | 1.16 | 0.0161 | 0.66 | 0.0017 | 1.32 | 0.0177 |
| UCK2 | 2.01 | 0.0001 | 0.70 | 0.0004 | 1.41 | 0.0129 | 0.64 | 0.0001 | 1.28 | 0.0510 |
| LEPROTL1 | 0.50 | 0.0000 | 1.45 | 0.0013 | 0.72 | 0.0004 | 1.47 | 0.0011 | 0.73 | 0.0005 |
| COPS7A | 0.49 | 0.0000 | 1.35 | 0.0645 | 0.66 | 0.0039 | 1.49 | 0.0026 | 0.73 | 0.0016 |
| PRM17 | 0.48 | 0.0001 | 1.51 | 0.2023 | 0.73 | 0.1585 | 1.34 | 0.0445 | 0.65 | 0.0002 |
| LDB3 | 0.47 | 0.0000 | 1.55 | 0.0550 | 0.73 | 0.0607 | 1.57 | 0.0010 | 0.74 | 0.0055 |
| LOC100046120 | 0.47 | 0.0000 | 1.31 | 0.0077 | 0.61 | 0.0000 | 1.27 | 0.0381 | 0.60 | 0.0002 |
| LOC677317 | 0.45 | 0.0001 | 1.49 | 0.0004 | 0.68 | 0.0012 | 1.93 | 0.0011 | 0.88 | 0.2082 |
| LDB2 | 0.45 | 0.0000 | 1.73 | 0.0424 | 0.78 | 0.1234 | 1.23 | 0.0817 | 0.56 | 0.0000 |
| SUM03 | 0.44 | 0.0000 | 1.70 | 0.0123 | 0.74 | 0.0223 | 1.37 | 0.0960 | 0.60 | 0.0023 |
| LRRC24 | 0.43 | 0.0001 | 1.89 | 0.0009 | 0.82 | 0.0212 | 1.42 | 0.0898 | 0.61 | 0.0041 |
| HNRPH1 | 0.42 | 0.0000 | 1.64 | 0.0077 | 0.69 | 0.0094 | 1.70 | 0.0057 | 0.71 | 0.0144 |
| ARMETL1 | 0.38 | 0.0000 | 2.58 | 0.0000 | 0.98 | 0.7666 | 2.70 | 0.0000 | 1.02 | 0.7109 |
| LOC100041504 | 0.37 | 0.0000 | 2.02 | 0.0001 | 0.75 | 0.0061 | 1.84 | 0.0040 | 0.68 | 0.0094 |
| MMP9 | 0.32 | 0.0000 | 2.40 | 0.0006 | 0.77 | 0.0340 | 1.37 | 0.1834 | 0.44 | 0.0009 |
| CBFB | 0.28 | 0.0000 | 2.66 | 0.0304 | 0.75 | 0.1852 | 1.94 | 0.0056 | 0.55 | 0.0004 |
| MDH2 | 0.24 | 0.0000 | 1.20 | 0.0473 | 0.29 | 0.0000 | 1.12 | 0.1037 | 0.27 | 0.0000 |
| APCDD1 | 0.20 | 0.0000 | 1.98 | 0.2157 | 0.39 | 0.0059 | 4.55 | 0.0001 | 0.90 | 0.2873 |
| LOC654842 | 0.19 | 0.0000 | 1.28 | 0.1712 | 0.24 | 0.0000 | 1.07 | 0.8807 | 0.20 | 0.0001 |
| F2RL3 | 0.15 | 0.0000 | 5.78 | 0.0001 | 0.86 | 0.1901 | 4.92 | 0.0004 | 0.73 | 0.0310 |
| EIF3H | 0.13 | 0.0000 | 1.99 | 0.2185 | 0.26 | 0.0001 | 1.86 | 0.1997 | 0.24 | 0.0000 |
| AVIL | 0.12 | 0.0000 | 4.22 | 0.0156 | 0.52 | 0.0081 | 1.88 | 0.2270 | 0.23 | 0.0001 |
| ACTC1 | 0.08 | 0.0000 | 1.42 | 0.0346 | 0.11 | 0.0000 | 6.07 | 0.0098 | 0.48 | 0.0087 |

1.-124. (canceled)

125. A compound comprising a modified oligonucleotide consisting of 12 to 30 linked nucleosides wherein the nucleobase sequence of the modified oligonucleotide is at least 85% complementary to at least 12 contiguous nucleobases of:
an equal length portion of nucleobases 178-230 of SEQ ID NO: 1;
an equal length portion of nucleobases 232-253 of SEQ ID NO: 1;
an equal length portion of nucleobases 279-299 of SEQ ID NO: 1;
an equal length portion of nucleobases 366-399 of SEQ ID NO: 1;
an equal length portion of nucleobases 519-541 of SEQ ID NO: 1;
an equal length portion of nucleobases 923-975 of SEQ ID NO: 1;
an equal length portion of nucleobases 1073-1105 of SEQ ID NO: 1;
an equal length portion of nucleobases 1171-1196 of SEQ ID NO: 1;
an equal length portion of nucleobases 1178-1206 of SEQ ID NO: 1;
an equal length portion of nucleobases 1215-1246 of SEQ ID NO: 1;
an equal length portion of nucleobases 1263-1324 of SEQ ID NO: 1;
an equal length portion of nucleobases 1706-1734 of SEQ ID NO: 1;
an equal length portion of nucleobases 1743-1763 of SEQ ID NO: 1;
an equal length portion of nucleobases 1932-1979 of SEQ ID NO: 1;
an equal length portion of nucleobases 1981-2003 of SEQ ID NO: 1;
an equal length portion of nucleobases 2077-2108 of SEQ ID NO: 1;
an equal length portion of nucleobases 2152-2173 of SEQ ID NO: 1;
an equal length portion of nucleobases 2159-2182 of SEQ ID NO: 1;
an equal length portion of nucleobases 2174-2196 of SEQ ID NO: 1;
an equal length portion of nucleobases 2426-2447 of SEQ ID NO: 1;
an equal length portion of nucleobases 2450-2518 of SEQ ID NO: 1;
an equal length portion of nucleobases 2679-2725 of SEQ ID NO: 1;
an equal length portion of nucleobases 1251-1303 of SEQ ID NO:2;
an equal length portion of nucleobases 1305-1326 of SEQ ID NO:2;
an equal length portion of nucleobases 1352-1372 of SEQ ID NO:2;
an equal length portion of nucleobases 3762-3795 of SEQ ID NO:2;
an equal length portion of nucleobases 4170-4192 of SEQ ID NO:2;
an equal length portion of nucleobases 5800-5852 of SEQ ID NO:2;
an equal length portion of nucleobases 6124-6149 of SEQ ID NO:2;
an equal length portion of nucleobases 6168-6199 of SEQ ID NO:2;
an equal length portion of nucleobases 6216-6277 of SEQ ID NO:2;
an equal length portion of nucleobases 11979-12007 of SEQ ID NO:2;
an equal length portion of nucleobases 12016-12036 of SEQ ID NO:2;
an equal length portion of nucleobases 12993-13042 of SEQ ID NO:2;
an equal length portion of nucleobases 13044-13066 of SEQ ID NO:2;
an equal length portion of nucleobases 13140-13171 of SEQ ID NO:2; or
an equal length portion of nucleobases 13215-13236 of SEQ ID NO:2;
wherein the modified oligonucleotide comprises at least one modified sugar moiety and/or at least one modified internucleoside linkage.

126. A compound comprising a modified oligonucleotide consisting of 12 to 30 linked nucleosides, wherein the nucleobase sequence of the modified oligonucleotide comprises at least 12 contiguous nucleobases of any of the sequences recited in SEQ ID NOs: 12-156, 160-697, or 802-837, and wherein the modified oligonucleotide comprises at least one modified sugar moiety and/or at least one modified internucleoside linkage.

127. The compound of claim 125, wherein the modified oligonucleotide is a single-stranded oligonucleotide.

128. The compound of claim 125, wherein the modified oligonucleotide is a double-stranded oligonucleotide.

129. The compound of claim 125, wherein the nucleobase sequence of the modified oligonucleotide is 90% or 100% complementary to SEQ ID NO: 1 or SEQ ID NO: 2.

130. The compound of claim 125, wherein at least one internucleoside linkage is a modified internucleoside linkage.

131. The compound of claim 125, wherein at least one internucleoside linkage is a phosphorothioate internucleoside linkage.

132. The compound of claim 125, wherein each internucleoside linkage is a phosphorothioate internucleoside linkage.

133. The compound of claim 125, wherein at least one nucleoside comprises a modified sugar.

134. The compound of claim 133, wherein the modified sugar is a bicyclic sugar.

135. The compound of claim 133, wherein the modified sugar comprises a 2′-O-methoxyethyl.

136. The compound of claim 125, wherein at least one nucleoside comprises a modified nucleobase.

137. The compound of claim 136, wherein the modified nucleobase is a 5-methylcytosine.

138. The compound of claim 125, wherein the modified oligonucleotide comprises:
a gap segment consisting of 6 to 14 linked deoxynucleosides;
a 5′ wing segment consisting of 3 to 8 linked nucleosides;
a 3′ wing segment consisting of 3 to 8 linked nucleosides;
wherein the gap segment is positioned between the 5′ wing segment and the 3′ wing segment, and
wherein each nucleoside of each wing segment comprises a modified sugar.

139. The compound of claim 125, wherein the modified oligonucleotide consists of 20 linked nucleosides and comprises:
a gap segment consisting of ten linked deoxynucleosides;
a 5′ wing segment consisting of five linked nucleosides;
a 3′ wing segment consisting of five linked nucleosides;
wherein the gap segment is positioned between the 5′ wing segment and the 3′ wing segment,
wherein each nucleoside of each wing segment comprises a 2′-O-methoxyethyl sugar, and
wherein each internucleoside linkage is a phosphorothioate linkage.

140. The compound of claim 125, wherein the modified oligonucleotide consists of 18 to 22 linked nucleosides.

141. The compound of claim 125, further comprising a conjugate group.

142. A composition comprising the compound of claim 125 and a pharmaceutically acceptable carrier or diluent.

143. A method of treating a DMPK related disease or a symptom thereof in an animal, wherein the method comprises administering to said animal a therapeutically effective amount of the compound of claim 125.

144. The method of claim 143, wherein the DMPK related disease is type 1 myotonic dystrophy.

145. The method of claim 143, wherein the DMPK related disease is MBNL dependent spliceopathy.


SEQUENCE LISTING (837 sequences)

SEQ ID NO: 1 - DNA, 2877 bp, Homo sapiens
  source 1..2877: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaaaaaaaaaaaaaaa

SEQ ID NO: 2 - DNA, 14411 bp, Homo sapiens
  source 1..14411: mol_type = genomic DNA
ctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaag

SEQ ID NO: 3 - DNA, 15000 bp, Mus musculus
  source 1..15000: mol_type = genomic DNA
cagtgtccccactgcccaaggctggctccatcacgtaccgctttggctcagctggccaggacacacagttctgcctgtgggacctcacagaagatgtgctctcccctcatccgtctctggcccgtacccgcacccttccgggcacacctggtgccaccccaccagcttctggtagttctcgggccggagagacaggtgcaggccccctgccccgctccctgtctcgttccaacagtctcccacacccagctggtggtggcaaggctggtgggcctagtgcatcgatggagcctggcataccattcagcattggccgctttgccacactgaccctgcaggagcggcgggaccggggagctgagaaggaacacaaacgctaccatagcctgggaaacatcagccgcggtggcagtgggggcaatagcagcaatgacaagctcagtggtcctgccccccgaagccgattggacccagctaaggtgctgggcacggcactgtgccctcggatccatgaggtgccactgctggagcctctcgtgtgcaagaagattgctcaggaacgcctgaccgtgctactgttcctggaggattgtatcatcactgcctgccaagagggcctcatctgcacctgggcccggccaggcaaggcggtgagtccgcacctgcccaagcgctgaggggcaccagttctgtccctaccggatgccagttatccgtcagcagaaaggtcaggtataggagacagaatggggggaaccacagctaacgtctttagagcctctgctggcccatatggctcatccttagtacttcacactcaaggcagaacctgtgtttataggaaatctgaagtgtagatggtgaaactttattcaggtctagggatgtgattgagctgggggcccacttctggcctgcctcttagacactgtttctgagccagctgctgaaggcctggatgggaattagccagggtccaggcctgcacttcctcttgctgctgtgtggtcctggtcattgggtctcacagatgggctgtgcagtggctgtgctcttagttggtgaggtgcaggcctgtcacctggtcaggcttgagcatgtggtctcagtgtctaggaccctactctgccctcagtccttcagtcccttgctttggaaggctagagtccagaagccttagaacgtcaggcagttgcagagccactgccaggctagtagggctgcgggagttgactgagttctcacagacacccctctgtctccctagttcacagacgaggagaccgaggcccaggcagggcaagcaagttggcccaggtcacccagcaagtcagttgtagaggtaggacaacccctgaagctgcaagtggaccccagtttcttttctctccactgtcgtcccctgtatgcccaggacacctggggccacattactgtggaagtgctactctgggtcagtggagacggccgagctgtttgttcctagctaggacagcagctttaggcctggggggcagatcccagctggggcagcagctccaaggcctttgggtggctccttctccgggttctggcagaagcccaggtgctgtctaatccacctttctcctcttgttctccccagggcatctcctcccaaccaggcagctcccccagtggcactgtggtgtgaaatgtggatgtcccatgttcccggcctcctagccataaccctccccgctgacctcaagaatcactgtattaacaagactaatcatgatggaaggactgctccaagccccacgctgcacacatactgggggtcccctaggttggcccagccatggggatgtagtgtcctgtgtggccttggccctgtcctccacccactgccaagtacaatgacctgttctctgaaacatcagtgttaaccatatccctgtcccagcatgtgactgttcactcctgggagagacttagcccacagtacccctgggtgagagggcagggcaggggccatccccactcctgcccaaactccaccccttgctatggtctgtgattttgaaagtgttaaattatggaagccctgagggccctccttgttcccctggacctcttatttatactaaagtccttgtttgcacagtgtttctgttccctggggcagggtagggtgggggttgcagtacttggcctccaagctgtgctctgaccaaaggaagcccaatcttagctgtttccccatccctagccccgagcagagagccctctgaaagatgagtctcgacccccaaagtcaagaggctgagatggccttcctactaggtccttggagatgtttgaaacttgttttaaacaccaggactatccaagcatgctctccttggggagaggaggatgctggaattgactgcactccctgcctcctctgaacatgcctttgcagtctgctgcccctggcccatttatgactggccatctagtgccagctggaggtcatgatttcctccccagagaactggccaccctagaaagaagctaacttgtcgcctggcttgctgtccaggcagctccgccctcaacccctaaaatgtttctgtctctaatcctagcccaggcaggaatgtggctgccccggcctgtggccaaggagctattttggggttctcttttgcttaaggagggcctggatccaccacttgcctcccccaggctggggccagcaggtcacccctggccctggcggctgagcaaactctctcctgatcttccttctacctcctgccaaaaaatgggggggcgggtaatacagcaggcacaggggctaaatttaactgtcccaaagtcggaatccattgctgagtcacgaagaagctgcccctggcctttgccccccccactaccccctcaccccctgttgcccaggcatcagccctttcccccaacccctcccagctctgagtctatagactggctctcctgggcactgacacctcccacctgtaactccctgtgctctctttatgggtgggtagagtcaatgggggggggcaaccctggagtattactctgtcccctgacattgggctctgaagagttttgaggggccctggaagaagggagttggggtgttggctcaggaggggttaaaaactgggaggcgggaggggggctgggccaaggggtggagaaaagaggaggaggccttaagcatagaactggccagagagacccaagggatagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtggggtgagtatggataggaagcctggggttgggtgcaaggcagaggtgggtctacagggcaagaatgggctatggaggggcaggagggcctggaaagggctttttgtaagggagccaagcagagctcatgacctgaccccaagctcccctggtgaggcaccagggtcagtgaggccacctatgactcagccagtgcaggctggggtgggcatagcctcctgctatctcagcacccacactaggacctggcagctttctcttttaggacccttggctcctcaaactggcttcatagccctccccagtttcccagagtgtggggagggacagcgtggggcagctgccagggtgtggcccataggcaggtgtttggcgtctgcctccccagctgccctgacaggtgtccaggagctatgagggcactgtgactcacagaggccctgggggagaaccagcccggcagacaggcgccaccgagcaccctttctgttccccaaattaagaggaaggaacaacttcagcttctgagtgtgcccatccctagcactctgatcccgcccagcctttgtgggccagattggtcatccctcctggcttctcatctgcttttgtggttctagctcaagacctctaattcctctgctgacttaaatgcccttccccagaggtcttctcaggcctagtggacaagcttggagccttatctgctcctgcccaacattgagccaaagctccagcttaccccagcttccttacaagtaacgacctgttttgttgctctgtgcctattattaagggtccaggtcttgattcttggctgtctgcccatgtgtgtgaccctagtgcattctcccctcctcccccgtttcacagatggaaaggttgaggccatcggttagactgctaagcctgtgaaagactttttctcctctccagtctttagtgtctccctcaacctttcttttgaaggatggggtttgggctggcagggtaaactgagaactggggtgggggcagggggtctgaccctctgggaaggagcagtccttttgtggcctgagcagcatcctgtgggcccctcccctgccaggcctgggcgggggagggggcctgggttcccgctgccttaaaagggctcaacgccttggctctctcctcctccccaccccccagccttggccctagctgtatcttccccggctgcccactttcccaaacccctttcttctctgtgaccccatctccccgcttccccacacgtccctcctccatccttactccccggccttagaacttccctaagggagatctgacctccctctgcccaccccgcacccccagtcgccagcctcagacctagctgctctcccctctggctgaaccaccctagcacaggaccttataccctggagctttggttataagaagactctccttcaccctttggaaaccaagaaagcccttccaacagtgtccaggatgctggagggcagtgaccctcccccacttcttcttcgtgctggctgtgctgacacagctccagttcgaggttgtggcccgagacattaagtgagagccccgggtgacctgacttagcaccctgatcatcacatgggagtgaaaggcctgatgcgccagcttctcccactgcctccctttctgccctgcaaccctgtggaaacaggcagttctgggtcccacaaacatcacagaggttttgaaagcagaatcctaaagccgatttaaggggcagaaggaaggaggctataaagtcactacccttaccgctagtgttctgatgacccttggttcttcttcccccacccccgcccagtggagcccattgcagcaaggcttaaggaggtccgactgcagagggatgattttgagattttgaaggtgatcgggcgtggggcgttcagcgaggtgagtcttcagtggcctgggaatggaactttacttgatgtgggtggggcataacagctggggcagagccttaaaaattgatgaatgagcttgaatttaaggctggaggggtgggggcggagcttgtggtcagtgggcggtgtgcacgtgagggcggggctaaggttgggtggagataagggtggagtcctgtctgggtgagccttgctggttttccctgccacctcttgctgtcatctcggttccgtatttaggtagcggtggtgaagatgaaacagacgggccaagtgtatgccatgaagattatgaataagtgggacatgctgaagagaggcgaggtgagggccagggattagggcagcgccctcatctctccaactcacctcctgtagcttctctcctacctcacaggtgtcgtgcttccgggaagaaagggatgtattagtgaaaggggaccggcgctggatcacacagctgcactttgccttccaggatgagaactacctggtaagctccgggttcaggtgactaggaaagagtgacagttacatcgccccaagtcaagaaggctggagaagggagaagctgctgtagatcgggggggtgggggtgggggggacacacacaggggatgggggacgggggtaggattgtgtctcaagtataggagagaccttccttgagacaggagtgatatctggtttggcctttggatggggcgctctctcactgtgcgggggtcctctgtgcttgggaacggggtgtctttgggagtcttgggggctaccaaacccctgtgacacacccgctcccagtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacaggtgggcgtggcggggcccttggagggttagcagaatttgtgtgggaaggaagggtacctgaaggtcagatcccattggggacagaatcggggtctagaattgtagaatcctgggtggggtggaagtggatcgagctgacgggccctaagagggaaggttttcaagaaagcacactttccctcttctctctatgcacagggacatcaaaccagataacattctgctggaccgatgtgggcacattcgcctggcagacttcggctcctgcctcaaactgcagcctgatggaatggtaagaagagcctggcgaaactctcctcattggtgaaggaccggattagggggcggggctgggttgaggagcaggaggggagcttggtctgggatgtcctgcgcaccatatttggacagtcaagggaaaggttttaagcattcaggtctgattggcacaggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggtgagcacaagcaccatgcaggggggctgacttagtggcttgtgctcccagactgtcttttttaaaagatatttatttatatgtgtgtgttttctgtgtatgtatatctgtgcactgagtaggtgtgcgaaggtcagagggcatgggatcccctggaactggagtcacagactattgtgtgctgccatgctgagtgctgggaaacagaaccttgatcactctgcaagagcagccagtgcactgaaacgacagagccagctctgcagcccagggctaactgttgcttttctttctaaataggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggtgagcgggggcggggtacgtacctgcagttcctgatccgttgaggggacttccctagcctcttccataaaattggggtgattggccaggtgtggtggtgcatacctttaatcgtagaacttcataggcagaggcaggtggctctctggtaaatcaaggccatcttggtctacatagtgacttctaggccagtcaggagtgagatcctcccttgaaaaataaaaaagggggtgttgaccttcctgggtcccaaattattatcctagagcactgctatgtatccactcaggtatgaggacacacaggtgaccagtcccaaagacagtgagtgaggcctcactcttggcagtactaaaattgattgtagggggctgggctcttgacccagcctggaaagtgctggagggcttcctggaggaggagactagctgagcccagaaggatgcaggagatcctttctcgggtgagtgctctcagcattttaacaagctctaggccctgcagagagaagtctggtgtgggcagagccccaatagaaagcaacaagatagaagagaaaatggtggagtttgttagtgggggcagttatgccgtgaacatagaggggcgaagggccatctcggataactgctagccacaagagccctgtctgtcttcctaggagacgctgtcagacatgcaggaagacatgccccttggggtgcgcctgcccttcgtgggctactcctactgctgcatggccttcaggtgagcacgactgccccctgctggggcctgtgtgcaggcccaccacagccactcaattgaaggctcagtcttcaaaccaagtattcctaggagctgtctaagttaggctttctgctgctgcgatgaaccctgactaaaagcaagctggggaggaaaaggcttatcgggcttacgtttccacatgggagcccatcactgaaggaagccaggacaggaactcacagcggggcaggaacgtggagctgatgcagaggcaatggaggggagctgcttactgacttgatccttatgtcttcctcagcctgtttccttgtagagcccaggaccaccaggccagtgagggctccactcacaatgggctgagctctcatctatgatcactagttatgaaaatgcccgataggcttgcctgcagcttcagtttttgaggcactttccttccttccttccttccttccttccttccttccttcctttctttctttctttctttctttctttctttctttctttctttctttctttctttctttcttagtcttttagagacagggtctttctatgtagctctggctgtcttggaattcattctgtagaccaggctggtcttatttatttattttatgtatgtgagtccactatcactgtcctcagacacaccagaagagggcatcagatcccattacagatggctgtgagccaccatgtggttgctgggaattgaactcaggacctctggaagagcagccagtgctcctgccctgtagaggcattttcttcatgaaggctgtctcctctctgatgacttgatgactctagcttgttgtgtcaagtggacataagactaggaaagcagctacacatgcactttgtttatttttgttttgctttttgagactgggtctctccatctcatagctctggccatcctgcctggtgacattccagtccagttgtataacctaagaatctgagactcagccttgcagaatcctgctattaacgggtctaggacactccatagaatccaggatcttagaaaaacaaacctgaagtgtgacagtttattttaagaacacaattggagcacataacaataatacaacttttcagttttaaaaagttttctgtcttgttttttgaggcaggagctccttaatatagtctaagccgccctgcgagtgctgtgattgatgggcatgtaccaccatgcctagtcaataaagcctttaaaaagcatccgttatgctggctgtggtgccacaaacctgtaatcccagcacttagaaggtagaggcaagattatcagaaattcaaggccatcctgggctatacagtaatctaaggctagcctggtctacaagagactctgtctaaagaaacaaaagataaatagcacccactattgctaggcaatataaccctataaccccaccattgaggaggctgaggctggagcatcactgcaaatttgaggccaggatggtcaacaaataagtcccagagctggcatagaggaactctgtctcaacaataaagagaacttatctagcatttatgagggtaaataaaaatttaccattgccacaaaaaatgtaaatgaagagactgcttttaggagtgaactgggaagcagggaacacttagaggatgctcactcacacaggtatccaccatcaggcatgcctcaggcctgcacagggaaggacaacttgtttcatgatttgcaagcagcatcccatgctccttagagcgggttgggcccagcccaccctctgtggagttatcgctcagccaggcagcaaggcagccaaggtgctgaggccctggcagtctgctctcttctctgctctgaacctcctttagctttagcctaggagcctggcctggtgcccacaggctagggagtccctagcctcttcctcttctcagagacaatcaggtcccggaccccacccctatggaactagaggccctgcagttgcctgtgtcagacttgcaagggcttgacttgcagcccccagtgtccccaccggatcaagtggtgagtagactgagaggtgggcaaagcttcctgggtgggtgtacctgcagtgccaactgccaggctgttaattcagtaggacactgtccccaactggcccaactgcacatcctgtagtcaggaggcacaggcagaaaaatcccaaattcaaggcttgctcccgttatgtaatgagatcctgtcttggagtaaaaaacaaagaagagaactagggatagctcagaggtagatgctctcctggcatgggggtggggtcagaaagcaacaccaaccggggcctgggagggagggactgccaaccacctggaggagtctggggtagacttggtgaacaaagttcagaggccatcaggtgggatgctggtttcttaaaagccacagataggtgggtagcattggaaagaggagtggggggttgcagaaagtgacaagacacaaactggggaggcctaagggtaaagccagggttgtctgaagcactgtggagctgggaggaacacgctaaacttctgacttcagcccttcagttcccctgttgactacactgtccccagggacccagggatggggagaggtggacgggggagggaagtacgggactgatccagctccaggtcccaactctgatccccaccgacaggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccaggtcagggtcacagtgctggggtgaggggagaggagagcagcaaccctcgcagtctcctcaccgataggtcggctcactcccctatctttcccagccaactacaggaggccgaggtccgaaaccgagacctggaggcgcatgttcggcagctacaggaacggatggagatgctgcaggccccaggagccgcaggcgagtccctcacctgcttccagccaagggggcactgggtggagatggggggcatgttgggtgtgtgaaccctcggggcaggggaggagtccaggctggggcaccgcagccgcgccactgcctttctcctccatcctccacactccatacacctctctcttctccttccagccatcacgggggtccccagtccccgggccacggatccaccttcccatgtaagacccctctctcccctccccgatccccatcttagatatgctacccacagcccttctcccgtccacgtttagggtccattctccttgggggttccagaagaaagctgcccttcactcatccattcagcatgcactatctaccagctctccctcgtttcaggcttctcgccaaatcctccccaagggaactccctatactcccgttctggcctcgactagattcccgcactgcctctcgccctgctgctgggctccgatcgggtcacctgtcccttctctctccagctagatggccccccggccgtggctgtgggccagtgcccgctggtggggccaggccccatgcaccgccgtcacctgctgctccctgccagggtatgtcccacgtccgcccaccacgggcctctgcctagctctgcccactgagtgtcaccactgcttgctgtgcctctgtggagctcggcccaccgcagggagggggggtattcgggcggccaatcaacacaggctgctgctaagtagccaatgacgagttccaacaggagctctttcttgcgagcagaccaactttagctgcgggctgtggggaccagagatgcgctcagaggcccatctatgggtataggctgggcggctcccaggagccagtgggcccctgtagcctagtgctaatccaaccttctctcctgcagatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcgccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaatcccgcgcaatatctctgttgtggaaaggaaaccgccccgcaggccaatggagagtccaatagagacaaccaatggcttgagtgggagctagaggggaggcaaagcgcacgaatcaggttgaagggtggggcttaggcatccagccagtaggagagaagcaacaagccaccagagacaccaccgccccccaccctcccccccagctgtgacccagctgtgccactcaagtttggaaaaaagtagggggttgggccagcagcgggcacaccatcttcccactgcgcctgcgcaagccacgcgcatccgctttttggaccgacactccagaaaagttgctgcaaactttctagcgcgattccccgcccctcctcccagctagatccaccgcctacccgcggggccgggaattccgaggggcggagcacggcgcggagatgggaagggagggggcccttcaagggacccgggagatgggagcggcttcgcgcccttaaccctccggacggcccattaccttctccgttgctctgatagggaaactgaggccctgagtcagaggcacacaaggggggaaggccaaaagcgcggccagagacggagggaaaacaaagaatcctgacagcccgggaggggggcggacacacagggacaaggacagacccgagtgcagagctgggtctagtctttgggagggggccagaagactgcaaggggaccgggggggggggcggcgaggaggactgggcggaggagggggctggggaagcccgcgggaggcggcaaaggagggaggaactttccaaagttgccaaacatggctacctcgcctgcggagccgagcgcggggcccgcggctcggggggaggcggcggcggcgaccgaggagcaggaggaggaagcgcgccagcttctgcagactctgcaggcagccgagggggaggcggcggcggccggggcgggagatgcggcggcggcggcggactctgggtccccgagtggcccggggtctccccgggagaccgtgaccgaggtgcccactggccttcgcttctcgcccgaacaggtggcatgcgtgtgcgaggcgctgctgcaggcgggccacgccggccgcttgagccgcttcctgggcgcgctgcccccggccgagcgcctacgtggcagcgatccggtgctgcgcgcgcgggccctagtggccttccagcggggtgaatacgccgagctctaccaacttctcgagagccgccctttccccgccgcccaccacgccttcctgcaggacctctacctgcgcgcgcgctaccacgaggccgagcgggcccgtggccgtgcgctgggcgctgtggacaaataccggctgcgcaagaagttccctctgcccaagaccatctgggatggcgaggagaccgtctattgcttcaaggagcgctcgcgagcggcgctcaaggcctgctaccgcggcaaccgctatcccacgcctgacgagaagcgccgcctggccacgctcaccggcctctcgcttacacaggtcagcaactggttcaagaaccggcgacagcgcgaccgcactgggaccggcggtggagcgccttgcaaaaggtgaggggggaaccgaccctccttcctcggtggccgctggagtctgcgcaagtgacccttcacatccctcttcggtggcgtcggcgagtgcataggctgagcgtggagagaccaggcacaccctgggttctctgggcatcactgcctcaggggcagaggttgttccagctacttctaagctgggaacgcagtgccaggaatgggggggggggcgggggcgggacgggcagtga

SEQ ID NO: 4 - DNA, 1896 bp, Mus musculus
  source 1..1896: mol_type = genomic DNA
atgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggccgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctga

SEQ ID NO: 5 - DNA, 771 bp, Mus musculus
  misc_feature 89: note = n is a, c, g, or t
  misc_feature 238: note = n is a, c, g, or t
  misc_feature 506: note = n is a, c, g, or t
  source 1..771: mol_type = genomic DNA
cctgcccctgtggctgaggcagagaccacggtaacgctgcagcagctccaggaagccctggaagaagaggttctcacccggcagagctngagccgcgagctggaggccatccggaccgccaaccagaacttctccagccaactacaggaggccgaggtccgaaaccgagacctggaggcgcatgttcggcagctacaggaacggatggagatgctgcaggccccaggagccgccggantccctcacctgcttccagccaagggggcactgggtggagatggggggcatgttgggtgtgtgaaccctcggggcaggggaggagtccaggctggggcaccgcgccgcgccactgcctttctcctccatcctccacactccatacacctctctcttctccttccagccatcacgggggtcccagtccccgggccacggatccaccttcccatctagatggccccccggcggtggctgtgggccagtgcccgctggtggggccaggacantgtcaccgccgtcacctgctgctccctgccaggattcctaggcctggctatccgaggcgcgttgctgctcctgttcgccgctgctctggctgctgcgccacactgggctgcactgggttggttggctataccggcggtcttcacccagtctggtgtttcccgtgagcacccttcgcccctgaaacctaagacttcaagccatctttcatttaggccttctaggaaggtcgagcgacaggggagcgacccaaagcgtctctgtgcc

SEQ ID NO: 6 - DNA, 434 bp, Mus musculus
  source 1..434: mol_type = genomic DNA
gagagacccaaggggtagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacacacggagtctttggcttcggaca

SEQ ID NO: 7 - DNA, 2688 bp, Mus musculus
  source 1..2688: mol_type = genomic DNA
ccacgcgtccgcccacgcgtccggggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcacctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 2862 bp, Mus musculus
  source 1..2862: mol_type = genomic DNA
gggatagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattacgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcacctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatgcttctcgccaaatcctccccaagggaactccctagactcccgttctggcctcgactagattcccgcactgcctctcgccctgctgctgggctccgatcgggtcacctgtcccttctctctccagctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaaaaaaaaaaaaaaa

SEQ ID NO: 9 - DNA, 18 bp, synthetic construct
  misc_feature 1..18: note = Primer
  source 1..18: mol_type = other DNA
agcctgagccgggagatg

SEQ ID NO: 10 - DNA, 21 bp, synthetic construct
  misc_feature 1..21: note = Primer
  source 1..21: mol_type = other DNA
gcgtagttgactggcgaagtt

SEQ ID NO: 11 - DNA, 21 bp, synthetic construct
  misc_feature 1..21: note = Probe
  source 1..21: mol_type = other DNA
aggccatccgcacggacaacc

SEQ ID NO: 12 - DNA, 20 bp, synthetic construct
  misc_feature 1..20: note = Synthetic oligonucleotide
  source 1..20: mol_type = other DNA
ctggctgcatgtctgcctgt

SEQ ID NO: 13 - DNA, 20 bp, synthetic construct
  misc_feature 1..20: note = Synthetic oligonucleotide
  source 1..20: mol_type = other DNA
ccaggagaaggtcgagcagg

SEQ ID NO: 14 - DNA, 20 bp, synthetic construct
  misc_feature 1..20: note = Synthetic oligonucleotide
  source 1..20: mol_type = other DNA
tctatggccatgacaatctc

SEQ ID NO: 15 - DNA, 20 bp, synthetic construct
  misc_feature 1..20: note = Synthetic oligonucleotide
  source 1..20: mol_type = other DNA
atgtccctgtgcacgtagcc

SEQ ID NO: 16 - DNA, 20 bp, synthetic construct
  misc_feature 1..20: note = Synthetic oligonucleotide
  source 1..20: mol_type = other DNA
atgtgtccggaagtcgcctg

SEQ ID NO: 17 - DNA, 20 bp, synthetic construct
  misc_feature 1..20: note = Synthetic oligonucleotide
  source 1..20: mol_type = other DNA
ctcaggctctgccgggtgag

SEQ ID NO: 18 - DNA, 20 bp, synthetic construct
  misc_feature 1..20: note = Synthetic oligonucleotide
  source 1..20: mol_type = other DNA
ggcactggcccacagccacg

SEQ ID NO: 19 - DNA, 20 bp, synthetic construct
  misc_feature 1..20: note = Synthetic oligonucleotide
  source 1..20: mol_type = other DNA
cctggccgaaagaaagaaat

SEQ ID NO: 20 - DNA, 20 bp, synthetic construct
  misc_feature 1..20: note = Synthetic oligonucleotide
  source 1..20: mol_type = other DNA
aaagaaatggtctgtgatcc

SEQ ID NO: 21 - DNA, 20 bp, synthetic construct
  misc_feature 1..20: note = Synthetic oligonucleotide
  source 1..20: mol_type = other DNA
aagaaagaaatggtctgtga

SEQ ID NO: 22 - DNA, 20 bp, synthetic construct
  misc_feature 1..20: note = Synthetic oligonucleotide
  source 1..20: mol_type = other DNA
ggccgaaagaaagaaatggt

SEQ ID NO: 23 - DNA, 20 bp, synthetic construct
  misc_feature 1..20: note = Synthetic oligonucleotide
  source 1..20: mol_type = other DNA
cctcagcctggccgaaagaa

SEQ ID NO: 24 - DNA, 20 bp, synthetic construct
  misc_feature 1..20: note = Synthetic oligonucleotide
  source 1..20: mol_type = other DNA
gggcctcagcctggccgaaa

SEQ ID NO: 25 - DNA, 20 bp, synthetic construct
  misc_feature 1..20: note = Synthetic oligonucleotide
  source 1..20: mol_type = other DNA
tcagggcctcagcctggccg

SEQ ID NO: 26 - DNA, 20 bp, synthetic construct
  misc_feature 1..20: note = Synthetic oligonucleotide
  source 1..20: mol_type = other DNA
ctgcagtttgcccatccacg

SEQ ID NO: 27 - DNA, 20 bp, synthetic construct
  misc_feature 1..20: note = Synthetic oligonucleotide
  source 1..20: mol_type = other DNA
ggcctgcagtttgcccatcc

SEQ ID NO: 28 - DNA, 20 bp, synthetic construct
  misc_feature 1..20: note = Synthetic oligonucleotide
  source 1..20: mol_type = other DNA
ccaggcctgcagtttgccca

SEQ ID NO: 29 - DNA, 20 bp, synthetic construct
  misc_feature 1..20: note = Synthetic oligonucleotide
  source 1..20: mol_type = other DNA
gccttcccaggcctgcagtt

SEQ ID NO: 30 - DNA, 20 bp, synthetic construct
  misc_feature 1..20: note = Synthetic oligonucleotide
  source 1..20: mol_type = other DNA
gctgccttcccaggcctgca

SEQ ID NO: 31 - DNA, 20 bp, synthetic construct
  misc_feature 1..20: note = Synthetic oligonucleotide
  source 1..20: mol_type = other DNA
cttgctgccttcccaggcct

SEQ ID NO: 32 - DNA, 20 bp, synthetic construct
  misc_feature 1..20: note = Synthetic oligonucleotide
  source 1..20: mol_type = other DNA
gcccggcttgctgccttccc

SEQ ID NO: 33 - DNA, 20 bp, synthetic construct
  misc_feature 1..20: note = Synthetic oligonucleotide
  source 1..20: mol_type = other DNA
acggcccggcttgctgcctt

SEQ ID NO: 34 - DNA, 20 bp, synthetic construct
  misc_feature 1..20: note = Synthetic oligonucleotide
  source 1..20: mol_type = other DNA
cggacggcccggcttgctgc

SEQ ID NO: 35 - DNA, 20 bp, synthetic construct
  misc_feature 1..20: note = Synthetic oligonucleotide
  source 1..20: mol_type = other DNA
acacggacggcccggcttgc

SEQ ID NO: 36 - DNA, 20 bp, synthetic construct
  misc_feature 1..20: note = Synthetic oligonucleotide
  source 1..20: mol_type = other DNA
gatggaacacggacggcccg

SEQ ID NO: 37 - DNA, 20 bp, synthetic construct
  misc_feature 1..20: note = Synthetic oligonucleotide
  source 1..20: mol_type = other DNA
gaggatggaacacggacggc

SEQ ID NO: 38 - DNA, 20 bp, synthetic construct
  misc_feature 1..20: note = Synthetic oligonucleotide
  source 1..20: mol_type = other DNA
gtggaggatggaacacggac

SEQ ID NO: 39 - DNA, 20 bp, synthetic construct
  misc_feature 1..20: note = Synthetic oligonucleotide
  source 1..20: mol_type = other DNA
gcgaaccaacgataggtggg

SEQ ID NO: 40 - DNA, 20 bp, synthetic construct
  misc_feature 1..20: note = Synthetic oligonucleotide
  source 1..20: mol_type = other DNA
tttgcgaaccaacgataggt

SEQ ID NO: 41 - DNA, 20 bp, synthetic construct
  misc_feature 1..20: note = Synthetic oligonucleotide
  source 1..20: mol_type = other DNA
ttgcactttgcgaaccaacg

SEQ ID NO: 42 - DNA, 20 bp, synthetic construct
  misc_feature 1..20: note = Synthetic oligonucleotide
  source 1..20: mol_type = other DNA
gctttgcactttgcgaacca

SEQ ID NO: 43 - DNA, 20 bp, synthetic construct
  misc_feature 1..20: note = Synthetic oligonucleotide
  source 1..20: mol_type = other DNA
aaagctttgcactttgcgaa

SEQ ID NO: 44 - DNA, 20 bp, synthetic construct
  misc_feature 1..20: note = Synthetic oligonucleotide
  source 1..20: mol_type = other DNA
aagaaagctttgcactttgc

SEQ ID NO: 45 - DNA, 20 bp, synthetic construct
  misc_feature 1..20: note = Synthetic oligonucleotide
  source 1..20: mol_type = other DNA
cacaagaaagctttgcactt

SEQ ID NO: 46 - DNA, 20 bp, synthetic construct
  misc_feature 1..20: note = Synthetic oligonucleotide
  source 1..20: mol_type = other DNA
gtcatgcacaagaaagcttt

SEQ ID NO: 47 - DNA, 20 bp, synthetic construct
  misc_feature 1..20: note = Synthetic oligonucleotide
  source 1..20: mol_type = other DNA
acgctccccagagcagggcg

SEQ ID NO: 48 - DNA, 20 bp, synthetic construct
  misc_feature 1..20: note = Synthetic oligonucleotide
  source 1..20: mol_type = other DNA
gcagagatcgcgccagacgc

SEQ ID NO: 49 - DNA, 20 bp, synthetic construct
  misc_feature 1..20: note = Synthetic oligonucleotide
  source 1..20: mol_type = other DNA
caggcagagatcgcgccaga

SEQ ID NO: 50 - DNA, 20 bp, synthetic construct
  misc_feature 1..20: note = Synthetic oligonucleotide
  source 1..20: mol_type = other DNA
aagcaggcagagatcgcgcc

SEQ ID NO: 51 - DNA, 20 bp, synthetic construct
  misc_feature 1..20: note = Synthetic oligonucleotide
  source 1..20: mol_type = other DNA
ccgagtaagcaggcagagat

SEQ ID NO: 52 - DNA, 20 bp, synthetic construct
  misc_feature 1..20: note = Synthetic oligonucleotide
  source 1..20: mol_type = other DNA
ttcccgagtaagcaggcaga

SEQ ID NO: 53 - DNA, 20 bp, synthetic construct
  misc_feature 1..20: note = Synthetic oligonucleotide
  source 1..20: mol_type = other DNA
gcaaatttcccgagtaagca

SEQ ID NO: 54 - DNA, 20 bp, synthetic construct
  misc_feature 1..20: note = Synthetic oligonucleotide
  source 1..20: mol_type = other DNA
aaagcaaatttcccgagtaa

SEQ ID NO: 55 - DNA, 20 bp, synthetic construct
  misc_feature 1..20: note = Synthetic oligonucleotide
  source 1..20: mol_type = other DNA
ttggcaaaagcaaatttccc

SEQ ID NO: 56 - DNA, 20 bp, synthetic construct
  misc_feature 1..20: note = Synthetic oligonucleotide
  source 1..20: mol_type = other DNA
ggtttggcaaaagcaaattt

SEQ ID NO: 57 - DNA, 20 bp, synthetic construct
  misc_feature 1..20: note = Synthetic oligonucleotide
  source 1..20: mol_type = other DNA
gcgggtttggcaaaagcaaa

SEQ ID NO: 58 - DNA, 20 bp, synthetic construct
  misc_feature 1..20: note = Synthetic oligonucleotide
  source 1..20: mol_type = other DNA
aaagcgggtttggcaaaagc

SEQ ID NO: 59 - DNA, 20 bp, synthetic construct
  misc_feature 1..20: note = Synthetic oligonucleotide
  source 1..20: mol_type = other DNA
cccgaaaaagcgggtttggc

SEQ ID NO: 60 - DNA, 20 bp, synthetic construct
  misc_feature 1..20: note = Synthetic oligonucleotide
  source 1..20: mol_type = other DNA
atccccgaaaaagcgggttt

SEQ ID NO: 61 - DNA, 20 bp, synthetic construct
  misc_feature 1..20: note = Synthetic oligonucleotide
  source 1..20: mol_type = other DNA
cgggatccccgaaaaagcgg

SEQ ID NO: 62 - DNA, 20 bp, synthetic construct
  misc_feature 1..20: note = Synthetic oligonucleotide
  source 1..20: mol_type = other DNA
gcgcgggatccccgaaaaag

SEQ ID NO: 63 - DNA, 20 bp, synthetic construct
  misc_feature 1..20: note = Synthetic oligonucleotide
  source 1..20: mol_type = other DNA
gagagcagcgcaagtgagga

SEQ ID NO: 64 - DNA, 20 bp, synthetic construct
  misc_feature 1..20: note = Synthetic oligonucleotide
  source 1..20: mol_type = other DNA
tccgagagcagcgcaagtga

SEQ ID NO: 65 - DNA, 20 bp, synthetic construct
  misc_feature 1..20: note = Synthetic oligonucleotide
  source 1..20: mol_type = other DNA
ggctccgagagcagcgcaag

SEQ ID NO: 66 - DNA, 20 bp, synthetic construct
  misc_feature 1..20: note = Synthetic oligonucleotide
  source 1..20: mol_type = other DNA
aagcgggcggagccggctgg

SEQ ID NO: 67 - DNA, 20 bp, synthetic construct
  misc_feature 1..20: note = Synthetic oligonucleotide
  source 1..20: mol_type = other DNA
ccgaagcgggcggagccggc

SEQ ID NO: 68 - DNA, 20 bp, synthetic construct
  misc_feature 1..20: note = Synthetic oligonucleotide
  source 1..20: mol_type = other DNA
aaaccgccgaagcgggcgga

SEQ ID NO: 69 - DNA, 20 bp, synthetic construct
  misc_feature 1..20: note = Synthetic oligonucleotide
  source 1..20: mol_type = other DNA
tccaaaccgccgaagcgggc

SEQ ID NO: 70 - DNA, 20 bp, synthetic construct
  misc_feature 1..20: note = Synthetic oligonucleotide
  source 1..20: mol_type = other DNA
atatccaaaccgccgaagcg

SEQ ID NO: 71 - DNA, 20 bp, synthetic construct
  misc_feature 1..20: note = Synthetic oligonucleotide
  source 1..20: mol_type = other DNA
taaatatccaaaccgccgaa

SEQ ID NO: 72 - DNA, 20 bp, synthetic construct
  misc_feature 1..20: note = Synthetic oligonucleotide
  source 1..20: mol_type = other DNA
caataaatatccaaaccgcc

SEQ ID NO: 73 - DNA, 20 bp, synthetic construct
  misc_feature 1..20: note = Synthetic oligonucleotide
  source 1..20: mol_type = other DNA
cgaggtcaataaatatccaa

SEQ ID NO: 74 - DNA, 20 bp, synthetic construct
  misc_feature 1..20: note = Synthetic oligonucleotide
  source 1..20: mol_type = other DNA
ggacgaggtcaataaatatc

SEQ ID NO: 75 - DNA, 20 bp, synthetic construct
  misc_feature 1..20: note = Synthetic oligonucleotide
  source 1..20: mol_type = other DNA
ggaggacgaggtcaataaat

SEQ ID NO: 76 - DNA, 20 bp, synthetic construct
  misc_feature 1..20: note = Synthetic oligonucleotide
  source 1..20: mol_type = other DNA
gtcggaggacgaggtcaata

SEQ ID NO: 77 - DNA, 20 bp, synthetic construct
  misc_feature 1..20: note = Synthetic oligonucleotide
  source 1..20: mol_type = other DNA
cgagtcggaggacgaggtca

SEQ ID NO: 78 - DNA, 20 bp, synthetic construct
  misc_feature 1..20: note = Synthetic oligonucleotide
  source 1..20: mol_type = other DNA
tgtcagcgagtcggaggacg

SEQ ID NO: 79 - DNA, 20 bp, synthetic construct
  misc_feature 1..20: note = Synthetic oligonucleotide
  source 1..20: mol_type = other DNA
gcctgtcagcgagtcggagg

SEQ ID NO: 80 - DNA, 20 bp, synthetic construct
  misc_feature 1..20: note = Synthetic oligonucleotide
  source 1..20: mol_type = other DNA
gtagcctgtcagcgagtcgg

SEQ ID NO: 81 - DNA, 20 bp, synthetic construct
  misc_feature 1..20: note = Synthetic oligonucleotide
  source 1..20: mol_type = other DNA
cctgtagcctgtcagcgagt

SEQ ID NO: 82 - DNA, 20 bp, synthetic construct
  misc_feature 1..20: note = Synthetic oligonucleotide
  source 1..20: mol_type = other DNA
ggtcctgtagcctgtcagcg

SEQ ID NO: 83 - DNA, 20 bp, synthetic construct
  misc_feature 1..20: note = Synthetic oligonucleotide
  source 1..20: mol_type = other DNA
aaataccgaggaatgtcggg

SEQ ID NO: 84 - DNA, 20 bp, synthetic construct
  misc_feature 1..20: note = Synthetic oligonucleotide
  source 1..20: mol_type = other DNA
aataaataccgaggaatgtc

SEQ ID NO: 85 - DNA, 20 bp, synthetic construct
  misc_feature 1..20: note = Synthetic oligonucleotide
  source 1..20: mol_type = other DNA
gacaataaataccgaggaat

SEQ ID NO: 86 - DNA, 20 bp, synthetic construct
  misc_feature 1..20: note = Synthetic oligonucleotide
  source 1..20: mol_type = other DNA
cggggccccggagtcgaaga

SEQ ID NO: 87 - DNA, 20 bp, synthetic construct
  misc_feature 1..20: note = Synthetic oligonucleotide
  source 1..20: mol_type = other DNA
ccaacggggccccggagtcg

SEQ ID NO: 88 - DNA, 20 bp, synthetic construct
  misc_feature 1..20: note = Synthetic oligonucleotide
  source 1..20: mol_type = other DNA
ttccaacggggccccggagt

SEQ ID NO: 89 - DNA, 20 bp, synthetic construct
  misc_feature 1..20: note = Synthetic oligonucleotide
  source 1..20: mol_type = other DNA
gtcttccaacggggccccgg

SEQ ID NO: 90 - DNA, 20 bp, synthetic construct
  misc_feature 1..20: note = Synthetic oligonucleotide
  source 1..20: mol_type = other DNA
cagtcttccaacggggcccc

SEQ ID NO: 91 - DNA, 20 bp, synthetic construct
  misc_feature 1..20: note = Synthetic oligonucleotide
  source 1..20: mol_type = other DNA
ctcagtcttccaacggggcc

SEQ ID NO: 92 - DNA, 20 bp, synthetic construct
  misc_feature 1..20: note = Synthetic oligonucleotide
  source 1..20: mol_type = other DNA
gcactcagtcttccaacggg

SEQ ID NO: 93 - DNA, 20 bp, synthetic construct
  misc_feature 1..20: note = Synthetic oligonucleotide
  source 1..20: mol_type = other DNA
ccccgggcactcagtcttcc

SEQ ID NO: 94 - DNA, 20 bp, synthetic construct
  misc_feature 1..20: note = Synthetic oligonucleotide
  source 1..20: mol_type = other DNA
tgccccgggcactcagtctt

SEQ ID NO: 95 - DNA, 20 bp, synthetic construct
  misc_feature 1..20: note = Synthetic oligonucleotide
  source 1..20: mol_type = other DNA
cgtgccccgggcactcagtc

SEQ ID NO: 96 - DNA, 20 bp, synthetic construct
  misc_feature 1..20: note = Synthetic oligonucleotide
  source 1..20: mol_type = other DNA
gtgccgtgccccgggcactc

SEQ ID NO: 97 - DNA, 20 bp, synthetic construct
  misc_feature 1..20: note = Synthetic oligonucleotide
  source 1..20: mol_type = other DNA
tctgtgccgtgccccgggca

SEQ ID NO: 98 - DNA, 20 bp, synthetic construct
  misc_feature 1..20: note = Synthetic oligonucleotide
  source 1..20: mol_type = other DNA
gcttctgtgccgtgccccgg

SEQ ID NO: 99 - DNA, 20 bp, synthetic construct
  misc_feature 1..20: note = Synthetic oligonucleotide
  source 1..20: mol_type = other DNA
gcggcttctgtgccgtgccc

SEQ ID NO: 100 - DNA, 20 bp, synthetic construct
  misc_feature 1..20: note = Synthetic oligonucleotide
  source 1..20: mol_type = other DNA
gcgcggcttctgtgccgtgc

SEQ ID NO: 101 - DNA, 20 bp, synthetic construct
  misc_feature 1..20: note = Synthetic oligonucleotide
  source 1..20: mol_type = other DNA
gggcgcggcttctgtgccgt

SEQ ID NO: 102 - DNA, 20 bp, synthetic construct
  misc_feature 1..20: note = Synthetic oligonucleotide
  source 1..20: mol_type = other DNA
ggcggtgggcgcggcttctg

SEQ ID NO: 103 - DNA, 20 bp, synthetic construct
  misc_feature 1..20: note = Synthetic oligonucleotide
  source 1..20: mol_type = other DNA
ggcaggcggtgggcgcggct

SEQ ID NO: 104 - DNA, 20 bp, synthetic construct
  misc_feature 1..20: note = Synthetic oligonucleotide
  source 1..20: mol_type = other DNA
ctggcaggcggtgggcgcgg

SEQ ID NO: 105 - DNA, 20 bp, synthetic construct
  misc_feature 1..20: note = Synthetic oligonucleotide
  source 1..20: mol_type = other DNA
aactggcaggcggtgggcgc

SEQ ID NO: 106 - DNA, 20 bp, synthetic construct
  misc_feature 1..20: note = Synthetic oligonucleotide
  source 1..20: mol_type = other DNA
gtgaactggcaggcggtggg

SEQ ID NO: 107 - DNA, 20 bp, synthetic construct
  misc_feature 1..20: note = Synthetic oligonucleotide
  source 1..20: mol_type = other DNA
ggttgtgaactggcaggcgg

SEQ ID NO: 108 - DNA, 20 bp, synthetic construct
  misc_feature 1..20: note = Synthetic oligonucleotide
  source 1..20: mol_type = other DNA
gcggttgtgaactggcaggc

SEQ ID NO: 109 - DNA, 20 bp, synthetic construct
  misc_feature 1..20: note = Synthetic oligonucleotide
  source 1..20: mol_type = other DNA
cggagcggttgtgaactggc

SEQ ID NO: 110 - DNA, 20 bp, synthetic construct
  misc_feature 1..20: note = Synthetic oligonucleotide
  source 1..20: mol_type = other DNA
cgctcggagcggttgtgaac

SEQ ID NO: 111 - DNA, 20 bp, synthetic construct
  misc_feature 1..20: note = Synthetic oligonucleotide
  source 1..20: mol_type = other DNA
cccacgctcggagcggttgt

SEQ ID NO: 112 - DNA, 20 bp, synthetic construct
  misc_feature 1..20: note = Synthetic oligonucleotide
  source 1..20: mol_type = other DNA
agacccacgctcggagcggt

SEQ ID NO: 113 - DNA, 20 bp, synthetic construct
  misc_feature 1..20: note = Synthetic oligonucleotide
  source 1..20: mol_type = other DNA
cggagacccacgctcggagc

SEQ ID NO: 114 - DNA, 20 bp, synthetic construct
  misc_feature 1..20: note = Synthetic oligonucleotide
  source 1..20: mol_type = other DNA
gggcggagacccacgctcgg

SEQ ID NO: 115 - DNA, 20 bp, synthetic construct
  misc_feature 1..20: note = Synthetic oligonucleotide
  source 1..20: mol_type = other DNA
gctgggcggagacccacgct

SEQ ID NO: 116 - DNA, 20 bp, synthetic construct
  misc_feature 1..20: note = Synthetic oligonucleotide
  source 1..20: mol_type = other DNA
ggagctgggcggagacccac

SEQ ID NO: 117 - DNA, 20 bp, synthetic construct
  misc_feature 1..20: note = Synthetic oligonucleotide
  source 1..20: mol_type = other DNA
ctggagctgggcggagaccc

SEQ ID NO: 118 - DNA, 20 bp, synthetic construct
  misc_feature 1..20: note = Synthetic oligonucleotide
  source 1..20: mol_type = other DNA
ggactggagctgggcggaga

SEQ ID NO: 119 - DNA, 20 bp, synthetic construct
  misc_feature 1..20: note = Synthetic oligonucleotide
  source 1..20: mol_type = other DNA
caggactggagctgggcgga

SEQ ID NO: 120 - DNA, 20 bp, synthetic construct
  misc_feature 1..20: note = Synthetic oligonucleotide
  source 1..20: mol_type = other DNA
atcacaggactggagctggg

SEQ ID NO: 121 - DNA, 20 bp, synthetic construct
  misc_feature 1..20: note = Synthetic oligonucleotide
  source 1..20: mol_type = other DNA
gggcgggcccggatcacagg

SEQ ID NO: 122 - DNA, 20 bp, synthetic construct
  misc_feature 1..20: note = Synthetic oligonucleotide
  source 1..20: mol_type = other DNA
gggggcgggcccggatcaca

SEQ ID NO: 123 - DNA, 20 bp, synthetic construct
  misc_feature 1..20: note = Synthetic oligonucleotide
  source 1..20: mol_type = other DNA
aggcagcaccatggcccctc

SEQ ID NO: 124 - DNA, 20 bp, synthetic construct
  misc_feature 1..20: note = Synthetic oligonucleotide
  source 1..20: mol_type = other DNA
ggtccaacaccagctgctgg

SEQ ID NO: 125 - DNA, 20 bp, synthetic construct
  misc_feature 1..20: note = Synthetic oligonucleotide
  source 1..20: mol_type = other DNA
cgatcaccttcagaatctcg

SEQ ID NO: 126 - DNA, 20 bp, synthetic construct
  misc_feature 1..20: note = Synthetic oligonucleotide
  source 1..20: mol_type = other DNA
cttgttcatgatcttcatgg

SEQ ID NO: 127 - DNA, 20 bp, synthetic construct
  misc_feature 1..20: note = Synthetic oligonucleotide
  source 1..20: mol_type = other DNA
ccccattcaccaacacgtcc

SEQ ID NO: 128 - DNA, 20 bp, synthetic construct
  misc_feature 1..20: note = Synthetic oligonucleotide
  source 1..20: mol_type = other DNA
gcgtgatccaccgccggtcc

SEQ ID NO: 129 - DNA, 20 bp, synthetic construct
  misc_feature 1..20: note = Synthetic oligonucleotide
  source 1..20: mol_type = other DNA
gtaatactccatgaccaggt

SEQ ID NO: 130 - DNA, 20 bp, synthetic construct
  misc_feature 1..20: note = Synthetic oligonucleotide
  source 1..20: mol_type = other DNA
gcagtgtcagcaggtccccg

SEQ ID NO: 131 - DNA, 20 bp, synthetic construct
  misc_feature 1..20: note = Synthetic oligonucleotide
  source 1..20: mol_type = other DNA
caccgagtctatggccatga

SEQ ID NO: 132 - DNA, 20 bp, synthetic construct
  misc_feature 1..20: note = Synthetic oligonucleotide
  source 1..20: mol_type = other DNA
acgtagccaagccggtgcac

SEQ ID NO: 133 - DNA, 20 bp, synthetic construct
  misc_feature 1..20: note = Synthetic oligonucleotide
  source 1..20: mol_type = other DNA
atgtggccacagcggtccag

SEQ ID NO: 134 - DNA, 20 bp, synthetic construct
  misc_feature 1..20: note = Synthetic oligonucleotide
  source 1..20: mol_type = other DNA
cttcgtccaccagcggcaga

SEQ ID NO: 135 - DNA, 20 bp, synthetic construct
  misc_feature 1..20: note = Synthetic oligonucleotide
  source 1..20: mol_type = other DNA
gaccccttcgtccaccagcg

SEQ ID NO: 136 - DNA, 20 bp, synthetic construct
  misc_feature 1..20: note = Synthetic oligonucleotide
  source 1..20: mol_type = other DNA
cctgctccaccccggcccag

SEQ ID NO: 137 - DNA, 20 bp, synthetic construct
  misc_feature 1..20: note = Synthetic oligonucleotide
  source 1..20: mol_type = other DNA
cggaagtcgcctgctccacc

SEQ ID NO: 138 - DNA, 20 bp, synthetic construct
  misc_feature 1..20: note = Synthetic oligonucleotide
  source 1..20: mol_type = other DNA
cggagaccatcccagtcgag

SEQ ID NO: 139 - DNA, 20 bp, synthetic construct
  misc_feature 1..20: note = Synthetic oligonucleotide
  source 1..20: mol_type = other DNA
tgagggccatgcaggagtag

SEQ ID NO: 140 - DNA, 20 bp, synthetic construct
  misc_feature 1..20: note = Synthetic oligonucleotide
  source 1..20: mol_type = other DNA
ctccagttccatgggtgtgg

SEQ ID NO: 141 - DNA, 20 bp, synthetic construct
  misc_feature 1..20: note = Synthetic oligonucleotide
  source 1..20: mol_type = other DNA
gcgcttgcacgtgtggctca

SEQ ID NO: 142 - DNA, 20 bp, synthetic construct
  misc_feature 1..20: note = Synthetic oligonucleotide
  source 1..20: mol_type = other DNA
gccacttcagctgtttcatc

SEQ ID NO: 143 - DNA, 20 bp, synthetic construct
  misc_feature 1..20: note = Synthetic oligonucleotide
  source 1..20: mol_type = other DNA
gcctcagcctctgccgcagg

SEQ ID NO: 144 - DNA, 20 bp, synthetic construct
  misc_feature 1..20: note = Synthetic oligonucleotide
  source 1..20: mol_type = other DNA
gcagcgtcacctcggcctca

SEQ ID NO: 145 - DNA, 20 bp, synthetic construct
  misc_feature 1..20: note = Synthetic oligonucleotide
  source 1..20: mol_type = other DNA
ggctcaggctctgccgggtg

SEQ ID NO: 146 - DNA, 20 bp, synthetic construct
  misc_feature 1..20: note = Synthetic oligonucleotide
  source 1..20: mol_type = other DNA
ttccgagcctctgcctcgcg

SEQ ID NO: 147 - DNA, 20 bp, synthetic construct
  misc_feature 1..20: note = Synthetic oligonucleotide
  source 1..20: mol_type = other DNA
ggtcccggttccgagcctct

SEQ ID NO: 148 - DNA, 20 bp, synthetic construct
  misc_feature 1..20: note = Synthetic oligonucleotide
  source 1..20: mol_type = other DNA
atccgctcctgcaactgccg

SEQ ID NO: 149 - DNA, 20 bp, synthetic construct
  misc_feature 1..20: note = Synthetic oligonucleotide
  source 1..20: mol_type = other DNA
gcaactccatccgctcctgc

SEQ ID NO: 150 - DNA, 20 bp, synthetic construct
  misc_feature 1..20: note = Synthetic oligonucleotide
  source 1..20: mol_type = other DNA
aggtggatccgtggcccggg

SEQ ID NO: 151 - DNA, 20 bp, synthetic construct
  misc_feature 1..20: note = Synthetic oligonucleotide
  source 1..20: mol_type = other DNA
cgcggcttctgtgccgtgcc

SEQ ID NO: 152 - DNA, 20 bp, synthetic construct
  misc_feature 1..20: note = Synthetic oligonucleotide
  source 1..20: mol_type = other DNA
ttgctgccttcccaggcctg

SEQ ID NO: 153 - DNA, 20 bp, synthetic construct
  misc_feature 1..20: note = Synthetic oligonucleotide
  source 1..20: mol_type = other DNA
tgctcccgacaagctccaga

SEQ ID NO: 154 - DNA, 20 bp, synthetic construct
  misc_feature 1..20: note = Synthetic oligonucleotide
  source 1..20: mol_type = other DNA
agaacctgcccattgctgaa

SEQ ID NO: 155 - DNA, 20 bp, synthetic construct
  misc_feature 1..20: note = Synthetic oligonucleotide
  source 1..20: mol_type = other DNA
cactgagggccagacatatg

SEQ ID NO: 156 - DNA, 20 bp, synthetic construct
  misc_feature 1..20: note = Synthetic oligonucleotide
  source 1..20: mol_type = other DNA
ctctagattcagatgcaggt

SEQ ID NO: 157 - DNA, 15 bp, synthetic construct
  misc_feature 1..15: note = Primer
  source 1..15: mol_type = other DNA
cgggccgtccgtgtt

SEQ ID NO: 158 - DNA, 20 bp, synthetic construct
  misc_feature 1..20: note = Primer
  source 1..20: mol_type = other DNA
ctttgcactttgcgaaccaa

SEQ ID NO: 159 - DNA, 21 bp, synthetic construct
  misc_feature 1..21: note = Probe
  source 1..21: mol_type = other DNA
catcctccacgcacccccacc

SEQ ID NO: 160 - DNA, 20 bp, synthetic construct
  misc_feature 1..20: note = Synthetic oligonucleotide
  source 1..20: mol_type = other DNA
gcctggcagcccctgtccag

SEQ ID NO: 161 - DNA, 20 bp, synthetic construct
  misc_feature 1..20: note = Synthetic oligonucleotide
  source 1..20: mol_type = other DNA
ggcctggcagcccctgtcca

SEQ ID NO: 162 - DNA, 20 bp, synthetic construct
  misc_feature 1..20: note = Synthetic oligonucleotide
  source 1..20: mol_type = other DNA
gggcctggcagcccctgtcc

SEQ ID NO: 163 - DNA, 20 bp, synthetic construct
  misc_feature 1..20: note = Synthetic oligonucleotide
  source 1..20: mol_type = other DNA
atggcccctccccgggccgg

SEQ ID NO: 164 - DNA, 20 bp, synthetic construct
  misc_feature 1..20: note = Synthetic oligonucleotide
  source 1..20: mol_type = other DNA
catggcccctccccgggccg

SEQ ID NO: 165 - DNA, 20 bp, synthetic construct
  misc_feature 1..20: note = Synthetic oligonucleotide
  source 1..20: mol_type = other DNA
ccatggcccctccccgggcc

SEQ ID NO: 166 - DNA, 20 bp, synthetic construct
  misc_feature 1..20: note = Synthetic oligonucleotide
  source 1..20: mol_type = other DNA
accatggcccctccccgggc

SEQ ID NO: 167 - DNA, 20 bp, synthetic construct
  misc_feature 1..20: note = Synthetic oligonucleotide
  source 1..20: mol_type = other DNA
caccatggcccctccccggg

SEQ ID NO: 168 - DNA, 20 bp, synthetic construct
  misc_feature 1..20: note = Synthetic oligonucleotide
  source 1..20: mol_type = other DNA
gcaccatggcccctccccgg

SEQ ID NO: 169 - DNA, 20 bp, synthetic construct
  misc_feature 1..20: note = Synthetic oligonucleotide
  source 1..20: mol_type = other DNA
agcaccatggcccctccccg

SEQ ID NO: 170 - DNA, 20 bp, synthetic construct
  misc_feature 1..20: note = Synthetic oligonucleotide
  source 1..20: mol_type = other DNA
cagcaccatggcccctcccc

SEQ ID NO: 171 - DNA, 20 bp, synthetic construct
  misc_feature 1..20: note = Synthetic oligonucleotide
  source 1..20: mol_type = other DNA
gcagcaccatggcccctccc

SEQ ID NO: 172 - DNA, 20 bp, synthetic construct
  misc_feature 1..20: note = Synthetic oligonucleotide
  source 1..20: mol_type = other DNA
ggcagcaccatggcccctcc

SEQ ID NO: 173 - DNA, 20 bp, synthetic construct
  misc_feature 1..20: note = Synthetic oligonucleotide
  source 1..20: mol_type = other DNA
caggcagcaccatggcccct

SEQ ID NO: 174 - DNA, 20 bp, synthetic construct
  misc_feature 1..20: note = Synthetic oligonucleotide
  source 1..20: mol_type = other DNA
acaggcagcaccatggcccc

SEQ ID NO: 175 - DNA, 20 bp, synthetic construct
  misc_feature 1..20: note = Synthetic oligonucleotide
  source 1..20: mol_type = other DNA
ggacaggcagcaccatggcc

SEQ ID NO: 176 - DNA, 20 bp, synthetic construct
  misc_feature 1..20: note = Synthetic oligonucleotide
  source 1..20: mol_type = other DNA
tggacaggcagcaccatggc

SEQ ID NO: 177 - DNA, 20 bp, synthetic construct
  misc_feature 1..20: note = Synthetic oligonucleotide
  source 1..20: mol_type = other DNA
ttggacaggcagcaccatgg

SEQ ID NO: 178 - DNA, 20 bp, synthetic construct
  misc_feature 1..20: note = Synthetic oligonucleotide
  source 1..20: mol_type = other DNA
gttggacaggcagcaccatg

SEQ ID NO: 179 - DNA, 20 bp, synthetic construct
  misc_feature 1..20: note = Synthetic oligonucleotide
  source 1..20: mol_type = other DNA
tgttggacaggcagcaccat

SEQ ID NO: 180 - DNA, 20 bp, synthetic construct
  misc_feature 1..20: note = Synthetic oligonucleotide
  source 1..20: mol_type = other DNA
atgttggacaggcagcacca

SEQ ID NO: 181 - DNA, 20 bp, synthetic construct
  misc_feature 1..20: note = Synthetic oligonucleotide
  source 1..20: mol_type = other DNA
catgttggacaggcagcacc

SEQ ID NO: 182 - DNA, 20 bp, synthetic construct
  misc_feature 1..20: note = Synthetic oligonucleotide
  source 1..20: mol_type = other DNA
acatgttggacaggcagcac

SEQ ID NO: 183 - DNA, 20 bp, synthetic construct
  misc_feature 1..20: note = Synthetic oligonucleotide
  source 1..20: mol_type = other DNA
gacatgttggacaggcagca

SEQ ID NO: 184 - DNA, 20 bp, synthetic construct
  misc_feature 1..20: note = Synthetic oligonucleotide
  source 1..20: mol_type = other DNA
tgacatgttggacaggcagc

SEQ ID NO: 185 - DNA, 20 bp, synthetic construct
  misc_feature 1..20: note = Synthetic oligonucleotide
  source 1..20: mol_type = other DNA
ctgacatgttggacaggcag

SEQ ID NO: 186 - DNA, 20 bp, synthetic construct
  misc_feature 1..20: note = Synthetic oligonucleotide
  source 1..20: mol_type = other DNA
gctgacatgttggacaggca

SEQ ID NO: 187 - DNA, 20 bp, synthetic construct
  misc_feature 1..20: note = Synthetic oligonucleotide
  source 1..20: mol_type = other DNA
ggctgacatgttggacaggc

SEQ ID NO: 188 - DNA, 20 bp, synthetic construct
  misc_feature 1..20: note = Synthetic oligonucleotide
  source 1..20: mol_type = other DNA
cggctgacatgttggacagg

SEQ ID NO: 189 - DNA, 20 bp, synthetic construct
  misc_feature 1..20: note = Synthetic oligonucleotide
  source 1..20: mol_type = other DNA
tcggctgacatgttggacag

SEQ ID NO: 190 - DNA, 20 bp, synthetic construct
  misc_feature 1..20: note = Synthetic oligonucleotide
  source 1..20: mol_type = other DNA
ctcggctgacatgttggaca

SEQ ID NO: 191 - DNA, 20 bp, synthetic construct
  misc_feature 1..20: note = Synthetic oligonucleotide
  source 1..20: mol_type = other DNA
cctcggctgacatgttggac

SEQ ID NO: 192 - DNA, 20 bp, synthetic construct
  misc_feature 1..20: note = Synthetic oligonucleotide
  source 1..20: mol_type = other DNA
acctcggctgacatgttgga

SEQ ID NO: 193 - DNA, 20 bp, synthetic construct
  misc_feature 1..20: note = Synthetic oligonucleotide
  source 1..20: mol_type = other DNA
cacctcggctgacatgttgg

SEQ ID NO: 194 - DNA, 20 bp, synthetic construct
  misc_feature 1..20: note = Synthetic oligonucleotide
  source 1..20: mol_type = other DNA
gcacctcggctgacatgttg

SEQ ID NO: 195 - DNA, 20 bp, synthetic construct
  misc_feature 1..20: note = Synthetic oligonucleotide
  source 1..20: mol_type = other DNA
cgcacctcggctgacatgtt

SEQ ID NO: 196 - DNA, 20 bp, synthetic construct
  misc_feature 1..20: note = Synthetic oligonucleotide
  source 1..20: mol_type = other DNA
ccgcacctcggctgacatgt

SEQ ID NO: 197 - DNA, 20 bp, synthetic construct
  misc_feature 1..20: note = Synthetic oligonucleotide
  source 1..20: mol_type = other DNA
gccgcacctcggctgacatg

SEQ ID NO: 198 - DNA, 20 bp, synthetic construct
  misc_feature 1..20: note = Synthetic oligonucleotide
  source 1..20: mol_type = other DNA
agccgcacctcggctgacat

SEQ ID NO: 199 - DNA, 20 bp, synthetic construct
  misc_feature 1..20: note = Synthetic oligonucleotide
  source 1..20: mol_type = other DNA
cagccgcacctcggctgaca

SEQ ID NO: 200 - DNA, 20 bp, synthetic construct
  misc_feature 1..20: note = Synthetic oligonucleotide
  source 1..20: mol_type = other DNA
tcagccgcacctcggctgac

SEQ ID NO: 201 - DNA, 20 bp, synthetic construct
  misc_feature 1..20: note = Synthetic oligonucleotide
  source 1..20: mol_type = other DNA
ctcagccgcacctcggctga

SEQ ID NO: 202 - DNA, 20 bp, synthetic construct
  misc_feature 1..20: note = Synthetic oligonucleotide
  source 1..20: mol_type = other DNA
cctcagccgcacctcggctg

SEQ ID NO: 203 - DNA, 20 bp, synthetic construct
  misc_feature 1..20: note = Synthetic oligonucleotide
  source 1..20: mol_type = other DNA
gcctcagccgcacctcggct

SEQ ID NO: 204 - DNA, 20 bp, synthetic construct
  misc_feature 1..20: note = Synthetic oligonucleotide
  source 1..20: mol_type = other DNA
ccaacaccagctgctggagc

SEQ ID NO: 205 - DNA, 20 bp, synthetic construct
  misc_feature 1..20: note = Synthetic oligonucleotide
  source 1..20: mol_type = other DNA
tccaacaccagctgctggag

SEQ ID NO: 206 - DNA, 20 bp, synthetic construct
  misc_feature 1..20: note = Synthetic oligonucleotide
  source 1..20: mol_type = other DNA
gtccaacaccagctgctgga

SEQ ID NO: 207 - DNA, 20 bp, synthetic construct
  misc_feature 1..20: note = Synthetic oligonucleotide
  source 1..20: mol_type = other DNA
gggtccaacaccagctgctg

SEQ ID NO: 208 - DNA, 20 bp, synthetic construct
  misc_feature 1..20: note = Synthetic oligonucleotide
  source 1..20: mol_type = other DNA
ggctccagccccaggaagcc

SEQ ID NO: 209 - DNA, 20 bp, synthetic construct
  misc_feature 1..20: note = Synthetic oligonucleotide
  source 1..20: mol_type = other DNA
gggctccagccccaggaagc

SEQ ID NO: 210 - DNA, 20 bp, synthetic construct
  misc_feature 1..20: note = Synthetic oligonucleotide
  source 1..20: mol_type = other DNA
caggagaaggtcgagcaggg

SEQ ID NO: 211 - DNA, 20 bp, synthetic construct
  misc_feature 1..20: note = Synthetic oligonucleotide
  source 1..20: mol_type = other DNA
cccaggagaaggtcgagcag

SEQ ID NO: 212 - DNA, 20 bp, synthetic construct
  misc_feature 1..20: note = Synthetic oligonucleotide
  source 1..20: mol_type = other DNA
gcccaggagaaggtcgagca

SEQ ID NO: 213 - DNA, 20 bp, synthetic construct
  misc_feature 1..20: note = Synthetic oligonucleotide
  source 1..20: mol_type = other DNA
cgcccaggagaaggtcgagc

SEQ ID NO: 214 - DNA, 20 bp, synthetic construct
  misc_feature 1..20: note = Synthetic oligonucleotide
  source 1..20: mol_type = other DNA
acgcccaggagaaggtcgag

SEQ ID NO: 215 - DNA, 20 bp, synthetic construct
  misc_feature 1..20: note = Synthetic oligonucleotide
  source 1..20: mol_type = other DNA
tcctgggccagttcggaggc

SEQ ID NO: 216 - DNA, 20 bp, synthetic construct
  misc_feature 1..20: note = Synthetic oligonucleotide
  source 1..20: mol_type = other DNA
gtcctgggccagttcggagg

SEQ ID NO: 217 - DNA, 20 bp, synthetic construct
  misc_feature 1..20: note = Synthetic oligonucleotide
  source 1..20: mol_type = other DNA
tgtcctgggccagttcggag

SEQ ID NO: 218 - DNA, 20 bp, synthetic construct
  misc_feature 1..20: note = Synthetic oligonucleotide
  source 1..20: mol_type = other DNA
ttgtcctgggccagttcgga

SEQ ID NO: 219 - DNA, 20 bp, synthetic construct
  misc_feature 1..20: note = Synthetic oligonucleotide
  source 1..20: mol_type = other DNA
cttgtcctgggccagttcgg

SEQ ID NO: 220 - DNA, 20 bp, synthetic construct
  misc_feature 1..20: note = Synthetic oligonucleotide
  source 1..20: mol_type = other DNA
acttgtcctgggccagttcg

SEQ ID NO: 221 - DNA, 20 bp, synthetic construct
  misc_feature 1..20: note = Synthetic oligonucleotide
  source 1..20: mol_type = other DNA
tacttgtcctgggccagttc

SEQ ID NO: 222 - DNA, 20 bp, synthetic construct
  misc_feature 1..20: note = Synthetic oligonucleotide
  source 1..20: mol_type = other DNA
gtacttgtcctgggccagtt

SEQ ID NO: 223 - DNA, 20 bp, synthetic construct
  misc_feature 1..20: note = Synthetic oligonucleotide
  source 1..20: mol_type = other DNA
cgtacttgtcctgggccagt

SEQ ID NO: 224 - DNA, 20 bp, synthetic construct
  misc_feature 1..20: note = Synthetic oligonucleotide
  source 1..20: mol_type = other DNA
actgcaagaagtcggccacg

SEQ ID NO: 225 - DNA, 20 bp, synthetic construct
  misc_feature 1..20: note = Synthetic oligonucleotide
  source 1..20: mol_type = other DNA
ccactgcaagaagtcggcca

SEQ ID NO: 226 - DNA, 20 bp, synthetic construct
  misc_feature 1..20: note = Synthetic oligonucleotide
  source 1..20: mol_type = other DNA
cccactgcaagaagtcggcc

SEQ ID NO: 227 - DNA, 20 bp, synthetic construct
  misc_feature 1..20: note = Synthetic oligonucleotide
  source 1..20: mol_type = other DNA
gcccactgcaagaagtcggc

SEQ ID NO: 228 - DNA, 20 bp, synthetic construct
  misc_feature 1..20: note = Synthetic oligonucleotide
  source 1..20: mol_type = other DNA
cgcccactgcaagaagtcgg

SEQ ID NO: 229 - DNA, 20 bp, synthetic construct
  misc_feature 1..20: note = Synthetic oligonucleotide
  source 1..20: mol_type = other DNA
ccgcccactgcaagaagtcg

SEQ ID NO: 230 - DNA, 20 bp, synthetic construct
  misc_feature 1..20: note = Synthetic oligonucleotide
  source 1..20: mol_type = other DNA
tccgcccactgcaagaagtc

SEQ ID NO: 231 - DNA, 20 bp, synthetic construct
  misc_feature 1..20: note = Synthetic oligonucleotide
  source 1..20: mol_type = other DNA
ctccgcccactgcaagaagt

SEQ ID NO: 232 - DNA, 20 bp, synthetic construct
  misc_feature 1..20: note = Synthetic oligonucleotide
  source 1..20: mol_type = other DNA
gctccgcccactgcaagaag

SEQ ID NO: 233 - DNA, 20 bp, synthetic construct
  misc_feature 1..20: note = Synthetic oligonucleotide
  source 1..20: mol_type = other DNA
ggctccgcccactgcaagaa

SEQ ID NO: 234 - DNA, 20 bp, synthetic construct
  misc_feature 1..20: note = Synthetic oligonucleotide
  source 1..20: mol_type = other DNA
gggctccgcccactgcaaga

SEQ ID NO: 235 - DNA, 20 bp, synthetic construct
  misc_feature 1..20: note = Synthetic oligonucleotide
  source 1..20: mol_type = other DNA
tgggctccgcccactgcaag

SEQ ID NO: 236 - DNA, 20 bp, synthetic construct
  misc_feature 1..20: note = Synthetic oligonucleotide
  source 1..20: mol_type = other DNA
atgggctccgcccactgcaa

SEQ ID NO: 237 - DNA, 20 bp, synthetic construct
  misc_feature 1..20: note = Synthetic oligonucleotide
  source 1..20: mol_type = other DNA
gatgggctccgcccactgca

SEQ ID NO: 238 - DNA, 20 bp, synthetic construct
  misc_feature 1..20: note = Synthetic oligonucleotide
  source 1..20: mol_type = other DNA
cgatgggctccgcccactgc

SEQ ID NO: 239 - DNA, 20 bp, synthetic construct
  misc_feature 1..20: note = Synthetic oligonucleotide
  source 1..20: mol_type = other DNA
acgatgggctccgcccactg

SEQ ID NO: 240 - DNA, 20 bp, synthetic construct
  misc_feature 1..20: note = Synthetic oligonucleotide
  source 1..20: mol_type = other DNA
cacgatgggctccgcccact

SEQ ID NO: 241 - DNA, 20 bp, synthetic construct
  misc_feature 1..20: note = Synthetic oligonucleotide
  source 1..20: mol_type = other DNA
ccacgatgggctccgcccac

SEQ ID NO: 242 - DNA, 20 bp, synthetic construct
  misc_feature 1..20: note = Synthetic oligonucleotide
  source 1..20: mol_type = other DNA
accacgatgggctccgccca

SEQ ID NO: 243 - DNA, 20 bp, synthetic construct
  misc_feature 1..20: note = Synthetic oligonucleotide
  source 1..20: mol_type = other DNA
caccacgatgggctccgccc

SEQ ID NO: 244 - DNA, 20 bp, synthetic construct
  misc_feature 1..20: note = Synthetic oligonucleotide
  source 1..20: mol_type = other DNA
tcaccacgatgggctccgcc

SEQ ID NO: 245 - DNA, 20 bp, synthetic construct
  misc_feature 1..20: note = Synthetic oligonucleotide
  source 1..20: mol_type = other DNA
ctcaccacgatgggctccgc

SEQ ID NO: 246 - DNA, 20 bp, synthetic construct
  misc_feature 1..20: note = Synthetic oligonucleotide
  source 1..20: mol_type = other DNA
cctcaccacgatgggctccg

SEQ ID NO: 247 - DNA, 20 bp, synthetic construct
  misc_feature 1..20: note = Synthetic oligonucleotide
  source 1..20: mol_type = other DNA
gcctcaccacgatgggctcc

SEQ ID NO: 248 - DNA, 20 bp, synthetic construct
  misc_feature 1..20: note = Synthetic oligonucleotide
  source 1..20: mol_type = other DNA
agcctcaccacgatgggctc

SEQ ID NO: 249 - DNA, 20 bp, synthetic construct
  misc_feature 1..20: note = Synthetic oligonucleotide
  source 1..20: mol_type = other DNA
aagcctcaccacgatgggct

SEQ ID NO: 250 - DNA, 20 bp, synthetic construct
  misc_feature 1..20: note = Synthetic oligonucleotide
  source 1..20: mol_type = other DNA
taagcctcaccacgatgggc

SEQ ID NO: 251 - DNA, 20 bp, synthetic construct
  misc_feature 1..20: note = Synthetic oligonucleotide
  source 1..20: mol_type = other DNA
ttaagcctcaccacgatggg

SEQ ID NO: 252 - DNA, 20 bp, synthetic construct
  misc_feature 1..20: note = Synthetic oligonucleotide
  source 1..20: mol_type = other DNA
cttaagcctcaccacgatgg

SEQ ID NO: 253 - DNA, 20 bp, synthetic construct
  misc_feature 1..20: note = Synthetic oligonucleotide
  source 1..20: mol_type = other DNA
ccttaagcctcaccacgatg

SEQ ID NO: 254 - DNA, 20 bp, synthetic construct
  misc_feature 1..20: note = Synthetic oligonucleotide
  source 1..20: mol_type = other DNA
tccttaagcctcaccacgat

SEQ ID NO: 255 - DNA, 20 bp, synthetic construct
  misc_feature 1..20: note = Synthetic oligonucleotide
  source 1..20: mol_type = other DNA
ctccttaagcctcaccacga

SEQ ID NO: 256 - DNA, 20 bp, synthetic construct
  misc_feature 1..20: note = Synthetic oligonucleotide
  source 1..20: mol_type = other DNA
cctccttaagcctcaccacg

SEQ ID NO: 257 - DNA, 20 bp, synthetic construct
  misc_feature 1..20: note = Synthetic oligonucleotide
  source 1..20: mol_type = other DNA
acctccttaagcctcaccac

SEQ ID NO: 258 - DNA, 20 bp, synthetic construct
  misc_feature 1..20: note = Synthetic oligonucleotide
  source 1..20: mol_type = other DNA
gacctccttaagcctcacca

SEQ ID NO: 259 - DNA, 20 bp, synthetic construct
  misc_feature 1..20: note = Synthetic oligonucleotide
  source 1..20: mol_type = other DNA
ggacctccttaagcctcacc

SEQ ID NO: 260 - DNA, 20 bp, synthetic construct
  misc_feature 1..20: note = Synthetic oligonucleotide
  source 1..20: mol_type = other DNA
cggacctccttaagcctcac

SEQ ID NO: 261 - DNA, 20 bp, synthetic construct
  misc_feature 1..20: note = Synthetic oligonucleotide
  source 1..20: mol_type = other DNA
tcggacctccttaagcctca

SEQ ID NO: 262 - DNA, 20 bp, synthetic construct
  misc_feature 1..20: note = Synthetic oligonucleotide
  source 1..20: mol_type = other DNA
gtcggacctccttaagcctc

SEQ ID NO: 263 - DNA, 20 bp, synthetic construct
  misc_feature 1..20: note = Synthetic oligonucleotide
  source 1..20: mol_type = other DNA
cagtcggacctccttaagcc

SEQ ID NO: 264 - DNA, 20 bp, synthetic construct
  misc_feature 1..20: note = Synthetic oligonucleotide
  source 1..20: mol_type = other DNA
gcagtcggacctccttaagc

SEQ ID NO: 265 - DNA, 20 bp, synthetic construct
  misc_feature 1..20: note = Synthetic oligonucleotide
  source 1..20: mol_type = other DNA
tgcagtcggacctccttaag

SEQ ID NO: 266 - DNA, 20 bp, synthetic construct
  misc_feature 1..20: note = Synthetic oligonucleotide
  source 1..20: mol_type = other DNA
ccttcagaatctcgaagtcg

SEQ ID NO: 267 - DNA, 20 bp, synthetic construct
  misc_feature 1..20: note = Synthetic oligonucleotide
  source 1..20: mol_type = other DNA
accttcagaatctcgaagtc

SEQ ID NO: 268 - DNA, 20 bp, synthetic construct
  misc_feature 1..20: note = Synthetic oligonucleotide
  source 1..20: mol_type = other DNA
tcaccttcagaatctcgaag

SEQ ID NO: 269 - DNA, 20 bp, synthetic construct
  misc_feature 1..20: note = Synthetic oligonucleotide
  source 1..20: mol_type = other DNA
atcaccttcagaatctcgaa

SEQ ID NO: 270 - DNA, 20 bp, synthetic construct
  misc_feature 1..20: note = Synthetic oligonucleotide
  source 1..20: mol_type = other DNA
gatcaccttcagaatctcga

SEQ ID NO: 271 - DNA, 20 bp, synthetic construct
  misc_feature 1..20: note = Synthetic oligonucleotide
  source 1..20: mol_type = other DNA
ccgatcaccttcagaatctc

SEQ ID NO: 272 - DNA, 20 bp, synthetic construct
  misc_feature 1..20: note = Synthetic oligonucleotide
  source 1..20: mol_type = other DNA
tccgatcaccttcagaatct

SEQ ID NO: 273 - DNA, 20 bp, synthetic construct
  misc_feature 1..20: note = Synthetic oligonucleotide
  source 1..20: mol_type = other DNA
gtccgatcaccttcagaatc

SEQ ID NO: 274 - DNA, 20 bp, synthetic construct
  misc_feature 1..20: note = Synthetic oligonucleotide
  source 1..20: mol_type = other DNA
cgtccgatcaccttcagaat

SEQ ID NO: 275 - DNA, 20 bp, synthetic construct
  misc_feature 1..20: note = Synthetic oligonucleotide
  source 1..20: mol_type = other DNA
cccgtctgcttcatcttcac

SEQ ID NO: 276 - DNA, 20 bp, synthetic construct
  misc_feature 1..20: note = Synthetic oligonucleotide
  source 1..20: mol_type = other DNA
gcccgtctgcttcatcttca

SEQ ID NO: 277 - DNA, 20 bp, synthetic construct
  misc_feature 1..20: note = Synthetic oligonucleotide
  source 1..20: mol_type = other DNA
ggcccgtctgcttcatcttc

SEQ ID NO: 278 - DNA, 20 bp, synthetic construct
  misc_feature 1..20: note = Synthetic oligonucleotide
  source 1..20: mol_type = other DNA
tggcccgtctgcttcatctt

SEQ ID NO: 279 - DNA, 20 bp, synthetic construct
  misc_feature 1..20: note = Synthetic oligonucleotide
  source 1..20: mol_type = other DNA
ctggcccgtctgcttcatct

SEQ ID NO: 280 - DNA, 20 bp, synthetic construct
  misc_feature 1..20: note = Synthetic oligonucleotide
  source 1..20: mol_type = other DNA
cctggcccgtctgcttcatc

SEQ ID NO: 281 - DNA, 20 bp, synthetic construct
  misc_feature 1..20: note = Synthetic oligonucleotide
  source 1..20: mol_type = other DNA
acctggcccgtctgcttcat

SEQ ID NO: 282 - DNA, 20 bp, synthetic construct
  misc_feature 1..20: note = Synthetic oligonucleotide
  source 1..20: mol_type = other DNA
cacctggcccgtctgcttca

SEQ ID NO: 283 - DNA, 20 bp, synthetic construct
  misc_feature 1..20: note = Synthetic oligonucleotide
  source 1..20: mol_type = other DNA
acacctggcccgtctgcttc

SEQ ID NO: 284 - DNA, 20 bp, synthetic construct
  misc_feature 1..20: note = Synthetic oligonucleotide
  source 1..20: mol_type = other DNA
tacacctggcccgtctgctt

SEQ ID NO: 285 - DNA, 20 bp, synthetic construct
  misc_feature 1..20: note = Synthetic oligonucleotide
  source 1..20: mol_type = other DNA
ttgttcatgatcttcatggc

SEQ ID NO: 286 - DNA, 20 bp, synthetic construct
  misc_feature 1..20: note = Synthetic oligonucleotide
  source 1..20: mol_type = other DNA
acttgttcatgatcttcatg

SEQ ID NO: 287 - DNA, 20 bp, synthetic construct
  misc_feature 1..20: note = Synthetic oligonucleotide
  source 1..20: mol_type = other DNA
cacttgttcatgatcttcat

SEQ ID NO: 288 - DNA, 20 bp, synthetic construct
  misc_feature 1..20: note = Synthetic oligonucleotide
  source 1..20: mol_type = other DNA
ccacttgttcatgatcttca

SEQ ID NO: 289 - DNA, 20 bp, synthetic construct
  misc_feature 1..20: note = Synthetic oligonucleotide
  source 1..20: mol_type = other DNA
cccacttgttcatgatcttc

SEQ ID NO: 290 - DNA, 20 bp, synthetic construct
  misc_feature 1..20: note = Synthetic oligonucleotide
  source 1..20: mol_type = other DNA
tcccacttgttcatgatctt

SEQ ID NO: 291 - DNA, 20 bp, synthetic construct
  misc_feature 1..20: note = Synthetic oligonucleotide
  source 1..20: mol_type = other DNA
gtcccacttgttcatgatct

SEQ ID NO: 292 - DNA, 20 bp, synthetic construct
  misc_feature 1..20: note = Synthetic oligonucleotide
  source 1..20: mol_type = other DNA
tgtcccacttgttcatgatc

SEQ ID NO: 293 - DNA, 20 bp, synthetic construct
  misc_feature 1..20: note = Synthetic oligonucleotide
  source 1..20: mol_type = other DNA
atgtcccacttgttcatgat

SEQ ID NO: 294 - DNA, 20 bp, synthetic construct
  misc_feature 1..20: note = Synthetic oligonucleotide
  source 1..20: mol_type = other DNA
catgtcccacttgttcatga

SEQ ID NO: 295 - DNA, 20 bp, synthetic construct
  misc_feature 1..20: note = Synthetic oligonucleotide
  source 1..20: mol_type = other DNA
gcatgtcccacttgttcatg

SEQ ID NO: 296 - DNA, 20 bp, synthetic construct
  misc_feature 1..20: note = Synthetic oligonucleotide
  source 1..20: mol_type = other DNA
agcatgtcccacttgttcat

SEQ ID NO: 297 - DNA, 20 bp, synthetic construct
  misc_feature 1..20: note = Synthetic oligonucleotide
  source 1..20: mol_type = other DNA
cagcatgtcccacttgttca

SEQ ID NO: 298 - DNA, 20 bp, synthetic construct
  misc_feature 1..20: note = Synthetic oligonucleotide
  source 1..20: mol_type = other DNA
tcagcatgtcccacttgttc

SEQ ID NO: 299 - DNA, 20 bp, synthetic construct
  misc_feature 1..20: note = Synthetic oligonucleotide
  source 1..20: mol_type = other DNA
ttcagcatgtcccacttgtt

SEQ ID NO: 300 - DNA, 20 bp, synthetic construct
  misc_feature 1..20: note = Synthetic oligonucleotide
  source 1..20: mol_type = other DNA
cttcagcatgtcccacttgt

SEQ ID NO: 301 - DNA, 20 bp, synthetic construct
  misc_feature 1..20: note = Synthetic oligonucleotide
  source 1..20: mol_type = other DNA
tcttcagcatgtcccacttg

SEQ ID NO: 302 - DNA, 20 bp, synthetic construct
  misc_feature 1..20: note = Synthetic oligonucleotide
  source 1..20: mol_type = other DNA
cctcttcagcatgtcccact

SEQ ID NO: 303 - DNA, 20 bp, synthetic construct
  misc_feature 1..20: note = Synthetic oligonucleotide
  source 1..20: mol_type = other DNA
ccctcttcagcatgtcccac

SEQ ID NO: 304 - DNA, 20 bp, synthetic construct
  misc_feature 1..20: note = Synthetic oligonucleotide
  source 1..20: mol_type = other DNA
cccctcttcagcatgtccca

SEQ ID NO: 305 - DNA, 20 bp, synthetic construct
  misc_feature 1..20: note = Synthetic oligonucleotide
  source 1..20: mol_type = other DNA
gcccctcttcagcatgtccc

SEQ ID NO: 306 - DNA, 20 bp, synthetic construct
  misc_feature 1..20: note = Synthetic oligonucleotide
  source 1..20: mol_type = other DNA
cgcccctcttcagcatgtcc

SEQ ID NO: 307 - DNA, 20 bp, synthetic construct
  misc_feature 1..20: note = Synthetic oligonucleotide
  source 1..20: mol_type = other DNA
tcgcccctcttcagcatgtc

SEQ ID NO: 308 - DNA, 20 bp, synthetic construct
  misc_feature 1..20: note = Synthetic oligonucleotide
  source 1..20: mol_type = other DNA
ctcgcccctcttcagcatgt

SEQ ID NO: 309 - DNA, 20 bp, synthetic construct
  misc_feature 1..20: note = Synthetic oligonucleotide
  source 1..20: mol_type = other DNA
cctcgcccctcttcagcatg

SEQ ID NO: 310 - DNA, 20 bp, synthetic construct
  misc_feature 1..20: note = Synthetic oligonucleotide
  source 1..20: mol_type = other DNA
acctcgcccctcttcagcat

SEQ ID NO: 311 - DNA, 20 bp, synthetic construct
  misc_feature 1..20: note = Synthetic oligonucleotide
  source 1..20: mol_type = other DNA
cacctcgcccctcttcagca

SEQ ID NO: 312 - DNA, 20 bp, synthetic construct
  misc_feature 1..20: note = Synthetic oligonucleotide
  source 1..20: mol_type = other DNA
acacctcgcccctcttcagc

SEQ ID NO: 313 - DNA, 20 bp, synthetic construct
  misc_feature 1..20: note = Synthetic oligonucleotide
  source 1..20: mol_type = other DNA
gacacctcgcccctcttcag

SEQ ID NO: 314 - DNA, 20 bp, synthetic construct
  misc_feature 1..20: note = Synthetic oligonucleotide
  source 1..20: mol_type = other DNA
gccaggcggatgtggccaca

SEQ ID NO: 315 - DNA, 20 bp, synthetic construct
  misc_feature 1..20: note = Synthetic oligonucleotide
  source 1..20: mol_type = other DNA
accgcaccgttccatctgcc

SEQ ID NO: 316 - DNA, 20 bp, synthetic construct
  misc_feature 1..20: note = Synthetic oligonucleotide
  source 1..20: mol_type = other DNA
gaccgcaccgttccatctgc

SEQ ID NO: 317 - DNA, 20 bp, synthetic construct
  misc_feature 1..20: note = Synthetic oligonucleotide
  source 1..20: mol_type = other DNA
acagcctgcaggatctcggg

SEQ ID NO: 318 - DNA, 20 bp, synthetic construct
  misc_feature 1..20: note = Synthetic oligonucleotide
  source 1..20: mol_type = other DNA
cacagcctgcaggatctcgg

SEQ ID NO: 319 - DNA, 20 bp, synthetic construct
  misc_feature 1..20: note = Synthetic oligonucleotide
  source 1..20: mol_type = other DNA
ccacagcctgcaggatctcg

SEQ ID NO: 320 - DNA, 20 bp, synthetic construct
  misc_feature 1..20: note = Synthetic oligonucleotide
  source 1..20: mol_type = other DNA
cccacagcctgcaggatctc

SEQ ID NO: 321 - DNA, 20 bp, synthetic construct
  misc_feature 1..20: note = Synthetic oligonucleotide
  source 1..20: mol_type = other DNA
gcccacagcctgcaggatct

SEQ ID NO: 322 - DNA, 20 bp, synthetic construct
  misc_feature 1..20: note = Synthetic oligonucleotide
  source 1..20: mol_type = other DNA
cgcccacagcctgcaggatc

SEQ ID NO: 323 - DNA, 20 bp, synthetic construct
  misc_feature 1..20: note = Synthetic oligonucleotide
  source 1..20: mol_type = other DNA
ccgcccacagcctgcaggat

SEQ ID NO: 324 - DNA, 20 bp, synthetic construct
  misc_feature 1..20: note = Synthetic oligonucleotide
  source 1..20: mol_type = other DNA
accgcccacagcctgcagga

SEQ ID NO: 325 - DNA, 20 bp, synthetic construct
  misc_feature 1..20: note = Synthetic oligonucleotide
  source 1..20: mol_type = other DNA
caccgcccacagcctgcagg

SEQ ID NO: 326 - DNA, 20 bp, synthetic construct
  misc_feature 1..20: note = Synthetic oligonucleotide
  source 1..20: mol_type = other DNA
ccaccgcccacagcctgcag

SEQ ID NO: 327 - DNA, 20 bp, synthetic construct
  misc_feature 1..20: note = Synthetic oligonucleotide
  source 1..20: mol_type = other DNA
cccaccgcccacagcctgca

SEQ ID NO: 328 - DNA, 20 bp, synthetic construct
  misc_feature 1..20: note = Synthetic oligonucleotide
  source 1..20: mol_type = other DNA
gcccaccgcccacagcctgc

SEQ ID NO: 329 - DNA, 20 bp, synthetic construct
  misc_feature 1..20: note = Synthetic oligonucleotide
  source 1..20: mol_type = other DNA
ggcccaccgcccacagcctg

SEQ ID NO: 330 - DNA, 20 bp, synthetic construct
  misc_feature 1..20: note = Synthetic oligonucleotide
  source 1..20: mol_type = other DNA
aggcccaccgcccacagcct

SEQ ID NO: 331 - DNA, 20 bp, synthetic construct
  misc_feature 1..20: note = Synthetic oligonucleotide
  source 1..20: mol_type = other DNA
caggcccaccgcccacagcc

SEQ ID NO: 332 - DNA, 20 bp, synthetic construct
  misc_feature 1..20: note = Synthetic oligonucleotide
  source 1..20: mol_type = other DNA
ccaggcccaccgcccacagc

SEQ ID NO: 333 - DNA, 20 bp, synthetic construct
  misc_feature 1..20: note = Synthetic oligonucleotide
  source 1..20: mol_type = other DNA
cccaggcccaccgcccacag

SEQ ID NO: 334 - DNA, 20 bp, synthetic construct
  misc_feature 1..20: note = Synthetic oligonucleotide
  source 1..20: mol_type = other DNA
tcccaggcccaccgcccaca

SEQ ID NO: 335 - DNA, 20 bp, synthetic construct
  misc_feature 1..20: note = Synthetic oligonucleotide
  source 1..20: mol_type = other DNA
gtcccaggcccaccgcccac

SEQ ID NO: 336 - DNA, 20 bp, synthetic construct
  misc_feature 1..20: note = Synthetic oligonucleotide
  source 1..20: mol_type = other DNA
tgtcccaggcccaccgccca

SEQ ID NO: 337 - DNA, 20 bp, synthetic construct
  misc_feature 1..20: note = Synthetic oligonucleotide
  source 1..20: mol_type = other DNA
ctgtcccaggcccaccgccc

SEQ ID NO: 338 - DNA, 20 bp, synthetic construct
  misc_feature 1..20: note = Synthetic oligonucleotide
  source 1..20: mol_type = other DNA
cctgtcccaggcccaccgcc

SEQ ID NO: 339 - DNA, 20 bp, synthetic construct
  misc_feature 1..20: note = Synthetic oligonucleotide
  source 1..20: mol_type = other DNA
gcctgtcccaggcccaccgc

SEQ ID NO: 340 - DNA, 20 bp, synthetic construct
  misc_feature 1..20: note = Synthetic oligonucleotide
  source 1..20: mol_type = other DNA
tgcctgtcccaggcccaccg

SEQ ID NO: 341 - DNA, 20 bp, synthetic construct
  misc_feature 1..20: note = Synthetic oligonucleotide
  source 1..20: mol_type = other DNA
ctgcctgtcccaggcccacc

SEQ ID NO: 342 - DNA, 20 bp, synthetic construct
  misc_feature 1..20: note = Synthetic oligonucleotide
  source 1..20: mol_type = other DNA
gctgcctgtcccaggcccac

SEQ ID NO: 343 - DNA, 20 bp, synthetic construct
  misc_feature 1..20: note = Synthetic oligonucleotide
  source 1..20: mol_type = other DNA
agctgcctgtcccaggccca

SEQ ID NO: 344 - DNA, 20 bp, synthetic construct
  misc_feature 1..20: note = Synthetic oligonucleotide
  source 1..20: mol_type = other DNA
tagctgcctgtcccaggccc

SEQ ID NO: 345 - DNA, 20 bp, synthetic construct
  misc_feature 1..20: note = Synthetic oligonucleotide
  source 1..20: mol_type = other DNA
gtagctgcctgtcccaggcc

SEQ ID NO: 346 - DNA, 20 bp, synthetic construct
  misc_feature 1..20: note = Synthetic oligonucleotide
  source 1..20: mol_type = other DNA
cgtagctgcctgtcccaggc

SEQ ID NO: 347 - DNA, 20 bp, synthetic construct
  misc_feature 1..20: note = Synthetic oligonucleotide
  source 1..20: mol_type = other DNA
ccgtagctgcctgtcccagg

SEQ ID NO: 348 - DNA, 20 bp, synthetic construct
  misc_feature 1..20: note = Synthetic oligonucleotide
  source 1..20: mol_type = other DNA
cccgtagctgcctgtcccag

SEQ ID NO: 349 - DNA, 20 bp, synthetic construct
  misc_feature 1..20: note = Synthetic oligonucleotide
  source 1..20: mol_type = other DNA
gcccgtagctgcctgtccca

SEQ ID NO: 350 - DNA, 20 bp, synthetic construct
  misc_feature 1..20: note = Synthetic oligonucleotide
  source 1..20: mol_type = other DNA
ggcccgtagctgcctgtccc

SEQ ID NO: 351 - DNA, 20 bp, synthetic construct
  misc_feature 1..20: note = Synthetic oligonucleotide
  source 1..20: mol_type = other DNA
tagaacatttcataggcgaa

SEQ ID NO: 352 - DNA, 20 bp, synthetic construct
  misc_feature 1..20: note = Synthetic oligonucleotide
  source 1..20: mol_type = other DNA
tctccgccgtggaatccgcg

SEQ ID NO: 353 - DNA, 20 bp, synthetic construct
  misc_feature 1..20: note = Synthetic oligonucleotide
  source 1..20: mol_type = other DNA
gtctccgccgtggaatccgc

SEQ ID NO: 354 - DNA, 20 bp, synthetic construct
  misc_feature 1..20: note = Synthetic oligonucleotide
  source 1..20: mol_type = other DNA
ggtctccgccgtggaatccg

SEQ ID NO: 355 - DNA, 20 bp, synthetic construct
  misc_feature 1..20: note = Synthetic oligonucleotide
  source 1..20: mol_type = other DNA
aggtctccgccgtggaatcc

SEQ ID NO: 356 - DNA, 20 bp, synthetic construct
  misc_feature 1..20: note = Synthetic oligonucleotide
  source 1..20: mol_type = other DNA
taggtctccgccgtggaatc

SEQ ID NO: 357 - DNA, 20 bp, synthetic construct
  misc_feature 1..20: note = Synthetic oligonucleotide
  source 1..20: mol_type = other DNA
ttgtagtggacgatcttgcc

SEQ ID NO: 358 - DNA, 20 bp, synthetic construct
  misc_feature 1..20: note = Synthetic oligonucleotide
  source 1..20: mol_type = other DNA
cttgtagtggacgatcttgc

SEQ ID NO: 359 - DNA, 20 bp, synthetic construct
  misc_feature 1..20: note = Synthetic oligonucleotide
  source 1..20: mol_type = other DNA
ccttgtagtggacgatcttg

SEQ ID NO: 360 - DNA, 20 bp, synthetic construct
  misc_feature 1..20: note = Synthetic oligonucleotide
  source 1..20: mol_type = other DNA
tccttgtagtggacgatctt

SEQ ID NO: 361 - DNA, 20 bp, synthetic construct
  misc_feature 1..20: note = Synthetic oligonucleotide
  source 1..20: mol_type = other DNA
ctccttgtagtggacgatct

SEQ ID NO: 362 - DNA, 20 bp, synthetic construct
  misc_feature 1..20: note = Synthetic oligonucleotide
  source 1..20: mol_type = other DNA
gctccttgtagtggacgatc

SEQ ID NO: 363 - DNA, 20 bp, synthetic construct
  misc_feature 1..20: note = Synthetic oligonucleotide
  source 1..20: mol_type = other DNA
tgctccttgtagtggacgat

SEQ ID NO: 364 - DNA, 20 bp, synthetic construct
  misc_feature 1..20: note = Synthetic oligonucleotide
  source 1..20: mol_type = other DNA
gtgctccttgtagtggacga

SEQ ID NO: 365 - DNA, 20 bp, synthetic construct
  misc_feature 1..20: note = Synthetic oligonucleotide
  source 1..20: mol_type = other DNA
ggtgctccttgtagtggacg

SEQ ID NO: 366 - DNA, 20 bp, synthetic construct
  misc_feature 1..20: note = Synthetic oligonucleotide
  source 1..20: mol_type = other DNA
aggtgctccttgtagtggac

SEQ ID NO: 367 - DNA, 20 bp, synthetic construct
  misc_feature 1..20: note = Synthetic oligonucleotide
  source 1..20: mol_type = other DNA
gaggtgctccttgtagtgga

SEQ ID NO: 368 - DNA, 20 bp, synthetic construct
  misc_feature 1..20: note = Synthetic oligonucleotide
  source 1..20: mol_type = other DNA
agaggtgctccttgtagtgg

SEQ ID NO: 369 - DNA, 20 bp, synthetic construct
  misc_feature 1..20: note = Synthetic oligonucleotide
  source 1..20: mol_type = other DNA
gagaggtgctccttgtagtg

SEQ ID NO: 370 - DNA, 20 bp, synthetic construct
  misc_feature 1..20: note = Synthetic oligonucleotide
  source 1..20: mol_type = other DNA
agagaggtgctccttgtagt

SEQ ID NO: 371 - DNA, 20 bp, synthetic construct
  misc_feature 1..20: note = Synthetic oligonucleotide
  source 1..20: mol_type = other DNA
gagagaggtgctccttgtag

SEQ ID NO: 372 - DNA, 20 bp, synthetic construct
  misc_feature 1..20: note = Synthetic oligonucleotide
  source 1..20: mol_type = other DNA
agagagaggtgctccttgta

SEQ ID NO: 373 - DNA, 20 bp, synthetic construct
  misc_feature 1..20: note = Synthetic oligonucleotide
  source 1..20: mol_type = other DNA
cagagagaggtgctccttgt

SEQ ID NO: 374 - DNA, 20 bp, synthetic construct
  misc_feature 1..20: note = Synthetic oligonucleotide
  source 1..20: mol_type = other DNA
ggcagagagaggtgctcctt

SEQ ID NO: 375 - DNA, 20 bp, synthetic construct
  misc_feature 1..20: note = Synthetic oligonucleotide
  source 1..20: mol_type = other DNA
cggcagagagaggtgctcct

SEQ ID NO: 376 - DNA, 20 bp, synthetic construct
  misc_feature 1..20: note = Synthetic oligonucleotide
  source 1..20: mol_type = other DNA
gcggcagagagaggtgctcc

SEQ ID NO: 377 - DNA, 20 bp, synthetic construct
  misc_feature 1..20: note = Synthetic oligonucleotide
  source 1..20: mol_type = other DNA
agcggcagagagaggtgctc

SEQ ID NO: 378 - DNA, 20 bp, synthetic construct
  misc_feature 1..20: note = Synthetic oligonucleotide
  source 1..20: mol_type = other DNA
cagcggcagagagaggtgct

SEQ ID NO: 379 - DNA, 20 bp, synthetic construct
  misc_feature 1..20: note = Synthetic oligonucleotide
  source 1..20: mol_type = other DNA
ccagcggcagagagaggtgc

SEQ ID NO: 380 - DNA, 20 bp, synthetic construct
  misc_feature 1..20: note = Synthetic oligonucleotide
  source 1..20: mol_type = other DNA
ggcccagccgtgtctccggg

SEQ ID NO: 381 - DNA, 20 bp, synthetic construct
  misc_feature 1..20: note = Synthetic oligonucleotide
  source 1..20: mol_type = other DNA
cggcccagccgtgtctccgg

SEQ ID NO: 382 - DNA, 20 bp, synthetic construct
  misc_feature 1..20: note = Synthetic oligonucleotide
  source 1..20: mol_type = other DNA
ccggcccagccgtgtctccg

SEQ ID NO: 383 - DNA, 20 bp, synthetic construct
  misc_feature 1..20: note = Synthetic oligonucleotide
  source 1..20: mol_type = other DNA
cccggcccagccgtgtctcc

SEQ ID NO: 384 - DNA, 20 bp, synthetic construct
  misc_feature 1..20: note = Synthetic oligonucleotide
  source 1..20: mol_type = other DNA
ccccggcccagccgtgtctc

SEQ ID NO: 385 - DNA, 20 bp, synthetic construct
  misc_feature 1..20: note = Synthetic oligonucleotide
  source 1..20: mol_type = other DNA
accccggcccagccgtgtct

SEQ ID NO: 386 - DNA, 20 bp, synthetic construct
  misc_feature 1..20: note = Synthetic oligonucleotide
  source 1..20: mol_type = other DNA
caccccggcccagccgtgtc

SEQ ID NO: 387 - DNA, 20 bp, synthetic construct
  misc_feature 1..20: note = Synthetic oligonucleotide
  source 1..20: mol_type = other DNA
ccaccccggcccagccgtgt

SEQ ID NO: 388 - DNA, 20 bp, synthetic construct
  misc_feature 1..20: note = Synthetic oligonucleotide
  source 1..20: mol_type = other DNA
tccaccccggcccagccgtg

SEQ ID NO: 389 - DNA, 20 bp, synthetic construct
  misc_feature 1..20: note = Synthetic oligonucleotide
  source 1..20: mol_type = other DNA
ctccaccccggcccagccgt

SEQ ID NO: 390 - DNA, 20 bp, synthetic construct
  misc_feature 1..20: note = Synthetic oligonucleotide
  source 1..20: mol_type = other DNA
gctccaccccggcccagccg

SEQ ID NO: 391 - DNA, 20 bp, synthetic construct
  misc_feature 1..20: note = Synthetic oligonucleotide
  source 1..20: mol_type = other DNA
tgctccaccccggcccagcc

SEQ ID NO: 392 - DNA, 20 bp, synthetic construct
  misc_feature 1..20: note = Synthetic oligonucleotide
  source 1..20: mol_type = other DNA
ctgctccaccccggcccagc

SEQ ID NO: 393 - DNA, 20 bp, synthetic construct
  misc_feature 1..20: note = Synthetic oligonucleotide
  source 1..20: mol_type = other DNA
aagggatgtgtccggaagtc

SEQ ID NO: 394 - DNA, 20 bp, synthetic construct
  misc_feature 1..20: note = Synthetic oligonucleotide
  source 1..20: mol_type = other DNA
gaagggatgtgtccggaagt

SEQ ID NO: 395 - DNA, 20 bp, synthetic construct
  misc_feature 1..20: note = Synthetic oligonucleotide
  source 1..20: mol_type = other DNA
agaagggatgtgtccggaag

SEQ ID NO: 396 - DNA, 20 bp, synthetic construct
  misc_feature 1..20: note = Synthetic oligonucleotide
  source 1..20: mol_type = other DNA
aagaagggatgtgtccggaa

SEQ ID NO: 397 - DNA, 20 bp, synthetic construct
  misc_feature 1..20: note = Synthetic oligonucleotide
  source 1..20: mol_type = other DNA
gaagaagggatgtgtccgga

SEQ ID NO: 398 - DNA, 20 bp, synthetic construct
  misc_feature 1..20: note = Synthetic oligonucleotide
  source 1..20: mol_type = other DNA
agaagaagggatgtgtccgg

SEQ ID NO: 399 - DNA, 20 bp, synthetic construct
  misc_feature 1..20: note = Synthetic oligonucleotide
  source 1..20: mol_type = other DNA
aagaagaagggatgtgtccg

SEQ ID NO: 400 - DNA, 20 bp, synthetic construct
  misc_feature 1..20: note = Synthetic oligonucleotide
  source 1..20: mol_type = other DNA
aaagaagaagggatgtgtcc

SEQ ID NO: 401 - DNA, 20 bp, synthetic construct
  misc_feature 1..20: note = Synthetic oligonucleotide
  source 1..20: mol_type = other DNA
caaagaagaagggatgtgtc

SEQ ID NO: 402 - DNA, 20 bp, synthetic construct
  misc_feature 1..20: note = Synthetic oligonucleotide
  source 1..20: mol_type = other DNA
ccaaagaagaagggatgtgt

SEQ ID NO: 403 - DNA, 20 bp, synthetic construct
  misc_feature 1..20: note = Synthetic oligonucleotide
  source 1..20: mol_type = other DNA
ggccaaagaagaagggatgt

SEQ ID NO: 404 - DNA, 20 bp, synthetic construct
  misc_feature 1..20: note = Synthetic oligonucleotide
  source 1..20: mol_type = other DNA
aggccaaagaagaagggatg

SEQ ID NO: 405 - DNA, 20 bp, synthetic construct
  misc_feature 1..20: note = Synthetic oligonucleotide
  source 1..20: mol_type = other DNA
gaggccaaagaagaagggat

SEQ ID NO: 406 - DNA, 20 bp, synthetic construct
  misc_feature 1..20: note = Synthetic oligonucleotide
  source 1..20: mol_type = other DNA
cgaggccaaagaagaaggga

SEQ ID NO: 407 - DNA, 20 bp, synthetic construct
  misc_feature 1..20: note = Synthetic oligonucleotide
  source 1..20: mol_type = other DNA
tcgaggccaaagaagaaggg

SEQ ID NO: 408 - DNA, 20 bp, synthetic construct
  misc_feature 1..20: note = Synthetic oligonucleotide
  source 1..20: mol_type = other DNA
gtcgaggccaaagaagaagg

SEQ ID NO: 409 - DNA, 20 bp, synthetic construct
  misc_feature 1..20: note = Synthetic oligonucleotide
  source 1..20: mol_type = other DNA
agtcgaggccaaagaagaag

SEQ ID NO: 410 - DNA, 20 bp, synthetic construct
  misc_feature 1..20: note = Synthetic oligonucleotide
  source 1..20: mol_type = other DNA
cagtcgaggccaaagaagaa

SEQ ID NO: 411 - DNA, 20 bp, synthetic construct
  misc_feature 1..20: note = Synthetic oligonucleotide
  source 1..20: mol_type = other DNA
ccagtcgaggccaaagaaga

SEQ ID NO: 412 - DNA, 20 bp, synthetic construct
  misc_feature 1..20: note = Synthetic oligonucleotide
  source 1..20: mol_type = other DNA
cccagtcgaggccaaagaag

SEQ ID NO: 413 - DNA, 20 bp, synthetic construct
  misc_feature 1..20: note = Synthetic oligonucleotide
  source 1..20: mol_type = other DNA
tcccagtcgaggccaaagaa

SEQ ID NO: 414 - DNA, 20 bp, synthetic construct
  misc_feature 1..20: note = Synthetic oligonucleotide
  source 1..20: mol_type = other DNA
atcccagtcgaggccaaaga

SEQ ID NO: 415 - DNA, 20 bp, synthetic construct
  misc_feature 1..20: note = Synthetic oligonucleotide
  source 1..20: mol_type = other DNA
catcccagtcgaggccaaag

SEQ ID NO: 416 - DNA, 20 bp, synthetic construct
  misc_feature 1..20: note = Synthetic oligonucleotide
  source 1..20: mol_type = other DNA
ccatcccagtcgaggccaaa

SEQ ID NO: 417 - DNA, 20 bp, synthetic construct
  misc_feature 1..20: note = Synthetic oligonucleotide
  source 1..20: mol_type = other DNA
accatcccagtcgaggccaa

SEQ ID NO: 418 - DNA, 20 bp, synthetic construct
  misc_feature 1..20: note = Synthetic oligonucleotide
  source 1..20: mol_type = other DNA
gaccatcccagtcgaggcca

SEQ ID NO: 419 - DNA, 20 bp, synthetic construct
  misc_feature 1..20: note = Synthetic oligonucleotide
  source 1..20: mol_type = other DNA
agaccatcccagtcgaggcc

SEQ ID NO: 420 - DNA, 20 bp, synthetic construct
  misc_feature 1..20: note = Synthetic oligonucleotide
  source 1..20: mol_type = other DNA
gagaccatcccagtcgaggc

SEQ ID NO: 421 - DNA, 20 bp, synthetic construct
  misc_feature 1..20: note = Synthetic oligonucleotide
  source 1..20: mol_type = other DNA
ggagaccatcccagtcgagg

SEQ ID NO: 422 - DNA, 20 bp, synthetic construct
  misc_feature 1..20: note = Synthetic oligonucleotide
  source 1..20: mol_type = other DNA
ttcgaaatccggtgtaaagg

SEQ ID NO: 423 - DNA, 20 bp, synthetic construct
  misc_feature 1..20: note = Synthetic oligonucleotide
  source 1..20: mol_type = other DNA
cttcgaaatccggtgtaaag

SEQ ID NO: 424 - DNA, 20 bp, synthetic construct
  misc_feature 1..20: note = Synthetic oligonucleotide
  source 1..20: mol_type = other DNA
ccttcgaaatccggtgtaaa

SEQ ID NO: 425 - DNA, 20 bp, synthetic construct
  misc_feature 1..20: note = Synthetic oligonucleotide
  source 1..20: mol_type = other DNA
accttcgaaatccggtgtaa

SEQ ID NO: 426 - DNA, 20 bp, synthetic construct
  misc_feature 1..20: note = Synthetic oligonucleotide
  source 1..20: mol_type = other DNA
caccttcgaaatccggtgta

SEQ ID NO: 427 - DNA, 20 bp, synthetic construct
  misc_feature 1..20: note = Synthetic oligonucleotide
  source 1..20: mol_type = other DNA
gcaccttcgaaatccggtgt

SEQ ID NO: 428 - DNA, 20 bp, synthetic construct
  misc_feature 1..20: note = Synthetic oligonucleotide
  source 1..20: mol_type = other DNA
ggcaccttcgaaatccggtg

SEQ ID NO: 429 - DNA, 20 bp, synthetic construct
  misc_feature 1..20: note = Synthetic oligonucleotide
  source 1..20: mol_type = other DNA
tggcaccttcgaaatccggt

SEQ ID NO: 430 - DNA, 20 bp, synthetic construct
  misc_feature 1..20: note = Synthetic oligonucleotide
  source 1..20: mol_type = other DNA
gtggcaccttcgaaatccgg

SEQ ID NO: 431 - DNA, 20 bp, synthetic construct
  misc_feature 1..20: note = Synthetic oligonucleotide
  source 1..20: mol_type = other DNA
ggtggcaccttcgaaatccg

SEQ ID NO: 432 - DNA, 20 bp, synthetic construct
  misc_feature 1..20: note = Synthetic oligonucleotide
  source 1..20: mol_type = other DNA
cggtggcaccttcgaaatcc

SEQ ID NO: 433 - DNA, 20 bp, synthetic construct
  misc_feature 1..20: note = Synthetic oligonucleotide
  source 1..20: mol_type = other DNA
tcggtggcaccttcgaaatc

SEQ ID NO: 434 - DNA, 20 bp, synthetic construct
  misc_feature 1..20: note = Synthetic oligonucleotide
  source 1..20: mol_type = other DNA
gtcggtggcaccttcgaaat

SEQ ID NO: 435 - DNA, 20 bp, synthetic construct
  misc_feature 1..20: note = Synthetic oligonucleotide
  source 1..20: mol_type = other DNA
tgtcggtggcaccttcgaaa

SEQ ID NO: 436 - DNA, 20 bp, synthetic construct
  misc_feature 1..20: note = Synthetic oligonucleotide
  source 1..20: mol_type = other DNA
gtgtcggtggcaccttcgaa

SEQ ID NO: 437 - DNA, 20 bp, synthetic construct
  misc_feature 1..20: note = Synthetic oligonucleotide
  source 1..20: mol_type = other DNA
tgtgtcggtggcaccttcga

SEQ ID NO: 438 - DNA, 20 bp, synthetic construct
  misc_feature 1..20: note = Synthetic oligonucleotide
  source 1..20: mol_type = other DNA
atgtgtcggtggcaccttcg

SEQ ID NO: 439 - DNA, 20 bp, synthetic construct
  misc_feature 1..20: note = Synthetic oligonucleotide
  source 1..20: mol_type = other DNA
catgtgtcggtggcaccttc

SEQ ID NO: 440 - DNA, 20 bp, synthetic construct
  misc_feature 1..20: note = Synthetic oligonucleotide
  source 1..20: mol_type = other DNA
gcatgtgtcggtggcacctt

SEQ ID NO: 441 - DNA, 20 bp, synthetic construct
  misc_feature 1..20: note = Synthetic oligonucleotide
  source 1..20: mol_type = other DNA
tgcatgtgtcggtggcacct

SEQ ID NO: 442 - DNA, 20 bp, synthetic construct
  misc_feature 1..20: note = Synthetic oligonucleotide
  source 1..20: mol_type = other DNA
ttgcatgtgtcggtggcacc

SEQ ID NO: 443 - DNA, 20 bp, synthetic construct
  misc_feature 1..20: note = Synthetic oligonucleotide
  source 1..20: mol_type = other DNA
gttgcatgtgtcggtggcac

SEQ ID NO: 444 - DNA, 20 bp, synthetic construct
  misc_feature 1..20: note = Synthetic oligonucleotide
  source 1..20: mol_type = other DNA
agttgcatgtgtcggtggca

SEQ ID NO: 445 - DNA, 20 bp, synthetic construct
  misc_feature 1..20: note = Synthetic oligonucleotide
  source 1..20: mol_type = other DNA
aagttgcatgtgtcggtggc

SEQ ID NO: 446 - DNA, 20 bp, synthetic construct
  misc_feature 1..20: note = Synthetic oligonucleotide
  source 1..20: mol_type = other DNA
gaagttgcatgtgtcggtgg

SEQ ID NO: 447 - DNA, 20 bp, synthetic construct
  misc_feature 1..20: note = Synthetic oligonucleotide
  source 1..20: mol_type = other DNA
cgaagttgcatgtgtcggtg

SEQ ID NO: 448 - DNA, 20 bp, synthetic construct
  misc_feature 1..20: note = Synthetic oligonucleotide
  source 1..20: mol_type = other DNA
gtcgaagttgcatgtgtcgg

SEQ ID NO: 449 - DNA, 20 bp, synthetic construct
  misc_feature 1..20: note = Synthetic oligonucleotide
  source 1..20: mol_type = other DNA
agtcgaagttgcatgtgtcg

SEQ ID NO: 450 - DNA, 20 bp, synthetic construct
  misc_feature 1..20: note = Synthetic oligonucleotide
  source 1..20: mol_type = other DNA
aagtcgaagttgcatgtgtc

SEQ ID NO: 451 - DNA, 20 bp, synthetic construct
  misc_feature 1..20: note = Synthetic oligonucleotide
  source 1..20: mol_type = other DNA
caagtcgaagttgcatgtgt

SEQ ID NO: 452 - DNA, 20 bp, synthetic construct
  misc_feature 1..20: note = Synthetic oligonucleotide
  source 1..20: mol_type = other DNA
ccaagtcgaagttgcatgtg

SEQ ID NO: 453 - DNA, 20 bp, synthetic construct
  misc_feature 1..20: note = Synthetic oligonucleotide
  source 1..20: mol_type = other DNA
accaagtcgaagttgcatgt

SEQ ID NO: 454 - DNA, 20 bp, synthetic construct
  misc_feature 1..20: note = Synthetic oligonucleotide
  source 1..20: mol_type = other DNA
caccaagtcgaagttgcatg

SEQ ID NO: 455 - DNA, 20 bp, synthetic construct
  misc_feature 1..20: note = Synthetic oligonucleotide
  source 1..20: mol_type = other DNA
ccaccaagtcgaagttgcat

SEQ ID NO: 456 - DNA, 20 bp, synthetic construct
  misc_feature 1..20: note = Synthetic oligonucleotide
  source 1..20: mol_type = other DNA
tccaccaagtcgaagttgca

SEQ ID NO: 457 - DNA, 20 bp, synthetic construct
  misc_feature 1..20: note = Synthetic oligonucleotide
  source 1..20: mol_type = other DNA
ctccaccaagtcgaagttgc

SEQ ID NO: 458 - DNA, 20 bp, synthetic construct
  misc_feature 1..20: note = Synthetic oligonucleotide
  source 1..20: mol_type = other DNA
cctccaccaagtcgaagttg

SEQ ID NO: 459 - DNA, 20 bp, synthetic construct
  misc_feature 1..20: note = Synthetic oligonucleotide
  source 1..20: mol_type = other DNA
tcctccaccaagtcgaagtt

SEQ ID NO: 460 - DNA, 20 bp, synthetic construct
  misc_feature 1..20: note = Synthetic oligonucleotide
  source 1..20: mol_type = other DNA
gtcctccaccaagtcgaagt

SEQ ID NO: 461 - DNA, 20 bp, synthetic construct
  misc_feature 1..20: note = Synthetic oligonucleotide
  source 1..20: mol_type = other DNA
cgtcctccaccaagtcgaag

SEQ ID NO: 462 - DNA, 20 bp, synthetic construct
  misc_feature 1..20: note = Synthetic oligonucleotide
  source 1..20: mol_type = other DNA
ccgtcctccaccaagtcgaa

SEQ ID NO: 463 - DNA, 20 bp, synthetic construct
  misc_feature 1..20: note = Synthetic oligonucleotide
  source 1..20: mol_type = other DNA
cccgtcctccaccaagtcga

SEQ ID NO: 464 - DNA, 20 bp, synthetic construct
  misc_feature 1..20: note = Synthetic oligonucleotide
  source 1..20: mol_type = other DNA
gcccgtcctccaccaagtcg

SEQ ID NO: 465 - DNA, 20 bp, synthetic construct
  misc_feature 1..20: note = Synthetic oligonucleotide
  source 1..20: mol_type = other DNA
agcccgtcctccaccaagtc

SEQ ID NO: 466 - DNA, 20 bp, synthetic construct
  misc_feature 1..20: note = Synthetic oligonucleotide
  source 1..20: mol_type = other DNA
gagcccgtcctccaccaagt

SEQ ID NO: 467 - DNA, 20 bp, synthetic construct
  misc_feature 1..20: note = Synthetic oligonucleotide
  source 1..20: mol_type = other DNA
tgagcccgtcctccaccaag

SEQ ID NO: 468 - DNA, 20 bp, synthetic construct
  misc_feature 1..20: note = Synthetic oligonucleotide
  source 1..20: mol_type = other DNA
ggttccgagcctctgcctcg

SEQ ID NO: 469 - DNA, 20 bp, synthetic construct
  misc_feature 1..20: note = Synthetic oligonucleotide
  source 1..20: mol_type = other DNA
cggttccgagcctctgcctc

SEQ ID NO: 470 - DNA, 20 bp, synthetic construct
  misc_feature 1..20: note = Synthetic oligonucleotide
  source 1..20: mol_type = other DNA
ccggttccgagcctctgcct

SEQ ID NO: 471 - DNA, 20 bp, synthetic construct
  misc_feature 1..20: note = Synthetic oligonucleotide
  source 1..20: mol_type = other DNA
cccggttccgagcctctgcc

SEQ ID NO: 472 - DNA, 20 bp, synthetic construct
  misc_feature 1..20: note = Synthetic oligonucleotide
  source 1..20: mol_type = other DNA
tcccggttccgagcctctgc

SEQ ID NO: 473 - DNA, 20 bp, synthetic construct
  misc_feature 1..20: note = Synthetic oligonucleotide
  source 1..20: mol_type = other DNA
gtcccggttccgagcctctg

SEQ ID NO: 474 - DNA, 20 bp, synthetic construct
  misc_feature 1..20: note = Synthetic oligonucleotide
  source 1..20: mol_type = other DNA
aggtcccggttccgagcctc

SEQ ID NO: 475 - DNA, 20 bp, synthetic construct
  misc_feature 1..20: note = Synthetic oligonucleotide
  source 1..20: mol_type = other DNA
taggtcccggttccgagcct

SEQ ID NO: 476 - DNA, 20 bp, synthetic construct
  misc_feature 1..20: note = Synthetic oligonucleotide
  source 1..20: mol_type = other DNA
ctaggtcccggttccgagcc

SEQ ID NO: 477 - DNA, 20 bp, synthetic construct
  misc_feature 1..20: note = Synthetic oligonucleotide
  source 1..20: mol_type = other DNA
tctaggtcccggttccgagc

SEQ ID NO: 478 - DNA, 20 bp, synthetic construct
  misc_feature 1..20: note = Synthetic oligonucleotide
  source 1..20: mol_type = other DNA
ctctaggtcccggttccgag

SEQ ID NO: 479 - DNA, 20 bp, synthetic construct
  misc_feature 1..20: note = Synthetic oligonucleotide
  source 1..20: mol_type = other DNA
cctctaggtcccggttccga

SEQ ID NO: 480 - DNA, 20 bp, synthetic construct
  misc_feature 1..20: note = Synthetic oligonucleotide
  source 1..20: mol_type = other DNA
gcctctaggtcccggttccg

SEQ ID NO: 481 - DNA, 20 bp, synthetic construct
  misc_feature 1..20: note = Synthetic oligonucleotide
  source 1..20: mol_type = other DNA
catccgctcctgcaactgcc

SEQ ID NO: 482 - DNA, 20 bp, synthetic construct
  misc_feature 1..20: note = Synthetic oligonucleotide
  source 1..20: mol_type = other DNA
ccatccgctcctgcaactgc

SEQ ID NO: 483 - DNA, 20 bp, synthetic construct
  misc_feature 1..20: note = Synthetic oligonucleotide
  source 1..20: mol_type = other DNA
tccatccgctcctgcaactg

SEQ ID NO: 484 - DNA, 20 bp, synthetic construct
  misc_feature 1..20: note = Synthetic oligonucleotide
  source 1..20: mol_type = other DNA
ctccatccgctcctgcaact

SEQ ID NO: 485 - DNA, 20 bp, synthetic construct
  misc_feature 1..20: note = Synthetic oligonucleotide
  source 1..20: mol_type = other DNA
actccatccgctcctgcaac

SEQ ID NO: 486 - DNA, 20 bp, synthetic construct
  misc_feature 1..20: note = Synthetic oligonucleotide
  source 1..20: mol_type = other DNA
aactccatccgctcctgcaa

SEQ ID NO: 487 - DNA, 20 bp, synthetic construct
  misc_feature 1..20: note = Synthetic oligonucleotide
  source 1..20: mol_type = other DNA
caactccatccgctcctgca

SEQ ID NO: 488 - DNA, 20 bp, synthetic construct
  misc_feature 1..20: note = Synthetic oligonucleotide
  source 1..20: mol_type = other DNA
agcaactccatccgctcctg

SEQ ID NO: 489 - DNA, 20 bp, synthetic construct
  misc_feature 1..20: note = Synthetic oligonucleotide
  source 1..20: mol_type = other DNA
cagcaactccatccgctcct

SEQ ID NO: 490 - DNA, 20 bp, synthetic construct
  misc_feature 1..20: note = Synthetic oligonucleotide
  source 1..20: mol_type = other DNA
gcagcaactccatccgctcc

SEQ ID NO: 491 - DNA, 20 bp, synthetic construct
  misc_feature 1..20: note = Synthetic oligonucleotide
  source 1..20: mol_type = other DNA
cagctgtggctccctctgcc

SEQ ID NO: 492 - DNA, 20 bp, synthetic construct
  misc_feature 1..20: note = Synthetic oligonucleotide
  source 1..20: mol_type = other DNA
acagctgtggctccctctgc

SEQ ID NO: 493 - DNA, 20 bp, synthetic construct
  misc_feature 1..20: note = Synthetic oligonucleotide
  source 1..20: mol_type = other DNA
gacagctgtggctccctctg

SEQ ID NO: 494 - DNA, 20 bp, synthetic construct
  misc_feature 1..20: note = Synthetic oligonucleotide
  source 1..20: mol_type = other DNA
tgacagctgtggctccctct

SEQ ID NO: 495 - DNA, 20 bp, synthetic construct
  misc_feature 1..20: note = Synthetic oligonucleotide
  source 1..20: mol_type = other DNA
gtgacagctgtggctccctc

SEQ ID NO: 496 - DNA, 20 bp, synthetic construct
  misc_feature 1..20: note = Synthetic oligonucleotide
  source 1..20: mol_type = other DNA
cgtgacagctgtggctccct

SEQ ID NO: 497 - DNA, 20 bp, synthetic construct
  misc_feature 1..20: note = Synthetic oligonucleotide
  source 1..20: mol_type = other DNA
ccgtgacagctgtggctccc

SEQ ID NO: 498 - DNA, 20 bp, synthetic construct
  misc_feature 1..20: note = Synthetic oligonucleotide
  source 1..20: mol_type = other DNA
cccgtgacagctgtggctcc

SEQ ID NO: 499 - DNA, 20 bp, synthetic construct
  misc_feature 1..20: note = Synthetic oligonucleotide
  source 1..20: mol_type = other DNA
ccccgtgacagctgtggctc

SEQ ID NO: 500 - DNA, 20 bp, synthetic construct
  misc_feature 1..20: note = Synthetic oligonucleotide
  source 1..20: mol_type = other DNA
cccccgtgacagctgtggct

SEQ ID NO: 501 - DNA, 20 bp, synthetic construct
  misc_feature 1..20: note = Synthetic oligonucleotide
  source 1..20: mol_type = other DNA
acccccgtgacagctgtggc

SEQ ID NO: 502 - DNA, 20 bp, synthetic construct
  misc_feature 1..20: note = Synthetic oligonucleotide
  source 1..20: mol_type = other DNA
gacccccgtgacagctgtgg

SEQ ID NO: 503 - DNA, 20 bp, synthetic construct
  misc_feature 1..20: note = Synthetic oligonucleotide
  source 1..20: mol_type = other DNA
ggacccccgtgacagctgtg

SEQ ID NO: 504 - DNA, 20 bp, synthetic construct
  misc_feature 1..20: note = Synthetic oligonucleotide
  source 1..20: mol_type = other DNA
gggacccccgtgacagctgt

SEQ ID NO: 505 - DNA, 20 bp, synthetic construct
  misc_feature 1..20: note = Synthetic oligonucleotide
  source 1..20: mol_type = other DNA
gaaggtggatccgtggcccg

SEQ ID NO: 506 - DNA, 20 bp, synthetic construct
  misc_feature 1..20: note = Synthetic oligonucleotide
  source 1..20: mol_type = other DNA
ggaaggtggatccgtggccc

SEQ ID NO: 507 - DNA, 20 bp, synthetic construct
  misc_feature 1..20: note = Synthetic oligonucleotide
  source 1..20: mol_type = other DNA
gggaaggtggatccgtggcc

SEQ ID NO: 508 - DNA, 20 bp, synthetic construct
  misc_feature 1..20: note = Synthetic oligonucleotide
  source 1..20: mol_type = other DNA
tgggaaggtggatccgtggc

SEQ ID NO: 509 - DNA, 20 bp, synthetic construct
  misc_feature 1..20: note = Synthetic oligonucleotide
  source 1..20: mol_type = other DNA
atgggaaggtggatccgtgg

SEQ ID NO: 510 - DNA, 20 bp, synthetic construct
  misc_feature 1..20: note = Synthetic oligonucleotide
  source 1..20: mol_type = other DNA
gatgggaaggtggatccgtg

SEQ ID NO: 511 - DNA, 20 bp, synthetic construct
  misc_feature 1..20: note = Synthetic oligonucleotide
  source 1..20: mol_type = other DNA
tagatgggaaggtggatccg

SEQ ID NO: 512 - DNA, 20 bp, synthetic construct
  misc_feature 1..20: note = Synthetic oligonucleotide
  source 1..20: mol_type = other DNA
ctagatgggaaggtggatcc

SEQ ID NO: 513 - DNA, 20 bp, synthetic construct
  misc_feature 1..20: note = Synthetic oligonucleotide
  source 1..20: mol_type = other DNA
tctagatgggaaggtggatc

SEQ ID NO: 514 - DNA, 20 bp, synthetic construct
  misc_feature 1..20: note = Synthetic oligonucleotide
  source 1..20: mol_type = other DNA
atctagatgggaaggtggat

SEQ ID NO: 515 - DNA, 20 bp, synthetic construct
  misc_feature 1..20: note = Synthetic oligonucleotide
  source 1..20: mol_type = other DNA
ccatctagatgggaaggtgg

SEQ ID NO: 516 - DNA, 20 bp, synthetic construct
  misc_feature 1..20: note = Synthetic oligonucleotide
  source 1..20: mol_type = other DNA
gccatctagatgggaaggtg

SEQ ID NO: 517 - DNA, 20 bp, synthetic construct
  misc_feature 1..20: note = Synthetic oligonucleotide
  source 1..20: mol_type = other DNA
ggccatctagatgggaaggt

SEQ ID NO: 518 - DNA, 20 bp, synthetic construct
  misc_feature 1..20: note = Synthetic oligonucleotide
  source 1..20: mol_type = other DNA
caccagcgggcactggccca

SEQ ID NO: 519 - DNA, 20 bp, synthetic construct
  misc_feature 1..20: note = Synthetic oligonucleotide
  source 1..20: mol_type = other DNA
ccaccagcgggcactggccc

SEQ ID NO: 520 - DNA, 20 bp, synthetic construct
  misc_feature 1..20: note = Synthetic oligonucleotide
  source 1..20: mol_type = other DNA
cccaccagcgggcactggcc

SEQ ID NO: 521 - DNA, 20 bp, synthetic construct
  misc_feature 1..20: note = Synthetic oligonucleotide
  source 1..20: mol_type = other DNA
ccccaccagcgggcactggc

SEQ ID NO: 522 - DNA, 20 bp, synthetic construct
  misc_feature 1..20: note = Synthetic oligonucleotide
  source 1..20: mol_type = other DNA
ggccccaccagcgggcactg

SEQ ID NO: 523 - DNA, 20 bp, synthetic construct
  misc_feature 1..20: note = Synthetic oligonucleotide
  source 1..20: mol_type = other DNA
tggccccaccagcgggcact

SEQ ID NO: 524 - DNA, 20 bp, synthetic construct
  misc_feature 1..20: note = Synthetic oligonucleotide
  source 1..20: mol_type = other DNA
ctggccccaccagcgggcac

SEQ ID NO: 525 - DNA, 20 bp, synthetic construct
  misc_feature 1..20: note = Synthetic oligonucleotide
  source 1..20: mol_type = other DNA
cctggccccaccagcgggca

SEQ ID NO: 526 - DNA, 20 bp, synthetic construct
  misc_feature 1..20: note = Synthetic oligonucleotide
  source 1..20: mol_type = other DNA
gcctggccccaccagcgggc

SEQ ID NO: 527 - DNA, 20 bp, synthetic construct
  misc_feature 1..20: note = Synthetic oligonucleotide
  source 1..20: mol_type = other DNA
gggcctggccccaccagcgg

SEQ ID NO: 528 - DNA, 20 bp, synthetic construct
  misc_feature 1..20: note = Synthetic oligonucleotide
  source 1..20: mol_type = other DNA
aggtggcggcggtgcatggg

SEQ ID NO: 529 - DNA, 20 bp, synthetic construct
  misc_feature 1..20: note = Synthetic oligonucleotide
  source 1..20: mol_type = other DNA
caggtggcggcggtgcatgg

SEQ ID NO: 530 - DNA, 20 bp, synthetic construct
  misc_feature 1..20: note = Synthetic oligonucleotide
  source 1..20: mol_type = other DNA
gcaggtggcggcggtgcatg

SEQ ID NO: 531 - DNA, 20 bp, synthetic construct
  misc_feature 1..20: note = Synthetic oligonucleotide
  source 1..20: mol_type = other DNA
agcaggtggcggcggtgcat

SEQ ID NO: 532 - DNA, 20 bp, synthetic construct
  misc_feature 1..20: note = Synthetic oligonucleotide
  source 1..20: mol_type = other DNA
cagcaggtggcggcggtgca

SEQ ID NO: 533 - DNA, 20 bp, synthetic construct
  misc_feature 1..20: note = Synthetic oligonucleotide
  source 1..20: mol_type = other DNA
gcagcaggtggcggcggtgc

SEQ ID NO: 534 - DNA, 20 bp, synthetic construct
  misc_feature 1..20: note = Synthetic oligonucleotide
  source 1..20: mol_type = other DNA
agcagcaggtggcggcggtg

SEQ ID NO: 535 - DNA, 20 bp, synthetic construct
  misc_feature 1..20: note = Synthetic oligonucleotide
  source 1..20: mol_type = other DNA
gagcagcaggtggcggcggt

SEQ ID NO: 536 - DNA, 20 bp, synthetic construct
  misc_feature 1..20: note = Synthetic oligonucleotide
  source 1..20: mol_type = other DNA
ggagcagcaggtggcggcgg

SEQ ID NO: 537 - DNA, 20 bp, synthetic construct
  misc_feature 1..20: note = Synthetic oligonucleotide
  source 1..20: mol_type = other DNA
gggagcagcaggtggcggcg

SEQ ID NO: 538 - DNA, 20 bp, synthetic construct
  misc_feature 1..20: note = Synthetic oligonucleotide
  source 1..20: mol_type = other DNA
agggagcagcaggtggcggc

SEQ ID NO: 539 - DNA, 20 bp, synthetic construct
  misc_feature 1..20: note = Synthetic oligonucleotide
  source 1..20: mol_type = other DNA
cagggagcagcaggtggcgg

SEQ ID NO: 540 - DNA, 20 bp, synthetic construct
  misc_feature 1..20: note = Synthetic oligonucleotide
  source 1..20: mol_type = other DNA
gcagggagcagcaggtggcg

SEQ ID NO: 541 - DNA, 20 bp, synthetic construct
  misc_feature 1..20: note = Synthetic oligonucleotide
  source 1..20: mol_type = other DNA
ggcagggagcagcaggtggc

SEQ ID NO: 542 - DNA, 20 bp, synthetic construct
  misc_feature 1..20: note = Synthetic oligonucleotide
  source 1..20: mol_type = other DNA
tggcagggagcagcaggtgg

SEQ ID NO: 543 - DNA, 20 bp, synthetic construct
  misc_feature 1..20: note = Synthetic oligonucleotide
  source 1..20: mol_type = other DNA
ctggcagggagcagcaggtg

SEQ ID NO: 544 - DNA, 20 bp, synthetic construct
  misc_feature 1..20: note = Synthetic oligonucleotide
  source 1..20: mol_type = other DNA
ccctggcagggagcagcagg

SEQ ID NO: 545 - DNA, 20 bp, synthetic construct
  misc_feature 1..20: note = Synthetic oligonucleotide
  source 1..20: mol_type = other DNA
accctggcagggagcagcag

SEQ ID NO: 546 - DNA, 20 bp, synthetic construct
  misc_feature 1..20: note = Synthetic oligonucleotide
  source 1..20: mol_type = other DNA
gaccctggcagggagcagca

SEQ ID NO: 547 - DNA, 20 bp, synthetic construct
  misc_feature 1..20: note = Synthetic oligonucleotide
  source 1..20: mol_type = other DNA
ggaccctggcagggagcagc

SEQ ID NO: 548 - DNA, 20 bp, synthetic construct
  misc_feature 1..20: note = Synthetic oligonucleotide
  source 1..20: mol_type = other DNA
ggcctagggaccctggcagg

SEQ ID NO: 549 - DNA, 20 bp, synthetic construct
  misc_feature 1..20: note = Synthetic oligonucleotide
  source 1..20: mol_type = other DNA
aggcctagggaccctggcag

SEQ ID NO: 550 - DNA, 20 bp, synthetic construct
  misc_feature 1..20: note = Synthetic oligonucleotide
  source 1..20: mol_type = other DNA
ccaggcctagggaccctggc

SEQ ID NO: 551 - DNA, 20 bp, synthetic construct
  misc_feature 1..20: note = Synthetic oligonucleotide
  source 1..20: mol_type = other DNA
gccaggcctagggaccctgg

SEQ ID NO: 552 - DNA, 20 bp, synthetic construct
  misc_feature 1..20: note = Synthetic oligonucleotide
  source 1..20: mol_type = other DNA
ggccaggcctagggaccctg

SEQ ID NO: 553 - DNA, 20 bp, synthetic construct
  misc_feature 1..20: note = Synthetic oligonucleotide
  source 1..20: mol_type = other DNA
aggccaggcctagggaccct

SEQ ID NO: 554 - DNA, 20 bp, synthetic construct
  misc_feature 1..20: note = Synthetic oligonucleotide
  source 1..20: mol_type = other DNA
taggccaggcctagggaccc

SEQ ID NO: 555 - DNA, 20 bp, synthetic construct
  misc_feature 1..20: note = Synthetic oligonucleotide
  source 1..20: mol_type = other DNA
ataggccaggcctagggacc

SEQ ID NO: 556 - DNA, 20 bp, synthetic construct
  misc_feature 1..20: note = Synthetic oligonucleotide
  source 1..20: mol_type = other DNA
gataggccaggcctagggac

SEQ ID NO: 557 - DNA, 20 bp, synthetic construct
  misc_feature 1..20: note = Synthetic oligonucleotide
  source 1..20: mol_type = other DNA
cgataggccaggcctaggga

SEQ ID NO: 558 - DNA, 20 bp, synthetic construct
  misc_feature 1..20: note = Synthetic oligonucleotide
  source 1..20: mol_type = other DNA
ccgataggccaggcctaggg

SEQ ID NO: 559 - DNA, 20 bp, synthetic construct
  misc_feature 1..20: note = Synthetic oligonucleotide
  source 1..20: mol_type = other DNA
tccgataggccaggcctagg

SEQ ID NO: 560 - DNA, 20 bp, synthetic construct
  misc_feature 1..20: note = Synthetic oligonucleotide
  source 1..20: mol_type = other DNA
ctccgataggccaggcctag

SEQ ID NO: 561 - DNA, 20 bp, synthetic construct
  misc_feature 1..20: note = Synthetic oligonucleotide
  source 1..20: mol_type = other DNA
cctccgataggccaggccta

SEQ ID NO: 562 - DNA, 20 bp, synthetic construct
  misc_feature 1..20: note = Synthetic oligonucleotide
  source 1..20: mol_type = other DNA
gcctccgataggccaggcct

SEQ ID NO: 563 - DNA, 20 bp, synthetic construct
  misc_feature 1..20: note = Synthetic oligonucleotide
  source 1..20: mol_type = other DNA
gcgcctccgataggccaggc

SEQ ID NO: 564 - DNA, 20 bp, synthetic construct
  misc_feature 1..20: note = Synthetic oligonucleotide
  source 1..20: mol_type = other DNA
aacaggagcagggaaagcgc

SEQ ID NO: 565 - DNA, 20 bp, synthetic construct
  misc_feature 1..20: note = Synthetic oligonucleotide
  source 1..20: mol_type = other DNA
gaacaggagcagggaaagcg

SEQ ID NO: 566 - DNA, 20 bp, synthetic construct
  misc_feature 1..20: note = Synthetic oligonucleotide
  source 1..20: mol_type = other DNA
cgaacaggagcagggaaagc

SEQ ID NO: 567 - DNA, 20 bp, synthetic construct
  misc_feature 1..20: note = Synthetic oligonucleotide
  source 1..20: mol_type = other DNA
gcgaacaggagcagggaaag

SEQ ID NO: 568 - DNA, 20 bp, synthetic construct
  misc_feature 1..20: note = Synthetic oligonucleotide
  source 1..20: mol_type = other DNA
ggcgaacaggagcagggaaa

SEQ ID NO: 569 - DNA, 20 bp, synthetic construct
  misc_feature 1..20: note = Synthetic oligonucleotide
  source 1..20: mol_type = other DNA
cggcgaacaggagcagggaa

SEQ ID NO: 570 - DNA, 20 bp, synthetic construct
  misc_feature 1..20: note = Synthetic oligonucleotide
  source 1..20: mol_type = other DNA
acggcgaacaggagcaggga

SEQ ID NO: 571 - DNA, 20 bp, synthetic construct
  misc_feature 1..20: note = Synthetic oligonucleotide
  source 1..20: mol_type = other DNA
aacggcgaacaggagcaggg

SEQ ID NO: 572 - DNA, 20 bp, synthetic construct
  misc_feature 1..20: note = Synthetic oligonucleotide
  source 1..20: mol_type = other DNA
caacggcgaacaggagcagg

SEQ ID NO: 573 - DNA, 20 bp, synthetic construct
  misc_feature 1..20: note = Synthetic oligonucleotide
  source 1..20: mol_type = other DNA
gggcggcggcacgagacaga

SEQ ID NO: 574 - DNA, 20 bp, synthetic construct
  misc_feature 1..20: note = Synthetic oligonucleotide
  source 1..20: mol_type = other DNA
agggcggcggcacgagacag

SEQ ID NO: 575 - DNA, 20 bp, synthetic construct
  misc_feature 1..20: note = Synthetic oligonucleotide
  source 1..20: mol_type = other DNA
cagggcggcggcacgagaca

SEQ ID NO: 576 - DNA, 20 bp, synthetic construct
  misc_feature 1..20: note = Synthetic oligonucleotide
  source 1..20: mol_type = other DNA
ccagggcggcggcacgagac

SEQ ID NO: 577 - DNA, 20 bp, synthetic construct
  misc_feature 1..20: note = Synthetic oligonucleotide
  source 1..20: mol_type = other DNA
cccagggcggcggcacgaga

SEQ ID NO: 578 - DNA, 20 bp, synthetic construct
  misc_feature 1..20: note = Synthetic oligonucleotide
  source 1..20: mol_type = other DNA
gcccagggcggcggcacgag

SEQ ID NO: 579 - DNA, 20 bp, synthetic construct
  misc_feature 1..20: note = Synthetic oligonucleotide
  source 1..20: mol_type = other DNA
agcccagggcggcggcacga

SEQ ID NO: 580 - DNA, 20 bp, synthetic construct
  misc_feature 1..20: note = Synthetic oligonucleotide
  source 1..20: mol_type = other DNA
cagcccagggcggcggcacg

SEQ ID NO: 581 - DNA, 20 bp, synthetic construct
  misc_feature 1..20: note = Synthetic oligonucleotide
  source 1..20: mol_type = other DNA
gcagcccagggcggcggcac

SEQ ID NO: 582 - DNA, 20 bp, synthetic construct
  misc_feature 1..20: note = Synthetic oligonucleotide
  source 1..20: mol_type = other DNA
ctgcggtgagttggccggcg

SEQ ID NO: 583 - DNA, 20 bp, synthetic construct
  misc_feature 1..20: note = Synthetic oligonucleotide
  source 1..20: mol_type = other DNA
actgcggtgagttggccggc

SEQ ID NO: 584 - DNA, 20 bp, synthetic construct
  misc_feature 1..20: note = Synthetic oligonucleotide
  source 1..20: mol_type = other DNA
gactgcggtgagttggccgg

SEQ ID NO: 585 - DNA, 20 bp, synthetic construct
  misc_feature 1..20: note = Synthetic oligonucleotide
  source 1..20: mol_type = other DNA
agactgcggtgagttggccg

SEQ ID NO: 586 - DNA, 20 bp, synthetic construct
  misc_feature 1..20: note = Synthetic oligonucleotide
  source 1..20: mol_type = other DNA
cagactgcggtgagttggcc

SEQ ID NO: 587 - DNA, 20 bp, synthetic construct
  misc_feature 1..20: note = Synthetic oligonucleotide
  source 1..20: mol_type = other DNA
ccagactgcggtgagttggc

SEQ ID NO: 588 - DNA, 20 bp, synthetic construct
  misc_feature 1..20: note = Synthetic oligonucleotide
  source 1..20: mol_type = other DNA
gccagactgcggtgagttgg

SEQ ID NO: 589 - DNA, 20 bp, synthetic construct
  misc_feature 1..20: note = Synthetic oligonucleotide
  source 1..20: mol_type = other DNA
cgccagactgcggtgagttg

SEQ ID NO: 590 - DNA, 20 bp, synthetic construct
  misc_feature 1..20: note = Synthetic oligonucleotide
  source 1..20: mol_type = other DNA
aagacagttctagggttcag

SEQ ID NO: 591 - DNA, 20 bp, synthetic construct
  misc_feature 1..20: note = Synthetic oligonucleotide
  source 1..20: mol_type = other DNA
gaagacagttctagggttca

SEQ ID NO: 592 - DNA, 20 bp, synthetic construct
  misc_feature 1..20: note = Synthetic oligonucleotide
  source 1..20: mol_type = other DNA
cgaagacagttctagggttc

SEQ ID NO: 593 - DNA, 20 bp, synthetic construct
  misc_feature 1..20: note = Synthetic oligonucleotide
  source 1..20: mol_type = other DNA
tcgaagacagttctagggtt

SEQ ID NO: 594 - DNA, 20 bp, synthetic construct
  misc_feature 1..20: note = Synthetic oligonucleotide
  source 1..20: mol_type = other DNA
gtcgaagacagttctagggt

SEQ ID NO: 595 - DNA, 20 bp, synthetic construct
  misc_feature 1..20: note = Synthetic oligonucleotide
  source 1..20: mol_type = other DNA
agtcgaagacagttctaggg

SEQ ID NO: 596 - DNA, 20 bp, synthetic construct
  misc_feature 1..20: note = Synthetic oligonucleotide
  source 1..20: mol_type = other DNA
gagtcgaagacagttctagg

SEQ ID NO: 597 - DNA, 20 bp, synthetic construct
  misc_feature 1..20: note = Synthetic oligonucleotide
  source 1..20: mol_type = other DNA
ggagtcgaagacagttctag

SEQ ID NO: 598 - DNA, 20 bp, synthetic construct
  misc_feature 1..20: note = Synthetic oligonucleotide
  source 1..20: mol_type = other DNA
cggagtcgaagacagttcta

SEQ ID NO: 599 - DNA, 20 bp, synthetic construct
  misc_feature 1..20: note = Synthetic oligonucleotide
  source 1..20: mol_type = other DNA
ccggagtcgaagacagttct

SEQ ID NO: 600 - DNA, 20 bp, synthetic construct
  misc_feature 1..20: note = Synthetic oligonucleotide
  source 1..20: mol_type = other DNA
cccggagtcgaagacagttc

SEQ ID NO: 601 - DNA, 20 bp, synthetic construct
  misc_feature 1..20: note = Synthetic oligonucleotide
  source 1..20: mol_type = other DNA
ccccggagtcgaagacagtt

SEQ ID NO: 602 - DNA, 20 bp, synthetic construct
  misc_feature 1..20: note = Synthetic oligonucleotide
  source 1..20: mol_type = other DNA
gccccggagtcgaagacagt

SEQ ID NO: 603 - DNA, 20 bp, synthetic construct
  misc_feature 1..20: note = Synthetic oligonucleotide
  source 1..20: mol_type = other DNA
ggccccggagtcgaagacag

SEQ ID NO: 604 - DNA, 20 bp, synthetic construct
  misc_feature 1..20: note = Synthetic oligonucleotide
  source 1..20: mol_type = other DNA
gggccccggagtcgaagaca

SEQ ID NO: 605 - DNA, 20 bp, synthetic construct
  misc_feature 1..20: note = Synthetic oligonucleotide
  source 1..20: mol_type = other DNA
aggcggtgggcgcggcttct

SEQ ID NO: 606 - DNA, 20 bp, synthetic construct
  misc_feature 1..20: note = Synthetic oligonucleotide
  source 1..20: mol_type = other DNA
caggcggtgggcgcggcttc

SEQ ID NO: 607 - DNA, 20 bp, synthetic construct
  misc_feature 1..20: note = Synthetic oligonucleotide
  source 1..20: mol_type = other DNA
gcaggcggtgggcgcggctt

SEQ ID NO: 608 - DNA, 20 bp, synthetic construct
  misc_feature 1..20: note = Synthetic oligonucleotide
  source 1..20: mol_type = other DNA
tggcaggcggtgggcgcggc

SEQ ID NO: 609 - DNA, 20 bp, synthetic construct
  misc_feature 1..20: note = Synthetic oligonucleotide
  source 1..20: mol_type = other DNA
actggcaggcggtgggcgcg

SEQ ID NO: 610 - DNA, 20 bp, synthetic construct
  misc_feature 1..20: note = Synthetic oligonucleotide
  source 1..20: mol_type = other DNA
gaactggcaggcggtgggcg

SEQ ID NO: 611 - DNA, 20 bp, synthetic construct
  misc_feature 1..20: note = Synthetic oligonucleotide
  source 1..20: mol_type = other DNA
tgaactggcaggcggtgggc

SEQ ID NO: 612 - DNA, 20 bp, synthetic construct
  misc_feature 1..20: note = Synthetic oligonucleotide
  source 1..20: mol_type = other DNA
tgtgaactggcaggcggtgg

SEQ ID NO: 613 - DNA, 20 bp, synthetic construct
  misc_feature 1..20: note = Synthetic oligonucleotide
  source 1..20: mol_type = other DNA
tggagctgggcggagaccca

SEQ ID NO: 614 - DNA, 20 bp, synthetic construct
  misc_feature 1..20: note = Synthetic oligonucleotide
  source 1..20: mol_type = other DNA
actggagctgggcggagacc

SEQ ID NO: 615 - DNA, 20 bp, synthetic construct
  misc_feature 1..20: note = Synthetic oligonucleotide
  source 1..20: mol_type = other DNA
gactggagctgggcggagac

SEQ ID NO: 616 - DNA, 20 bp, synthetic construct
  misc_feature 1..20: note = Synthetic oligonucleotide
  source 1..20: mol_type = other DNA
aggactggagctgggcggag

SEQ ID NO: 617 - DNA, 20 bp, synthetic construct
  misc_feature 1..20: note = Synthetic oligonucleotide
  source 1..20: mol_type = other DNA
acaggactggagctgggcgg

SEQ ID NO: 618 - DNA, 20 bp, synthetic construct
  misc_feature 1..20: note = Synthetic oligonucleotide
  source 1..20: mol_type = other DNA
cacaggactggagctgggcg

SEQ ID NO: 619 - DNA, 20 bp, synthetic construct
  misc_feature 1..20: note = Synthetic oligonucleotide
  source 1..20: mol_type = other DNA
tcacaggactggagctgggc

SEQ ID NO: 620 - DNA, 20 bp, synthetic construct
  misc_feature 1..20: note = Synthetic oligonucleotide
  source 1..20: mol_type = other DNA
gcctcagcctggccgaaaga

SEQ ID NO: 621 - DNA, 20 bp, synthetic construct
  misc_feature 1..20: note = Synthetic oligonucleotide
  source 1..20: mol_type = other DNA
ggcctcagcctggccgaaag

SEQ ID NO: 622 - DNA, 20 bp, synthetic construct
  misc_feature 1..20: note = Synthetic oligonucleotide
  source 1..20: mol_type = other DNA
tggtggagccaagccctccc

SEQ ID NO: 623 - DNA, 20 bp, synthetic construct
  misc_feature 1..20: note = Synthetic oligonucleotide
  source 1..20: mol_type = other DNA
gggcaccctcagagcctgaa

SEQ ID NO: 624 - DNA, 20 bp, synthetic construct
  misc_feature 1..20: note = Synthetic oligonucleotide
  source 1..20: mol_type = other DNA
accccactgcaagaagtcgg

SEQ ID NO: 625 - DNA, 20 bp, synthetic construct
  misc_feature 1..20: note = Synthetic oligonucleotide
  source 1..20: mol_type = other DNA
gccccaggatgggaggatct

SEQ ID NO: 626 - DNA, 20 bp, synthetic construct
  misc_feature 1..20: note = Synthetic oligonucleotide
  source 1..20: mol_type = other DNA
cataggacagagaaatgttg

SEQ ID NO: 627 - DNA, 20 bp, synthetic construct
  misc_feature 1..20: note = Synthetic oligonucleotide
  source 1..20: mol_type = other DNA
tgctgaccttactctgcccc

SEQ ID NO: 628 - DNA, 20 bp, synthetic construct
  misc_feature 1..20: note = Synthetic oligonucleotide
  source 1..20: mol_type = other DNA
taagccatggctctgagtca

SEQ ID NO: 629 - DNA, 20 bp, synthetic construct
  misc_feature 1..20: note = Synthetic oligonucleotide
  source 1..20: mol_type = other DNA
agagaggccatgggaggctg

SEQ ID NO: 630 - DNA, 20 bp, synthetic construct
  misc_feature 1..20: note = Synthetic oligonucleotide
  source 1..20: mol_type = other DNA
ctggccctcctggcttgccc

SEQ ID NO: 631 - DNA, 20 bp, synthetic construct
  misc_feature 1..20: note = Synthetic oligonucleotide
  source 1..20: mol_type = other DNA
agctgccccatgctggccct

SEQ ID NO: 632 - DNA, 20 bp, synthetic construct
  misc_feature 1..20: note = Synthetic oligonucleotide
  source 1..20: mol_type = other DNA
gcccctggcagctgccccat

SEQ ID NO: 633 - DNA, 20 bp, synthetic construct
  misc_feature 1..20: note = Synthetic oligonucleotide
  source 1..20: mol_type = other DNA
ctgtcggctgcgcccctggc

SEQ ID NO: 634 - DNA, 20 bp, synthetic construct
  misc_feature 1..20: note = Synthetic oligonucleotide
  source 1..20: mol_type = other DNA
cgccgaacacctgcctgtcg

SEQ ID NO: 635 - DNA, 20 bp, synthetic construct
  misc_feature 1..20: note = Synthetic oligonucleotide
  source 1..20: mol_type = other DNA
cctcccagtgcctgggcacc

SEQ ID NO: 636 - DNA, 20 bp, synthetic construct
  misc_feature 1..20: note = Synthetic oligonucleotide
  source 1..20: mol_type = other DNA
gcgcctgtctgcaaagctgg

SEQ ID NO: 637 - DNA, 20 bp, synthetic construct
  misc_feature 1..20: note = Synthetic oligonucleotide
  source 1..20: mol_type = other DNA
cccaaagttgtccctcctgg

SEQ ID NO: 638 - DNA, 20 bp, synthetic construct
  misc_feature 1..20: note = Synthetic oligonucleotide
  source 1..20: mol_type = other DNA
acacccagaagaacccaaag

SEQ ID NO: 639 - DNA, 20 bp, synthetic construct
  misc_feature 1..20: note = Synthetic oligonucleotide
  source 1..20: mol_type = other DNA
ctgacccacacggctcatag

SEQ ID NO: 640 - DNA, 20 bp, synthetic construct
  misc_feature 1..20: note = Synthetic oligonucleotide
  source 1..20: mol_type = other DNA
tggccccaggccctggaaag

SEQ ID NO: 641 - DNA, 20 bp, synthetic construct
  misc_feature 1..20: note = Synthetic oligonucleotide
  source 1..20: mol_type = other DNA
gacaaggcagctggcagaag

SEQ ID NO: 642 - DNA, 20 bp, synthetic construct
  misc_feature 1..20: note = Synthetic oligonucleotide
  source 1..20: mol_type = other DNA
aagaaaccagtgaccagtga

SEQ ID NO: 643 - DNA, 20 bp, synthetic construct
  misc_feature 1..20: note = Synthetic oligonucleotide
  source 1..20: mol_type = other DNA
ctgtgaaatgggaggaggag

SEQ ID NO: 644 - DNA, 20 bp, synthetic construct
  misc_feature 1..20: note = Synthetic oligonucleotide
  source 1..20: mol_type = other DNA
gaaggtttttccagaggctg

SEQ ID NO: 645 - DNA, 20 bp, synthetic construct
  misc_feature 1..20: note = Synthetic oligonucleotide
  source 1..20: mol_type = other DNA
ggccaggagagtcattaggg

SEQ ID NO: 646 - DNA, 20 bp, synthetic construct
  misc_feature 1..20: note = Synthetic oligonucleotide
  source 1..20: mol_type = other DNA
ccacaaaaggagtgctcctc

SEQ ID NO: 647 - DNA, 20 bp, synthetic construct
  misc_feature 1..20: note = Synthetic oligonucleotide
  source 1..20: mol_type = other DNA
ccttttaaggcagcaggaac

SEQ ID NO: 648 - DNA, 20 bp, synthetic construct
  misc_feature 1..20: note = Synthetic oligonucleotide
  source 1..20: mol_type = other DNA
ctaggactgtctgcttccca

SEQ ID NO: 649 - DNA, 20 bp, synthetic construct
  misc_feature 1..20: note = Synthetic oligonucleotide
  source 1..20: mol_type = other DNA
gtcattcatcaatttctaag

SEQ ID NO: 650 - DNA, 20 bp, synthetic construct
  misc_feature 1..20: note = Synthetic oligonucleotide
  source 1..20: mol_type = other DNA
ggaggagctgcagccggaga

SEQ ID NO: 651 - DNA, 20 bp, synthetic construct
  misc_feature 1..20: note = Synthetic oligonucleotide
  source 1..20: mol_type = other DNA
gcacccggaggagctgcagc

SEQ ID NO: 652 - DNA, 20 bp, synthetic construct
  misc_feature 1..20: note = Synthetic oligonucleotide
  source 1..20: mol_type = other DNA
gcacgacacctgcagggcac

SEQ ID NO: 653 - DNA, 20 bp, synthetic construct
  misc_feature 1..20: note = Synthetic oligonucleotide
  source 1..20: mol_type = other DNA
agctcaccaggtagttctca

SEQ ID NO: 654 - DNA, 20 bp, synthetic construct
  misc_feature 1..20: note = Synthetic oligonucleotide
  source 1..20: mol_type = other DNA
gcttcctctccccacctcct

SEQ ID NO: 655 - DNA, 20 bp, synthetic construct
  misc_feature 1..20: note = Synthetic oligonucleotide
  source 1..20: mol_type = other DNA
gcagcacccccaatcctaga

SEQ ID NO: 656 - DNA, 20 bp, synthetic construct
  misc_feature 1..20: note = Synthetic oligonucleotide
  source 1..20: mol_type = other DNA
gcccctcatccacctgacac

SEQ ID NO: 657 - DNA, 20 bp, synthetic construct
  misc_feature 1..20: note = Synthetic oligonucleotide
  source 1..20: mol_type = other DNA
ttccaggtaagagacccccc

SEQ ID NO: 658 - DNA, 20 bp, synthetic construct
  misc_feature 1..20: note = Synthetic oligonucleotide
  source 1..20: mol_type = other DNA
agaataggtcccagacactc

SEQ ID NO: 659 - DNA, 20 bp, synthetic construct
  misc_feature 1..20: note = Synthetic oligonucleotide
  source 1..20: mol_type = other DNA
ctccccctgagatgttctgg

SEQ ID NO: 660 - DNA, 20 bp, synthetic construct
  misc_feature 1..20: note = Synthetic oligonucleotide
  source 1..20: mol_type = other DNA
ccccagcccagagataacca

SEQ ID NO: 661 - DNA, 20 bp, synthetic construct
  misc_feature 1..20: note = Synthetic oligonucleotide
  source 1..20: mol_type = other DNA
cctgatccatcacggatggc

SEQ ID NO: 662 - DNA, 20 bp, synthetic construct
  misc_feature 1..20: note = Synthetic oligonucleotide
  source 1..20: mol_type = other DNA
tactccatgaccaggtactg

SEQ ID NO: 663 - DNA, 20 bp, synthetic construct
  misc_feature 1..20: note = Synthetic oligonucleotide
  source 1..20: mol_type = other DNA
gctctgaccttccaagaacc

SEQ ID NO: 664 - DNA, 20 bp, synthetic construct
  misc_feature 1..20: note = Synthetic oligonucleotide
  source 1..20: mol_type = other DNA
ctcccttctgtggtcccacc

SEQ ID NO: 665 - DNA, 20 bp, synthetic construct
  misc_feature 1..20: note = Synthetic oligonucleotide
  source 1..20: mol_type = other DNA
gtcgggtttgatgtccctgc

SEQ ID NO: 666 - DNA, 20 bp, synthetic construct
  misc_feature 1..20: note = Synthetic oligonucleotide
  source 1..20: mol_type = other DNA
agggcactggctcaccgttc

SEQ ID NO: 667 - DNA, 20 bp, synthetic construct
  misc_feature 1..20: note = Synthetic oligonucleotide
  source 1..20: mol_type = other DNA
gggccctccttccaaccact

SEQ ID NO: 668 - DNA, 20 bp, synthetic construct
  misc_feature 1..20: note = Synthetic oligonucleotide
  source 1..20: mol_type = other DNA
gcccacccctctgggcccac

SEQ ID NO: 669 - DNA, 20 bp, synthetic construct
  misc_feature 1..20: note = Synthetic oligonucleotide
  source 1..20: mol_type = other DNA
aggagcagagcgaggcttgg

SEQ ID NO: 670 - DNA, 20 bp, synthetic construct
  misc_feature 1..20: note = Synthetic oligonucleotide
  source 1..20: mol_type = other DNA
caccttgtagtggacgatct

SEQ ID NO: 671 - DNA, 20 bp, synthetic construct
  misc_feature 1..20: note = Synthetic oligonucleotide
  source 1..20: mol_type = other DNA
ctaccccgcccccgctcacc

SEQ ID NO: 672 - DNA, 20 bp, synthetic construct
  misc_feature 1..20: note = Synthetic oligonucleotide
  source 1..20: mol_type = other DNA
ctaggtcactgctgggtcct

SEQ ID NO: 673 - DNA, 20 bp, synthetic construct
  misc_feature 1..20: note = Synthetic oligonucleotide
  source 1..20: mol_type = other DNA
ctcagatagctccccactcc

SEQ ID NO: 674 - DNA, 20 bp, synthetic construct
  misc_feature 1..20: note = Synthetic oligonucleotide
  source 1..20: mol_type = other DNA
aattctctaattctctagac

SEQ ID NO: 675 - DNA, 20 bp, synthetic construct
  misc_feature 1..20: note = Synthetic oligonucleotide
  source 1..20: mol_type = other DNA
tacctgagggccatgcagga

SEQ ID NO: 676 - DNA, 20 bp, synthetic construct
  misc_feature 1..20: note = Synthetic oligonucleotide
  source 1..20: mol_type = other DNA
gttccaagactgatcctgca

SEQ ID NO: 677 - DNA, 20 bp, synthetic construct
  misc_feature 1..20: note = Synthetic oligonucleotide
  source 1..20: mol_type = other DNA
aggagggcggtggcgcggcg

SEQ ID NO: 678 - DNA, 20 bp, synthetic construct
  misc_feature 1..20: note = Synthetic oligonucleotide
  source 1..20: mol_type = other DNA
tgacagctggaaggagaaga

SEQ ID NO: 679 - DNA, 20 bp, synthetic construct
  misc_feature 1..20: note = Synthetic oligonucleotide
  source 1..20: mol_type = other DNA
catgggaaggtggatccgtg

SEQ ID NO: 680 - DNA, 20 bp, synthetic construct
  misc_feature 1..20: note = Synthetic oligonucleotide
  source 1..20: mol_type = other DNA
ggaggttatctagggagatc

SEQ ID NO: 681 - DNA, 20 bp, synthetic construct
  misc_feature 1..20: note = Synthetic oligonucleotide
  source 1..20: mol_type = other DNA
gaagggacaggtgacccgat

SEQ ID NO: 682 - DNA, 20 bp, synthetic construct
  misc_feature 1..20: note = Synthetic oligonucleotide
  source 1..20: mol_type = other DNA
cgtaccctggcagggagcag

SEQ ID NO: 683 - DNA, 20 bp, synthetic construct
  misc_feature 1..20: note = Synthetic oligonucleotide
  source 1..20: mol_type = other DNA
ggactcgccccgcctacgcc

SEQ ID NO: 684 - DNA, 20 bp, synthetic construct
  misc_feature 1..20: note = Synthetic oligonucleotide
  source 1..20: mol_type = other DNA
ctcctgggactcgccccgcc

SEQ ID NO: 685 - DNA, 20 bp, synthetic construct
  misc_feature 1..20: note = Synthetic oligonucleotide
  source 1..20: mol_type = other DNA
gctcctgggactcgccccgc

SEQ ID NO: 686 - DNA, 20 bp, synthetic construct
  misc_feature 1..20: note = Synthetic oligonucleotide
  source 1..20: mol_type = other DNA
attggctcctgggactcgcc

SEQ ID NO: 687 - DNA, 20 bp, synthetic construct
  misc_feature 1..20: note = Synthetic oligonucleotide
  source 1..20: mol_type = other DNA
gattggctcctgggactcgc

SEQ ID NO: 688 - DNA, 20 bp, synthetic construct
  misc_feature 1..20: note = Synthetic oligonucleotide
  source 1..20: mol_type = other DNA
gcctctgattggctcctggg

SEQ ID NO: 689 - DNA, 20 bp, synthetic construct
  misc_feature 1..20: note = Synthetic oligonucleotide
  source 1..20: mol_type = other DNA
gcatgggcctctgattggct

SEQ ID NO: 690 - DNA, 20 bp, synthetic construct
  misc_feature 1..20: note = Synthetic oligonucleotide
  source 1..20: mol_type = other DNA
cacccggcatgggcctctga

SEQ ID NO: 691 - DNA, 20 bp, synthetic construct
  misc_feature 1..20: note = Synthetic oligonucleotide
  source 1..20: mol_type = other DNA
gccaggcctagggacctgcg

SEQ ID NO: 692 - DNA, 20 bp, synthetic construct
  misc_feature 1..20: note = Synthetic oligonucleotide
  source 1..20: mol_type = other DNA
ttcctcccccaaccctgatt

SEQ ID NO: 693 - DNA, 20 bp, synthetic construct
  misc_feature 1..20: note = Synthetic oligonucleotide
  source 1..20: mol_type = other DNA
aagtttgcagcaacttttct

SEQ ID NO: 694 - DNA, 20 bp, synthetic construct
  misc_feature 1..20: note = Synthetic oligonucleotide
  source 1..20: mol_type = other DNA
gcccctcggaattcccggct

SEQ ID NO: 695 - DNA, 20 bp, synthetic construct
  misc_feature 1..20: note = Synthetic oligonucleotide
  source 1..20: mol_type = other DNA
catctcggcctgcgctccgc

SEQ ID NO: 696 - DNA, 20 bp, synthetic construct
  misc_feature 1..20: note = Synthetic oligonucleotide
  source 1..20: mol_type = other DNA
gcaggcccccacattcccca

SEQ ID NO: 697 - DNA, 20 bp, synthetic construct
  misc_feature 1..20: note = Synthetic oligonucleotide
  source 1..20: mol_type = other DNA
cttctgcacgcctccgtctc

SEQ ID NO: 698 - DNA, 20 bp, synthetic construct
  misc_feature 1..20: note = Synthetic oligonucleotide
  source 1..20: mol_type = other DNA
tggcccacagccacggccgg

SEQ ID NO: 699 - DNA, 20 bp, synthetic construct
  misc_feature 1..20: note = Synthetic oligonucleotide
  source 1..20: mol_type = other DNA
ggcctggccccaccagcggg

SEQ ID NO: 700 - DNA, 20 bp, synthetic construct
  misc_feature 1..20: note = Synthetic oligonucleotide
  source 1..20: mol_type = other DNA
cctggcagggagcagcaggt

SEQ ID NO: 701 - DNA, 20 bp, synthetic construct
  misc_feature 1..20: note = Synthetic oligonucleotide
  source 1..20: mol_type = other DNA
cagccgcacttcggctgaca

SEQ ID NO: 702 - DNA, 20 bp, synthetic construct
  misc_feature 1..20: note = Synthetic oligonucleotide
  source 1..20: mol_type = other DNA
gcctgggtccagcaccagct

SEQ ID NO: 703 - DNA, 20 bp, synthetic construct
  misc_feature 1..20: note = Synthetic oligonucleotide
  source 1..20: mol_type = other DNA
gtcccaggaagcctgggtcc

SEQ ID NO: 704 - DNA, 20 bp, synthetic construct
  misc_feature 1..20: note = Synthetic oligonucleotide
  source 1..20: mol_type = other DNA
cgttagcaggtccccgccca

SEQ ID NO: 705 - DNA, 20 bp, synthetic construct
  misc_feature 1..20: note = Synthetic oligonucleotide
  source 1..20: mol_type = other DNA
gtctatggccatgacaatct

SEQ ID NO: 706 - DNA, 20 bp, synthetic construct
  misc_feature 1..20: note = Synthetic oligonucleotide
  source 1..20: mol_type = other DNA
gtagcccagccggtgcacgg

SEQ ID NO: 707 - DNA, 20 bp, synthetic construct
  misc_feature 1..20: note = Synthetic oligonucleotide
  source 1..20: mol_type = other DNA
gggtgcccacagccaccagc

SEQ ID NO: 708 - DNA, 20 bp, synthetic construct
  misc_feature 1..20: note = Synthetic oligonucleotide
  source 1..20: mol_type = other DNA
tggcccgtagctgcctgccc

SEQ ID NO: 709 - DNA, 20 bp, synthetic construct
  misc_feature 1..20: note = Synthetic oligonucleotide
  source 1..20: mol_type = other DNA
ggaaatcacctgccccacct

SEQ ID NO: 710 - DNA, 20 bp, synthetic construct
  misc_feature 1..20: note = Synthetic oligonucleotide
  source 1..20: mol_type = other DNA
ggatgtttctggaaatcacc

SEQ ID NO: 711 - DNA, 20 bp, synthetic construct
  misc_feature 1..20: note = Synthetic oligonucleotide
  source 1..20: mol_type = other DNA
gtggcaccctcgaagtctgg

SEQ ID NO: 712 - DNA, 20 bp, synthetic construct
  misc_feature 1..20: note = Synthetic oligonucleotide
  source 1..20: mol_type = other DNA
ccccgctcaccatggcagtg

SEQ ID NO: 713 - DNA, 20 bp, synthetic construct
  misc_feature 1..20: note = Synthetic oligonucleotide
  source 1..20: mol_type = other DNA
ggtccgggacctgattgtct

SEQ ID NO: 714 - DNA, 20 bp, synthetic construct
  misc_feature 1..20: note = Synthetic oligonucleotide
  source 1..20: mol_type = other DNA
gctgcatgtctgcccgtccc

SEQ ID NO: 715 - DNA, 20 bp, synthetic construct
  misc_feature 1..20: note = Synthetic oligonucleotide
  source 1..20: mol_type = other DNA
ggccccagaaccctagctgc

SEQ ID NO: 716 - DNA, 20 bp, synthetic construct
  misc_feature 1..20: note = Synthetic oligonucleotide
  source 1..20: mol_type = other DNA
tcacagggcctggctgcccc

SEQ ID NO: 717 - DNA, 20 bp, synthetic construct
  misc_feature 1..20: note = Synthetic oligonucleotide
  source 1..20: mol_type = other DNA
ggctgacatgttgggcaggc

SEQ ID NO: 718 - DNA, 20 bp, synthetic construct
  misc_feature 1..20: note = Synthetic oligonucleotide
  source 1..20: mol_type = other DNA
tgtccaggccccagaaccct

SEQ ID NO: 719 - DNA, 20 bp, synthetic construct
  misc_feature 1..20: note = Synthetic oligonucleotide
  source 1..20: mol_type = other DNA
ggccaggcctagggatctgc

SEQ ID NO: 720 - DNA, 20 bp, synthetic construct
  misc_feature 1..20: note = Synthetic oligonucleotide
  source 1..20: mol_type = other DNA
cgcctcggataggccaggcc

SEQ ID NO: 721 - DNA, 20 bp, synthetic construct
  misc_feature 1..20: note = Synthetic oligonucleotide
  source 1..20: mol_type = other DNA
ggcttggagtcttagggttc

SEQ ID NO: 722 - DNA, 20 bp, synthetic construct
  misc_feature 1..20: note = Synthetic oligonucleotide
  source 1..20: mol_type = other DNA
tccccggccgccaggtggca

SEQ ID NO: 723 - DNA, 20 bp, synthetic construct
  misc_feature 1..20: note = Synthetic oligonucleotide
  source 1..20: mol_type = other DNA
ggtgctgggcacgagccctg

SEQ ID NO: 724 - DNA, 20 bp, synthetic construct
  misc_feature 1..20: note = Synthetic oligonucleotide
  source 1..20: mol_type = other DNA
gcccagctgctgcagcagcg

SEQ ID NO: 725 - DNA, 20 bp, synthetic construct
  misc_feature 1..20: note = Synthetic oligonucleotide
  source 1..20: mol_type = other DNA
ccgtgtgtgctggcagaggt

SEQ ID NO: 726 - DNA, 20 bp, synthetic construct
  misc_feature 1..20: note = Synthetic oligonucleotide
  source 1..20: mol_type = other DNA
ataaataccgaggaatgtcg

SEQ ID NO: 727 - DNA, 20 bp, synthetic construct
  misc_feature 1..20: note = Synthetic oligonucleotide
  source 1..20: mol_type = other DNA
gggacagacaataaataccg

SEQ ID NO: 728 - DNA, 20 bp, synthetic construct
  misc_feature 1..20: note = Synthetic oligonucleotide
  source 1..20: mol_type = other DNA
gtgcagcccagtgtggcggc

SEQ ID NO: 729 - DNA, 20 bp, synthetic construct
  misc_feature 1..20: note = Synthetic oligonucleotide
  source 1..20: mol_type = other DNA
cctggagaagttctggttgg

SEQ ID NO: 730 - DNA, 20 bp, synthetic construct
  misc_feature 1..20: note = Synthetic oligonucleotide
  source 1..20: mol_type = other DNA
ggtgacccgatcggagccca

SEQ ID NO: 731 - DNA, 20 bp, synthetic construct
  misc_feature 1..20: note = Synthetic oligonucleotide
  source 1..20: mol_type = other DNA
agctggagagagaagggaca

SEQ ID NO: 732 - DNA, 20 bp, synthetic construct
  misc_feature 1..20: note = Synthetic oligonucleotide
  source 1..20: mol_type = other DNA
gtgagggactcgcctgcggc

SEQ ID NO: 733 - DNA, 20 bp, synthetic construct
  misc_feature 1..20: note = Synthetic oligonucleotide
  source 1..20: mol_type = other DNA
gcggctgcggtgccccagcc

SEQ ID NO: 734 - DNA, 20 bp, synthetic construct
  misc_feature 1..20: note = Synthetic oligonucleotide
  source 1..20: mol_type = other DNA
gggccatctagctggagaga

SEQ ID NO: 735 - DNA, 20 bp, synthetic construct
  misc_feature 1..20: note = Synthetic oligonucleotide
  source 1..20: mol_type = other DNA
ccccactgcaagaagtcggc

SEQ ID NO: 736 - DNA, 20 bp, synthetic construct
  misc_feature 1..20: note = Synthetic oligonucleotide
  source 1..20: mol_type = other DNA
ttgagcccttttaaggcagc

SEQ ID NO: 737 - DNA, 20 bp, synthetic construct
  misc_feature 1..20: note = Synthetic oligonucleotide
  source 1..20: mol_type = other DNA
tgaccaggtactgggagcgg

SEQ ID NO: 738 - DNA, 20 bp, synthetic construct
  misc_feature 1..20: note = Synthetic oligonucleotide
  source 1..20: mol_type = other DNA
cctggagctggatcagtccc

SEQ ID NO: 739 - DNA, 20 bp, synthetic construct
  misc_feature 1..20: note = Synthetic oligonucleotide
  source 1..20: mol_type = other DNA
acatgggaaggtggatccgt

SEQ ID NO: 740 - DNA, 20 bp, synthetic construct
  misc_feature 1..20: note = Synthetic oligonucleotide
  source 1..20: mol_type = other DNA
gtgggacataccctggcagg

SEQ ID NO: 741 - DNA, 20 bp, synthetic construct
  misc_feature 1..20: note = Synthetic oligonucleotide
  source 1..20: mol_type = other DNA
gccaggcctagggatctgca

SEQ ID NO: 742 - DNA, 20 bp, synthetic construct
  misc_feature 1..20: note = Synthetic oligonucleotide
  source 1..20: mol_type = other DNA
ggaagcacgacacctcgcct

SEQ ID NO: 743 - DNA, 20 bp, synthetic construct
  misc_feature 1..20: note = Synthetic oligonucleotide
  source 1..20: mol_type = other DNA
cctcaccattccatcaggct

SEQ ID NO: 744 - DNA, 20 bp, synthetic construct
  misc_feature 1..20: note = Synthetic oligonucleotide
  source 1..20: mol_type = other DNA
cggcagcgacaagtgttccc

SEQ ID NO: 745 - DNA, 20 bp, synthetic construct
  misc_feature 1..20: note = Synthetic oligonucleotide
  source 1..20: mol_type = other DNA
gtctctgaaggccatgcagc

SEQ ID NO: 746 - DNA, 20 bp, synthetic construct
  misc_feature 1..20: note = Synthetic oligonucleotide
  source 1..20: mol_type = other DNA
cagccacttgatccggtggg

SEQ ID NO: 747 - DNA, 20 bp, synthetic construct
  misc_feature 1..20: note = Synthetic oligonucleotide
  source 1..20: mol_type = other DNA
aggtcggcctcttcagccac

SEQ ID NO: 748 - DNA, 20 bp, synthetic construct
  misc_feature 1..20: note = Synthetic oligonucleotide
  source 1..20: mol_type = other DNA
gttggctggagaagttctgg

SEQ ID NO: 749 - DNA, 20 bp, synthetic construct
  misc_feature 1..20: note = Synthetic oligonucleotide
  source 1..20: mol_type = other DNA
ccccgtgatggctgcggctc

SEQ ID NO: 750 - DNA, 20 bp, synthetic construct
  misc_feature 1..20: note = Synthetic oligonucleotide
  source 1..20: mol_type = other DNA
aggccaggcctagggatcct

SEQ ID NO: 751 - DNA, 20 bp, synthetic construct
  misc_feature 1..20: note = Synthetic oligonucleotide
  source 1..20: mol_type = other DNA
ggcgcggtgccccagcctgg

SEQ ID NO: 752 - DNA, 20 bp, synthetic construct
  misc_feature 1..20: note = Synthetic oligonucleotide
  source 1..20: mol_type = other DNA
gtcctggccccaccagcggg

SEQ ID NO: 753 - DNA, 20 bp, synthetic construct
  misc_feature 1..20: note = Synthetic oligonucleotide
  source 1..20: mol_type = other DNA
ccaggcctaggaatcctggc

SEQ ID NO: 754 - DNA, 20 bp, synthetic construct
  misc_feature 1..20: note = Synthetic oligonucleotide
  source 1..20: mol_type = other DNA
gcgcctcggatagccaggcc

SEQ ID NO: 755 - DNA, 20 bp, synthetic construct
  misc_feature 1..20: note = Synthetic oligonucleotide
  source 1..20: mol_type = other DNA
cccagtgtggcgcagcagcc

SEQ ID NO: 756 - DNA, 20 bp, synthetic construct
  misc_feature 1..20: note = Synthetic oligonucleotide
  source 1..20: mol_type = other DNA
gtgtttcatcttcaccaccg

SEQ ID NO: 757 - DNA, 20 bp, synthetic construct
  misc_feature 1..20: note = Synthetic oligonucleotide
  source 1..20: mol_type = other DNA
aggtcagcctcttcagccac

SEQ ID NO: 758 - DNA, 20 bp, synthetic construct
  misc_feature 1..20: note = Synthetic oligonucleotide
  source 1..20: mol_type = other DNA
ggccatatgggaaggtggat

SEQ ID NO: 759 - DNA, 20 bp, synthetic construct
  misc_feature 1..20: note = Synthetic oligonucleotide
  source 1..20: mol_type = other DNA
ggaggatttggcgagaagca

SEQ ID NO: 760 - DNA, 20 bp, synthetic construct
  misc_feature 1..20: note = Synthetic oligonucleotide
  source 1..20: mol_type = other DNA
cgaagtctgccccacctcga

SEQ ID NO: 761 - DNA, 20 bp, synthetic construct
  misc_feature 1..20: note = Synthetic oligonucleotide
  source 1..20: mol_type = other DNA
gtggcaccctcgaagtctgc

SEQ ID NO: 762 - DNA, 20 bp, synthetic construct
  misc_feature 1..20: note = Synthetic oligonucleotide
  source 1..20: mol_type = other DNA
gggtccattgtaaggaagct

SEQ ID NO: 763 - DNA, 20 bp, synthetic construct
  misc_feature 1..20: note = Synthetic oligonucleotide
  source 1..20: mol_type = other DNA
ggtgcccacagccaccaggg

SEQ ID NO: 764 - DNA, 20 bp, synthetic construct
  misc_feature 1..20: note = Synthetic oligonucleotide
  source 1..20: mol_type = other DNA
tccatggcagtgagccggtc

SEQ ID NO: 765 - DNA, 20 bp, synthetic construct
  misc_feature 1..20: note = Synthetic oligonucleotide
  source 1..20: mol_type = other DNA
gggaccacttgatccggtgg

SEQ ID NO: 766 - DNA, 20 bp, synthetic construct
  misc_feature 1..20: note = Synthetic oligonucleotide
  source 1..20: mol_type = other DNA
ggatcagagttgggaccact

SEQ ID NO: 767 - DNA, 20 bp, synthetic construct
  misc_feature 1..20: note = Synthetic oligonucleotide
  source 1..20: mol_type = other DNA
ccccgtgatggctgcggttc

SEQ ID NO: 768 - DNA, 20 bp, synthetic construct
  misc_feature 1..20: note = Synthetic oligonucleotide
  source 1..20: mol_type = other DNA
gtgtgtcctcataccccgcc

SEQ ID NO: 769 - DNA, 20 bp, synthetic construct
  misc_feature 1..20: note = Synthetic oligonucleotide
  source 1..20: mol_type = other DNA
gcaccctcgaagtctcgacc

SEQ ID NO: 770 - DNA, 20 bp, synthetic construct
  misc_feature 1..20: note = Synthetic oligonucleotide
  source 1..20: mol_type = other DNA
gctctgaaggccatgcagca

SEQ ID NO: 771 - DNA, 25 bp, synthetic construct
  misc_feature 1..25: note = Primer
  source 1..25: mol_type = other DNA
gacatatgccaagattgtgcactac

SEQ ID NO: 772 - DNA, 21 bp, synthetic construct
  misc_feature 1..21: note = Primer
  source 1..21: mol_type = other DNA
cacgaatgaggtcctgagctt

SEQ ID NO: 773 - DNA, 22 bp, synthetic construct
  misc_feature 1..22: note = Probe
  source 1..22: mol_type = other DNA
aacacttgtcgctgccgctggc

SEQ ID NO: 774 - DNA, 20 bp, synthetic construct
  misc_feature 1..20: note = Synthetic oligonucleotide
  source 1..20: mol_type = other DNA
agcgaggcttcacttggcgc

SEQ ID NO: 775 - DNA, 20 bp, synthetic construct
  misc_feature 1..20: note = Synthetic oligonucleotide
  source 1..20: mol_type = other DNA
gggaagcgaggcttcacttg

SEQ ID NO: 776 - DNA, 20 bp, synthetic construct
  misc_feature 1..20: note = Synthetic oligonucleotide
  source 1..20: mol_type = other DNA
gcggtcagcgatcccagggt

SEQ ID NO: 777 - DNA, 20 bp, synthetic construct
  misc_feature 1..20: note = Synthetic oligonucleotide
  source 1..20: mol_type = other DNA
gggtgccagcgcggtgatct

SEQ ID NO: 778 - DNA, 20 bp, synthetic construct
  misc_feature 1..20: note = Synthetic oligonucleotide
  source 1..20: mol_type = other DNA
tgttacaaagaaagtgactg

SEQ ID NO: 779 - DNA, 20 bp, synthetic construct
  misc_feature 1..20: note = Synthetic oligonucleotide
  source 1..20: mol_type = other DNA
cgatggcagcaacggaagtt

SEQ ID NO: 780 - DNA, 20 bp, synthetic construct
  misc_feature 1..20: note = Synthetic oligonucleotide
  source 1..20: mol_type = other DNA
gtcagtttacgatggcagca

SEQ ID NO: 781 - DNA, 20 bp, synthetic construct
  misc_feature 1..20: note = Synthetic oligonucleotide
  source 1..20: mol_type = other DNA
cagggctttgtttcgaaaaa

SEQ ID NO: 782 - DNA, 20 bp, synthetic construct
  misc_feature 1..20: note = Synthetic oligonucleotide
  source 1..20: mol_type = other DNA
ccattttcttccacagggct

SEQ ID NO: 783 - DNA, 20 bp, synthetic construct
  misc_feature 1..20: note = Synthetic oligonucleotide
  source 1..20: mol_type = other DNA
atgcttcttcaagttttcca

SEQ ID NO: 784 - DNA, 20 bp, synthetic construct
  misc_feature 1..20: note = Synthetic oligonucleotide
  source 1..20: mol_type = other DNA
cagaatgactttaatgcttc

SEQ ID NO: 785 - DNA, 21 bp, synthetic construct
  misc_feature 1..21: note = Primer
  source 1..21: mol_type = other DNA
ccaccgcaaatgcttctagac

SEQ ID NO: 786 - DNA, 20 bp, synthetic construct
  misc_feature 1..20: note = Primer
  source 1..20: mol_type = other DNA
cccccccattgagaagattc

SEQ ID NO: 787 - DNA, 24 bp, synthetic construct
  misc_feature 1..24: note = Probe
  source 1..24: mol_type = other DNA
ctccacctccagcacgcgacttct

SEQ ID NO: 788 - DNA, 20 bp, synthetic construct
  misc_feature 1..20: note = Synthetic oligonucleotide
  source 1..20: mol_type = other DNA
gcggtcagcgatcccagggt

SEQ ID NO: 789 - DNA, 25 bp, synthetic construct
  misc_feature 1..25: note = Synthetic oligonucleotide
  source 1..25: mol_type = other DNA
agcagcagcagcagcagcagcagca

SEQ ID NO: 790 - DNA, 20 bp, synthetic construct
  misc_feature 1..20: note = Synthetic oligonucleotide
  source 1..20: mol_type = other DNA
agcagcagcagcagcagcag

SEQ ID NO: 791 - DNA, 15 bp, synthetic construct
  misc_feature 1..15: note = Synthetic oligonucleotide
  source 1..15: mol_type = other DNA
gcagcagcagcagca

SEQ ID NO: 792 - skipped

SEQ ID NO: 793 - DNA, 2611 bp, Mus musculus
  source 1..2611: mol_type = genomic DNA
cgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcacctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaaaaaaaaaaaaaaaaaaaaaaa

SEQ ID NO: 794 - DNA, 988 bp, Mus musculus
  misc_feature 531: note = n is a, c, g, or t
  misc_feature 942: note = n is a, c, g, or t
  source 1..988: mol_type = genomic DNA
gctggaccggtccggaattctccggatcgccagcctttgtgggccatattcgtcatccctcctggcttctcatctgcttttgtggtcctagctcaagacctctaattcctctgctgacttaaatgcccttccccagaggtcttctcaggcctagtggacaagcttggagccttatctgctcctgcccaacattgagccaaagctccagcttaccccagcttccttacaatggaccccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggngacctgctaacgctgctgagcaagttttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctggcctcaactgcagcctgatggaatggtggagtcccctggtggctgtgggcacccccggactacctgtctcctgaaattctgcagggccttggtgggaggccctggggaaggcaactacgggccaaaagttggaaggggggggcctggggggggttcccctatgaaaagttctatggggaggaccccctttaagcggaatcccaggccgaaaaatatgcccangattgggccctaacagggaaaacttttcccctgcccctgggacaat

SEQ ID NO: 795 - DNA, 649 bp, Mus musculus
  source 1..649: mol_type = genomic DNA
ggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggtcccaactctgatccccaccgacaggctgaagaggctgacctagtggctgtccctgcccctgtggctgaggcagagccacggtaacgctgcagcagctccaggaagccctg

SEQ ID NO: 796 - DNA, 527 bp, Mus musculus
  source 1..527: mol_type = genomic DNA
atttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccaggaggccgaggtccgaaaccgagacctggaggcgcatgttcggcagctacaggaacggatggagatgctgcaggccccaggaaccgcagccatcacgggggtccccagtcccccgg

SEQ ID NO: 797 - DNA, 567 bp, Mus musculus
  source 1..567: mol_type = genomic DNA
atggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggtgagcgggggcggggtatgaggacacacaggtgaccagtccccaagacagtgagtgaggcttcactcttggcagtactaaaattgaatgtagggggctgggctcttg

SEQ ID NO: 798 - DNA, 2474 bp, Mus musculus
  source 1..2474: mol_type = genomic DNA
ccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggccgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcaggcgagtccctcacctgcttccagccaagggggcactgggtggagatggggggcatgttgggtgtgtgaaccctcggggcaggggaggagtccaggctggggcaccgcagccgcgccactgcctttctcctccatcctccacactccatacacctctctcttctccttccagccatcacgggggtccccagtccccgggccacggatccaccttcccatgcttctcgccaaatcctccccaagggaactccctagactcccgttctggcctcgactagattcccgcactgcctctcgccctgctgctgggctccgatcgggtcacctgtcccttctctctccagctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaagatcgagcgaccagggagcgacccaaagcgtctctgtgcccatcgcccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccctgacgtggatgggctaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgcccta

SEQ ID NO: 799 - DNA, 2135 bp, Mus musculus
  source 1..2135: mol_type = genomic DNA
ccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcaggtgatttccagaaacatcctttcttctttggccttgattgggagggtctccgagacagtgtacccccctttacaccagacttcgagggtgccacggacacatgcaatttcgatgtggtggaggaccggctcactgccatggagacgctgtcagacatgcaggaagacatgccccttggggtgcgcctgcccttcgtgggctactcctactgctgcatggccttcagagctgaagaggccgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaagatcgagcgaccagggagcgacccaaagcgtctctgtgcccatcgcccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccctgacgtggatgggctaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgcccta

SEQ ID NO: 800 - DNA, 2873 bp, Homo sapiens
  source 1..2873: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaaaaaaaaaaaaaaa

SEQ ID NO: 801 - DNA, 1509 bp, Homo sapiens
  source 1..1509: mol_type = genomic DNA
ccaccgcagcggacagcgccaagtgaagcctcgcttcccctccgcggcgaccagggcccgagccgagagtagcagttgtagctacccgcccagaaactagacacaatgtgcgacgaagacgagaccaccgccctcgtgtgcgacaatggctccggcctggtgaaagccggcttcgccggggatgacgcccctagggccgtgttcccgtccatcgtgggccgcccccgacaccagggcgtcatggtcggtatgggtcagaaagattcctacgtgggcgacgaggctcagagcaagagaggtatcctgaccctgaagtaccctatcgagcacggcatcatcaccaactgggatgacatggagaagatctggcaccacaccttctacaacgagcttcgcgtggctcccgaggagcaccccaccctgctcaccgaggcccccctcaatcccaaggccaaccgcgagaagatgacccagatcatgtttgagaccttcaacgtgcccgccatgtacgtggccatccaggccgtgctgtccctctacgcctccggcaggaccaccggcatcgtgctggactccggcgacggcgtcacccacaacgtgcccatttatgagggctacgcgctgccgcacgccatcatgcgcctggacctggcgggccgcgatctcaccgactacctgatgaagatcctcactgagcgtggctactccttcgtgaccacagctgagcgcgagatcgtgcgcgacatcaaggagaagctgtgctacgtggccctggacttcgagaacgagatggcgacggccgcctcctcctcctccctggaaaagagctacgagctgccagacgggcaggtcatcaccatcggcaacgagcgcttccgctgcccggagacgctcttccagccctccttcatcggtatggagtcggcgggcattcacgagaccacctacaacagcatcatgaagtgtgacatcgacatcaggaaggacctgtatgccaacaacgtcatgtcggggggcaccacgatgtaccctgggatcgctgaccgcatgcagaaagagatcaccgcgctggcacccagcaccatgaagatcaagatcatcgccccgccggagcgcaaatactcggtgtggatcggcggctccatcctggcctcgctgtccaccttccagcagatgtggatcaccaagcaggagtacgacgaggccggcccttccatcgtccaccgcaaatgcttctagacacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaaaaaaaaaaaaaaaaaa

SEQ ID NO: 802 - DNA, 19 bp, synthetic construct
  misc_feature 1..19: note = Synthetic oligonucleotide
  source 1..19: mol_type = other DNA
gcagcagcagcagcagcag

SEQ ID NO: 803 - DNA, 19 bp, synthetic construct
  misc_feature 1..19: note = Synthetic oligonucleotide
  source 1..19: mol_type = other DNA
gcagcagcagcagcagcag

SEQ ID NO: 804 - DNA, 20 bp, synthetic construct
  misc_feature 1..20: note = Synthetic oligonucleotide
  source 1..20: mol_type = other DNA
agcagcagcagcagcagcag

SEQ ID NO: 805 - DNA, 18 bp, synthetic construct
  misc_feature 1..18: note = Synthetic oligonucleotide
  source 1..18: mol_type = other DNA
gcagcagcagcagcagca

SEQ ID NO: 806 - DNA, 19 bp, synthetic construct
  misc_feature 1..19: note = Synthetic oligonucleotide
  source 1..19: mol_type = other DNA
agcagcagcagcagcagca

SEQ ID NO: 807 - DNA, 16 bp, synthetic construct
  misc_feature 1..16: note = Synthetic oligonucleotide
  source 1..16: mol_type = other DNA
agcagcagcagcagca

SEQ ID NO: 808 - DNA, 16 bp, synthetic construct
  misc_feature 1..16: note = Synthetic oligonucleotide
  source 1..16: mol_type = other DNA
ctcccgacaagctcca

SEQ ID NO: 809 - DNA, 14 bp, synthetic construct
  misc_feature 1..14: note = Synthetic oligonucleotide
  source 1..14: mol_type = other DNA
tcccgacaagctcc

SEQ ID NO: 810 - DNA, 16 bp, synthetic construct
  misc_feature 1..16: note = Synthetic oligonucleotide
  source 1..16: mol_type = other DNA
gcttgcacgtgtggct

SEQ ID NO: 811 - DNA, 14 bp, synthetic construct
  misc_feature 1..14: note = Synthetic oligonucleotide
  source 1..14: mol_type = other DNA
cttgcacgtgtggc

SEQ ID NO: 812 - DNA, 16 bp, synthetic construct
  misc_feature 1..16: note = Synthetic oligonucleotide
  source 1..16: mol_type = other DNA
ggttgtgaactggcag

SEQ ID NO: 813 - DNA, 14 bp, synthetic construct
  misc_feature 1..14: note = Synthetic oligonucleotide
  source 1..14: mol_type = other DNA
gttgtgaactggca

SEQ ID NO: 814 - DNA, 16 bp, synthetic construct
  misc_feature 1..16: note = Synthetic oligonucleotide
  source 1..16: mol_type = other DNA
gagcggttgtgaactg

SEQ ID NO: 815 - DNA, 14 bp, synthetic construct
  misc_feature 1..14: note = Synthetic oligonucleotide
  source 1..14: mol_type = other DNA
agcggttgtgaact

SEQ ID NO: 816 - DNA, 16 bp, synthetic construct
  misc_feature 1..16: note = Synthetic oligonucleotide
  source 1..16: mol_type = other DNA
gctgccttcccaggcc

SEQ ID NO: 817 - DNA, 14 bp, synthetic construct
  misc_feature 1..14: note = Synthetic oligonucleotide
  source 1..14: mol_type = other DNA
ctgccttcccaggc

SEQ ID NO: 818 - DNA, 16 bp, synthetic construct
  misc_feature 1..16: note = Synthetic oligonucleotide
  source 1..16: mol_type = other DNA
gcactttgcgaaccaa

SEQ ID NO: 819 - DNA, 14 bp, synthetic construct
  misc_feature 1..14: note = Synthetic oligonucleotide
  source 1..14: mol_type = other DNA
cactttgcgaacca

SEQ ID NO: 820 - DNA, 16 bp, synthetic construct
  misc_feature 1..16: note = Synthetic oligonucleotide
  source 1..16: mol_type = other DNA
gaaagctttgcacttt

SEQ ID NO: 821 - DNA, 14 bp, synthetic construct
  misc_feature 1..14: note = Synthetic oligonucleotide
  source 1..14: mol_type = other DNA
aaagctttgcactt

SEQ ID NO: 822 - DNA, 16 bp, synthetic construct
  misc_feature 1..16: note = Synthetic oligonucleotide
  source 1..16: mol_type = other DNA
cggaggacgaggtcaa

SEQ ID NO: 823 - DNA, 14 bp, synthetic construct
  misc_feature 1..14: note = Synthetic oligonucleotide
  source 1..14: mol_type = other DNA
ggaggacgaggtca

SEQ ID NO: 824 - DNA, 16 bp, synthetic construct
  misc_feature 1..16: note = Synthetic oligonucleotide
  source 1..16: mol_type = other DNA
agcctgtcagcgagtc

SEQ ID NO: 825 - DNA, 14 bp, synthetic construct
  misc_feature 1..14: note = Synthetic oligonucleotide
  source 1..14: mol_type = other DNA
gcctgtcagcgagt

SEQ ID NO: 826 - DNA, 16 bp, synthetic construct
  misc_feature 1..16: note = Synthetic oligonucleotide
  source 1..16: mol_type = other DNA
tcctgtagcctgtcag

SEQ ID NO: 827 - DNA, 14 bp, synthetic construct
  misc_feature 1..14: note = Synthetic oligonucleotide
  source 1..14: mol_type = other DNA
cctgtagcctgtca

SEQ ID NO: 828 - DNA, 16 bp, synthetic construct
  misc_feature 1..16: note = Synthetic oligonucleotide
  source 1..16: mol_type = other DNA
gaagcgaggcttcact

SEQ ID NO: 829 - DNA, 14 bp, synthetic construct
  misc_feature 1..14: note = Synthetic oligonucleotide
  source 1..14: mol_type = other DNA
aagcgaggcttcac

SEQ ID NO: 830 - DNA, 16 bp, synthetic construct
  misc_feature 1..16: note = Synthetic oligonucleotide
  source 1..16: mol_type = other DNA
acctgcccgtctggca

SEQ ID NO: 831 - DNA, 14 bp, synthetic construct
  misc_feature 1..14: note = Synthetic oligonucleotide
  source 1..14: mol_type = other DNA
cctgcccgtctggc

SEQ ID NO: 832 - DNA, 16 bp, synthetic construct
  misc_feature 1..16: note = Synthetic oligonucleotide
  source 1..16: mol_type = other DNA
ggtcagcgatcccagg

SEQ ID NO: 833 - DNA, 14 bp, synthetic construct
  misc_feature 1..14: note = Synthetic oligonucleotide
  source 1..14: mol_type = other DNA
gtcagcgatcccag

SEQ ID NO: 834 - DNA, 16 bp, synthetic construct
  misc_feature 1..16: note = Synthetic oligonucleotide
  source 1..16: mol_type = other DNA
attttcttccacaggg

SEQ ID NO: 835 - DNA, 14 bp, synthetic construct
  misc_feature 1..14: note = Synthetic oligonucleotide
  source 1..14: mol_type = other DNA
ttttcttccacagg

SEQ ID NO: 836 - DNA, 16 bp, synthetic construct
  misc_feature 1..16: note = Synthetic oligonucleotide
  source 1..16: mol_type = other DNA
gaatgactttaatgct

SEQ ID NO: 837 - DNA, 14 bp, synthetic construct
  misc_feature 1..14: note = Synthetic oligonucleotide
  source 1..14: mol_type = other DNA
aatgactttaatgc